(19) United States
(12) Patent Application Publication
(10) Pub. No.: US 2024/0148744 A1
(43) Pub. Date: May 9, 2024

(54) SULFONAMIDES AND THEIR USE FOR TREATMENT OF HELMINTHIC INFECTIONS AND DISEASES

(71) Applicants: Celgene Corporation, Summit, NJ (US); Zoetis LLC, Kalamazoo, MI (US)

(72) Inventors: Natalie Hawryluk, San Diego, CA (US); Graham Kyne, Kalamazoo, MI (US); Sanjay MENON, Kalamazoo, MI (US); Matthew Bedore, Portage, MI (US)

(21) Appl. No.: 18/275,565

(22) PCT Filed: Feb. 8, 2022
(86) PCT No.: PCT/US2022/015596

§ 371 Date: Aug. 2, 2023

Related U.S. Application Data
(60) Provisional application No. 63/147,710, filed on Feb. 9, 2021.

(51) Int. Cl. A61K 31/5377; A61K 31/4155; A61K 31/4178; A61K 31/4439; A61K 31/4709; A61K 31/4725; A61K 31/497; A61K 31/4985; A61K 31/65; A61P 33/10; C07D 215/40; C07D 401/04; C07D 401/12; C07D 401/14; C07D 403/12; C07D 405/12; C07D 413/14; C07D 471/04

(52) U.S. Cl. CPC A61K 31/5377; A61K 31/4155; A61K 31/4178; A61K 31/4439; A61K 31/4709; A61K 31/4725; A61K 31/497; A61K 31/4985; A61K 31/65; A61P 33/10; C07D 215/40; C07D 401/04; C07D 401/12; C07D 401/14; C07D 403/12; C07D 405/12; C07D 413/14; C07D 471/04

(57) ABSTRACT

Provided herein are Sulfonamide compounds of Formula I: and pharmaceutically acceptable salts, tautomers, isotopologues, and stereoisomers thereof, wherein R¹, R², R, A, m, n, and p are as defined herein, compositions comprising an effective amount of a Sulfonamide Compound, and methods for treating or preventing animal and human filarial worm infections and diseases.

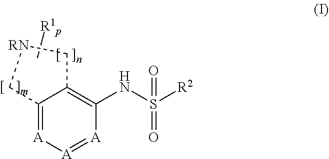

### CROSS REFERENCE TO RELATED APPLICATIONS

This application claims the benefit of U.S. Provisional Application No. 63/147,710, filed Feb. 9, 2021, the disclosure of which is incorporated herein by reference in its entirety.

### FIELD

Disclosed herein are compounds and methods for the prevention and/or treatment of helminthic infections and diseases caused by helminthic infection. Disclosed herein are compounds and methods for the prevention and/or treatment of helminthiasis. Also provided herein are such compounds for use in such methods. Also disclosed herein are pharmaceutical compositions comprising such compounds for use in such methods of preventing or treating helminthic infection and/or diseases associated with helminthic infection.

### BACKGROUND

There are several types of parasitic worms (helminths), with the most common worldwide the intestinal nematodes or soil-transmitted helminths (STH), schistosomes (parasites of schistosomiasis) and filarial worms, which cause lymphatic filariasis (LF) and onchocerciasis. Filariasis is a parasitic disease that is caused by thread-like filarial nematodes or roundworms. Filariasis is a vector-borne disease that is transmitted via insect bites. Infective larvae of the nematodes can be introduced into the human body via bites of blood sucking insects like mosquitoes or flies. Filariasis can also affect domestic animals like dogs. In dogs, dirofilariasis which is also called heartworm disease, is caused by nematodes called Dirofilaria immitis and Dirofilaria repens. Dirofilariasis is considered endemic in 49 states of the United States. The vectors as well are blood sucking insects like mosquitoes.

The major causes of human filariasis are the filarial nematodes Wuchereria bancrofti, Brugia malayi, Brugia timori, Onchocerca volvulus and Mansonella species that have human hosts. The nematodes Wuchereria bancrofti, Brugia malayi and Onchocerca volvulus are responsible for most of the debilitating filarial infections in more than 80 developing countries of the tropics and sub-tropics where 1.1 billion are at risk of infection and about 150 million are infected. All three species are a source of severe pathologies that result in high morbidity and increased mortality. The infection can cause severe morbidity in up to 50% of those infected with the nematodes.

W. bancrofti and B. malayi infections can develop into lymphatic filariasis, often seen as hydrocoele in men and/or lymphoedema and in extreme cases elephantiasis. O. volvulus infections can develop into severe dermatitis and/or onchocerciasis, the visual impairment giving the latter disease its common name River Blindness. Community directed mass drug administration programs are designed to control these infections and eliminate them as a public health problem.

Current efforts aim to eliminate these parasitic nematodes through the use of drugs like diethylcarbamazine, ivermectin, and albendazole that kill the larvae, but not the adult worms. The antihelminthic drug diethylcarbamazine is used to combat lymphatic filariasis in countries without co-endemic O. volvulus infections, i.e. outside of Africa. Ivermectin is used to combat onchocerciasis. The greatest efficacy of both drugs is against the first stage larvae found in the blood stream or in the dermis. Since the worms can live up to 14 years and are fecund for most of their lifespan, populations in endemic regions must be treated with high coverage (at least 65%) for many years to break transmission of the disease to uninfected persons.

Two of the major constraints of treatment of filarial diseases are (i) the absence of a macrofilaricidal drug (or for onchocerciasis, one which permanently sterilizes the worm) and (ii) the risk of worms developing drug-resistance. For example, currently available treatments for onchocerciasis include ivermectin, which kills worm larvae, but has little or no activity against adult Onchocerca volvulus parasites. Thus, infected patients must be retreated with ivermectin for several years until the adult worms die naturally. In addition, there are also potential signs of resistance to ivermectin within the parasite in a few areas. Osei-Atweneboana M Y, et al., Phenotypic Evidence of Emerging Ivermectin Resistance in Onchocerca volvulus, PLoS Negl Trop Dis 5(3): e998 (2011). In addition, there is a danger in treating patients co-infected with both (i) Wuchereria bancrofti, Brugia malayi, Brugia timori, and/or Onchocerca volvulus; and (ii) Loa loa with ivermectin. In such co-infected patients, ivermectin treatment can cause severe reactions, including encephalopathy, leading to coma or even death.

Heartworm infection, caused by the endoparasite Dirofilaria immitis (D. immitis), can be a severe and life-threatening disease in animals such as dogs and cats. Heartworm has a complicated life cycle involving several life stages before they mature into adults that will eventually infect the pulmonary artery of the host animal. Heartworm transmission also requires the mosquito to act as an intermediate host to complete this life cycle. For example, the beginning of the heartworm life cycle and transmission process involves a mosquito biting a previously infected dog and ingesting blood containing heartworm microfilariae (larva stage 1). Within the mosquito, the microfilariae will molt into infective larva stage 3 (L3) worms over a two week period. Once the mosquito bites another dog, infective L3 worms will move through the bite wound to enter the host and migrate into the tissues where they will begin molting into larva stage 4 (L4) worms, usually within 1 to 3 days post infection. Subsequently, L4 worms will continue their migration through tissues and molt into sexually immature or “adolescent” adults (larva stage 5, immature adult), approximately 50-70 days post infection. Sexually mature worms will eventually migrate to the heart and lungs of the dog, as early as 70 days post infection. Approximately 6-7 months post infection D. immitis adults reach maturity and sexually reproduce in the pulmonary artery leading to microfilaria (MF) production and circulation in the blood of the dog, thus completing the heartworm life cycle.

The most commonly used heartworm preventatives are the macrocyclic lactones (MLs) such as ivermectin, moxidectin and selamectin. These agents are administered on a monthly basis whereby they kill D. immitis L3 and L4 worms acquired by the host within the previous 30 days. Their primary action is to disrupt the heartworm life cycle by killing L3 and L4 worms thus preventing adult formation and subsequent disease. While very effective at preventing heartworm disease, owners are advised to test dogs for existing heartworm infections (i.e. heartworm positive dogs) prior to starting treatment with MLs due to their potential to kill circulating microfilariae. A rapid decrease in the numbers of microfilariae in the blood can lead to hypersensitivity-type reactions and circulatory shock (e.g. anaphylaxis), presumably due to dead or dying microfilariae. These potential adverse effects can be life-threatening to the dog and as such are presented as caution statements on many ML product labels. Therefore, the discovery of a novel heartworm preventative that would selectively target L3 and L4 stage worms versus microfilariae would offer a potential safety advantage. By not killing circulating microfilariae in heartworm positive dogs, a targeted treatment would prevent the adverse effects known to occur with other heartworm preventatives that lack D. immitis stage selectivity.

Thus, alternative, and more effective, treatments for filarial worm diseases are needed.

Citation or identification of any reference in this application is not to be construed as an admission that the reference is prior art to the present application.

### SUMMARY

Provided herein are Sulfonamide Compounds of formula (I).

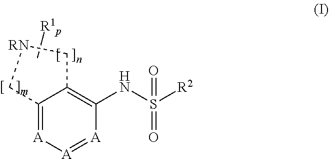

and pharmaceutically acceptable salts, tautomers, isotopologues, and stereoisomers thereof, wherein R¹, R², R, A, m, n, and p are as defined herein.

Provided herein are Sulfonamide Compounds of formula (Ia):

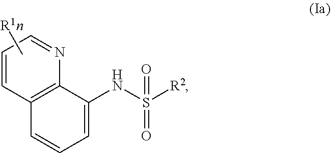

and pharmaceutically acceptable salts, tautomers, isotopologues, and stereoisomers thereof, wherein R¹, R² and n are as defined herein.

In one aspect, provided herein are Sulfonamide Compounds as described in the instant disclosure, such as, for example, a Sulfonamide Compound of formula (I), formula (Ia), or formula (II), or a compound from Table 1, Table 2, Table 3, or Table 4.

In one aspect, provided herein are Sulfonamide Compounds as described in the instant disclosure, such as, for example, a Sulfonamide Compound of formula (I), formula (Ia), or formula (II), or a compound from Table 1 or Table 2.

In one aspect, provided herein are pharmaceutical compositions comprising an effective amount of a Sulfonamide Compound, as described herein, and a pharmaceutically acceptable carrier, excipient or vehicle. In some embodiments the pharmaceutical composition is suitable for oral, parenteral, mucosal, transdermal or topical administration.

In one aspect, provided herein are methods of treating a subject infected with a helminth. In another aspect, provided herein are uses of Sulfonamide Compounds for treating or preventing helminthic infections, comprising administering to a subject affected by helminthic infections an effective amount of a Sulfonamide Compound as described herein. In one aspect the helminthic infection is a filarial infection.

In one aspect, provided herein are methods of treating a subject infected with a filarial worm. In another aspect, provided herein are uses of Sulfonamide Compounds for treating or preventing filarial infections, comprising administering to a subject affected by filarial infections an effective amount of a Sulfonamide Compound as described herein.

In certain embodiments, the methods described herein includes administering a therapeutically effective amount of a compound of formula (I), (Ia), (II), or a compound from Table 1 or Table 2, Table 3, or Table 4, or a pharmaceutically acceptable salt, tautomer, isotopologue, or stereoisomer thereof, to the subject.

In certain embodiments, the methods described herein includes administering a therapeutically effective amount of a compound of formula (I), (Ia), (II), or a compound from Table 1 or Table 2, or Table 3, or a pharmaceutically acceptable salt, tautomer, isotopologue, or stereoisomer thereof, to the subject.

The compounds of the present invention are useful for the treatment of helminthic diseases where the helminths are categorized as cestodes (tapeworms), nematodes (roundworms) and trematodes (flatworms or flukes). Non-limiting examples of filarial nematodes within the Onchocercidae family include the genus Brugia spp. (i.e., B. malayi, B. pahangi, B. timori, and the like), Wuchereria spp. (i.e., W. bancrofti, and the like), Diroflaria spp. (D. immitis, D. repens, D. ursi, D. tenuis, D. spectans, D. lutrae, and the like), Dipetalonema spp. (i.e., D reconditum, D. repens, and the like), Onchocerca spp. (i.e., O. gibsoni, O. gutturosa, O. volvulus, and the like), Elaeophora spp. (E. bohmi, E. elaphi, E. poeli, E. sagitta, E. schneideri, and the like), Mansonella spp. (i.e., M. ozzardi, M. perstans, and the like), and Loa spp. (i.e., L. loa). In certain embodiments, the filarial worm is Onchocerca volvulus. In certain embodiments, the filarial worm is Wuchereria bancrofti. In certain embodiments, the filarial worm is Brugia malayi. In certain embodiments, the filarial worm is Brugia timori. In certain embodiments, the filarial worm is Mansonella. In certain embodiments, the filarial worm is Dirofilaria immitis.

In one aspect, provided herein are uses of Sulfonamide Compounds for treating or preventing helminthic infections, comprising administering to a subject affected by helminthic infection an effective amount of a Sulfonamide Compound as described herein. In another aspect, provided herein are uses of Sulfonamide Compounds for treating or preventing filarial worm infections, wherein the methods comprise administering to a subject affected by filarial worm infections an effective amount of a Sulfonamide Compound as described herein.

In one aspect, provided herein is a Sulfonamide Compound for use as a medicament. In a particular embodiment, provided herein is the Sulfonamide Compound for use in a method for the treatment or prevention of a helminthic infection, the method comprising administering to a subject an effective amount of the Sulfonamide Compound. In a particular embodiment, provided herein is the Sulfonamide Compound for use in a method for the treatment or prevention of a filarial worm infection, the method comprising administering to a subject an effective amount of the Sulfonamide Compound.

In another aspect provided herein are methods for preparing Sulfonamide Compounds as described herein.

The present embodiments can be understood more fully by reference to the detailed description and examples, which are intended to exemplify non-limiting embodiments.

### BRIEF DESCRIPTION OF THE FIGURES

FIG. 1 shows the L. sigmodontis (a rodent filarial nematode) life cycle from microfilariae (L1) to adult stage.

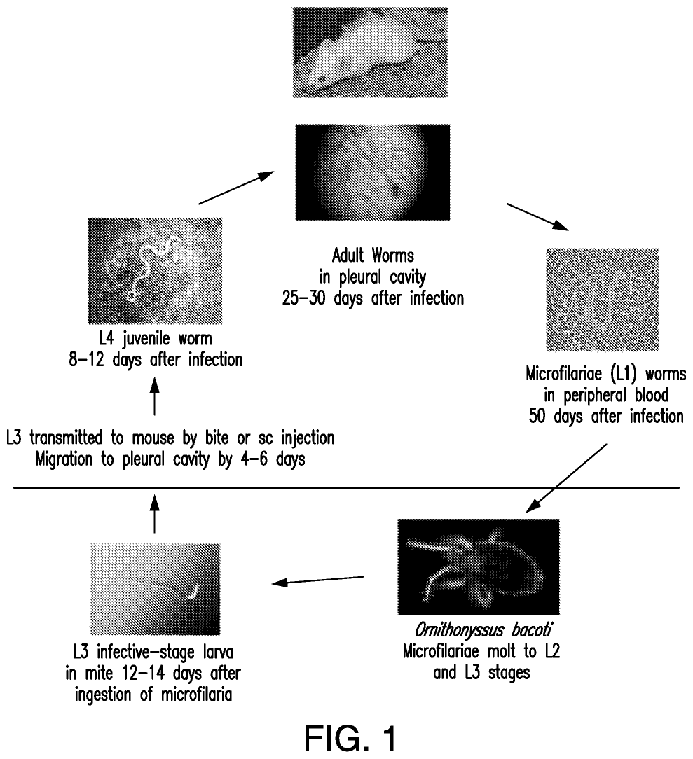

### DETAILED DESCRIPTION

### Definitions

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments of the invention.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

As used herein, the term “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

As used herein and unless otherwise specified, an “alkyl” group is a saturated, partially saturated, or unsaturated straight chain or branched non-cyclic hydrocarbon having from 1 to 10 carbon atoms, typically from 1 to 8 carbons or, in some embodiments, from 1 to 6, 1 to 4, or 2 to 6 or carbon atoms. Representative alkyl groups include -methyl, -ethyl, -n-propyl, -n-butyl, -n-pentyl and -n-hexyl; while saturated branched alkyls include -isopropyl, -sec-butyl, -isobutyl, -tert-butyl, -isopentyl, -neopentyl, tert-pentyl, -2-methylpentyl, -3-methylpentyl, -4-methylpentyl, -2,3-dimethylbutyl and the like. An “alkenyl” group is an alkyl group that contains one or more carbon-carbon double bonds. An “alkynyl” group is an alkyl group that contains one or more carbon-carbon triple bonds. Examples of unsaturated alkyl groups include, but are not limited to, vinyl, allyl, —CH═CH(CH₃), —CH═C(CH₃)₂, —C(CH₃)═CH₂, —C(CH₃)═CH(CH₃), —C(CH₂CH₃)═CH₂, —C≡CH, —C≡C(CH₃), —C≡C(CH₂CH₃), —CH₂C≡CH, —CH₂C≡C(CH₃) and —CH₂C≡C(CH₂CH₃), among others. An alkyl group can be substituted or unsubstituted. When the alkyl groups described herein are said to be “substituted,” they may be substituted with any substituent or substituents as those found in the exemplary compounds and embodiments disclosed herein, as well as halogen; hydroxy; alkoxy; cycloalkyloxy, aryloxy, heterocyclyloxy, heteroaryloxy, heterocycloalkyoxy, cycloalkylalkyloxy, aralkyloxy, heterocyclylalkyloxy, heteroarylalkyloxy, heterocycloalkyalkyloxy; oxo (═O); amino, alkylamino, cycloalkylamino, arylamino, heterocyclylamino, heteroarylamino, heterocycloalkylamino; imino; imido; amidino; guanidino; enamino; acylamino; sulfonylamino; urea, nitrourea; oxime; hydroxylamino; alkoxyamino; aralkoxyamino; hydrazino; hydrazido; hydrazono; azido; nitro; thio (—SH), alkylthio; ═S; sulfinyl; sulfonyl; aminosulfonyl; phosphonate; phosphinyl; acyl; formyl; carboxy; ester; carbamate; amido; cyano; isocyanato; isothiocyanato; cyanato; thiocyanato; or —B(OH)₂.

As used herein and unless otherwise specified, a “cycloalkyl” group is a saturated, or partially saturated cyclic alkyl group of from 3 to 10 carbon atoms having a single cyclic ring or multiple condensed or bridged rings which can be optionally substituted. In some embodiments, the cycloalkyl group has 3 to 8 ring members, whereas in other embodiments the number of ring carbon atoms ranges from 3 to 5, 3 to 6, or 3 to 7. Such cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as 1-bicyclo[1.1.1]pentyl, bicyclo[2.1.1]hexyl, bicyclo[2.2.1]heptyl, bicyclo[2.2.2]octyl, adamantyl and the like. Examples of unsaturared cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanol and the like.

As used herein and unless otherwise specified, an “aryl” group is an aromatic carbocyclic group of from 6 to 14 carbon atoms having a single ring (e.g., phenyl) or multiple condensed rings (e.g., naphthyl or anthryl). In some embodiments, aryl groups contain 6-14 carbons, and in others from 6 to 12 or even 6 to 10 carbon atoms in the ring portions of the groups. Particular aryl groups include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

As used herein and unless otherwise specified, a “heteroaryl” group is an aromatic ring system having one to four heteroatoms as ring atoms in a heteroaromatic ring system, wherein the remainder of the atoms are carbon atoms. In some embodiments, heteroaryl groups contain 3 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, benzisoxazolyl (e.g., benzo[d]isoxazolyl), thiazolyl, pyrolyl, pyridazinyl, pyrimidyl, pyrazinyl, thiophenyl, benzothiophenyl, furanyl, benzofuranyl, indolyl (e.g., indol-2-onyl), isoindolin-1-onyl, azaindolyl, pyrrolopyridyl (e.g., 1H-pyrrolo[2,3-b]pyridyl), indazolyl, benzimidazolyl (e.g., 1H-benzo[d]imidazolyl), azabenzimidazolyl, imidazopyridyl (e.g., 1H-imidazo[4,5-b]pyridyl), pyrazolopyridyl, triazolopyridyl, benzotriazolyl (e.g., 1H-benzo[d][1,2,3]triazolyl), benzoxazolyl (e.g., benzo[d]oxazolyl), benzothiazolyl, benzothiadiazolyl, isoxazolopyridyl, thianaphthalenyl, purinyl, xanthinyl, adeninyl, guaninyl, quinolinyl, isoquinolinyl, 3,4-dihydroisoquinolin-1(2H)-onyl, tetrahydroquinolinyl, quinoxalinyl, and quinazolinyl groups. A heteroaryl group can be substituted or unsubstituted.

As used herein and unless otherwise specified, a “heterocyclyl” is an aromatic ring system (also referred to as heteroaryl) or non-aromatic cycloalkyl (also referred to as heterocycloalkyl) in which one to four of the ring carbon atoms are independently replaced with a heteroatom. Suitable heteroatoms include oxygen, sulfur and nitrogen. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocyclyl group can be substituted or unsubstituted. Heterocyclyl groups encompass unsaturated, partially saturated and saturated ring systems, such as, for example, imidazolyl, imidazolinyl and imidazolidinyl (e.g., imidazolidin-4-onyl or imidazolidin-2,4-dionyl) groups. The phrase heterocyclyl includes fused ring species, including those comprising fused aromatic and non-aromatic groups, such as, for example, 1- and 2-aminotetraline, benzotriazolyl (e.g., 1H-benzo[d][1,2,3]triazolyl), benzimidazolyl (e.g., 1H-benzo[d]imidazolyl), 2,3-dihydrobenzo[1,4]dioxinyl, and benzo[1,3]dioxolyl. The phrase also includes bridged polycyclic ring systems containing a heteroatom such as, but not limited to, quinuclidyl. Representative examples of a heterocyclyl group include, but are not limited to, aziridinyl, azetidinyl, azepanyl, oxetanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, benzisoxazolyl (e.g., benzo[d]isoxazolyl), thiazolyl, thiazolinyl, isothiazolyl, thiadiazolyl, oxadiazolyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dioxyl, dithianyl, pyranyl, pyridyl, pyrimidyl, pyridazinyl, pyrazinyl, triazinyl, dihydropyridyl, dihydrodithiinyl, dihydrodithionyl, 1,4-dioxaspiro[4.5]decanyl, homopiperazinyl, quinuclidyl, indolyl (e.g., indol-2-onyl), isoindolin-1-onyl, indolinyl, isoindolyl, isoindolinyl, azaindolyl, pyrrolopyridyl (e.g, 1H-pyrrolo[2,3-b]pyridyl), indazolyl, indolizinyl, benzotriazolyl (e.g. 1H-benzo[d][1,2,3]triazolyl), benzimidazolyl (e.g., 1H-benzo[d]imidazolyl or 1H-benzo[d]imidazol-2(3H)-onyl), benzofuranyl, benzothiophenyl, benzothiazolyl, benzoxadiazolyl, benzoxazinyl, benzodithiinyl, benzoxathiinyl, benzothiazinyl, benzoxazolyl (e.g., benzo[d]oxazolyl), benzothiazolyl, benzothiadiazolyl, benzo[1,3]dioxolyl, pyrazolopyridyl (e.g., 1H-pyrazolo[3,4-b]pyridyl, 1H-pyrazolo[4,3-b]pyridyl), azabenzimidazolyl, imidazopyridyl (e.g., 1H-imidazo[4,5-b]pyridyl), triazolopyridyl, isoxazolopyridyl, purinyl, xanthinyl, adeninyl, guaninyl, quinolinyl, isoquinolinyl, 3,4-dihydroisoquinolin-1(2H)-onyl, quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, tetrahydropyrimidin-2(1H)-one and tetrahydroquinolinyl groups. Representative non-aromatic heterocyclyl groups do not include fused ring species that comprise a fused aromatic group. Examples of non-aromatic heterocyclyl groups include aziridinyl, azetidinyl, azepanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dithianyl, 1,4-dioxaspiro[4.5]decanyl, homopiperazinyl, quinuclidyl, or tetrahydropyrimidin-2(1H)-one. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed below.

As used herein and unless otherwise specified, a “cycloalkylalkyl” group is a radical of the formula: -alkyl-cycloalkyl, wherein alkyl and cycloalkyl are defined above. Substituted cycloalkylalkyl groups may be substituted at the alkyl, the cycloalkyl, or both the alkyl and the cycloalkyl portions of the group. Representative cycloalkylalkyl groups include but are not limited to cyclopropylmethyl, cyclobutylmethyl, cyclopentylmethyl, cyclohexylmethyl, cyclopropylethyl, cyclobutylethyl, cyclopentylethyl, cyclohexylethyl, cyclopentylpropyl, cyclohexylpropyl and the like.

As used herein and unless otherwise specified, an “aralkyl” group is a radical of the formula: -alkyl-aryl, wherein alkyl and aryl are defined above. Substituted aralkyl groups may be substituted at the alkyl, the aryl, or both the alkyl and the aryl portions of the group. Representative aralkyl groups include but are not limited to benzyl and phenethyl groups and aralkyl groups wherein the aryl group is fused to a cycloalkyl group such as indan-4-yl ethyl.

As used herein and unless otherwise specified, a “heterocyclylalkyl” group is a radical of the formula: -alkyl-heterocyclyl, wherein alkyl and heterocyclyl are defined above. A “heteroarylalkyl” group is a radical of the formula: -alkyl-heteroaryl, wherein alkyl and heteroaryl are defined above. A “heterocycloalkylalkyl” group is a radical of the formula: -alkyl-heterocycloalkyl, wherein alkyl and heterocycloalkyl are defined above. Substituted heterocyclylalkyl groups may be substituted at the alkyl, the heterocyclyl, or both the alkyl and the heterocyclyl portions of the group. Representative heterocylylalkyl groups include but are not limited to morpholin-4-yl ethyl, morpholin-4-yl propyl, furan-2-yl methyl, furan-3-yl methyl, pyridin-3-yl methyl, tetrahydrofuran-2-yl ethyl, and indol-2-yl propyl.

As used herein and unless otherwise specified, a “halogen” is fluorine, chlorine, bromine or iodine.

As used herein and unless otherwise specified, a “hydroxyalkyl” group is an alkyl group as described above substituted with one or more hydroxy groups.

As used herein and unless otherwise specified, an “alkoxy” group is —O-(alkyl), wherein alkyl is defined above. An “alkylthio” group is —S-(alkyl), wherein alkyl is defined above.

As used herein and unless otherwise specified, an “alkoxyalkyl” group is -(alkyl)-O-(alkyl), wherein alkyl is defined above.

As used herein and unless otherwise specified, a “cycloalkyloxy” group is —O-(cycloalkyl), wherein cycloalkyl is defined above.

As used herein and unless otherwise specified, an “aryloxy” group is —O-(aryl), wherein aryl is defined above.

As used herein and unless otherwise specified, a “heterocyclyloxy” group is —O-(heterocyclyl), wherein heterocyclyl is defined above. A “heteroaryloxy” group is —O-(heteroaryl), wherein heteroaryl is defined above. A “heterocycloalkyloxy” group is —O-(heterocycloalkyl), wherein heterocycloalkyl is defined above.

As used herein and unless otherwise specified, an “amino” group is a radical of the formula: —NH₂, —NH(R^{#}), or —N(R^{#})₂, wherein each R^{#} is independently an alkyl, cycloalkyl, cycloalkylalkyl, aryl, aralkyl, heterocyclyl (e.g., heteroaryl or heterocycloalkyl), or heterocyclylalkyl (e.g., heteroarylalkyl or heterocycloalkylalkyl) group defined above, each of which is independently substituted or unsubstituted.

In one embodiment, an “amino” group is an “alkylamino” group, which is a radical of the formula: —NH-alkyl or —N(alkyl)₂, wherein each alkyl is independently defined above. The term “cycloalkylamino”, “arylamino”, “heterocyclylamino”, “heteroarylamino”, “heterocycloalkylamino”, or the like, mirrors the above description for “alkylamino” where the term “alkyl” is replaced with “cycloalkyl”, “aryl”, “heterocyclyl”, “heteroaryl”, “heterocycloalkyl”, or the like, respectively.

As used herein and unless otherwise specified, a “carboxy” group is a radical of the formula: —C(O)OH.

As used herein and unless otherwise specified, an “acyl” group is a radical of the formula: —C(O)(R^{#}) or —C(O)H, wherein R^{#} is defined above. A “formyl” group is a radical of the formula: —C(O)H.

As used herein and unless otherwise specified, an “amido” group is a radical of the formula: —C(O)—NH₂, —C(O)—NH(R′), —C(O)—N(R^{#})₂, —NH—C(O)H, —NH—C(O)—(R^{#}), —N(R^{#})—C(O)H, or —N(R⁴)—C(O)—(R^{#}), wherein each R^{#} is independently defined above.

In one embodiment, an “amido” group is an “aminocarbonyl” group, which is a radical of the formula: —C(O)—NH₂, —C(O)—NH(R^{#}), —C(O)—N(R⁴)₂, wherein each R^{#} is independently defined above.

In one embodiment, an “amido” group is an “acylamino” group, which is a radical of the formula: —NH—C(O)H, —NH—C(O)—(R^{#}), —N(R^{#})—C(O)H, or —N(R^{#})—C(O)—(R^{#}), wherein each R^{#} is independently defined above.

As used herein and unless otherwise specified, a “sulfonylamino” group is a radical of the formula: —NHSO₂(R^{#}) or —N(R^{#})SO₂(R^{#}), wherein each R^{#} is defined above.

As used herein and unless otherwise specified, an “ester” group is a radical of the formula: —C(O)—O—(R⁴) or —O—C(O)—(R⁴), wherein R^{#} is defined above.

In one embodiment, an “ester” group is an “alkoxycarbonyl” group, which is a radical of the formula: —C(O)—O-(alkyl), wherein alkyl is defined above. The term “cycloalkyloxycarbonyl”, “aryloxycarbonyl”, “heterocyclyloxycarbonyl”, “heteroaryloxycarbonyl”, “heterocycloalkyloxycarbonyl”, or the like, mirrors the above description for “alkoxycarbonyl” where the term “alkoxy” is replaced with “cycloalkyloxy”, “aryloxy”, “heterocyclyloxy”, “heteroaryloxy”, “heterocycloalkyloxy”, or the like, respectively.

As used herein and unless otherwise specified, a “carbamate” group is a radical of the formula: —O—C(O)—NH₂, —O—C(O)—NH(R^{#}), —O—C(O)—N(R^{#})₂, —NH—C(O)—O—(R^{#}), or —N(R⁴)—C(O)—O—(R⁴), wherein each R^{#} is independently defined above.

As used herein and unless otherwise specified, a “urea” group is a radical of the formula: —NH(CO)NH₂, —NHC(O)NH(R^{#}), —NHC(O)N(R⁴)₂, —N(R^{#})C(O)NH₂, —N(R^{#})C(O)NH(R^{#}), or —N(R^{#})C(O)N(R^{#})₂, wherein each R^{#} is independently defined above.

As used herein and unless otherwise specified, a “sulfinyl” group is a radical of the formula: —S(O)R^{#}, wherein R^{#} is defined above.

As used herein and unless otherwise specified, a “sulfonyl” group is a radical of the formula: —S(O)₂R^{#}, wherein R^{#} is defined above.

As used herein and unless otherwise specified, an “aminosulfonyl” group is a radical of the formula: —SO₂NH₂, —SO₂NH(R^{#}), or —SO₂N(R^{#})₂, wherein each R^{#} is independently defined above.

When the groups described herein, with the exception of alkyl groups, are said to be “substituted,” they may be substituted with any appropriate substituent or substituents. Illustrative examples of substituents are those found in the exemplary compounds and embodiments disclosed herein, as well as halogen; alkyl, alkenyl, alkynyl, cycloalkyl, aryl, heterocyclyl, heteroaryl, heterocycloalky, cycloalkylalkyl, aralkyl, heterocyclylalkyl, heteroarylalkyl, heterocycloalkyalkyl, optionally further substituted; hydroxy; alkoxy; cycloalkyloxy, aryloxy, heterocyclyloxy, heteroaryloxy, heterocycloalkyoxy, cycloalkylalkyloxy, aralkyloxy, heterocyclylalkyloxy, heteroarylalkyloxy, heterocycloalkyalkyloxy; oxo (═O); oxide (e.g., a nitrogen atom substituted with an oxide is called N-oxide); amino, alkylamino, cycloalkylamino, arylamino, heterocyclylamino, heteroarylamino, heterocycloalkylamino; imino; imido; amidino; guanidino; enamino; acylamino; sulfonylamino; urea, nitrourea; oxime; hydroxylamino; alkoxyamino; aralkoxyamino; hydrazino; hydrazido; hydrazono; azido; nitro; thio (—SH), alkylthio; ═S; sulfinyl; sulfonyl; aminosulfonyl; phosphonate; phosphinyl; acyl; formyl; carboxy; ester; carbamate; amido; cyano; isocyanato; isothiocyanato; cyanato; thiocyanato; or —B(OH)₂.

As used herein, the term “Sulfonamide Compound” includes compounds of formula (I) formula (Ia), formula (II), as well as to further embodiments of compounds of formula (I) formula (Ia), and formula (II), provided herein. For example, the term “Sulfonamide Compound” includes deuterated compounds of formula (I), formula (Ia), formula (II), Table 1, Table 2, Table 3, or Table 4. In one embodiment, a “Sulfonamide Compound” is a compound set forth in Table 1, Table 2, Table 3, or Table 4. In certain embodiments, the term “Sulfonamide Compound” includes pharmaceutically acceptable salts, tautomers, isotopologues, and/or stereoisomers of the Sulfonamide Compounds provided herein.

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the compounds of formula (I), formula (Ia), formula (II), Table 1, Table 2, Table 3, or Table 4, include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methyl-glucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, maleic, phosphoric, sulfuric, and methanesulfonic acids. Examples of specific salts thus include hydrochloride and mesylate salts. Others are well-known in the art, see for example, Remington's Pharmaceutical Sciences, 18^{th} eds., Mack Publishing, Easton PA (1990) or Remington: The Science and Practice of Pharmacy, 19^{th} eds., Mack Publishing, Easton PA (1995).

As used herein and unless otherwise indicated, the term “stereoisomer” or “stereomerically pure” means one stereoisomer of a Sulfonamide Compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The Sulfonamide Compounds can have chiral centers and can occur as racemates, individual enantiomers or diastereomers, and mixtures thereof. All such isomeric forms are included within the embodiments disclosed herein, including mixtures thereof.

The use of stereomerically pure forms of such Sulfonamide Compounds, as well as the use of mixtures of those forms, are encompassed by the embodiments disclosed herein. For example, mixtures comprising equal or unequal amounts of the enantiomers of a particular Sulfonamide Compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33.2725 (1977); Eliel, E. L., Stereochemistry of Carbon Compounds (McGraw-Hill, N Y, 1962); and Wilen, S. H., Tables of Resolving Agents and Optical Resolutions p. 268 (E. L. Eliel, Ed., Univ. of Notre Dame Press, Notre Dame, IN, 1972).

It should also be noted the Sulfonamide Compounds can include E and Z isomers, or a mixture thereof, and cis and trans isomers or a mixture thereof. In certain embodiments, the Sulfonamide Compounds are isolated as either the E or Z isomer. In other embodiments, the Sulfonamide Compounds are a mixture of the E and Z isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms which are referred to as tautomers of each other:

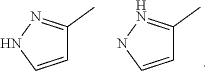

As readily understood by one skilled in the art, a wide variety of functional groups and other structures may exhibit tautomerism and all tautomers of compounds of formula (I), formula (Ia) and formula (II) are within the scope of the present invention.

It should also be noted the Sulfonamide Compounds can contain unnatural proportions of atomic isotopes at least one of the atoms. For example, the compounds may be radiolabeled with radioactive isotopes, such as for example tritium (³H), iodine-125 (¹²⁵I), sulfur-35 (³⁵S), or carbon-14 (¹⁴C), or may be isotopically enriched, such as with carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer and inflammation therapeutic agents, research reagents, e.g., binding assay reagents, and diagnostic agents, e.g., in vivo imaging agents. All isotopic variations of the Sulfonamide Compounds as described herein, whether radioactive or not, are intended to be encompassed within the scope of the embodiments provided herein. In some embodiments, there are provided isotopologues of the Sulfonamide Compounds, for example, the isotopologues are carbon-13, or nitrogen-15 enriched Sulfonamide Compounds. As used herein, “deuterated”, means a compound wherein at least one hydrogen (H) has been replaced by deuterium (indicated by D or ²H), that is, the compound is enriched in deuterium in at least one position It should be noted that if there is a discrepancy between a depicted structure and a name for that structure, the depicted structure is to be accorded more weight.

As used herein, “inhibit” and “inhibition” mean that a specified response of a designated activity (e.g., worm motility) is comparatively decreased in the presence of a Sulfonamide Compound. Inhibition of worm motility, for example motility of Onchocerca volvulus, Brugia malayi and/or Brugia timori, can be determined by the assays described herein.

“Treating” as used herein, means an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself. In one embodiment, the disorder, disorder or condition is a helminthic infection.

“Preventing” as used herein, means a method of delaying and/or precluding the onset, recurrence or spread, in whole or in part, of a disorder, disease or condition; barring a subject from acquiring a disorder, disease, or condition; or reducing a subject's risk of acquiring a disorder, disease, or condition. In one embodiment, the disorder, disorder or condition is a helminthic infection.

The term “effective amount” in connection with a Sulfonamide Compound means an amount capable of treating or preventing a disorder, disease or condition, or symptoms thereof, disclosed herein. In one embodiment, the disorder, disorder or condition is a helminthic infection.

The term “subject” or “patient” includes humans and other primates as well as domesticated and semi-domesticated animals including, but not limited to, poultry, honeybees, cows, sheep, cattle, goats, pigs, horses, dogs, cats, rabbits, rats, mice and the like. The term “poultry” encompasses all types of domestic fowl, including, but not limited to chickens, turkey, ducks, geese, the ratite group of birds and game birds. In certain embodiments, the subject is a human. In certain embodiments, the subject is a dog. In certain embodiments, the subject is a cat. In certain embodiments, the subject is a livestock. In certain embodiments, the subject is a cow. In certain embodiments, the subject is a sheep. In another embodiment, the subject is a goat.

The term “combination” or administration “in combination” includes administration as a mixture, simultaneous administration using separate formulations, and consecutive administration in any order.

The term “helminthic infections” or “helminth infection” as used herein refers to infections that are caused by parasitic worms. An infection caused by a helminth, known as “helminthiasis” (plural “helminthiases”), is any macroparasitic disease of humans and other animals in which a part of the body is infected with parasitic worms, known as helminths. There are numerous species of these parasites, which are broadly classified into tapeworms, flukes, and roundworms.

The term “filariasis” as used herein refers to helminth infections that are caused by filarial nematodes. Non-limiting examples of filarial nematodes within the Onchocercidae family include the genus Brugia spp. (i.e., B. malayi, B. pahangi, B. timori, and the like), Wuchereria spp. (i.e., W. bancrofti, and the like), Dirofilaria spp. (D. immitis, D. repens, D. ursi, D. tenuis, D. spectans, D. lutrae, and the like), Dipetalonema spp. (i.e., D. reconditum, D. repens, and the like), Onchocerca spp. (i.e., O. gibsoni, O. gutturosa, O. volvulus, and the like), Elaeophora spp. (E. bohmi, E. elaphi, E. poeli, E. sagitta, E. schneideri, and the like), Mansonella spp. (i.e., M. ozzardi, M. perstans, and the like), and Loa spp. (i.e., L. loa). An infection is the colonization of a host organism by parasite species. Infections with human filarial nematodes can cause lymphatic filariasis or onchocerciasis. The term “lymphatic filariasis” refers to an infection with the nematodes Wuchereria hancrofti, Brugia malayi or Brugia timori. The term “onchocerciasis” refers to an infection with the nematode Onchocerca volvulus, Lymphatic filariasis may cause hydrocoele, lymphoedema, and elephantiasis. Onchocerciasis may cause skin inflammation and blindness, so called River Blindness. In dogs, an infection with nematode species called Dirofilaria immitis or Dirofilaria repens causes dirofilariasis. In sheep and goats and infection with a nematoide species called Haemonchus contortus causes haemonchosis.

The term “worm” or “nematode” as used interchangeably herein refers to all life stages of the organism, such as an egg, an unfertilized egg, a fertilized egg, a larva or juvenile worm, a larva in any one of four larval stages (L1, L2, L3, L4), a worm in sexually immature stage (stage L5), a worm in mature stage, a worm in fully mature stage, an adult worm, a worm in pre-parasitic stage, or a worm in parasitic stage.

The term “microfilaria” or “mf” as used herein refers to an early stage in the life cycle of certain parasitic nematodes. Microfilaria is considered to be the first larval stage also referred to as L1. The terms “microfilaria,” “mf,” or “L1” are used alternatively and/or interchangeably.

The term “macrofilaria” as used herein refers to the adult stage in the life cycle of certain parasitic nematodes.

Unless otherwise defined, the technical and scientific terms used herein have the same meaning as commonly understood by one of ordinary skill in the art to which this application belongs.

Surprisingly, it was found that the compounds disclosed herein are effective in the treatment of helminthic infections, for example, filarial infections. In vitro and in vivo results demonstrated that the compounds disclosed herein are effective against filarial nematodes. In some embodiments, the compounds disclosed herein surprisingly presented distinct activity between parasitic nematodes in adult and juvenile stage. In some such embodiments, the compounds disclosed herein are selectively effective against adult filarial nematodes (also referred to as macrofilaricidal activity). In other embodiments, the compounds disclosed herein are selectively effective against the juvenile stage filarial nematodes (also referred to as microfilaricidal activity). Therefore, the compounds disclosed herein have the potential to be potent anti-filarial drugs.

### Compounds

Provided herein are Sulfonamide Compounds having the following formula (I):

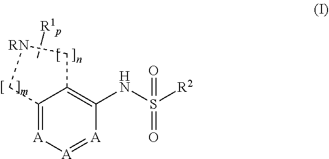

and pharmaceutically acceptable salts, tautomers, isotopologues, and stereoisomers thereof,
wherein:
- - - is a single or double bond;
each A is independently N or CR¹;
each R¹ is independently H, halogen, alkyl, alkenyl, alkynyl, cycloalkyl, aryl, heterocyclyl, heteroaryl, aralkyl, heterocyclylalkyl, heteroarylalkyl; hydroxy; alkoxy; cycloalkyloxy, aryloxy, heterocyclyloxy, heteroaryloxy, cycloalkylalkyloxy, aralkyloxy, heterocyclylalkyloxy, heteroarylalkyloxy; amino, alkylamino, cycloalkylamino, arylamino, heterocyclylamino, heteroarylamino; imino; imido; amidino; enamino; acylamino; sulfonylamino; urea, alkoxyamino; aralkoxyamino; thio (—SH) sulfonyl; aminosulfonyl; acyl; formyl; carboxy; ester; carbamate; amido; or cyano; each optionally further substituted;
R² is substituted or unsubstituted alkyl, cycloalkyl, heterocyclyl, aryl or heteroaryl;
R is absent, H, substituted or unsubstituted alkyl, cycloalkyl, heterocyclyl, or CO (substituted or unsubstituted alkyl, cycloalkyl, aryl, heterocyclyl, or heteroaryl);
m is 0-3;
n is 0-3; and
p is 0-3;
provided that m and n are not both 0; and
wherein when a group described above is said to be “substituted,” it may be substituted with one or more substituents selected from: halogen; alkyl, alkenyl, alkynyl, cycloalkyl, aryl, heterocyclyl, heteroaryl, cycloalkylalkyl, aralkyl, heterocyclylalkyl, heteroarylalkyl, hydroxy; alkoxy; cycloalkyloxy, aryloxy, heterocyclyloxy, heteroaryloxy, cycloalkylalkyloxy, aralkyloxy, heterocyclylalkyloxy, heteroarylalkyloxy; oxo (═O); oxide; amino, alkylamino, cycloalkylamino, arylamino, heterocyclylamino, heteroarylamino; imino; imido; amidino; guanidino; enamino; acylamino; sulfonylamino; urea, nitrourea; oxime; hydroxylamino; alkoxyamino; aralkoxyamino; hydrazino; hydrazido; hydrazono; azido; nitro; thio (—SH), alkylthio; ═S; sulfinyl; sulfonyl; aminosulfonyl; phosphonate; phosphinyl; acyl; formyl; carboxy; ester; carbamate; amido; cyano; isocyanato; isothiocyanato; cyanato; thiocyanato; and —B(OH)₂; each optionally further substituted.

Further provided herein are Sulfonamide Compounds having the following formula (I):

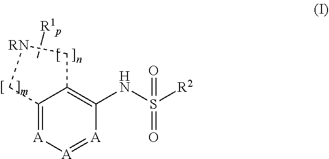

and pharmaceutically acceptable salts, tautomers, isotopologues, and stereoisomers thereof,
wherein:
- - - is a single or double bond;
each A is independently N or CR¹;
each R¹ is independently H, halogen, alkyl, alkenyl, alkynyl, cycloalkyl, aryl, heterocyclyl, heteroaryl, aralkyl, heterocyclylalkyl, heteroarylalkyl; hydroxy; alkoxy; cycloalkyloxy, aryloxy, heterocyclyloxy, heteroaryloxy, cycloalkylalkyloxy, aralkyloxy, heterocyclylalkyloxy, heteroarylalkyloxy; amino, alkylamino, cycloalkylamino, arylamino, heterocyclylamino, heteroarylamino; imino; imido; amidino; enamino; acylamino; sulfonylamino; urea, alkoxyamino; aralkoxyamino; thio (—SH) sulfonyl; alkylsulfonyl, aminosulfonyl; acyl; formyl; carboxy; ester; carbamate; amido; or cyano; each optionally further substituted;
R² is substituted or unsubstituted alkyl, cycloalkyl, heterocyclyl, aryl or heteroaryl;
R is absent, H, substituted or unsubstituted alkyl, cycloalkyl, heterocyclyl, or CO (substituted or unsubstituted alkyl, cycloalkyl, aryl, heterocyclyl, or heteroaryl);
m is 0-3;
n is 0-3; and
p is 0-3;
provided that m and n are not both 0; and
wherein when a group described above is said to be “substituted,” it may be substituted with one or more substituents selected from: halogen; alkyl, alkenyl, alkynyl, cycloalkyl, aryl, heterocyclyl, heteroaryl, cycloalkylalkyl, aralkyl, heterocyclylalkyl, heteroarylalkyl, hydroxy; alkoxy; cycloalkyloxy, aryloxy, heterocyclyloxy, heteroaryloxy, cycloalkylalkyloxy, aralkyloxy, heterocyclylalkyloxy, heteroarylalkyloxy; oxo (═O); oxide; amino, alkylamino, cycloalkylamino, arylamino, heterocyclylamino, heteroarylamino; imino; imido; amidino; guanidino; enamino; acylamino; sulfonylamino; urea, nitrourea; oxime; hydroxylamino; alkoxyamino; aralkoxyamino; hydrazino; hydrazido; hydrazono; azido; nitro; thio (—SH), alkylthio; ═S; sulfinyl; sulfonyl; aminosulfonyl; phosphonate; phosphinyl; acyl; formyl; carboxy; ester; carbamate; amido; cyano; isocyanato; isothiocyanato; cyanato; thiocyanato; and —B(OH)₂; each optionally further substituted.

Provided herein are Sulfonamide Compounds having the following formula (Ia):

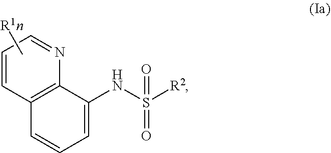

and pharmaceutically acceptable salts, tautomers, isotopologues, and stereoisomers thereof,
wherein:
each R¹ is independently halogen, substituted or unsubstituted C₁₋₄ alkyl, substituted or unsubstituted C₃₋₇ cycloalkyl, substituted or unsubstituted 3-6 membered heterocyclyl, substituted or unsubstituted aryl, or —OR; R² is
a. 2-pyridyl or 3-pyridyl, substituted with one or more substituents independently selected from halogen, CN, substituted or unsubstituted C₁₋₄ alkyl, substituted or unsubstituted C₃₋₆ cycloalkyl, —OR, and —NR₂;
b. 2-imidazolyl or 5-imidazolyl, substituted with one or more substituents independently selected from substituted or unsubstituted C₁₋₄ alkyl, substituted or unsubstituted C₃₋₆ cycloalkyl, and (C₁₋₃ alkyl) (substituted or unsubstituted C₃₋₆ cycloalkyl);
c. pyrazyl, substituted with one or more substituents independently selected from substituted or unsubstituted C₁₋₄ alkyl, substituted or unsubstituted 3-6 membered heterocyclyl, —OR, and NR₂;
d. pyrazolyl, unsubstituted or substituted with one or more substituted or unsubstituted C₁₋₄ alkyl;
e. 2-furanyl unsubstituted or substituted with one or more C₁₋₄ alkyl;
each R is independently H and substituted, unsubstituted C₁₋₄ alkyl, or (C₁₋₃ alkyl) (substituted or unsubstituted C₃₋₆ cycloalkyl);
n is 1-3;
provided the compound is not 5-cyano-N-[5-(trifluoromethyl)-8-quinolinyl]-2-pyridinesulfonamide:

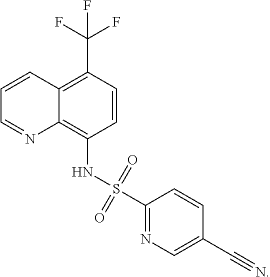 [SMILES: N#Cc1ccc(S(=O)(=O)Nc2ccc(C(F)(F)F)c3cccnc23)nc1]

Further provided herein are Sulfonamide Compounds having the following formula (Ia).

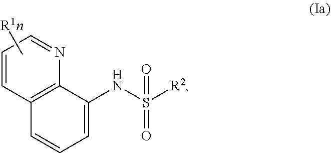

and pharmaceutically acceptable salts, tautomers, isotopologues, and stereoisomers thereof,
wherein:
each R¹ is independently halogen, —CN, substituted or unsubstituted C₁₋₄ alkyl, substituted or unsubstituted C₃₋₇ cycloalkyl, substituted or unsubstituted acyl, substituted or unsubstituted C₁₋₄ sulfonyl, substituted or unsubstituted 3-6 membered heterocyclyl, substituted or unsubstituted aryl, or —OR;
R² is
a. 2-pyridyl or 3-pyridyl, substituted with one or more substituents independently selected from halogen, CN, substituted or unsubstituted C₁₋₄ alkyl, substituted or unsubstituted C₃₋₆ cycloalkyl, —OR, and —NR₂;
b. 2-imidazolyl substituted with one or more substituents independently selected from substituted or unsubstituted C₁₋₄ alkyl, substituted or unsubstituted C₃₋₆ cycloalkyl, (C₁₋₃ alkyl) (substituted or unsubstituted C₃₋₆ cycloalkyl), and substituted or unsubstituted aryl;
c. 5-imidazolyl, substituted with one or more substituents independently selected from substituted or unsubstituted C₁₋₄ alkyl, substituted or unsubstituted C₃₋₆ cycloalkyl, and (C₁₋₃ alkyl) (substituted or unsubstituted C₃₋₆ cycloalkyl);
d. pyrazyl, substituted with one or more substituents independently selected from substituted or unsubstituted C₁₋₄ alkyl, substituted or unsubstituted 3-6 membered heterocyclyl, —OR, and NR₂;
e. pyrazolyl, unsubstituted or substituted with one or more substituted or unsubstituted C₁₋₄ alkyl;
f. 2-furanyl unsubstituted or substituted with one or more C₁₋₄ alkyl;
each R is independently H and substituted or unsubstituted C₁₋₄ alkyl, (C₁₋₃ alkyl), (substituted or unsubstituted C₃₋₆ cycloalkyl), or substituted or unsubstituted aryloxy;
n is 1-3;
provided the compound is not 5-cyano-N-[5-(trifluoromethyl)-8-quinolinyl]-2-pyridinesulfonamide:

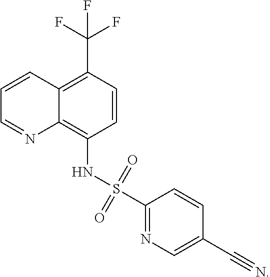 [SMILES: N#Cc1ccc(S(=O)(=O)Nc2ccc(C(F)(F)F)c3cccnc23)nc1]

In one embodiment of compounds of formula (I), m is 2, n is 1, and A is CR¹.

In one embodiment of compounds of formula (I), m is 1, n is 1, and A is CR¹.

In one embodiment of compounds of formula (I), m is 3, n is 0, and A is CR¹.

In one embodiment of compounds of formula (I), m is 2, n is 1, and A is CR1.

In one embodiment of compounds of formula (I), m is 2, n is 0, and A is CR1.

In one embodiment of compounds of formula (I), m is 2, n is 1, and A is CR¹. In one such embodiment, - - - is a single bond. In one embodiment of compounds of formula (I), each R¹ is independently H, halogen, alkyl, alkenyl, alkynyl, cycloalkyl, aryl, heterocyclyl, heteroaryl, aralkyl, heterocyclylalkyl, heteroarylalkyl; hydroxy; alkoxy; cycloalkyloxy, aryloxy, heterocyclyloxy, heteroaryloxy, cycloalkylalkyloxy, aralkyloxy, heterocyclylalkyloxy, heteroarylalkyloxy; amino, alkylamino, cycloalkylamino, arylamino, heterocyclylamino, heteroarylamino; imino; imido; amidino; enamino; acylamino; sulfonylamino; urea, alkoxyamino; aralkoxyamino; thio (—SH) sulfonyl; aminosulfonyl; acyl; formyl; carboxy; ester; carbamate; amido; or cyano; each optionally further substituted; and R² is 2-pyridyl, substituted with one or more substituents independently selected from halogen, CN, substituted or unsubstituted C₁₋₄ alkyl, substituted or unsubstituted C₃₋₆ cycloalkyl, —OR, and —NR₂.

In one embodiment of compounds of formula (I), m is 2, n is 1, and A is CR¹. In one such embodiment, - - - is a single bond. In one such embodiment, p is 0. In one such embodiment, R is independently H or substituted or unsubstituted C₁₋₄ alkyl or substituted or unsubstituted C₃₋₆ cycloalkyl. In one embodiment of compounds of formula (I), each R¹ is independently H, halogen, alkyl, alkenyl, alkynyl, cycloalkyl, aryl, heterocyclyl, heteroaryl, aralkyl, heterocyclylalkyl, heteroarylalkyl; hydroxy; alkoxy; cycloalkyloxy, aryloxy, heterocyclyloxy, heteroaryloxy, cycloalkylalkyloxy, aralkyloxy, heterocyclylalkyloxy, heteroarylalkyloxy; amino, alkylamino, cycloalkylamino, arylamino, heterocyclylamino, heteroarylamino; imino; imido; amidino; enamino; acylamino; sulfonylamino; urea, alkoxyamino; aralkoxyamino; thio (—SH) sulfonyl; aminosulfonyl; acyl; formyl; carboxy; ester; carbamate; amido; or cyano; each optionally further substituted; and R² is 2-pyridyl, substituted with one or more substituents independently selected from halogen, CN, substituted or unsubstituted C₁₋₄ alkyl, substituted or unsubstituted C₃₋₆ cycloalkyl, —OR, and —NR₂.

In one embodiment of compounds of formula (I), m is 2, n is 1, and A is CR¹. In one such embodiment, - - - is a single bond. In one such embodiment, p is 0. In one such embodiment, R is independently H or substituted or unsubstituted C₁₋₄ alkyl or substituted or unsubstituted C₃₋₆ cycloalkyl. In one embodiment of compounds of formula (I), each R¹ is independently H, F, Cl, —CH₃, —CH₂CH₃, —CH₂CH₂CH₃, —CH(CH₃)₂, —CF₃, cyclopropyl, cylobutyl, cyclopentyl, cyclohexyl, —OCH₃, —OCH₂CH₃, —OCH(CH₃)₂, phenyl, pyrrolidyl, piperidyl, piperazinyl or morpholinyl; and R² is 2-pyridyl, substituted with one or more substituents independently selected from F, Cl, —CN, —CH₃, —CH₂CH₃, —CH₂CH₂CH₃, —CH(CH₃)₂, cyclopropyl, cyclobutyl, cyclopentyl, —OCH₃, —OCH₂CH₂CH₃, and —N(CH₃)₂.

In one embodiment of compounds of formula (I), m is 2, n is 1, and A is CR¹. In one such embodiment, - - - is a single bond. In one such embodiment, p is 0. In one such embodiment, R is independently —CH₃, —CH₂CH₃, —CH₂CH₂CH₃, cyclopropyl, cylobutyl, or cyclopentyl. In one embodiment of compounds of formula (I), each R¹ is independently H, C₁, —CH₃, —CH₂CH₃, —CH₂CH₂CH₃, cyclopropyl, cylobutyl, cyclopentyl, —OCH₃, —OCH₂CH₃, -phenyl, pyrrolidyl, piperidyl, piperazinyl or morpholinyl; and R² is 2-pyridyl, substituted with one or more substituents independently selected from —CH₃, —CH₂CH₃, —CH₂CH₂CH₃, -cyclopropyl, cyclobutyl, cyclopentyl, —OCH₃, —OCH₂CH₂CH₃, and —N(CH₃)₂.

In one embodiment of compounds of formula (I), wherein when m is 2, n is 1, and A is CR¹ each R¹ is independently H, or C₁; R² is 2-pyridyl, substituted with one or more substituents independently selected from —CH₃, or -cyclopropyl. In one such embodiment, - - - is a single bond; p is 0; and R is —CH₃ or cyclopropyl.

In one embodiment of compounds of formula (I), m is 2, n is 1, and A is CR¹. In one such embodiment, - - - is a single bond. In one embodiment of compounds of formula (I), each R¹ is independently H, halogen, alkyl, alkenyl, alkynyl, cycloalkyl, aryl, heterocyclyl, heteroaryl, aralkyl, heterocyclylalkyl, heteroarylalkyl; hydroxy; alkoxy; cycloalkyloxy, aryloxy, heterocyclyloxy, heteroaryloxy, cycloalkylalkyloxy, aralkyloxy, heterocyclylalkyloxy, heteroarylalkyloxy; amino, alkylamino, cycloalkylamino, arylamino, heterocyclylamino, heteroarylamino; imino; imido; amidino; enamino; acylamino; sulfonylamino; urea, alkoxyamino; aralkoxyamino; thio (—SH) sulfonyl; aminosulfonyl; acyl; formyl; carboxy; ester; carbamate; amido; or cyano; each optionally further substituted; and R² is 2-imidazolyl, substituted with one or more substituted or unsubstituted C₁₋₄ alkyl.

In one embodiment of compounds of formula (I), m is 2, n is 1, and A is CR¹. In one such embodiment, - - - is a single bond. In one such embodiment, p is 0. In one such embodiment, R is H or substituted or unsubstituted C₁₋₄ alkyl. In one embodiment of compounds of formula (I), each R¹ is independently H, halogen, alkyl, alkenyl, alkynyl, cycloalkyl, aryl, heterocyclyl, heteroaryl, aralkyl, heterocyclylalkyl, heteroarylalkyl; hydroxy; alkoxy; cycloalkyloxy, aryloxy, heterocyclyloxy, heteroaryloxy, cycloalkylalkyloxy, aralkyloxy, heterocyclylalkyloxy, heteroarylalkyloxy; amino, alkylamino, cycloalkylamino, arylamino, heterocyclylamino, heteroarylamino; imino; imido; amidino; enamino; acylamino; sulfonylamino; urea, alkoxyamino; aralkoxyamino; thio (—SH) sulfonyl; aminosulfonyl; acyl; formyl; carboxy; ester; carbamate; amido; or cyano; each optionally further substituted; and R² is 2-imidazolyl, substituted with one or more substituted or unsubstituted C₁₋₄ alkyl;

In one embodiment of compounds of formula (I), m is 2, n is 1, and A is CR¹. In one such embodiment, - - - is a single bond. In one such embodiment, p is 0. In one such embodiment, R is independently —CH₃, —CH₂CH₃, —CH₂CH₂CH₃, cyclopropyl, cylobutyl, or cyclopentyl. In one embodiment of compounds of formula (I), each R¹ is independently H, F, C₁, —CH₃, —CH₂CH₃, —CH₂CH₂CH₃, —CH(CH₃)₂, —CF₃, cyclopropyl, cylobutyl, cyclopentyl, cyclohexyl, —OCH₃, —OCH₂CH₃, —OCH(CH₃)₂, phenyl, pyrrolidyl, piperidyl, piperazinyl or morpholinyl; and R² is 2-imidazolyl, substituted with one or more substituents independently selected from —CH₃, —CH₂CH₃, —CH₂CH₂CH₃, —CH(CH₃)₂, and —CF₃.

In one embodiment of compounds of formula (I), wherein when m is 2, n is 1, and A is CR¹ each R¹ is independently H; R² is 2-imidazolyl, substituted with —CH(CH₃)₂; - - - is a single bond; p is 0; and R is —CH₃.

In one embodiment of compounds of formula (I), m is 2, n is 1, and A is CR¹. In one such embodiment, - - - is a single bond. In one embodiment of compounds of formula (I), each R¹ is independently H, halogen, alkyl, alkenyl, alkynyl, cycloalkyl, aryl, heterocyclyl, heteroaryl, aralkyl, heterocyclylalkyl, heteroarylalkyl; hydroxy; alkoxy; cycloalkyloxy, aryloxy, heterocyclyloxy, heteroaryloxy, cycloalkylalkyloxy, aralkyloxy, heterocyclylalkyloxy, heteroarylalkyloxy; amino, alkylamino, cycloalkylamino, arylamino, heterocyclylamino, heteroarylamino; imino; imido; amidino; enamino; acylamino; sulfonylamino; urea, alkoxyamino; aralkoxyamino; thio (—SH) sulfonyl; aminosulfonyl; acyl; formyl; carboxy; ester; carbamate; amido; or cyano; each optionally further substituted; and R² is pyrazolyl, substituted with one or more substituents independently selected from substituted or unsubstituted C₁₋₄ alkyl, substituted or unsubstituted 3-6 membered heterocyclyl, and —OR.

In one embodiment of compounds of formula (I), m is 2, n is 1, and A is CR¹. In one such embodiment, - - - is a single bond. In one such embodiment, p is 0. In one such embodiment, R is H or substituted or unsubstituted C₁₋₄ alkyl. In one embodiment of compounds of formula (I), each R¹ is independently H, halogen, alkyl, alkenyl, alkynyl, cycloalkyl, aryl, heterocyclyl, heteroaryl, aralkyl, heterocyclylalkyl, heteroarylalkyl; hydroxy; alkoxy; cycloalkyloxy, aryloxy, heterocyclyloxy, heteroaryloxy, cycloalkylalkyloxy, aralkyloxy, heterocyclylalkyloxy, heteroarylalkyloxy; amino, alkylamino, cycloalkylamino, arylamino, heterocyclylamino, heteroarylamino; imino; imido; amidino; enamino; acylamino; sulfonylamino; urea, alkoxyamino; aralkoxyamino; thio (—SH) sulfonyl; aminosulfonyl; acyl; formyl; carboxy; ester; carbamate; amido; or cyano; each optionally further substituted; and R² is pyrazolyl, substituted with one or more substituents independently selected from substituted or unsubstituted C₁₋₄ alkyl, substituted or unsubstituted 3-6 membered heterocyclyl, and —OR.

In one embodiment of compounds of formula (I), m is 2, n is 1, and A is CR¹. In one such embodiment, - - - is a single bond. In one such embodiment, p is 0. In one such embodiment, R is independently —CH₃, —CH₂CH₃, —CH₂CH₂CH₃, cyclopropyl, cylobutyl, or cyclopentyl. In one embodiment of compounds of formula (I), each R¹ is independently H, F, C₁, —CH₃, —CH₂CH₃, —CH₂CH₂CH₃, —CH(CH₃)₂, —CF₃, cyclopropyl, cylobutyl, cyclopentyl, cyclohexyl, —OCH₃, —OCH₂CH₃, —OCH(CH₃)₂, phenyl, pyrrolidyl, piperidyl, piperazinyl or morpholinyl; and R² is pyrazolyl, substituted with one or more substituents independently selected from —CH₃, —CH₂CH₃, —CH₂CH₂CH₃, —CH(CH₃)₂, and —CF₃.

In one embodiment of compounds of formula (I), wherein when m is 2, n is 1, and A is CR¹ each R¹ is independently H; R² is pyrazolyl, substituted with —CH(CH₃)₂; - - - is a single bond; p is 0; and R is —CH₃.

In one embodiment of compounds of formula (I), m is 1, n is 1, and A is CR¹. In one such embodiment, - - - is a single bond. In one embodiment of compounds of formula (I), each R¹ is independently H, halogen, alkyl, alkenyl, alkynyl, cycloalkyl, aryl, heterocyclyl, heteroaryl, aralkyl, heterocyclylalkyl, heteroarylalkyl; hydroxy; alkoxy; cycloalkyloxy, aryloxy, heterocyclyloxy, heteroaryloxy, cycloalkylalkyloxy, aralkyloxy, heterocyclylalkyloxy, heteroarylalkyloxy; amino, alkylamino, cycloalkylamino, arylamino, heterocyclylamino, heteroarylamino; imino; imido; amidino; enamino; acylamino; sulfonylamino; urea, alkoxyamino; aralkoxyamino; thio (—SH) sulfonyl; aminosulfonyl; acyl; formyl; carboxy; ester; carbamate; amido; or cyano; each optionally further substituted; and R² is 2-pyridyl, substituted with one or more substituents independently selected from halogen, CN, substituted or unsubstituted Ct-4 alkyl, substituted or unsubstituted C₃₋₆ cycloalkyl, —OR, and —NR₂.

In one embodiment of compounds of formula (I), m is 1, n is 1, and A is CR¹. In one such embodiment, - - - is a single bond. In one such embodiment, R is independently H or substituted or unsubstituted C₁₋₄ alkyl or substituted or unsubstituted C₃₋₆ cycloalkyl. In one embodiment of compounds of formula (I), each R¹ is independently H, halogen, alkyl, alkenyl, alkynyl, cycloalkyl, aryl, heterocyclyl, heteroaryl, aralkyl, heterocyclylalkyl, heteroarylalkyl; hydroxy; alkoxy; cycloalkyloxy, aryloxy, heterocyclyloxy, heteroaryloxy, cycloalkylalkyloxy, aralkyloxy, heterocyclylalkyloxy, heteroarylalkyloxy; amino, alkylamino, cycloalkylamino, arylamino, heterocyclylamino, heteroarylamino; imino; imido; amidino; enamino; acylamino; sulfonylamino; urea, alkoxyamino; aralkoxyamino; thio (—SH) sulfonyl; aminosulfonyl; acyl; formyl; carboxy; ester; carbamate; amido; or cyano; each optionally further substituted; and R² is 2-pyridyl, substituted with one or more substituents independently selected from halogen, CN, substituted or unsubstituted C₁₋₄ alkyl, substituted or unsubstituted C₃₋₆ cycloalkyl, —OR, and —NR₂.

In one embodiment of compounds of formula (I), m is 1, n is 1, and A is CR¹. In one such embodiment, - - - is a single bond. In one such embodiment, p is 0. In one such embodiment, R is independently H or substituted or unsubstituted C₁₋₄ alkyl or substituted or unsubstituted C₃₋₆ cycloalkyl. In one embodiment of compounds of formula (I), each R¹ is independently H, halogen, alkyl, alkenyl, alkynyl, cycloalkyl, aryl, heterocyclyl, heteroaryl, aralkyl, heterocyclylalkyl, heteroarylalkyl; hydroxy; alkoxy; cycloalkyloxy, aryloxy, heterocyclyloxy, heteroaryloxy, cycloalkylalkyloxy, aralkyloxy, heterocyclylalkyloxy, heteroarylalkyloxy; amino, alkylamino, cycloalkylamino, arylamino, heterocyclylamino, heteroarylamino; imino; imido; amidino; enamino; acylamino; sulfonylamino; urea, alkoxyamino; aralkoxyamino; thio (—SH) sulfonyl; aminosulfonyl; acyl; formyl; carboxy; ester; carbamate; amido; or cyano; each optionally further substituted; and R² is 2-pyridyl, substituted with one or more substituents independently selected from halogen, CN, substituted or unsubstituted C₁₋₄ alkyl, substituted or unsubstituted C₃₋₆ cycloalkyl, —OR, and —NR₂.

In one embodiment of compounds of formula (I), m is 1, n is 1, and A is CR¹. In one such embodiment, - - - is a single bond. In one such embodiment, p is 0. In one such embodiment, R is independently H or substituted or unsubstituted C₁₋₄ alkyl or substituted or unsubstituted C₃₋₆ cycloalkyl. In one embodiment of compounds of formula (I), each R¹ is independently H, F, Cl, —CH₃, —CH₂CH₃, —CH₂CH₂CH₃, —CH(CH₃)₂, —CF₃, cyclopropyl, cylobutyl, cyclopentyl, cyclohexyl, —OCH₃, —OCH₂CH₃, —OCH(CH₃)₂, phenyl, pyrrolidyl, piperidyl, piperazinyl or morpholinyl; and R² is 2-pyridyl, substituted with one or more substituents independently selected from F, Cl, —CN, —CH₃, —CH₂CH₃, —CH₂CH₂CH₃, —CH(CH₃)₂, cyclopropyl, cyclobutyl, cyclopentyl, —OCH₃, —OCH₂CH₂CH₃, and —N(CH₃)₂.

In one embodiment of compounds of formula (I), m is 1, n is 1, and A is CR¹. In one such embodiment, - - - is a single bond. In one such embodiment, p is 0. In one such embodiment, R is independently —CH₃, —CH₂CH₃, —CH₂CH₂CH₃, cyclopropyl, cylobutyl, or cyclopentyl. In one embodiment of compounds of formula (I), each R¹ is independently H, C₁, —CH₃, —CH₂CH₃, —CH₂CH₂CH₃, cyclopropyl, cylobutyl, cyclopentyl, —OCH₃, —OCH₂CH₃, -phenyl, pyrrolidyl, piperidyl, piperazinyl or morpholinyl; and R² is 2-pyridyl, substituted with one or more substituents independently selected from —CH₃, —CH₂CH₃, —CH₂CH₂CH₃, -cyclopropyl, cyclobutyl, cyclopentyl, —OCH₃, —OCH₂CH₂CH₃, and —N(CH₃)₂.

In one embodiment of compounds of formula (I), wherein when m is 1, n is 1, and A is CR¹ each R¹ is independently H; R² is 2-pyridyl, substituted with one or more substituents independently selected from —CH₃, or —N(CH₃)₂. In one such embodiment, - - - is a single bond; p is 0; and R is cyclopropyl.

In one embodiment of compounds of formula (I), m is 1, n is 1, and A is CR¹. In one such embodiment, - - - is a single bond. In one embodiment of compounds of formula (I), each R¹ is independently H, halogen, alkyl, alkenyl, alkynyl, cycloalkyl, aryl, heterocyclyl, heteroaryl, aralkyl, heterocyclylalkyl, heteroarylalkyl; hydroxy; alkoxy; cycloalkyloxy, aryloxy, heterocyclyloxy, heteroaryloxy, cycloalkylalkyloxy, aralkyloxy, heterocyclylalkyloxy, heteroarylalkyloxy; amino, alkylamino, cycloalkylamino, arylamino, heterocyclylamino, heteroarylamino; imino; imido; amidino; enamino; acylamino; sulfonylamino; urea, alkoxyamino; aralkoxyamino; thio (—SH) sulfonyl; aminosulfonyl; acyl; formyl; carboxy; ester; carbamate; amido; or cyano; each optionally further substituted; and R² is 2-imidazolyl, substituted with one or more substituted or unsubstituted C₁₋₄ alkyl.

In one embodiment of compounds of formula (I), m is 1, n is 1, and A is CR¹. In one such embodiment, - - - is a single bond. In one such embodiment, p is 0. In one such embodiment, R is H, substituted or unsubstituted C₁₋₄ alkyl, or substituted or unsubstituted C₃₋₆ cycloalkyl. In one embodiment of compounds of formula (I), each R¹ is independently H, halogen, alkyl, alkenyl, alkynyl, cycloalkyl, aryl, heterocyclyl, heteroaryl, aralkyl, heterocyclylalkyl, heteroarylalkyl; hydroxy; alkoxy; cycloalkyloxy, aryloxy, heterocyclyloxy, heteroaryloxy, cycloalkylalkyloxy, aralkyloxy, heterocyclylalkyloxy, heteroarylalkyloxy; amino, alkylamino, cycloalkylamino, arylamino, heterocyclylamino, heteroarylamino; imino; imido; amidino; enamino; acylamino; sulfonylamino; urea, alkoxyamino; aralkoxyamino; thio (—SH) sulfonyl; aminosulfonyl; acyl; formyl; carboxy; ester; carbamate; amido; or cyano; each optionally further substituted; and R² is 2-imidazolyl, substituted with one or more substituted or unsubstituted C₁₋₄ alkyl;

In one embodiment of compounds of formula (I), m is 1, n is 1, and A is CR¹. In one such embodiment, - - - is a single bond. In one such embodiment, p is 0. In one such embodiment, R is, H, substituted or unsubstituted C₁₋₄ alkyl, or substituted or unsubstituted C₃₋₆ cycloalkyl. In one embodiment of compounds of formula (I), each R¹ is independently H, F, Cl, —CH₃, —CH₂CH₃, —CH₂CH₂CH₃, —CH(CH₃)₂, —CF₃, cyclopropyl, cylobutyl, cyclopentyl, cyclohexyl, —OCH₃, —OCH₂CH₃, —OCH(CH₃)₂, phenyl, pyrrolidyl, piperidyl, piperazinyl or morpholinyl; and R² is 2-imidazolyl, substituted with one or more substituents independently selected from —CH₃, —CH₂CH₃, —CH₂CH₂CH₃, —CH(CH₃)₂, and —CF₃.

In one embodiment of compounds of formula (I), wherein when m is 1, n is 1, and A is CR¹ each R¹ is independently H; R² is 2-imidazolyl, substituted with —CH(CH₃)₂; - - - is a single bond; p is 0; and R is —CH₃.

In one embodiment of compounds of formula (I), m is 1, n is 1, and A is CR¹. In one such embodiment, - - - is a single bond. In one embodiment of compounds of formula (I), each R¹ is independently H, halogen, alkyl, alkenyl, alkynyl, cycloalkyl, aryl, heterocyclyl, heteroaryl, aralkyl, heterocyclylalkyl, heteroarylalkyl; hydroxy; alkoxy; cycloalkyloxy, aryloxy, heterocyclyloxy, heteroaryloxy, cycloalkylalkyloxy, aralkyloxy, heterocyclylalkyloxy, heteroarylalkyloxy; amino, alkylamino, cycloalkylamino, arylamino, heterocyclylamino, heteroarylamino; imino; imido; amidino; enamino; acylamino; sulfonylamino; urea, alkoxyamino; aralkoxyamino; thio (—SH) sulfonyl; aminosulfonyl; acyl; formyl; carboxy; ester; carbamate; amido; or cyano; each optionally further substituted; and R² is pyrazolyl, substituted with one or more substituents independently selected from substituted or unsubstituted C₁₋₄ alkyl, substituted or unsubstituted 3-6 membered heterocyclyl, and —OR.

In one embodiment of compounds of formula (I), m is 1, n is 1, and A is CR¹. In one such embodiment, - - - is a single bond. In one such embodiment, p is 0. In one such embodiment, R is H, substituted or unsubstituted C₁₋₄ alkyl, or substituted or unsubstituted C₃₋₆ cycloalkyl. In one embodiment of compounds of formula (I), each R¹ is independently H, halogen, alkyl, alkenyl, alkynyl, cycloalkyl, aryl, heterocyclyl, heteroaryl, aralkyl, heterocyclylalkyl, heteroarylalkyl; hydroxy; alkoxy; cycloalkyloxy, aryloxy, heterocyclyloxy, heteroaryloxy, cycloalkylalkyloxy, aralkyloxy, heterocyclylalkyloxy, heteroarylalkyloxy; amino, alkylamino, cycloalkylamino, arylamino, heterocyclylamino, heteroarylamino; imino; imido; amidino; enamino; acylamino; sulfonylamino; urea, alkoxyamino; aralkoxyamino; thio (—SH) sulfonyl; aminosulfonyl; acyl; formyl; carboxy; ester; carbamate; amido; or cyano; each optionally further substituted; and R² is pyrazolyl, substituted with one or more substituents independently selected from substituted or unsubstituted C₁₋₄ alkyl, substituted or unsubstituted 3-6 membered heterocyclyl, and —OR.

In one embodiment of compounds of formula (I), m is 1, n is 1, and A is CR¹. In one such embodiment, - - - is a single bond. In one such embodiment, p is 0. In one such embodiment, R is independently —CH₃, —CH₂CH₃, —CH₂CH₂CH₃, cyclopropyl, cylobutyl, or cyclopentyl. In one embodiment of compounds of formula (I), each R¹ is independently H, F, C₁, —CH₃, —CH₂CH₃, —CH₂CH₂CH₃, —CH(CH₃)₂, —CF₃, cyclopropyl, cylobutyl, cyclopentyl, cyclohexyl, —OCH₃, —OCH₂CH₃, —OCH(CH₃)₂, phenyl, pyrrolidyl, piperidyl, piperazinyl or morpholinyl; and R² is pyrazolyl, substituted with one or more substituents independently selected from —CH₃, —CH₂CH₃, —CH₂CH₂CH₃, —CH(CH₃)₂, and —CF₃.

In one embodiment of compounds of formula (I), wherein when m is 1, n is 1, and A is CR¹ each R¹ is independently H; R² is pyrazolyl, substituted with —CH(CH₃)₂; - - - is a single bond; p is 0; and R is cyclopropyl.

In one embodiment of compounds of formula (I), m is 3, n is 0, and A is CR¹. In one such embodiment, - - - is a single bond. In one embodiment of compounds of formula (I), each R¹ is independently H, halogen, alkyl, alkenyl, alkynyl, cycloalkyl, aryl, heterocyclyl, heteroaryl, aralkyl, heterocyclylalkyl, heteroarylalkyl; hydroxy; alkoxy; cycloalkyloxy, aryloxy, heterocyclyloxy, heteroaryloxy, cycloalkylalkyloxy, aralkyloxy, heterocyclylalkyloxy, heteroarylalkyloxy; amino, alkylamino, cycloalkylamino, arylamino, heterocyclylamino, heteroarylamino; imino; imido; amidino; enamino; acylamino; sulfonylamino; urea, alkoxyamino; aralkoxyamino; thio (—SH) sulfonyl; aminosulfonyl; acyl; formyl; carboxy; ester; carbamate; amido; or cyano; each optionally further substituted; and R² is 2-pyridyl, substituted with one or more substituents independently selected from halogen, CN, substituted or unsubstituted C₁₋₄ alkyl, substituted or unsubstituted C₃₋₆ cycloalkyl, —OR, and —NR₂.

In one embodiment of compounds of formula (I), m is 3, n is 0, and A is CR¹. In one such embodiment, - - - is a single bond. In one such embodiment, R is independently H or substituted or unsubstituted C₁₋₄ alkyl or substituted or unsubstituted C₃₋₆ cycloalkyl. In one embodiment of compounds of formula (I), each R¹ is independently H, halogen, alkyl, alkenyl, alkynyl, cycloalkyl, aryl, heterocyclyl, heteroaryl, aralkyl, heterocyclylalkyl, heteroarylalkyl; hydroxy; alkoxy; cycloalkyloxy, aryloxy, heterocyclyloxy, heteroaryloxy, cycloalkylalkyloxy, aralkyloxy, heterocyclylalkyloxy, heteroarylalkyloxy; amino, alkylamino, cycloalkylamino, arylamino, heterocyclylamino, heteroarylamino; imino; imido; amidino; enamino; acylamino; sulfonylamino; urea, alkoxyamino; aralkoxyamino; thio (—SH) sulfonyl; aminosulfonyl; acyl; formyl; carboxy; ester; carbamate; amido; or cyano; each optionally further substituted; and R² is 2-pyridyl, substituted with one or more substituents independently selected from halogen, CN, substituted or unsubstituted C₁₋₄ alkyl, substituted or unsubstituted C₃₋₆ cycloalkyl, —OR, and —NR₂.

In one embodiment of compounds of formula (I), m is 3, n is 0, and A is CR¹. In one such embodiment, - - - is a single bond. In one such embodiment, p is 0. In one such embodiment, R is independently H or substituted or unsubstituted C₁₋₄ alkyl or substituted or unsubstituted C₃₋₆ cycloalkyl. In one embodiment of compounds of formula (I), each R¹ is independently H, halogen, alkyl, alkenyl, alkynyl, cycloalkyl, aryl, heterocyclyl, heteroaryl, aralkyl, heterocyclylalkyl, heteroarylalkyl; hydroxy; alkoxy; cycloalkyloxy, aryloxy, heterocyclyloxy, heteroaryloxy, cycloalkylalkyloxy, aralkyloxy, heterocyclylalkyloxy, heteroarylalkyloxy; amino, alkylamino, cycloalkylamino, arylamino, heterocyclylamino, heteroarylamino; imino; imido; amidino; enamino; acylamino; sulfonylamino; urea, alkoxyamino; aralkoxyamino; thio (—SH) sulfonyl; aminosulfonyl; acyl; formyl; carboxy; ester; carbamate; amido; or cyano; each optionally further substituted; and R² is 2-pyridyl, substituted with one or more substituents independently selected from halogen, CN, substituted or unsubstituted C₁₋₄ alkyl, substituted or unsubstituted C₃₋₆ cycloalkyl, —OR, and —NR₂.

In one embodiment of compounds of formula (I), m is 3, n is 0, and A is CR¹. In one such embodiment, - - - is a single bond. In one such embodiment, p is 0. In one such embodiment, R is independently H or substituted or unsubstituted C₁₋₄ alkyl or substituted or unsubstituted C₃₋₆ cycloalkyl. In one embodiment of compounds of formula (I), each R¹ is independently H, F, Cl, —CH₃, —CH₂CH₃, —CH₂CH₂CH₃, —CH(CH₃)₂, —CF₃, cyclopropyl, cylobutyl, cyclopentyl, cyclohexyl, —OCH₃, —OCH₂CH₃, —OCH(CH₃)₂, phenyl, pyrrolidyl, piperidyl, piperazinyl or morpholinyl; and R² is 2-pyridyl, substituted with one or more substituents independently selected from F, Cl, —CN, —CH₃, —CH₂CH₃, —CH₂CH₂CH₃, —CH(CH₃)₂, cyclopropyl, cyclobutyl, cyclopentyl, —OCH₃, —OCH₂CH₂CH₃, and —N(CH₃)₂.

In one embodiment of compounds of formula (I), m is 3, n is 0, and A is CR¹. In one such embodiment, - - - is a single bond. In one such embodiment, p is 0. In one such embodiment, R is independently H, —CH₃, —CH₂CH₃, —CH₂CH₂CH₃, cyclopropyl, cylobutyl, or cyclopentyl. In one embodiment of compounds of formula (I), each R¹ is independently H, C₁, —CH₃, —CH₂CH₃, —CH₂CH₂CH₃, cyclopropyl, cylobutyl, cyclopentyl, —OCH₃, —OCH₂CH₃, -phenyl, pyrrolidyl, piperidyl, piperazinyl or morpholinyl; and R² is 2-pyridyl, substituted with one or more substituents independently selected from F, —CH₃, —CH₂CH₃, —CH₂CH₂CH₃, -cyclopropyl, cyclobutyl, cyclopentyl, —OCH₃, —OCH₂CH₂CH₃, and —N(CH₃)₂.

In one embodiment of compounds of formula (I), wherein when m is 3, n is 0, and A is CR¹ each R¹ is independently H; R² is 2-pyridyl, substituted with one or more substituents independently selected from F, —CH₃, or —N(CH₃)₂. In one such embodiment, - - - is a single bond; p is 0; and R is H, —CH₃, or cyclopropyl.

In one embodiment of compounds of formula (I), m is 3, n is 0, and A is CR¹. In one such embodiment, - - - is a single bond. In one embodiment of compounds of formula (I), each R¹ is independently H, halogen, alkyl, alkenyl, alkynyl, cycloalkyl, aryl, heterocyclyl, heteroaryl, aralkyl, heterocyclylalkyl, heteroarylalkyl; hydroxy; alkoxy; cycloalkyloxy, aryloxy, heterocyclyloxy, heteroaryloxy, cycloalkylalkyloxy, aralkyloxy, heterocyclylalkyloxy, heteroarylalkyloxy; amino, alkylamino, cycloalkylamino, arylamino, heterocyclylamino, heteroarylamino; imino; imido; amidino; enamino; acylamino; sulfonylamino; urea, alkoxyamino; aralkoxyamino; thio (—SH) sulfonyl; alkyl sulfonyl, aminosulfonyl; acyl; formyl; carboxy; ester; carbamate; amido; or cyano; each optionally further substituted; and R² is 2-imidazolyl, substituted with one or more substituted or unsubstituted C₁₋₄ alkyl, or substituted or unsubstituted aryl.

In one embodiment of compounds of formula (I), m is 3, n is 0, and A is CR¹. In one such embodiment, - - - is a single bond. In one embodiment of compounds of formula (I), each R¹ is independently H, halogen, alkyl, alkenyl, alkynyl, cycloalkyl, aryl, heterocyclyl, heteroaryl, aralkyl, heterocyclylalkyl, heteroarylalkyl; hydroxy; alkoxy; cycloalkyloxy, aryloxy, heterocyclyloxy, heteroaryloxy, cycloalkylalkyloxy, aralkyloxy, heterocyclylalkyloxy, heteroarylalkyloxy; amino, alkylamino, cycloalkylamino, arylamino, heterocyclylamino, heteroarylamino; imino; imido; amidino; enamino; acylamino; sulfonylamino; urea, alkoxyamino; aralkoxyamino; thio (—SH) sulfonyl; aminosulfonyl; acyl; formyl; carboxy; ester; carbamate; amido; or cyano; each optionally further substituted; and R² is 2-imidazolyl, substituted with one or more substituted or unsubstituted C₁₋₄ alkyl.

In one embodiment of compounds of formula (I), m is 3, n is 0, and A is CR¹. In one such embodiment, - - - is a single bond. In one embodiment of compounds of formula (I), each R¹ is independently H, halogen, alkyl, alkenyl, alkynyl, cycloalkyl, aryl, heterocyclyl, heteroaryl, aralkyl, heterocyclylalkyl, heteroarylalkyl; hydroxy; alkoxy; cycloalkyloxy, aryloxy, heterocyclyloxy, heteroaryloxy, cycloalkylalkyloxy, aralkyloxy, heterocyclylalkyloxy, heteroarylalkyloxy; amino, alkylamino, cycloalkylamino, arylamino, heterocyclylamino, heteroarylamino; imino; imido; amidino; enamino; acylamino; sulfonylamino; urea, alkoxyamino; aralkoxyamino; thio (—SH) sulfonyl; aminosulfonyl; acyl; formyl; carboxy; ester; carbamate; amido; or cyano; each optionally further substituted; and R² is pyrazolyl, substituted with one or more substituents independently selected from substituted or unsubstituted C₁₋₄ alkyl, substituted or unsubstituted 3-6 membered heterocyclyl, and —OR.

In one embodiment of compounds of formula (I), m is 2, n is 0, and A is CR¹. In one such embodiment, - - - is a single bond. In one embodiment of compounds of formula (I), each R¹ is independently H, halogen, alkyl, alkenyl, alkynyl, cycloalkyl, aryl, heterocyclyl, heteroaryl, aralkyl, heterocyclylalkyl, heteroarylalkyl; hydroxy; alkoxy; cycloalkyloxy, aryloxy, heterocyclyloxy, heteroaryloxy, cycloalkylalkyloxy, aralkyloxy, heterocyclylalkyloxy, heteroarylalkyloxy; amino, alkylamino, cycloalkylamino, arylamino, heterocyclylamino, heteroarylamino; imino; imido; amidino; enamino; acylamino; sulfonylamino; urea, alkoxyamino; aralkoxyamino; thio (—SH) sulfonyl; aminosulfonyl; acyl; formyl; carboxy; ester; carbamate; amido; or cyano; each optionally further substituted; and R² is 2-pyridyl, substituted with one or more substituents independently selected from halogen, CN, substituted or unsubstituted C₁₋₄ alkyl, substituted or unsubstituted C₃₋₆ cycloalkyl, —OR, and —NR₂.

In one embodiment of compounds of formula (I), m is 2, n is 0, and A is CR¹. In one such embodiment, - - - is a single bond. In one such embodiment, R is independently H or substituted or unsubstituted C₁₋₄ alkyl or substituted or unsubstituted C₃₋₆ cycloalkyl. In one embodiment of compounds of formula (I), each R¹ is independently H, halogen, alkyl, alkenyl, alkynyl, cycloalkyl, aryl, heterocyclyl, heteroaryl, aralkyl, heterocyclylalkyl, heteroarylalkyl; hydroxy; alkoxy; cycloalkyloxy, aryloxy, heterocyclyloxy, heteroaryloxy, cycloalkylalkyloxy, aralkyloxy, heterocyclylalkyloxy, heteroarylalkyloxy; amino, alkylamino, cycloalkylamino, arylamino, heterocyclylamino, heteroarylamino; imino; imido; amidino; enamino; acylamino; sulfonylamino; urea, alkoxyamino; aralkoxyamino; thio (—SH) sulfonyl; aminosulfonyl; acyl; formyl; carboxy; ester; carbamate; amido; or cyano; each optionally further substituted; and R² is 2-pyridyl, substituted with one or more substituents independently selected from halogen, CN, substituted or unsubstituted C₁₋₄ alkyl, substituted or unsubstituted C₃₋₆ cycloalkyl, —OR, and —NR₂.

In one embodiment of compounds of formula (I), m is 2, n is 0, and A is CR¹. In one such embodiment, - - - is a single bond. In one such embodiment, p is 0. In one such embodiment, R is independently H or substituted or unsubstituted C₁₋₄ alkyl or substituted or unsubstituted C₃₋₆ cycloalkyl. In one embodiment of compounds of formula (I), each R¹ is independently H, halogen, alkyl, alkenyl, alkynyl, cycloalkyl, aryl, heterocyclyl, heteroaryl, aralkyl, heterocyclylalkyl, heteroarylalkyl; hydroxy; alkoxy; cycloalkyloxy, aryloxy, heterocyclyloxy, heteroaryloxy, cycloalkylalkyloxy, aralkyloxy, heterocyclylalkyloxy, heteroarylalkyloxy; amino, alkylamino, cycloalkylamino, arylamino, heterocyclylamino, heteroarylamino; imino; imido; amidino; enamino; acylamino; sulfonylamino; urea, alkoxyamino; aralkoxyamino; thio (—SH) sulfonyl; aminosulfonyl; acyl; formyl; carboxy; ester; carbamate; amido; or cyano; each optionally further substituted; and R² is 2-pyridyl, substituted with one or more substituents independently selected from halogen, CN, substituted or unsubstituted C₁₋₄ alkyl, substituted or unsubstituted C₃₋₆ cycloalkyl, —OR, and —NR₂.

In one embodiment of compounds of formula (I), m is 2, n is 0, and A is CR¹. In one such embodiment, - - - is a single bond. In one such embodiment, p is 0. In one such embodiment, R is independently H or substituted or unsubstituted C₁₋₄ alkyl or substituted or unsubstituted C₃₋₆ cycloalkyl. In one embodiment of compounds of formula (I), each R¹ is independently H, F, Cl, —CH₃, —CH₂CH₃, —CH₂CH₂CH₃, —CH(CH₃)₂, —CF₃, cyclopropyl, cylobutyl, cyclopentyl, cyclohexyl, —OCH₃, —OCH₂CH₃, —OCH(CH₃)₂, phenyl, pyrrolidyl, piperidyl, piperazinyl or morpholinyl; and R² is 2-pyridyl, substituted with one or more substituents independently selected from F, Cl, —CN, —CH₃, —CH₂CH₃, —CH₂CH₂CH₃, —CH(CH₃)₂, cyclopropyl, cyclobutyl, cyclopentyl, —OCH₃, —OCH₂CH₂CH₃, and —N(CH₃)₂.

In one embodiment of compounds of formula (I), m is 2, n is 0, and A is CR¹. In one such embodiment, - - - is a single bond. In one such embodiment, p is 0. In one such embodiment, R is independently H, —CH₃, —CH₂CH₃, —CH₂CH₂CH₃, cyclopropyl, cylobutyl, or cyclopentyl. In one embodiment of compounds of formula (I), each R¹ is independently H, C₁, —CH₃, —CH₂CH₃, —CH₂CH₂CH₃, cyclopropyl, cylobutyl, cyclopentyl, —OCH₃, —OCH₂CH₃, -phenyl, pyrrolidyl, piperidyl, piperazinyl or morpholinyl; and R² is 2-pyridyl, substituted with one or more substituents independently selected from F, —CH₃, —CH₂CH₃, —CH₂CH₂CH₃, -cyclopropyl, cyclobutyl, cyclopentyl, —OCH₃, —OCH₂CH₂CH₃, and —N(CH₃)₂.

In one embodiment of compounds of formula (I), wherein when m is 2, n is 0, and A is CR¹ each R¹ is independently H; R² is 2-pyridyl, substituted with —CH₃. In one such embodiment, - - - is a single bond; p is 0; and R is —CH₃.

In one embodiment of compounds of formula (I), m is 2, n is 0, and A is CR¹. In one such embodiment, - - - is a single bond. In one embodiment of compounds of formula (I), each R¹ is independently H, halogen, alkyl, alkenyl, alkynyl, cycloalkyl, aryl, heterocyclyl, heteroaryl, aralkyl, heterocyclylalkyl, heteroarylalkyl; hydroxy; alkoxy; cycloalkyloxy, aryloxy, heterocyclyloxy, heteroaryloxy, cycloalkylalkyloxy, aralkyloxy, heterocyclylalkyloxy, heteroarylalkyloxy; amino, alkylamino, cycloalkylamino, arylamino, heterocyclylamino, heteroarylamino; imino; imido; amidino; enamino; acylamino; sulfonylamino; urea, alkoxyamino; aralkoxyamino; thio (—SH) sulfonyl; aminosulfonyl; acyl; formyl; carboxy; ester; carbamate; amido; or cyano; each optionally further substituted; and R² is 2-imidazolyl, substituted with one or more substituted or unsubstituted C₁₋₄ alkyl.

In one embodiment of compounds of formula (I), m is 2, n is 0, and A is CR¹. In one such embodiment, - - - is a single bond. In one embodiment of compounds of formula (I), each R¹ is independently H, halogen, alkyl, alkenyl, alkynyl, cycloalkyl, aryl, heterocyclyl, heteroaryl, aralkyl, heterocyclylalkyl, heteroarylalkyl; hydroxy; alkoxy; cycloalkyloxy, aryloxy, heterocyclyloxy, heteroaryloxy, cycloalkylalkyloxy, aralkyloxy, heterocyclylalkyloxy, heteroarylalkyloxy; amino, alkylamino, cycloalkylamino, arylamino, heterocyclylamino, heteroarylamino; imino; imido; amidino; enamino; acylamino; sulfonylamino; urea, alkoxyamino; aralkoxyamino; thio (—SH) sulfonyl; aminosulfonyl; acyl; formyl; carboxy; ester; carbamate; amido; or cyano; each optionally further substituted; and R² is pyrazolyl, substituted with one or more substituents independently selected from substituted or unsubstituted C₁₋₄ alkyl, substituted or unsubstituted 3-6 membered heterocyclyl, and —OR.

In one embodiment of compounds of formula (Ia), each R¹ is independently halogen, substituted or unsubstituted C₁₋₄ alkyl, substituted or unsubstituted C₃₋₇ cycloalkyl, substituted or unsubstituted 3-6 membered heterocyclyl, substituted or unsubstituted aryl, or —OR; and R² is 2-pyridyl, substituted with one or more substituents independently selected from halogen, CN, substituted or unsubstituted C₁₋₄ alkyl, substituted or unsubstituted C₃₋₆ cycloalkyl, —OR, and —NR₂.

In one embodiment of compounds of formula (Ia), each R¹ is independently halogen, substituted or unsubstituted C₁₋₄ alkyl, substituted or unsubstituted C₃₋₇ cycloalkyl, substituted or unsubstituted 3-6 membered heterocyclyl, substituted or unsubstituted aryl, or —OR; and R² is 3-pyridyl, substituted with one or more substituents independently selected from halogen, —CN, substituted or unsubstituted C₁₋₄ alkyl, substituted or unsubstituted C₃₋₆ cycloalkyl, —OR, and —NR₂.

In one embodiment of compounds of formula (Ia), each R¹ is independently halogen, —CN, substituted or unsubstituted C₁₋₄ alkyl, substituted or unsubstituted acyl; substituted or unsubstituted C₁₋₄ alkyl amino, substituted or unsubstituted C₁₋₄ alkyl sulfonyl, substituted or unsubstituted C₃₋₇ cycloalkyl, substituted or unsubstituted 3-6 membered heterocyclyl, substituted or unsubstituted aryl, or —OR; and R² is 2-imidazolyl or 5-imidazolyl, substituted with one or more substituents independently selected from substituted or unsubstituted C₁₋₄ alkyl, substituted or unsubstituted C₃₋₆ cycloalkyl, (C₁₋₃ alkyl) (substituted or unsubstituted C₃₋₆ cycloalkyl), and substituted or unsubstituted aryl.

In one embodiment of compounds of formula (Ia), each R¹ is independently halogen, substituted or unsubstituted C₁₋₄ alkyl, substituted or unsubstituted C₃₋₇ cycloalkyl, substituted or unsubstituted 3-6 membered heterocyclyl, substituted or unsubstituted aryl, or —OR; and R² is 2-imidazolyl or 5-imidazolyl, substituted with one or more substituents independently selected from substituted or unsubstituted C₁₋₄ alkyl, substituted or unsubstituted C₃₋₆ cycloalkyl, and (C₁₋₃ alkyl) (substituted or unsubstituted C₃₋₆ cycloalkyl).

In one embodiment of compounds of formula (Ia), each R¹ is independently halogen, substituted or unsubstituted C₁₋₄ alkyl, substituted or unsubstituted C₃₋₇ cycloalkyl, substituted or unsubstituted 3-6 membered heterocyclyl, substituted or unsubstituted aryl, or —OR; and R² is pyrazyl, substituted with one or more substituents independently selected from substituted or unsubstituted C₁₋₄ alkyl, substituted or unsubstituted 3-6 membered heterocyclyl, and —OR, and NR₂. In one embodiment of compounds of formula (Ia), each R is independently —CH₃, —CH₂CH₃, —CH₂CH₂CH₃, —CH(CH₃)₂, —CH₂CH(CH₃)₂, or —CH₂(cyclopropyl). In some embodiments of compounds of formula (Ia), n is 1 or 2.

In one embodiment of compounds of formula (Ia), each R¹ is independently halogen, substituted or unsubstituted C₁₋₄ alkyl, substituted or unsubstituted C₃₋₇ cycloalkyl, substituted or unsubstituted 3-6 membered heterocyclyl, substituted or unsubstituted aryl, or —OR; and R² is pyrazolyl, unsubstituted or substituted with one or more substituted or unsubstituted C₁₋₄ alkyl. In some embodiments of compounds of formula (Ia), n is 1 or 2.

In one embodiment of compounds of formula (Ia), each R¹ is independently halogen, substituted or unsubstituted C₁₋₄ alkyl, substituted or unsubstituted C₃₋₇ cycloalkyl, substituted or unsubstituted 3-6 membered heterocyclyl, substituted or unsubstituted aryl, or —OR; and R² is 2-furanyl unsubstituted or substituted with one or more C₁₋₄ alkyl. In some embodiments of compounds of formula (Ia), n is 1 or 2.

In some embodiments of compounds of formula (Ia), each R¹ is independently F, C₁, —CH₃, —CH₂CH₃, —CH₂CH₂CH₃, —CH₂CH(CH₃)₂, CF₃, cyclopropyl, cylobutyl, cyclopentyl, cyclohexyl, —OCH₃, —OCH₂CH₃, —OCH(CH₃)₂, —OCH₂(cyclopropyl), azetidinyl, phenyl, pyrrolidyl, piperidyl, piperazinyl or morpholinyl.

In some embodiments of compounds of formula (Ia), each R¹ is independently F, Cl, —CH₃, —CH₂CH₃, CF₃, cyclopropyl, cyclohexyl, —OCH₃—OCH(CH₃)₂, —OCH₂(cyclopropyl), azetidinyl, phenyl, or morpholinyl.

In some embodiments of compounds of formula (Ia), R² is 2-pyridyl, substituted with one or more substituents independently selected from F, Cl, —CN, —CH₃, —CH₂CH₃, —CH₂CH₂CH₃, —CH(CH₃)₂—CH₂CH(CH₃)₂, cyclopropyl, cyclobutyl, cyclopentyl, —OCH₃, —OCH₂CH₂CH₃, —OCH₂CH(CH₃)₂, —NH₂, —NHCH₃, and —N(CH₃)₂. In certain embodiments of compounds of formula (Ia), R² is 2-pyridyl, substituted with one or more substituents independently selected from F, Cl, —CN, —CH₃, —CH(CH₃)₂, —CH₂CH(CH₃)₂, cyclopropyl, —OCH₃—OCH₂CH(CH₃)₂, and —N(CH₃)₂.

In some embodiments of compounds of formula (Ia), R² is 3-pyridyl substituted with one or more substituents independently selected from F, Cl, —CN, —CH₃, —CH₂CH₃, and —CF₃. In certain embodiments of compounds of formula (Ia), R² is 3-pyridyl substituted with —CF₃.

In certain embodiments of compounds of formula (Ia), R² is 2-imidazolyl, substituted with one or more substituents independently selected from —CH₃, —CH₂CH₃, —CH₂CF₃, cyclopropyl, —CH₂CH(CH₃)₂, phenyl, and p-trifluoromethyl phenyl.

In certain embodiments of compounds of formula (Ia), R² is 2-imidazolyl, substituted with one or more substituents independently selected from —CH₃, —CH₂CH₃, —CH₂CH₂CH₃, cyclopropyl, and —CH₂CH(CH₃)₂.

In certain embodiments of compounds of formula (I), R² is 2-imidazolyl, substituted with one or more substituents independently selected from —CH₃, —CH₂CH₃, —CH₂CF₃, cyclopropyl, —CH₂CH(CH₃)₂, phenyl, and p-trifluoromethyl phenyl.

In certain embodiments of compounds of formula (I), R² is 2-imidazolyl, substituted with one or more substituents independently selected from —CH₃, —CH₂CH₃, cyclopropyl, and —CH₂CH(CH₃)₂.

In certain embodiments of compounds of formula (Ia), R² is 5-imidazolyl, substituted with one or more substituents independently selected from —CH₃, —CH₂CH₃, —CH₂CH₂CH₃, —CH(CH₃)₂, cyclopropyl, and CH₂-cyclopropyl.

In certain embodiments of compounds of formula (Ia), R² is 5-imidazolyl, substituted with one or more substituents independently selected from —CH(CH₃)₂ and —CH₂-cyclopropyl.

In some embodiments of compounds of formula (Ia), R² is 2-pyrazyl, substituted with one or more substituents independently selected from —CH₃, —CH₂CH₃, —CH₂CH₂CH₃, —CH₂CH(CH₃), —OCH₃, —OCH₂CH₃, —OCH₂CH₂CH₃, —OCH₂CH(CH₃)₂, —N(CH₃)₂, pyrrolidyl, piperidyl, piperazinyl and morpholinyl.

In some embodiments of compounds of formula (Ia), R² is 2-pyrazyl, substituted with one or more substituents independently selected from —CH₃, —OCH₃, —N(CH₃)₂, and pyrrolidyl.

In one embodiment of compounds of formula (Ia), R² is pyrazolyl, unsubstituted or substituted with one or more substituents independently selected from —CH₃, —CH₂CH₃, —CH₂CH₂CH₃, and —CH(CH₃)₂.

In one embodiment of compounds of formula (Ia), R² is 2-furanyl, unsubstituted or substituted with one or more substituents independently selected from —CH₃, —CH₂CH₃, —CH₂CH₂CH₃, and —CH(CH₃)₂. In some embodiments of compounds of formula (Ia), n is 1 or 2.

In certain embodiments of compounds of formula (Ia), each R¹ is independently F, Cl, —CH₃, —CH₂CH₂CH₃, —CF₃, cyclopropyl, cyclohexyl, —OCH₃, —OCH(CH₃)₂, —OCH₂(cyclopropyl), azetidinyl, phenyl, or morpholinyl, and R² is 2-pyridyl, substituted with one or more substituents independently selected from F, Cl, CN, —CH₃, —CH₂CH₃, —CH₂CH₂CH₃, —CH(CH₃)₂, —CH₂CH(CH₃)₂, cyclopropyl, cyclobutyl, cyclopentyl, —OCH₃, —OCH₂CH₃, —OCH₂CH₂CH₃, —OCH₂CH(CH₃)₂, —NH₂, —NHCH₃, and —N(CH₃)₂. In some such embodiments, R² is 2-pyridyl, substituted with one or more substituents independently selected from F, Cl, CN, —CH₃, —CH(CH₃)₂, —CH₂CH(CH₃)₂, cyclopropyl, —OCH₃, —OCH₂CH(CH₃)₂, or —N(CH₃)₂. In some such embodiments, each R¹ is independently F, Cl, —CH₃, —CF₃, cyclopropyl, cyclohexyl, —OCH₃, —OCH(CH₃)₂, —OCH₂(cyclopropyl), azetidinyl, phenyl, or morpholinyl.

In certain embodiments of compounds of formula (Ia), each R¹ is independently F, Cl, —CH₃, —CH₂CH₃, CF₃, cyclohexyl, —OCH₃, or morpholinyl, and R² is 3-pyridyl substituted with one or more substituents independently selected from F, Cl, —CN, —CH₃, —CH₂CH₃, and —CF₃. In certain embodiments of compounds of formula (Ia), R² is 3-pyridyl substituted with —CF₃. In some such embodiments, each R¹ is independently F, —CH₃, or —OCH₃.

In certain embodiments of compounds of formula (Ia), each R¹ is independently F, Br, C₁, —CN, —CH₃, —CH₂CH₃, CF₃, cyclohexyl, —OCH₃, —N(CH₃)₂, —C(O)CH₃, benzoyl, methyl sulfonyl, morpholinyl, phenyl, —O-(m-trifluormethyl)phenyl, or p-fluorophenyl, and R² is 2-imidazolyl, substituted with one or more substituents independently selected from —CH₃, —CH₂CH₃, —CH₂CH₂CH₃, cyclopropyl, CH₂CH(CH₃)₂. In some such embodiments, R² is 2-imidazolyl, substituted with one or more substituents independently selected from —CH₃, —CH₂CH₃, —CH₂CF₃, cyclopropyl, —CH₂CH(CH₃)₂, phenyl, and p-trifluoromethyl phenyl. In some such embodiments, each R¹ is independently F, Br, C₁, —CN, —CH₃, —CH₂CH₃, CF₃, —N(CH₃)₂, —C(O)CH₃, benzoyl, methyl sulfonyl, morpholinyl, —OCH₃, phenyl, —O-(m-trifluormethyl)phenyl, or p-fluorophenyl.

In certain embodiments of compounds of formula (Ia), each R¹ is independently F, Cl, —CH₃, —CH₂CH₃, CF₃, cyclohexyl, —OCH₃, or morpholinyl, and R² is 2-imidazolyl, substituted with one or more substituents independently selected from —CH₃, —CH₂CH₃, —CH₂CH₂CH₃, cyclopropyl, and —CH₂CH(CH₃)₂. In some such embodiments, R² is 2-imidazolyl, substituted with one or more substituents independently selected from —CH₃, —CH₂CH₃, cyclopropyl, and —CH₂CH(CH₃)₂. In some such embodiments, each R¹ is independently F, Cl, —CH₃, —CH₂CH₃, CF₃, morpholinyl, or —OCH₃.

In certain embodiments of compounds of formula (Ia), each R¹ is independently F, Cl, —CH₃, —CH₂CH₃, CF₃, cyclohexyl, —OCH₃, or morpholinyl, and R² is 5-imidazolyl, substituted with one or more substituents independently selected from —CH₃, —CH₂CH₃, —CH₂CH₂CH₃, —CH(CH₃)₂, —CH₂CH(CH₃)₂, cyclopropyl, and —CH₂-cyclopropyl. In some such embodiments, R² is 5-imidazolyl, substituted with one or more substituents independently selected from —CH₃, —CH₂CH₃, —CH₂CH₂CH₃, —CH(CH₃)₂, cyclopropyl, and —CH₂-cyclopropyl. In some such embodiments, R² is 5-imidazolyl, substituted with one or more substituents independently selected from —CH(CH₃)₂ and —CH₂-cyclopropyl. In some such embodiments, each R¹ is independently Cl or morpholinyl.

In certain embodiments of compounds of formula (Ia), each R¹ is independently F, Cl, —CH₃, —CH₂CH₃, —CF₃, cyclohexyl, —OCH₃, or morpholinyl, and R² is 2-pyrazyl, substituted with one or more substituents independently selected from —CH₃, —CH₂CH₃, —CH₂CH₂CH₃, —CH₂CH(CH₃)₂, —OCH₃, —OCH₂CH₃, —OCH₂CH₂CH₃, —OCH₂CH(CH₃)₂, pyrrolidyl, piperidyl, piperazinyl, and morpholinyl. In some such embodiments, R² is 2-pyrazyl, substituted with one or more substituents independently selected from —CH₃, —OCH₃, —N(CH₃)₂, and pyrrolidyl. In some such embodiments, each R¹ is independently C₁ or morpholinyl.

In certain embodiments of compounds of formula (Ia), each R¹ is independently F, Cl, —CH₃, —CH₂CH₃, —CF₃, cyclohexyl, —OCH₃, or morpholinyl, and R² is pyrazolyl, unsubstituted or substituted with one or more substituents independently selected from —CH₃, —CH₂CH₃, —CH₂CH₂CH₃, and —CH(CH₃)₂. In some such embodiments, R² is pyrazolyl, unsubstituted or substituted with —CH₃ and —CH(CH₃)₂. In some such embodiments, each R¹ is independently F or morpholinyl.

In certain embodiments of compounds of formula (Ia), each R¹ is independently F, Cl, —CH₃, —CH₂CH₃, —CF₃, cyclohexyl, —OCH₃, or morpholinyl, and R² is 2-furanyl, unsubstituted or substituted with one or more substituents independently selected from —CH₃, —CH₂CH₃, —CH₂CH₂CH₃, and —CH(CH₃)₂. In some such embodiments, R² is unsubstituted 2-furanyl. In some such embodiments, each R¹ is independently F, —CH₃, —OCH₃, or morpholinyl.

Provided herein are Sulfonamide Compounds having the following formula (II):

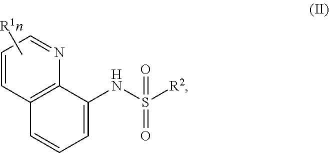

and pharmaceutically acceptable salts, tautomers, isotopologues, and stereoisomers thereof,
wherein:
each R¹ is independently H, halogen, substituted or unsubstituted C₁₋₄ alkyl, substituted or unsubstituted C₃₋₇ cycloalkyl, substituted or unsubstituted 3-6 membered heterocyclyl, substituted or unsubstituted aryl, or —OR;
R² is
a. pyridyl, unsubstituted or substituted with one or more substituents independently selected from halogen, CN, substituted or unsubstituted C₁₋₄ alkyl, substituted or unsubstituted C₃₋₆ cycloalkyl, substituted or unsubstituted C₆₋₁₀ aryl, substituted or unsubstituted monocyclic heteroaryl, —OR, and —NR₂;
b. 2-imidazolyl, unsubstituted or substituted with one or more substituents independently selected from substituted or unsubstituted C₁₋₄ alkyl, substituted or unsubstituted C₃₋₆ cycloalkyl, and (C₁₋₃ alkyl) (substituted or unsubstituted C₃₋₆ cycloalkyl);
c. pyrazyl, unsubstituted or substituted with one or more substituents independently selected from substituted or unsubstituted C₁₋₄ alkyl, substituted or unsubstituted 3-6 membered heterocyclyl, —OR, and NR₂;
d. pyrazolyl, unsubstituted or substituted with one or more substituents independently selected from substituted or unsubstituted C₁₋₄ alkyl and substituted or unsubstituted C₃₋₆ cycloalkyl;
e. pyrimidyl, unsubstituted or substituted with one or more substituents independently selected from substituted or unsubstituted C₁₋₄ alkyl and —OR;
each R is independently H, substituted or unsubstituted C₁₋₄ alkyl, or (C₁₋₃ alkyl) (substituted or unsubstituted C₃₋₆ cycloalkyl);
n is 1-3;
and provided that R and R² are not both unsubstituted.

Further provided herein are Sulfonamide Compounds having the following formula (II):

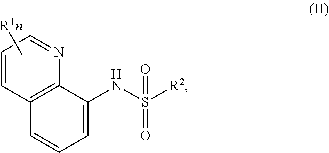

and pharmaceutically acceptable salts, tautomers, isotopologues, and stereoisomers thereof,
wherein:
each R¹ is independently halogen, —CN, substituted or unsubstituted C₁₋₄ alkyl, substituted or unsubstituted C₃₋₇ cycloalkyl, substituted or unsubstituted acyl, substituted or unsubstituted C₁₋₄ sulfonyl, substituted or unsubstituted 3-6 membered heterocyclyl, substituted or unsubstituted aryl, or —OR;
R² is
a. pyridyl, unsubstituted or substituted with one or more substituents independently selected from halogen, CN, substituted or unsubstituted C₁₋₄ alkyl, substituted or unsubstituted C₃₋₆ cycloalkyl, substituted or unsubstituted C₆₋₁₀ aryl, substituted or unsubstituted monocyclic heteroaryl, —OR, and —NR₂;
b. 2-imidazolyl, unsubstituted or substituted with one or more substituents independently selected from substituted or unsubstituted C₁₋₄ alkyl, substituted or unsubstituted C₃₋₆ cycloalkyl, (C₁₋₃ alkyl) (substituted or unsubstituted C₃₋₆ cycloalkyl), and substituted or unsubstituted aryl;
c. pyrazyl, unsubstituted or substituted with one or more substituents independently selected from substituted or unsubstituted C₁₋₄ alkyl, substituted or unsubstituted 3-6 membered heterocyclyl, —OR, and NR₂;
d. pyrazolyl, unsubstituted or substituted with one or more substituents independently selected from substituted or unsubstituted C₁₋₄ alkyl and substituted or unsubstituted C₃₋₆ cycloalkyl;
e. pyrimidyl, unsubstituted or substituted with one or more substituents independently selected from substituted or unsubstituted C₁₋₄ alkyl and —OR; each R is independently H and substituted or unsubstituted C₁₋₄ alkyl, (C₁₋₃ alkyl), (substituted or unsubstituted C₃₋₆ cycloalkyl), or substituted or unsubstituted aryloxy;
n is 1-3; [00167] and provided that R¹ and R² are not both unsubstituted.

In one embodiment of compounds of formula (II), each R¹ is independently H, halogen, substituted or unsubstituted C₁₋₄ alkyl, substituted or unsubstituted C₃₋₇ cycloalkyl, substituted or unsubstituted 3-6 membered heterocyclyl, substituted or unsubstituted aryl, or —OR; and R² is pyridyl, unsubstituted or substituted with one or more substituents independently selected from halogen, CN, substituted or unsubstituted C₁₋₄ alkyl, substituted or unsubstituted C₃₋₆ cycloalkyl, substituted or unsubstituted C₆₋₁₀ aryl, substituted or unsubstituted monocyclic heteroaryl, —OR, and —NR₂.

In one embodiment of compounds of formula (II), each R¹ is independently H, halogen, —CN, substituted or unsubstituted C₁₋₄ alkyl, substituted or unsubstituted C₃₋₇ cycloalkyl, substituted or unsubstituted acyl, substituted or unsubstituted C₁₋₄ sulfonyl, substituted or unsubstituted 3-6 membered heterocyclyl, substituted or unsubstituted aryl, or —OR; and R² is 2-imidazolyl, unsubstituted or substituted with one or more substituents independently selected from substituted or unsubstituted C₁₋₄ alkyl, substituted or unsubstituted C₃₋₆ cycloalkyl, (C₁₋₃ alkyl) (substituted or unsubstituted C₃₋₆ cycloalkyl), and substituted or unsubstituted aryl.

In one embodiment of compounds of formula (II), each R¹ is independently H, halogen, substituted or unsubstituted C₁₋₄ alkyl, substituted or unsubstituted C₃₋₇ cycloalkyl, substituted or unsubstituted 3-6 membered heterocyclyl, substituted or unsubstituted aryl, or —OR; and R² is 2-imidazolyl, unsubstituted or substituted with one or more substituents independently selected from substituted or unsubstituted C₁₋₄ alkyl, substituted or unsubstituted C₃₋₆ cycloalkyl, and (C₁₋₃ alkyl) (substituted or unsubstituted C₃₋₆ cycloalkyl).

In one embodiment of compounds of formula (II), each R¹ is independently H, halogen, substituted or unsubstituted C₁₋₄ alkyl, substituted or unsubstituted C₃₋₇ cycloalkyl, substituted or unsubstituted 3-6 membered heterocyclyl, substituted or unsubstituted aryl, or —OR; and R² is pyrazyl, unsubstituted or substituted with one or more substituents independently selected from substituted or unsubstituted C₁₋₄ alkyl, substituted or unsubstituted 3-6 membered heterocyclyl, and —OR, and NR₂. In one embodiment of compounds of formula (II), each R is independently H, —CH₃, —CH₂CH₃, —CH₂CH₂CH₃, or —CH₂CH(CH₃)₂. In some embodiments of compounds of formula (II), n is 1 or 2.

In one embodiment of compounds of formula (II), each R¹ is independently H, halogen, substituted or unsubstituted C₁₋₄ alkyl, substituted or unsubstituted C₃₋₇ cycloalkyl, substituted or unsubstituted 3-6 membered heterocyclyl, substituted or unsubstituted aryl, or —OR; and R² is pyrazolyl, unsubstituted or substituted with one or more substituents independently selected from substituted or unsubstituted C₁₋₄ alkyl and substituted or unsubstituted C₃₋₆ cycloalkyl.

In one embodiment of compounds of formula (II), each R¹ is independently H, halogen, substituted or unsubstituted C₁₋₄ alkyl, substituted or unsubstituted C₃₋₇ cycloalkyl, substituted or unsubstituted 3-6 membered heterocyclyl, substituted or unsubstituted aryl, or —OR; and R² is pyrimidyl, unsubstituted or substituted with one or more substituents independently selected from substituted or unsubstituted C₁₋₄ alkyl and —OR.

In some embodiments of compounds of formula (II), each R¹ is independently H, F, Cl, —CH₃, —CH₂CH₃, —CH₂CH₂CH₃, —CH₂CH(CH₃)₂, —CF₃, cyclopropyl, cylobutyl, cyclopentyl, cyclohexyl, —OCH₃, —OCH₂CH₃, pyrrolidyl, piperidyl, piperazinyl or morpholinyl. In some embodiments of compounds of formula (II), each R¹ is independently H, F, Cl, —CH₃, —CH₂CH₃, CF₃, cyclohexyl, —OCH₃, or morpholinyl.

In some embodiments of compounds of formula (II), R² is pyridyl, unsubstituted or substituted with one or more substituents independently selected from F, Cl, CN, —CH₃, —CH₂CH₃, —CH₂CH₂CH₃, —CH₂CH(CH₃)₂, —C(CH₃)₃, —(CH₂)CN, —CHF₂, —CF₃, cyclopropyl, cyclobutyl, cyclopentyl, phenyl, phenyl substituted with CN, phenyl substituted with F, oxadiazolyl, —OCH₃, —OCH₂CH₂CH₃, —OCH₂CH(CH₃)₂, —NH₂, —NHCH₃, and —N(CH₃)₂. In certain embodiments of compounds of formula (II), R² is pyridyl, unsubstituted or substituted with one or more substituents independently selected from F, Cl, CN, CH₃, —CH₂CH(CH₃)₂, —C(CH₃)₃, —(CH₂)CN, —CHF₂, —CF₃, cyclopropyl, phenyl, phenyl substituted with CN, phenyl substituted with F, oxadiazolyl, —OCH₃, —OCH₂CH(CH₃)₂, and —N(CH₃)₂.

In certain embodiments of compounds of formula (II), R² is 2-imidazolyl, unsubstituted or substituted with one or more substituents independently selected from —CH₃, —CH₂CH₃, —CH₂CF₃, —CH₂CH₂CH₃, —CH₂CH(CH₃)₂, substituted cyclopropyl, —CH₂-cyclopropyl, phenyl, and p-trifluoromethyl phenyl.

In certain embodiments of compounds of formula (II), R² is 2-imidazolyl, unsubstituted or substituted with one or more substituents independently selected from —CH₃, —CH₂CH₃, —CH₂CH₂CH₃, —CH₂CH(CH₃)₂, substituted cyclopropyl, and —CH₂-cyclopropyl. In certain embodiments of compounds of formula (II), R² is 2-imidazolyl, unsubstituted or substituted with one or more substituents independently selected from —CH₃, —CH₂CH₃, —CH₂CH(CH₃)₂, cyclopropyl substituted with one or more F, and —CH₂-cyclopropyl.

In some embodiments of compounds of formula (II), R² is 2-pyrazyl, unsubstituted or substituted with one or more substituents independently selected from —CH₃, —CH₂CH₃, —CH₂CH₂CH₃, —CH₂CH(CH₃), —OCH₃, —OCH₂CH₃, —OCH₂CH₂CH₃, —OCH₂CH(CH₃)₂, pyrrolidyl, piperidyl, piperazinyl and morpholinyl.

In some embodiments of compounds of formula (II), R² is 2-pyrazyl, unsubstituted or substituted with one or more substituents independently selected from —CH₃, —OCH₃, and pyrrolidyl.

In some embodiments of compounds of formula (II), R² is pyrazolyl, unsubstituted or substituted with one or more substituents independently selected from —CH₃, —CH₂CH₃, —CH₂CH₂CH₃, —CH₂CH(CH₃), —OCH₃, —OCH₂CH₃, —OCH₂CH₂CH₃, —OCH₂CH(CH₃)₂, pyrrolidyl, piperidyl, piperazinyl and morpholinyl.

In some embodiments of compounds of formula (II), R² is pyrazolyl, unsubstituted or substituted with one or more substituents independently selected from —CH₂CH₃ and morpholinyl.

In some embodiments of compounds of formula (II), R² is pyrimidyl, unsubstituted or substituted with one or more substituents independently selected from —CH₃, —CH₂CH₃, —CH₂CH₂CH₃, —CH₂CH(CH₃), —OCH₃, —OCH₂CH₃, —OCH₂CH₂CH₃, and —OCH₂CH(CH₃)₂.

In some embodiments of compounds of formula (II), R² is pyrimidyl, unsubstituted or substituted with one or more substituents independently selected from —CH₃ and —OCH₃.

In certain embodiments of compounds of formula (II), each R¹ is independently H, F, Cl, —CH₃, —CH₂CH₂CH₃, —CF₃, cyclohexyl, —OCH₃, or morpholinyl, and R² is 2-pyridyl, unsubstituted or substituted with one or more substituents independently selected from F, Cl, CN, —CH₃, —CH₂CH₃, —CH₂CH₂CH₃, —CH₂CH(CH₃)₂, cyclopropyl, cyclobutyl, cyclopentyl, —OCH₃, —OCH₂CH₃, —OCH₂CH₂CH₃, —OCH₂CH(CH₃)₂, —CF₃, phenyl substituted with CN, —NH₂, —NHCH₃, and —N(CH₃)₂. In some such embodiments, R² is 2-pyridyl, unsubstituted or substituted with one or more substituents independently selected from F, Cl, CN, —CH₃, —CH₂CH(CH₃)₂, cyclopropyl, —OCH₃, —OCH₂CH(CH₃)₂, —CF₃, phenyl substituted with CN, or —N(CH₃)₂. In some such embodiments, each R¹ is independently H, F, Cl, —CH₃, cyclohexyl, and —OCH₃.

In certain embodiments of compounds of formula (II), each R¹ is independently H, F, Cl, —CH₃, —CH₂CH₂CH₃, —CF₃, cyclohexyl, —OCH₃, or morpholinyl, and R² is 3-pyridyl, unsubstituted or substituted with one or more substituents independently selected from F, Cl, CN, —CH₃, —CH₂CH₃, —CH₂CH₂CH₃, —CH₂CH(CH₃)₂, —C(CH₃)₃, —CHF₂, —CF₃, cyclopropyl, cyclobutyl, cyclopentyl, —OCH₃, —OCH₂CH₃, —OCH₂CH₂CH₃, —OCH₂CH(CH₃)₂, phenyl, phenyl substituted with F, oxadiazolyl, —NH₂, —NHCH₃, and —N(CH₃)₂.

In certain embodiments of compounds of formula (II), each R¹ is independently H, F, Cl, —CH₃, —CH₂CH₂CH₃, —CF₃, cyclohexyl, —OCH₃, or morpholinyl, and R² is 3-pyridyl, unsubstituted or substituted with one or more substituents independently selected from F, Cl, CN, —CH₃, —C(CH₃)₃, —CHF₂, —CF₃, —OCH₃, cyclopropyl, phenyl, phenyl substituted with F, oxadiazolyl, and —NH₂.

In certain embodiments of compounds of formula (II), each R¹ is independently H, F, Cl, —CH₃, —CH₂CH₂CH₃, —CF₃, cyclohexyl, —OCH₃, or morpholinyl, and R² is 4-pyridyl, unsubstituted or substituted with one or more substituents independently selected from F, Cl, CN, —CH₃, —CH₂CH₃, —CH₂CH₂CH₃, —CH₂CH(CH₃)₂, —CF₃, cyclopropyl, cyclobutyl, cyclopentyl, —OCH₃, —OCH₂CH₃, —OCH₂CH₂CH₃, —OCH₂CH(CH₃)₂, phenyl, —NH₂, —NHCH₃, and —N(CH₃)₂.

In certain embodiments of compounds of formula (II), each R¹ is independently H, F, Cl, —CH₃, —CH₂CH₂CH₃, —CF₃, cyclohexyl, —OCH₃, or morpholinyl, and R² is is 4-pyridyl, unsubstituted or substituted with one or more substituents independently selected from —CH₃, cyclopropyl, and —CF₃.

In certain embodiments of compounds of formula (II), each R¹ is independently F, Cl, Br, —CN, —CH₃, —CH₂CH₃, —CF₃, cyclohexyl, —OCH₃, —N(CH₃)₂, —C(O)CH₃, phenyl, —O-(m-trifluoromethyl)phenyl, p-fluorophenyl, benzoyl, methyl sulfonyl, or morpholinyl, and R² is 2-imidazolyl, substituted with one or more substituents independently selected from —CH₃, —CH₂CH₃, —CH₂CF₃, —CH₂CH₂CH₃, —CH₂CH(CH₃)₂, substituted cyclopropyl, —CH₂-cyclopropyl, phenyl, and p-trifluoromethyl phenyl. In some such embodiments, R² is 2-imidazolyl, unsubstituted or substituted with one or more substituents independently selected from —CH₃, —CH₂CH₃, —CH₂CF₃, —CH₂CH(CH₃)₂, cyclopropyl substituted with one or more F, —CH₂-cyclopropyl, phenyl, and p-trifluoromethyl phenyl. In some such embodiments, each R¹ is independently selected from H, F, Br, C₁, —CN, —CH₃, —CH₂CH₃, CF₃, —OCH₃, —N(CH₃)₂, —C(O)CH₃, phenyl, —O-(m-trifluoromethyl)phenyl, p-fluorophenyl, benzoyl, and methyl sulfonyl.

In certain embodiments of compounds of formula (II), each R¹ is independently F, C₁, —CH₃, —CH₂CH₃, —CF₃, cyclohexyl, —OCH₃, or morpholinyl, and R² is 2-imidazolyl, substituted with one or more substituents independently selected from —CH₃, —CH₂CH₃, —CH₂CH₂CH₃, —CH₂CH(CH₃)₂, substituted cyclopropyl, and —CH₂-cyclopropyl. In some such embodiments, R² is 2-imidazolyl, unsubstituted or substituted with one or more substituents independently selected from —CH₃, —CH₂CH₃, —CH₂CH(CH₃)₂, cyclopropyl substituted with one or more F, and —CH₂-cyclopropyl. In some such embodiments, each R¹ is independently H, F, Cl, —CH₃, —CH₂CH₃, CF₃, and —OCH₃.

In certain embodiments of compounds of formula (II), each R¹ is independently H, F, Cl, —CH₃, —CH₂CH₃, CF₃, cyclohexyl, —OCH₃, or morpholinyl, and R² is 2-pyrazyl, unsubstituted or substituted with one or more substituents independently selected from —CH₃, —CH₂CH₃, —CH₂CH₂CH₃, —CH₂CH(CH₃)₂, —OCH₃, —OCH₂CH₃, —OCH₂CH₂CH₃, —OCH₂CH(CH₃)₂, pyrrolidyl, piperidyl, piperazinyl, and morpholinyl. In some such embodiments, R² is 2-pyrazyl, unsubstituted or substituted with one or more substituents independently selected from —CH₃, —OCH₃, and pyrrolidyl. In some such embodiments, each R¹ is independently H, C₁, —CH₃, —CH₂CH₃, —OCH₃, or morpholinyl.

In certain embodiments of compounds of formula (II), each R¹ is independently H, F, Cl, —CH₃, —CH₂CH₃, CF₃, cyclohexyl, —OCH₃, or morpholinyl, and R² is pyrazolyl, unsubstituted or substituted with one or more substituents independently selected from —CH₃, —CH₂CH₃, —CH₂CH₂CH₃, —CH₂CH(CH₃), —OCH₃, —OCH₂CH₃, —OCH₂CH₂CH₃, —OCH₂CH(CH₃)₂, pyrrolidyl, piperidyl, piperazinyl and morpholinyl. In some such embodiments, R² is pyrazolyl, unsubstituted or substituted with one or more substituents independently selected from —CH₂CH₃ and morpholinyl.

In certain embodiments of compounds of formula (II), each R¹ is independently H, F, Cl, —CH₃, —CH₂CH₃, CF₃, cyclohexyl, —OCH₃, or morpholinyl, and R² is pyrimidyl, unsubstituted or substituted with one or more substituents independently selected from —CH₃, —CH₂CH₃, —CH₂CH₂CH₃, —CH₂CH(CH₃), —OCH₃, —OCH₂CH₃, —OCH₂CH₂CH₃, —OCH₂CH(CH₃)₂, pyrrolidyl, piperidyl, piperazinyl and morpholinyl. In some such embodiments, R² is pyrimidyl, unsubstituted or substituted with one or more substituents independently selected from —CH₃ and —OCH₃.

Further embodiments provided herein include combinations of at least one of the particular embodiments set forth above.

Representative compounds of formula (I), formula (Ia), and formula (II) are set forth in Table 1 and Table 2.

Further representative compounds of formula (I), formula (Ia), and formula (II) are set forth in Table 1, Table 2, Table 3, and Table 4.

Each of the compounds in Table 1, Table 2, and Table 3 was tested in one or more of the in vitro parasitic motility assays and was found to have activity therein.

In a further embodiment, each of the compounds in Table 1, Table 2, Table 3, and Table 4 was tested in one or more of the in vitro parasitic motility assays and was found to have activity therein.

### Methods for Making Compounds

The Sulfonamide Compounds of formula (I), formula (Ia), and formula (II), Table 1, Table 2, and Table 3 can be made using conventional organic syntheses and commercially available starting materials. Further, the Sulfonamide Compounds of formula (I), formula (Ia), and formula (II), Table 1, Table 2, and Table 3, and Table 4 can be made using conventional organic syntheses and commercially available starting materials. By way of example and not limitation, Sulfonamide Compounds of formula (I), formula (Ia), and formula (II), Table 1, Table 2, Table 3, and Table 4 can be prepared as outlined in Scheme 1, shown below, as well as in the examples set forth herein. It should be noted that one skilled in the art would know how to modify the procedures set forth in the illustrative schemes and examples to arrive at the desired products.

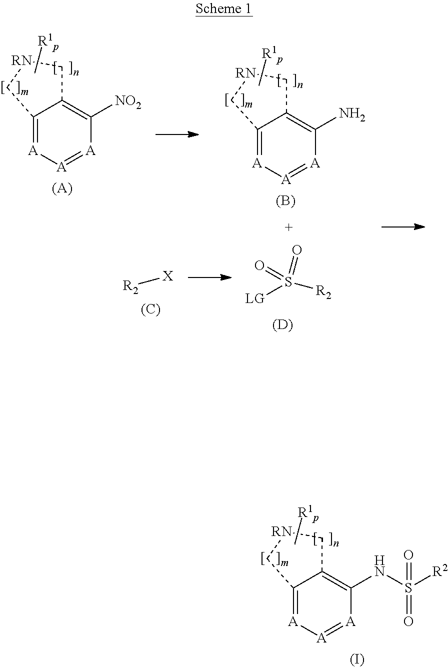

As shown in Scheme 1, compounds of formula (I), formula (Ia), formula (II), Table 1, Table 2, Table 3, and Table 4, wherein R¹, R², R, A, m, n, and p are as defined herein, can be prepared starting from an appropriately derivatized heterocyclicamine (B) and sulfonyls (D), wherein LG (leaving group) is Cl or Bt (1H-benzo[d][1,2,3]triazolyl). Compounds of formula I wherein (B) is quinolone-8-amine, (B) is commercially available or may be prepared according to known methods (see for example, J. Heterocyclic Chem., 39, 631 (2002)). Compounds wherein (B) is either hexahydroquinolin-amine or tetrahydro-indol amine, compounds are commercially available or may be prepared according to known methods (see for example, Org. Biomol. Chem., 2020, 18, 1214-1220). Appropriately substituted 8-nitroquinoline derivatives (A) are reduced, using for example, SnCl₂ or H₂ in the presence of a catalyst, such as Pd/C, in a solvent, such as MeOH or EtOH, and heating at temperatures ranging from 25 to 70° C., to provide derivatized quinolone-8-amines (B). Sulfonyl chlorides (D), wherein LG is C₁, are commercially available or may be prepared according to known methods (see for example, Bull. Korean Chem. Soc., 33, 383 (2012)). Thio-alkylation of the appropriately derivatized R² containing moiety (C), wherein X is Cl, with (4-methoxyphenyl)methanethiol or phenylmethanethiol in a solvent, such as THF or DMSO, in the presence of a base, such as NaH or CsF, at temperatures ranging from 0 to 80° C., provides the intermediate benzyl thiols. Subsequent oxidative chlorination of the benzyl thiol with 1,3-dichloro-5,5-dimethylimidazolidine-2,4-dione (DCH) or NaOCl in a solvent, such as DCM and water, in the presence of an acid, such as HOAc, at temperatures ranging from −10 to 25° C., provides sulfonyls (D), wherein LG is C₁. Alternatively, treatment of the appropriately derivatized R² containing moiety (C) wherein X is Br, with a base, such as nBuLi, in the presence of SO₂Cl₂ in a solvent such as THF, at temperatures ranging from −78 to 25° C. or in the presence of SO₂ gas in a solvent, such as Et₂O, at temperatures ranging from −70 to 25° C., and subsequent reaction with NCS in a solvent, such as CHCl₃ and water, at temperatures ranging from 0 to 25° C., provides sulfonyls (D), wherein LG is Cl. In another method, treatment of the appropriately derivatized R² moiety (C), wherein X is SH, with Cl₂ gas in a solvent, such as DCM and water, in the presence of an acid, such as HOAc, at temperatures ranging from 0 to 25° C., provides sulfonyls (D), wherein LG is Cl. Sulfonyls (D), wherein LG is Bt, are prepared according to known methods. Treatment of the appropriately derivatized R² moiety (C), wherein X is H, with a base, such as nBuLi, in the presence of S02 gas in a solvent, such as THF, at temperatures ranging from −78 to 0° C., followed by addition of N-chlorobenzotriazole in the presence of a base, such as TEA, at temperatures ranging from 0 to 25° C., provides sulfonyls (D), wherein LG is benzotriazole. Compounds of formula (I), formula (Ia), formula (II), Table 1, Table 2, Table 3, and Table 4 are obtained by treatment of quinolone-8-amines (B) and sulfonyls (D) with a base, such as NaHMDS or pyridine, in a solvent, such as THF or DCM, at temperatures ranging from −78 to 130° C.

In one aspect, provided herein are methods for preparing a Sulfonamide Compound of formula (I):

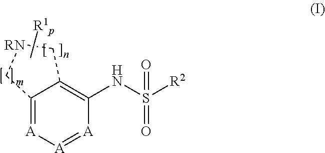

the methods comprising contacting a compound of formula (B):

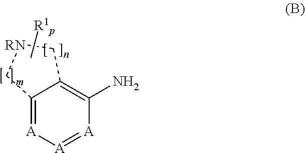

with a compound of formula (D):

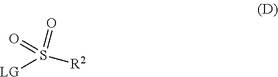

in a solvent, in presence of a base under conditions suitable to provide a Sulfonamide Compound of formula (I), wherein:
- - - is a single or double bond;
each A is independently N or CR¹;
each R¹ is independently H, halogen, alkyl, alkenyl, alkynyl, cycloalkyl, aryl, heterocyclyl, heteroaryl, aralkyl, heterocyclylalkyl, heteroarylalkyl; hydroxy; alkoxy; cycloalkyloxy, aryloxy, heterocyclyloxy, heteroaryloxy, cycloalkylalkyloxy, aralkyloxy, heterocyclylalkyloxy, heteroarylalkyloxy; amino, alkylamino, cycloalkylamino, arylamino, heterocyclylamino, heteroarylamino; imino; imido; amidino; enamino; acylamino; sulfonylamino; urea, alkoxyamino; aralkoxyamino; thio (—SH) sulfonyl; aminosulfonyl; acyl; formyl; carboxy; ester; carbamate; amido; or cyano; each optionally further substituted;
R² is substituted or unsubstituted alkyl, cycloalkyl, heterocyclyl, aryl or heteroaryl;
R is absent, H, substituted or unsubstituted alkyl, cycloalkyl, heterocyclyl, or CO (substituted or unsubstituted alkyl, cycloalkyl, aryl, heterocyclyl, or heteroaryl);
m is 0-3;
n is 0-3; and
p is 0-3;
provided that m and n are not both 0.

In some embodiments, the base is NaHMDS or pyridine. In some embodiments, the solvent is THF or DCM. In some embodiments, the contacting is performed at a temperature ranging from room temperature to −78 to 130° C. In some embodiments, LG is C₁ or 1H-benzo[d][1,2,3]triazolyl.

In some embodiments, the methods further comprise preparing a compound of formula (B):

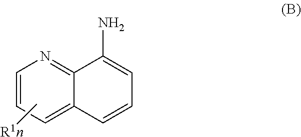

the method comprising reducing a compound of formula (A):

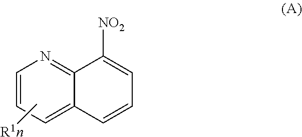

with a reducing agent, in a solvent, under conditions suitable to provide a compound of formula (A).

In some embodiments, the reducing agent is SnCl₂ or H₂ gas in the presence of a catalyst. In one embodiment, the catalyst is Pd/C. In one embodiment, the solvent is MeOH or EtOH. In some embodiments, the contacting is performed at a temperature ranging from 25 to 70° C.

In some embodiments, the methods further comprise preparing a compound of formula (D):

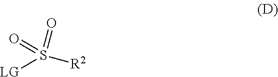

the method comprising:
a) contacting R²—X, wherein X is C₁, with (4-methoxyphenyl)methanethiol or phenylmethanethiol, in a first solvent;
b) contacting the product of step a) with 1,3-dichloro-5,5-dimethylimidazolidine-2,4-dione or NaOCl, in a second solvent, under conditions suitable to provide a compound of formula (D), wherein LG is C₁.

In some embodiments, the contacting in step (a) comprises the presence of a base. In one embodiment, the base in step (a) is NaH. In some embodiments, the first solvent is THF. In some embodiments, the contacting in step (a) is performed at a temperature ranging from 0 to 80 CC. In some embodiments, the base in step (a) is CsF and the first solvent is DMSO.

In some embodiments, the methods of step b) further comprise the presence of an acid. In one embodiment, the acid is HOAc. In some embodiments, the second solvent is DCM and water. In some embodiments, the contacting in step (b) is performed at a temperature ranging from −10 to 25° C.

In some embodiments, the methods further comprise preparing a compound of formula (D):

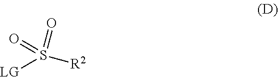

the methods comprising:
a) contacting R²—X, wherein X is Br, with a base in the presence of SO₂ gas, in a first solvent;
b) contacting the product of step a) with NCS, in a second solvent, under conditions suitable to provide a compound of formula (D), wherein LG is Cl.

In one embodiments, the base in step (a) is nBuLi. In one embodiment, the first solvent is Et₂O. In some embodiments, the contacting in step (a) is performed at a temperature ranging from −70 to 25° C.

In some embodiments, the second solvent is CHCl_{and} water. In some embodiments, the contacting in step (b) is performed at a temperature ranging from 0 to 25° C.

In some embodiments, the base in step (a) is nBuLi. In one embodiment, the first solvent is THF. In some embodiments, the contacting in step (a) is performed at a temperature ranging from −78 to 25° C. In some embodiments, contacting the product of step a) with SO₂Cl₂ is performed at a temperature ranging from −78 to 25° C.

In some embodiments, the methods further comprise preparing a compound of formula (D):

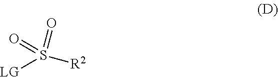

the method comprising contacting R²—X, wherein X is SH, with an acid in the presence of C₁₂ gas, in a solvent, under conditions suitable to provide a compound of formula (D), wherein LG is Cl.

In one embodiment the acid is HOAc. In one embodiment the solvent is DCM and water. In some embodiments, the contacting is performed at a temperature ranging from 0 to 25° C.

In some embodiments, the methods further comprise preparing a compound of formula (D):

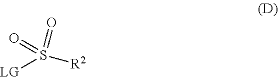

the methods comprising:
a) contacting R²—X, wherein X is H, with a base in the presence of SO₂ gas, in a first solvent;
b) contacting the product of step a) with N-chlorobenzotriazole, in a second solvent, under conditions suitable to provide a compound of formula (D), wherein LG is 1H-benzo[d][1,2,3]triazolyl.

In one embodiment the base in step (a) is nBuLi. In one embodiment the first solvent is Et₂O. In some embodiments, the contacting in step (a) is performed at a temperature ranging from −78 to 0° C.

In some embodiments, the methods further comprise the presence of a base in step (b). In some embodiments, the base is TEA. In some embodiments, the second solvent is THF. In some embodiments, the contacting in step (b) is performed at temperatures ranging from 0 to 25° C.

### Methods of Use

The Sulfonamide Compounds, including compounds of formula (I), formula (Ia), formula (II), Table 1, Table 2, and Table 3 have utility as pharmaceuticals to treat, prevent or improve conditions in animals and humans. Further, the Sulfonamide Compounds, including compounds of formula (I), formula (Ia), formula (II), Table 1, Table 2, Table 3, and Table 4 have utility as pharmaceuticals to treat, prevent or improve conditions in animals and humans. The Sulfonamide Compounds provided herein have utility for use in the treatment or prevention of all diseases, disorders or conditions disclosed herein.

In one aspect, provided herein is a method of treating a disease caused by a helminthic infection. In certain embodiments, a compound as described herein is used in human medical therapy, particularly in the treatment of helminthic infection. In certain embodiments, a compound as provided herein is used in animal medical therapy, particularly in the treatment of helminthic infections. In certain embodiments, the method includes administering a therapeutically effective amount of a compound as described to a subject having a disease caused by a helminthic infection.

In one aspect, provided herein is a method of treating a disease caused by a filarial worm infection. In certain embodiments, a compound as described herein is used in human medical therapy, particularly in the treatment of filarial worm infection. In certain embodiments, a compound as provided herein is used in animal medical therapy, particularly in the treatment of filarial worm infections. In certain embodiments, the method includes administering a therapeutically effective amount of a compound as described to a subject having a disease caused by a filarial worm infection.

In one embodiment, provided herein is a method for the treatment or prevention of helminthic infections and diseases, the methods comprising administering to a subject an effective amount of a Sulfonamide Compound, or a pharmaceutically acceptable salt, tautomer, isotopologue, or stereoisomer thereof. In some such embodiments, the helminthic infection is a filarial worm infection.

In one aspect, provided herein is a method of treating a disease caused by helminthic infection. In certain embodiments, a Sulfonamide Compound, or a pharmaceutically acceptable salt, tautomer, isotopologue, or stereoisomer thereof, is used in human medical therapy, particularly in the treatment of helminthic infections. In certain embodiments, a Sulfonamide Compound, or a pharmaceutically acceptable salt, tautomer, isotopologue, or stereoisomer thereof, is used in animal medical therapy, particularly in the treatment of helminthic infections. In certain embodiments, the method includes administering a therapeutically effective amount of a Sulfonamide Compound, or a pharmaceutically acceptable salt, tautomer, isotopologue, or stereoisomer thereof, to a subject having a disease caused by helminthic infection.

In one aspect, provided herein is a method of treating a disease caused by a filarial worm infection. In certain embodiments, a Sulfonamide Compound, or a pharmaceutically acceptable salt, tautomer, isotopologue, or stereoisomer thereof, is used in human medical therapy, particularly in the treatment of a filarial worm infection. In certain embodiments, a Sulfonamide Compound, or a pharmaceutically acceptable salt, tautomer, isotopologue, or stereoisomer thereof, is used in animal medical therapy, particularly in the treatment of a filarial worm infection. In certain embodiments, the method includes administering a therapeutically effective amount of a Sulfonamide Compound, or a pharmaceutically acceptable salt, tautomer, isotopologue, or stereoisomer thereof, to a subject having a disease caused by a filarial worm infection.

In another aspect, also provided is a method of preventing a disease caused by helminthic infection. In certain embodiments, a Sulfonamide Compound, or a pharmaceutically acceptable salt, tautomer, isotopologue, or stereoisomer thereof, is used in human medical therapy, particularly in the prevention of helminthic infection. In certain embodiments, a Sulfonamide Compound, or a pharmaceutically acceptable salt, tautomer, isotopologue, or stereoisomer thereof, is used in animal medical therapy, particularly in the prevention of helminthic infection. In certain embodiments, the method includes administering a therapeutically effective amount of a Sulfonamide Compound, or a pharmaceutically acceptable salt, tautomer, isotopologue, or stereoisomer thereof, to a subject to prevent a disease caused by helminthic infection.

In another aspect, also provided is a method of preventing a disease caused by a filarial worm infection. In certain embodiments, a Sulfonamide Compound, or a pharmaceutically acceptable salt, tautomer, isotopologue, or stereoisomer thereof, is used in human medical therapy, particularly in the prevention of a filarial worm infection. In certain embodiments, a Sulfonamide Compound, or a pharmaceutically acceptable salt, tautomer, isotopologue, or stereoisomer thereof, is used in animal medical therapy, particularly in the prevention of a filarial worm infection. In certain embodiments, the method includes administering a therapeutically effective amount of a Sulfonamide Compound, or a pharmaceutically acceptable salt, tautomer, isotopologue, or stereoisomer thereof, to a subject to prevent a disease caused by a filarial worm infection.

In another aspect, provided herein is a method of treating or preventing a parasitic disease. In certain embodiments, the parasitic disease is associated with a worm. In certain embodiments, the parasitic disease is caused by a worm. In certain embodiments, the parasitic worm is categorized as cestode (tapeworm), nematode (roundworm) and trematode (flatworm or fluke). In certain embodiments, the parasitic disease is associated with a helminth. In certain embodiments, the parasitic disease is associated with a nematode. In certain embodiments, the nematode is Wuchereria bancrofti. In certain embodiments, the nematode is Brugia malayi. In certain embodiments, the nematode is Brugia timori. In certain embodiments, the nematode is Onchocerca volvulus. In certain embodiments, the nematode is Dirofilaria immitis. In some embodiments, the nematode is Haemonchus contortus. In certain embodiments, the nematode is Ascaris lumbricoides. In certeain embodiments, the nematode is Necator americanus. In still another embodiments, the nematode is Ancylostoma duodenale. In yet other embodiments, the nematode is Trichuris trichiura. In certain embodiments, the parasitic disease is associated with a trematode. In certain embodiments, the parasitic disease is associated with Schistosoma. In certain embodiments, the parasitic disease is associated with Schistosoma mansoni. In certain embodiments, the parasitic disease is enterobiasis, oxyuriasis, ascariasis, ancylostomiasis, necatoriasis, dracunculiasis, filariasis, onchocerciasis, schistosomiasis, or trichuriasis. In certain embodiments, the parasitic disease is schistosomiasis. In certain embodiments, the parasitic disease is urinary schistosomiasis. In certain embodiments, the parasitic disease is intestinal schistosomiasis. In certain embodiments, the parasitic disease is Asian intestinal schistosomiasis. In certain embodiments, the parasitic disease is visceral schistosomiasis. In certain embodiments, the parasitic disease is acute schistosomiasis. In certain embodiments, the parasitic disease is lymphatic filariasis. In certain embodiments, the parasitic disease is bancroftian filariasis. In certain embodiments, the parasitic disease is subcutaneous filariasis. In certain embodiments, the parasitic disease is serious cavity filariasis. In certain embodiments, the parasitic disease is elephantiasis. In certain embodiments, the parasitic disease is elephantiasis tropica. In certain embodiments, the parasitic disease is onchocerciasis. In certain embodiments, the dirofilariasis is dirofilariasis in dogs. In some embodiments, the dirofilariasis is caused by Dirofilaria immitis or Dirofilaria repens. In certain embodiments, the parasitic disease is haemonchosis. In certain embodiments, the haemonchosis is haemonchosis in sheep and goats. In some embodiments, the haemonchosis is caused by Haemonchus contortus.

In certain aspects, the present methods comprise a step of administering a Sulfonamide Compound, or a pharmaceutically acceptable salt, tautomer, isotopologue, or stereoisomer thereof, to a subject. In certain embodiments, the methods comprise administering a Sulfonamide Compound, or a pharmaceutically acceptable salt, tautomer, isotopologue, or stereoisomer thereof, to a subject for no more than fourteen (14) days. In certain embodiments, the methods comprise administering a Sulfonamide Compound, or a pharmaceutically acceptable salt, tautomer, isotopologue, or stereoisomer thereof, to a subject for no more than seven (7) days. In certain embodiments, the subject is in need of treatment for an helminthic infection. In certain embodiments, the subject is in need of treatment for a filarial infection. In certain embodiments, the subject has an helminthic infection. In certain embodiments, the subject is at risk for having an helminthic infection. In certain embodiments, the subject has a filarial infection. In certain embodiments, the subject is at risk for having a filarial infection. In certain embodiments, the subject is a pediatric subject. In certain embodiments, the subject is less than nine (9) years of age. In certain embodiments, the subject is less than eight (8) years of age. In certain embodiments, the subject is a pregnant woman. In certain embodiments, the subject is a post-partum woman. In certain embodiments, the subject is a woman of childbearing potential. In certain embodiments, the subject is an individual attempting to conceive a child. In certain embodiments, the subject is a domestic animal. In certain embodiments, the subject is a dog.

The compounds disclosed herein exhibit potency against helminths, and, therefore, have the potential to kill and/or inhibit the growth, molt, or motility of such helminths. The compounds disclosed herein exhibit potency against filarial worms, and, therefore, have the potential to kill and/or inhibit the growth, molt, or motility of such filarial worms. Thus, in one aspect provided is a method of killing a filarial worm, comprising: contacting the filarial worm with a Sulfonamide Compound, or a pharmaceutically acceptable salt, tautomer, isotopologue, or stereoisomer thereof, in an amount effective to kill the filarial worm. In another aspect, provided herein is a method of inhibiting growth or molt of a filarial worm, comprising: contacting the filarial worm with a Sulfonamide Compound, or a pharmaceutically acceptable salt, tautomer, isotopologue, or stereoisomer thereof, in an amount effective to inhibit growth or molt of the filarial worm. In another aspect, provided herein is a method of inhibiting motility of a filarial worm, comprising: contacting the filarial worm with a Sulfonamide Compound, or a pharmaceutically acceptable salt, tautomer, isotopologue, or stereoisomer thereof, in an amount effective to inhibit motility of the filarial worm. In certain embodiments, the worm is an egg. In certain embodiments, the egg is an unfertilized egg. In certain embodiments, the egg is fertilized egg. In certain embodiments, the worm is a larva. In certain embodiments, the worm is in a larval or juvenile stage. In certain embodiments, the worm is a larva in any one of four larval stages (L1, L2, L3, L4). In certain embodiments, the worm is a larva of stage L1 or microfilaria. In certain embodiments, microfilaria is a larva of stage L1. In certain embodiments, the worm is a larva of stage L2. In certain embodiments, the worm is a larva of stage L3. In certain embodiments, the worm is a larva of stage L4. In certain embodiments the worm is in sexually immature stage (stage L5). In certain embodiments, the worm is mature. In certain embodiments, the worm is fully mature. In certain embodiments, the worm is in adult stage. In certain embodiments, the worm is in pre-parasitic stage. In certain embodiments, the worm is in parasitic stage. In certain embodiments, the worm is contacted with a Sulfonamide Compound, or a pharmaceutically acceptable salt, tautomer, isotopologue, or stereoisomer thereof, inside a subject. In certain embodiments, the worm is contacted with a Sulfonamide Compound, or a pharmaceutically acceptable salt, tautomer, isotopologue, or stereoisomer thereof, outside a subject.

As discussed herein, compounds provided herein are useful for treating and preventing certain diseases and disorders in humans and animals. In certain embodiments, a Sulfonamide Compound, or a pharmaceutically acceptable salt, tautomer, isotopologue, or stereoisomer thereof, is used to treat a disease caused by helminthic infection. In certain embodiments, a Sulfonamide Compound, or a pharmaceutically acceptable salt, tautomer, isotopologue, or stereoisomer thereof, is used to treat a disease caused by filarial worm infection, including, but not limited to, heartworm disease, ascariasis, trichuriasis, schistosomiasis, haemonchosis, onchocerciasis, and lymphatic filariasis. In certain embodiments, treatment or prevention of such diseases and disorders can be effected by administering a Sulfonamide Compound, or a pharmaceutically acceptable salt, tautomer, isotopologue, or stereoisomer thereof, either alone or in combination with another active agent as part of a combination therapy. The term “combination” as in the phrase “in combination with another active agent” includes co-administration of a first agent and a second agent, which for example may be dissolved or intermixed in the same pharmaceutically acceptable carrier, or administration of a first agent, followed by the second agent, or administration of the second agent, followed by the first agent. The present methods and compositions, therefore, include methods of combination therapeutic treatment and combination pharmaceutical compositions. The term “combination therapy” refers to the administration of two or more therapeutic substances, such as a compound described herein and another drug (e.g., an antihelminthic agent such as ivermectin, albendazole, flubendazole, diethylcarbamazine, or emodepside). The other drug(s) may be administered concomitant with, prior to, or following the administration of the macrolide antibiotic.

In one embodiment, provided is a method for the treatment or prevention of helminthic infections and diseases, the methods comprising administering to a subject an effective amount of a Sulfonamide Compound, or a pharmaceutically acceptable salt, tautomer, isotopologue, or stereoisomer thereof, in combination with one or more antihelminthic agent. In some such embodiments, the helminthic infection is a filarial worm infection. In one embodiment, the treatment of helminthic infections comprises administration of an antihelminthic agent such as benzimidazoles, for example, flubendazole, albendazole, mebendazole, thiabendazole, fenbendazole, or triclabendazole. In one embodiment, the treatment of helminthic infections comprises administration of one or more antihelminthic agents, for example, ivermectin, abamectin, diethylcarbamazine (DEC), suramin, pyrantel pamoate, levamisole, niclosamide, nitazoxanide, oxyclozanide, praziquantel, emodepside, monepantel, derquantel, or pelletierine sulphate. In certain embodiments, a Sulfonamide Compound, or a pharmaceutically acceptable salt, tautomer, isotopologue, or stereoisomer thereof, is used to treat helminthic infections in combination with one or more antihelminthic agents. In some embodiments, the antihelminthic agent is a benzimidazole, for example, flubendazole, albendazole, mebendazole, thiabendazole, fenbendazole, or triclabendazole. In some embodiments, the antihelminthic agent is one or more of ivermectin, abamectin, diethylcarbamazine (DEC), suramin, pyrantel pamoate, levamisole, niclosamide, nitazoxanide, oxyclozanide, praziquantel, emodepside, monepantel, derquantel, or pelletierine sulphate. In one embodiment, the antihelminthic agent is invermectin, moxidectin or selamectin. In certain embodiments, a Sulfonamide Compound, or a pharmaceutically acceptable salt, tautomer, isotopologue, or stereoisomer thereof, is used in a method of treatment or prevention of filarial worm infections and diseases, the method comprising administering to a subject an effective amount of a Sulfonamide Compound, or a pharmaceutically acceptable salt, tautomer, isotopologue, or stereoisomer thereof in combination with one or more antihelminthic agents. In some such embodiments, the antihelminthic agent is selected from flubendazole, albendazole, mebendazole, thiabendazole, fenbendazole, triclabendazole, ivermectin, abamectin, diethylcarbamazine (DEC), suramin, pyrantel pamoate, levamisole, niclosamide, nitazoxanide, oxyclozanide, praziquantel, emodepside, monepantel, derquantel, or pelletierine sulphate. In one embodiment, the antihelminthic agent is a Wolbachia targeting agent. In one embodiment, the Wolbachia targeting agent is doxycycline.

### Pharmaceutical Compositions and Routes of Administration

Provided herein are pharmaceutical compositions comprising an effective amount of a Sulfonamide Compound, as described herein, and a pharmaceutically acceptable carrier, excipient or vehicle. The Sulfonamide Compounds can be administered to a subject enterally (for example, orally, rectally), topically, or parenterally (for example, intravenously, intramuscularly, subcutaneously), in the conventional form of preparations, such as capsules, microcapsules, tablets, granules, powder, troches, pills, suppositories, injections, suspensions, syrups, patches, creams, lotions, ointments, gels, sprays, solutions and emulsions. Suitable formulations can be prepared by methods commonly employed using conventional, organic or inorganic additives, such as an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, polyethyleneglycol, sucrose or starch), a disintegrator (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g., sodium benzoate, sodium bisulfite, methylparaben or propylparaben), a stabilizer (e.g., citric acid, sodium citrate or acetic acid), a suspending agent (e.g., methylcellulose, polyvinyl pyrrolidone or aluminum stearate), a dispersing agent (e.g., hydroxypropylmethylcellulose), a diluent (e.g., water), a cosolvent (e.g., propylene glocyl/glycofurol), a buffer, a copolymer (e.g., poly(lactic-co-glycolic acid, i.e PLGA), and base wax (e.g., cocoa butter, white petrolatum or polyethylene glycol). The effective amount of the Sulfonamide Compound in the pharmaceutical composition may be at a level that will exercise the desired effect; for example, about 0.005 mg/kg of a subject's body weight to about 20 mg/kg of a subject's body weight in unit dosage for both oral and parenteral administration.

The dose of a Sulfonamide Compound to be administered to a subject is rather widely variable and can be subject to the judgment of a health-care practitioner. In general, the Sulfonamide Compound can be administered one to four times a day in a dose of about 0.5 mg/kg of a subject's body weight to about 20 mg/kg of a subject's body weight in a subject, but the above dosage may be properly varied depending on the age, body weight and medical condition of the subject and the type of administration. In one embodiment, the dose is about 0.1 mg/kg of a subject's body weight to about 3 mg/kg of a subject's body weight, about 0.5 mg/kg of a subject's body weight to about 2 mg/kg of a subject's body weight, about 1 mg/kg of a subject's body weight to about 2 mg/kg of a subject's body weight or about 1.5 mg/kg of a subject's body weight to about 2 mg/kg of a subject's body weight. In one embodiment, the dose is about 1 mg/kg of a subject's body weight to about 3 mg/kg of a subject's body weight. In one embodiment, the dose is about 0.5 mg/kg of a subject's body weight to about 1 mg/kg of a subject's body weight. In one embodiment, the dose is about 1 mg/kg of a subject's body weight to about 2 mg/kg of a subject's body weight. In one embodiment, the dose is about 1.5, 1.6, 1.7, 1.8, 1.9, 2.0, 2.1, 2.2, 2.3, 2.4, 2.5, 2.6, 2.7, 2.8, 2.9, 3.0 mg/kg of a subject's body weight. In one embodiment, one dose is given per day. In any given case, the amount of the Sulfonamide Compound administered will depend on such factors as the solubility of the active component, the formulation used and the route of administration. In one embodiment, application of a topical concentration provides intracellular exposures or concentrations of about 0.01-10 μM.

In another embodiment, provided herein are methods for the treatment or prevention of a disease or disorder comprising the administration of about 1 mg/day to about 1200 mg/day of a Sulfonamide Compound to a subject affected by helminthic infections. In another embodiment, provided herein are methods for the treatment or prevention of a disease or disorder comprising the administration of about 0.375 mg/day to about 750 mg/day, about 0.75 mg/day to about 375 mg/day, about 3.75 mg/day to about 75 mg/day, about 7.5 mg/day to about 55 mg/day or about 18 mg/day to about 37 mg/day of a Sulfonamide Compound to a subject affected by helminthic infections. In one embodiment, the methods for the treatment of a disease or disorder comprise the administration of about 0.375 mg/day to about 750 mg/day of a Sulfonamide Compound to a subject affected by helminthic infections. In one embodiment, the methods for the treatment of a disease or disorder comprise the administration of about 0.75 mg/day to about 375 mg/day of a Sulfonamide Compound to a subject affected by helminthic infections. In one embodiment, the methods for the treatment of a disease or disorder comprise the administration of about 3.75 mg/day to about 75 mg/day of a Sulfonamide Compound to a subject affected by helminthic infections. In one embodiment, the methods for the treatment of a disease or disorder comprise the administration of about 7.5 mg/day to about 55 mg/day of a Sulfonamide Compound to a subject affected by helminthic infections. In one embodiment, the methods for the treatment of a disease or disorder comprise the administration of about 18 mg/day to about 37 mg/day of a Sulfonamide Compound to a subject affected by helminthic infections.

In another embodiment, provided herein are unit dosage formulations that comprise between about 1 mg and 200 mg, about 35 mg and about 1400 mg, about 125 mg and about 1000 mg, about 250 mg and about 1000 mg, or about 500 mg and about 1000 mg of a Sulfonamide Compound. In one embodiment, the unit dosage formulations comprises between about 1 mg and 200 mg of a Sulfonamide Compound. In one embodiment, the unit dosage formulations comprises between about 35 mg and about 1400 mg of a Sulfonamide Compound. In one embodiment, the unit dosage formulations comprises between about 125 mg and about 1000 mg of a Sulfonamide Compound. In one embodiment, the unit dosage formulations comprises between about 250 mg and about 1000 mg of a Sulfonamide Compound. In one embodiment, the unit dosage formulations comprises between about 500 mg and about 1000 mg of a Sulfonamide Compound.

In a particular embodiment, provided herein are unit dosage formulations comprising about 100 mg or 400 mg of a Sulfonamide Compound.

In another embodiment, provided herein are unit dosage formulations that comprise 1 mg, 5 mg, 10 mg, 15 mg, 20 mg, 30 mg, 35 mg, 40 mg, 50 mg, 70 mg, 100 mg, 125 mg, 130 mg, 140 mg, 175 mg, 200 mg, 250 mg, 280 mg, 350 mg, 500 mg, 560 mg, 700 mg, 750 mg, 1000 mg or 1400 mg of a Sulfonamide Compound. In one embodiment, the unit dosage formulations comprise 1 mg of a Sulfonamide Compound. In one embodiment, the unit dosage formulations comprise 5 mg of a Sulfonamide Compound. In one embodiment, the unit dosage formulations comprise 10 mg of a Sulfonamide Compound. In one embodiment, the unit dosage formulations comprise 15 mg of a Sulfonamide Compound. In one embodiment, the unit dosage formulations comprise 20 mg of a Sulfonamide Compound. In one embodiment, the unit dosage formulations comprise 25 mg of a Sulfonamide Compound. In one embodiment, the unit dosage formulations comprise 30 mg of a Sulfonamide Compound. In one embodiment, the unit dosage formulations comprise 35 mg of a Sulfonamide Compound. In one embodiment, the unit dosage formulations comprise 40 mg of a Sulfonamide Compound. In one embodiment, the unit dosage formulations comprise 50 mg of a Sulfonamide Compound. In one embodiment, the unit dosage formulations comprise 70 mg of a Sulfonamide Compound. In one embodiment, the unit dosage formulations comprise 100 mg of a Sulfonamide Compound. In one embodiment, the unit dosage formulations comprise 125 mg of a Sulfonamide Compound. In one embodiment, the unit dosage formulations comprise 130 mg of a Sulfonamide Compound. In one embodiment, the unit dosage formulations comprise 140 mg of a Sulfonamide Compound. In one embodiment, the unit dosage formulations comprise 175 mg of a Sulfonamide Compound. In one embodiment, the unit dosage formulations comprise 200 mg of a Sulfonamide Compound. In one embodiment, the unit dosage formulations comprise 250 mg of a Sulfonamide Compound. In one embodiment, the unit dosage formulations comprise 280 mg of a Sulfonamide Compound. In one embodiment, the unit dosage formulations comprise 350 mg of a Sulfonamide Compound. In one embodiment, the unit dosage formulations comprise 500 mg of a Sulfonamide Compound. In one embodiment, the unit dosage formulations comprise 560 mg of a Sulfonamide Compound. In one embodiment, the unit dosage formulations comprise 700 mg of a Sulfonamide Compound. In one embodiment, the unit dosage formulations comprise 750 mg of a Sulfonamide Compound. In one embodiment, the unit dosage formulations comprise 1000 mg of a Sulfonamide Compound. In one embodiment, the unit dosage formulations comprise 1400 mg of a Sulfonamide Compound.

A Sulfonamide Compound can be administered once, twice, three, four or more times daily. In a particular embodiment, doses of 600 mg or less are administered as a once daily dose and doses of more than 600 mg are administered twice daily in an amount equal to one half of the total daily dose.

A Sulfonamide Compound can be administered orally for reasons of convenience. In one embodiment, when administered orally, a Sulfonamide Compound is administered with a meal and water. In another embodiment, the Sulfonamide Compound is dispersed in water or juice (e.g., apple juice or orange juice) and administered orally as a suspension.

The Sulfonamide Compound can also be administered intradermally, intramuscularly, intraperitoneally, percutaneously, intravenously, subcutaneously, intranasally, epidurally, sublingually, intracerebrally, intravaginally, transdermally, rectally, mucosally, by inhalation, topically to the ears, nose, eyes, or skin, or by local ocular (i.e., subconjunctival, intravitreal, retrobulbar, intracameral). The mode of administration is left to the discretion of the health-care practitioner, and can depend in-part upon the site of the medical condition.

In one embodiment, provided herein are capsules containing a Sulfonamide Compound without an additional carrier, excipient or vehicle.

In another embodiment, provided herein are compositions comprising an effective amount of a Sulfonamide Compound and a pharmaceutically acceptable carrier or vehicle, wherein a pharmaceutically acceptable carrier or vehicle can comprise an excipient, diluent, or a mixture thereof. In one embodiment, the composition is a pharmaceutical composition.

The compositions can be in the form of tablets, chewable tablets, capsules, solutions, parenteral solutions, troches, suppositories, suspensions, gels, intra-ruminal devices (e.g., for prolonged prophylaxis or controlled release), implants, topical pour-ons, transdermal delivery gels, spot-ons, implants (including devices, gels, liquids (e.g., PLGA), and the like. Compositions can be formulated to contain a daily dose, or a convenient fraction of a daily dose, in a dosage unit, which may be a single tablet or capsule or convenient volume of a liquid. In one embodiment, the solutions are prepared from water-soluble salts, such as the hydrochloride salt. In general, all of the compositions are prepared according to known methods in pharmaceutical chemistry. Capsules can be prepared by mixing a Sulfonamide Compound with a suitable carrier or diluent and filling the proper amount of the mixture in capsules. The usual carriers and diluents include, but are not limited to, inert powdered substances such as starch of many different kinds, powdered cellulose, especially crystalline and microcrystalline cellulose, sugars such as fructose, mannitol and sucrose, grain flours and similar edible powders.

Tablets can be prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant might be necessary in a tablet formulation to prevent the tablet and punches from sticking in the dye. The lubricant can be chosen from such slippery solids as talc, magnesium and calcium stearate, stearic acid and hydrogenated vegetable oils. Tablet disintegrators are substances that swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethyl cellulose, for example, can be used as well as sodium lauryl sulfate. Tablets can be coated with sugar as a flavor and sealant, or with film-forming protecting agents to modify the dissolution properties of the tablet. The compositions can also be formulated as chewable tablets, for example, by using substances such as mannitol in the formulation.

When it is desired to administer a Sulfonamide Compound as a suppository, typical bases can be used. Cocoa butter is a traditional suppository base, which can be modified by addition of waxes to raise its melting point slightly. Water-miscible suppository bases comprising, particularly, polyethylene glycols of various molecular weights are in wide use.

The effect of the Sulfonamide Compound can be delayed or prolonged by proper formulation. For example, a slowly soluble pellet of the Sulfonamide Compound can be prepared and incorporated in a tablet or capsule, or as a slow-release implantable device. The technique also includes making pellets of several different dissolution rates and filling capsules with a mixture of the pellets. Tablets or capsules can be coated with a film that resists dissolution for a predictable period of time. Even the parenteral preparations can be made long-acting, by dissolving or suspending the Sulfonamide Compound in oily or emulsified vehicles, or adding amounts of PLGA, that allow it to disperse slowly in the serum.

### EXAMPLES

The following Examples are presented by way of illustration, not limitation. Compounds are named using the automatic name generating tool provided in Chemdraw Ultra 17.0 (Cambridgesoft), which generates systematic names for chemical structures, with support for the Cahn-Ingold-Prelog rules for stereochemistry. One skilled in the art can modify the procedures set forth in the illustrative examples to arrive at the desired products.

Abbreviations used:

| | |
|---|---|
| AcOH | Acetic acid |
| nBuLi | n-Butyl lithium |
| rac-BINAP | Racemic 2,2′-Bis(diphenylphosphino)-1,1′- |
| | binaphthalene |
| CDCl₃ | Deuterated chloroform |
| CsF | Cesium fluoride |
| DMF | N,N-Dimethylformamide |
| DMSO | Dimethylsulfoxide |
| DCE | 1,2-dichloroethane |
| DCH | 1,3-dichloro-5,5-dimethylimidazolidine-2,4-dione |
| DCM | Dichloromethane |
| DIPEA | Diisopropylethylamine |
| DMSO-d6 | Deuterated Dimethylsulfoxide |
| ESI | Electrospray ionization |
| Et₂O | Diethyl ether |
| EtOH | Ethanol |
| EtOAc | Ethyl acetate |
| HEPES | (4-(2-hydroxyethyl)-1-piperazineethanesulfonic acid) |
| HBr | Hydrobromic acid |
| HCl | Hydrochloric acid |
| H₂O | Water |
| H₂O₂ | Hydrogen peroxide |
| H₂SO₄ | Sulfuric acid |
| HOBt | 1-Hydroxybenzotriazole |
| HPLC | High performance liquid chromatography |
| K₂CO₃ | Potassium carbonate |
| K₃PO₄ | Potassium phosphate |
| KHCO₃ | Potassium bicarbonate |
| LiEt₃BH | Lithium triethylborohydride (Superhydride) |
| LCMS | Liquid chromatography mass spectrometry |
| MeCN or | Acetonitrile |
| ACN | |
| MeOH | Methanol |
| MS | Mass spectrometry |
| NaHMDS | Sodium bis(trimethylsilyl)amide |
| NCS | N-Chlorosuccinimide, 1-Chloropyrrolidine-2,5-dione |
| Na₂CO₃ | Sodium carbonate |
| Na₂S | Sodium sulfide |
| NaH | Sodium hydride |
| NaHCO₃ | Sodium bicarbonate |
| NaOH | Sodium hydroxide |
| Na₂SO₄ | Sodium sulphate |
| NBS | N-Bromosuccinimide |
| NH₄Cl | Ammonium chloride |
| NMR | Nuclear magnetic resonance |
| NaOCl | Sodium hypochlorite |
| Pd/C | Palladium (0) on carbon |
| Pd(OAc)₂ | Palladium(II) acetate |
| Pd(PPh₃)₄ | Tetrakis(triphenylphosphine)palladium (0) |
| Pd₂(dba)₃ | Tris(dibenzylideneacetone)dipalladium (0) |
| Pd(dba)₂ | Bis(dibenzylideneacetone)palladium(0) |
| Pd(PPh₃)₂Cl₂ | Bis(triphenylphosphine)palladium(II) dichloride |
| Pd(dppf)Cl₂ | Bis(diphenylphosphino)ferrocene]dichloropalladium(II) |
| TEA | Triethylamine |
| TFA | Trifluoracetic acid |
| THF | Tetrahydrofuran |
| THP | Tetrahydropyran |
| TLC | Thin layer chromatography |
| TMEDA | Tetramethylethylenediamine |
| UPLC | Ultra Performance Liquid Chromatography |
| UHPLC | Ultra High Performance Liquid Chromatography |
| Xantphos | 4,5-Bis(diphenylphosphino)-9,9-dimethylxanthene |
| Xphos | Dicyclohexyl[2′,4′,6′-tris(propan-2-yl)[1,1′-biphenyl]- |
| | 2-yl]phosphane |

### Example 1. N-(7-chloroquinolin-8-yl)-3-cyclopropylpyridine-2-sulfonamide

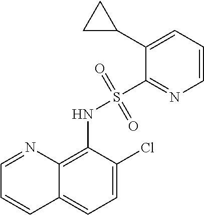 [SMILES: O=S(=O)(Nc1c(Cl)ccc2cccnc12)c1ncccc1C1CC1]

2-Chloro-3-cyclopropylpyridine. To a mixture of 2-chloro-3-iodopyridine (10.0 g, 41.8 mmol) and potassium cyclopropyltrifluoroborate (9.27 g, 62.7 mmol) in water (10 mL) and dioxane (100 mL) were added (1,1′-bis(diphenylphosphino)ferrocene)palladium(II) dichloride (3.06 g, 4.18 mmol) and potassium carbonate (20.2 g, 146 mmol) under nitrogen. The mixture was stirred at 100° C. for 24 h. The mixture concentrated in vacuo. The residue was filtered and the filter cake was washed with ethyl acetate. The filtrate was poured into water (100 mL). The aqueous phase was extracted with ethyl acetate. The combined organic phase was washed with brine, dried over anhydrous sodium sulfate, filtered and concentrated in vacuo. The residue was purified by flash column chromatography to get 2-chloro-3-cyclopropylpyridine (3.2 g, 20.83 mmol, 49.89% yield).

3-Cyclopropyl-2-((4-methoxybenzyl)thio)pyridine. To a mixture of (4-methoxyphenyl)methanethiol (4.82 g, 31.3 mmol) in DMSO (30 mL) were added cesium fluoride (6.33 g, 41.7 mmol) and 2-chloro-3-cyclopropylpyridine (3.20 g, 20.8 mmol). The mixture was stirred at 80° C. for 16 h. The mixture was poured into brine (40 mL). The aqueous phase was extracted with ethyl acetate. The combined organic phase was washed with brine and concentrated in vacuo. The residue was purified by prep-HPLC to get 3-cyclopropyl-2-((4-methoxybenzyl)thio)pyridine (5.6 g, crude).

3-Cyclopropylpyridine-2-sulfonyl chloride. To a solution of 3-cyclopropyl-2-((4-methoxybenzyl)thio)pyridine (700 mg, 2.58 mmol) in DCM (9.8 mL), acetic acid (1.4 mL) and water (2.8 mL) was added a solution of 1,3-dichloro-5,5-dimethylimidazolidine-2,4-dione (DCH) (1.52 g, 7.74 mmol) in DCM (5.6 mL) dropwise at 0° C. over 0.5 h. The mixture was stirred between 0-5° C. for 3 h. The mixture was poured into water. The aqueous phase was extracted with DCM. The combined organic phase was washed with saturated sodium bicarbonate and brine, dried over anhydrous sodium sulfate, filtered and concentrated in vacuo to get 3-cyclopropylpyridine-2-sulfonyl chloride (562 mg, crude).

N-(7-Chloroquinolin-8-yl)-3-cyclopropylpyridine-2-sulfonamide. To a mixture of 7-chloroquinolin-8-amine (138 mg, 0.774 mmol) in pyridine (5.6 mL) was added a solution of 3-cyclopropylpyridine-2-sulfonyl chloride (561 mg, 2.58 mmol) in DCM (5.6 mL) dropwise at 0° C. under nitrogen. The mixture was stirred at 25° C. for 16 h. The product was isolated and purified by standard methods to give N-(7-chloroquinolin-8-yl)-3-cyclopropylpyridine-2-sulfonamide (89.23 mg, 0.245 mmol, 10% yield, 98.9% purity). MS (ESI) m/z 360.1 [M+1]⁺.

### Example 2. N-(7-chloroquinolin-8-yl)-6-(pyrrolidin-1-yl)pyrazine-2-sulfonamide

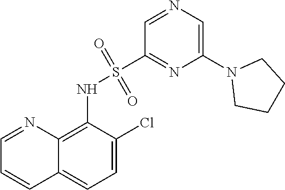 [SMILES: O=S(=O)(Nc1c(Cl)ccc2cccnc12)c1cncc(N2CCCC2)n1]

2-((4-Methoxybenzyl)thio)-6-(pyrrolidin-1-yl)pyrazine. To a mixture of 2-chloro-6-[(4-methoxyphenyl)methylsulfanyl]pyrazine (2.50 g, 9.37 mmol) and pyrrolidine (1.33 g, 18.74 mmol) in ACN (30.00 mL) was added potassium carbonate (2.59 g, 18.74 mmol). The mixture was stirred at 80° C. for 12 h. The mixture was diluted with ethyl acetate and the resulting mixture was filtered. The filtrate was concentrated. The residue was purified by silica gel chromatography to give 2-[(4-methoxyphenyl)methylsulfanyl]-6-pyrrolidin-1-yl-pyrazine (2.70 g, 8.96 mmol, 96% yield).

6-(Pyrrolidin-1-yl)pyrazine-2-sulfonyl chloride. To a mixture of 2-[(4-methoxyphenyl)methylsulfanyl]-6-pyrrolidin-1-yl-pyrazine (2.00 g, 6.64 mmol) in DCM (21 mL), water (6 mL) and acetic acid (3 mL) was added a solution of 1,3-dichloro-5,5-dimethyl-imidazolidine-2,4-dione (3.92 g, 19.9 mmol) in DCM (12 mL) dropwise at 0° C. over 1 h. The mixture was stirred at 0° C. for 1.5 h. The mixture was poured into water. The aqueous phase was extracted with ethyl acetate. The combined organic layers were washed with saturated sodium bicarbonate, dried over anhydrous sodium sulfate, filtered and concentrated in vacuo to give 6-pyrrolidin-1-ylpyrazine-2-sulfonyl chloride (1.62 g, crude).

N-(7-Chloroquinolin-8-yl)-6-(pyrrolidin-1-yl)pyrazine-2-sulfonamide. To a mixture of 7-chloroquinolin-8-amine (350.00 mg, 1.96 mmol) in THF (10 mL) was added sodium bis(trimethylsilyl)amide (1 M, 6.53 mL) at −65° C. under N₂ protection. The mixture was stirred at 25° C. for 0.5 h. The mixture was added a solution of 6-pyrrolidin-1-ylpyrazine-2-sulfonyl chloride (1.62 g, 6.53 mmol) in THF (5 mL) at −65° C. The mixture was stirred at 25° C. for 1 h. The mixture was quenched with water. The product was isolated and purified by standard methods to give N-(7-chloro-8-quinolyl)-6-pyrrolidin-1-yl-pyrazine-2-sulfonamide (229.10 mg, 0.580 mmol, 9% yield, 98.1% purity). MS (ESI) m/z 390.1 [M+1]⁺.

### Example 3. N-(7-chloroquinolin-8-yl)-3-(dimethylamino)pyrazine-2-sulfonamide

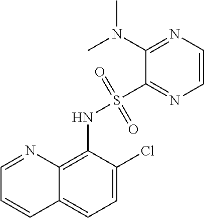 [SMILES: CN(C)c1nccnc1S(=O)(=O)Nc1c(Cl)ccc2cccnc12]

3-((4-Methoxybenzyl)thio)-N,N-dimethylpyrazin-2-amine. The mixture of 2-chloro-3-[(4-methoxyphenyl)methylsulfanyl]pyrazine (3.00 g, 11.3 mmol), N-methylmethanamine (1.38 g, 16.9 mmol, HCl salt) and potassium carbonate (4.66 g, 33.8 mmol) in ACN (20 mL) was stirred at 70° C. for 12 h. The resultant mixture was filtered and the filtrate was concentrated to give 3-[(4-methoxyphenyl)methylsulfanyl]-N,N-dimethyl-pyrazin-2-amine (3.30 g, crude).

3-(Dimethylamino)pyrazine-2-sulfonyl chloride. To a mixture of 3-[(4-methoxyphenyl)methylsulfanyl]-N,N-dimethyl- pyrazin-2-amine (3.10 g, 11.3 mmol) in DCM (21 mL), water (6 mL) and acetic acid (3 mL) was added a solution of 1,3-dichloro-5,5-dimethyl-imidazolidine-2,4-dione (6.65 g, 33.8 mmol) in DCM dropwise at 0° C. over 0.5 h. The mixture was stirred at 5° C. for 1 h. The mixture was poured into water. The aqueous phase was extracted with DCM. The combined organic layers were washed with saturated sodium bicarbonate, dried over anhydrous sodium sulfate, filtered and concentrated to remove most of solvent. The suspension was filtered and the filtrate was concentrated to give 3-(dimethylamino)pyrazine-2-sulfonyl chloride (3.10 g, crude).

N-(7-Chloroquinolin-8-yl)-3-(dimethylamino)pyrazine-2-sulfonamide. To a mixture of 7-chloroquinolin-8-amine (550.00 mg, 3.08 mmol) in THE (20 mL) was added sodium hexamethyldisilazane (1 M, 10.3 mL) at −65° C. under N₂ protection. The mixture was stirred at 25° C. for 0.5 h. Then to the mixture was added a solution of 3-(dimethylamino)pyrazine-2-sulfonyl chloride (2.28 g, 10.3 mmol) in THF (10 mL) at −65° C. The mixture was stirred at 25° C. for 1 h. The mixture was quenched with water. The product was isolated and purified by standard methods to give N-(7-chloro-8-quinolyl)-3-(dimethylamino)pyrazine-2-sulfonamide (303.00 mg, 0.816 mmol, 27% yield, 98% purity). MS (ESI) m/z 364 [M+1]⁺.

### Example 4. N-(7-chloroquinolin-8-yl)-1-isopropyl-1H-pyrazole-5-sulfonamide

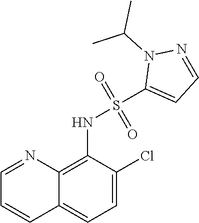 [SMILES: CC(C)n1nccc1S(=O)(=O)Nc1c(Cl)ccc2cccnc12]

Lithium 1-isopropyl-1H-pyrazole-5-sulfinate. To a solution of 1-isopropyl-1H-pyrazole (1.50 g, 13.6 mmol) in diethyl ether (50 mL) in the presence of S02 was added n-BuLi (2.5 M, 6.54 mL) at −70° C. The mixture was stirred at −70° C. for 0.5 h and 0° C. for 2 h. Excess SO₂ was purged and the mixture was stirred at −70° C. for 1 h. Then the mixture was warmed to 20° C. The mixture was filtered and the filter cake was collected and dried under vacuum to give lithium 1-isopropyl-1H-pyrazole-5-sulfinate (2.40 g, crude).

1-Isopropyl-1H-pyrazole-5-sulfonyl chloride. Lithium 1-isopropyl-1H-pyrazole-5-sulfinate (2.40 g, 13.3 mmol) was added into chloroform (40 mL) and water (50 mL) at 0° C., then N-chlorosuccinimide (2.67 g, 19.9 mmol) was added in portions. The mixture was stirred at 0° C. for 1 h. The mixture was diluted with cold water (20 mL). The aqueous phase was extracted with chloroform. The combined organic phase was dried over anhydrous sodium sulfate, filtered and concentrated under vacuum. The crude product was purified by silica gel chromatography to give 1-isopropyl-1H-pyrazole-5-sulfonyl chloride (1.20 g, 5.64 mmol, 42% yield, 98% purity). ¹H NMR (400 MHz, CDCl₃) δ 7.62 (d, J=1.9 Hz, 1H), 6.98 (d, J=2.0 Hz, 1H), 5.28-5.18 (m, 1H), 1.60 (s, 6H).

N-(7-chloroquinolin-8-yl)-1-isopropyl-1H-pyrazole-5-sulfonamide. To a solution of 7-chloroquinolin-8-amine (205 mg, 1.15 mmol) in pyridine (4.55 g, 57.5 mmol) was added a mixture of 1-isopropyl-1H-pyrazole-5-sulfonyl chloride (400 mg, 1.92 mmol) in DCM (5 mL) at 0° C. The mixture was stirred at 25° C. for 8 h. The product was isolated and purified by standard methods to give N-(7-chloroquinolin-8-yl)-1-isopropyl-1H-pyrazole-5- sulfonamide (89.25 mg, 0.251 mmol, 22% yield, 98.5% purity). MS (ESI) m/z 351.1 [M+1]⁺.

### Example 5. N-(7-chloroquinolin-8-yl)-5-methoxypyrazine-2-sulfonamide

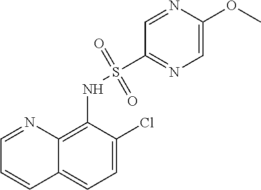 [SMILES: COc1cnc(S(=O)(=O)Nc2c(Cl)ccc3cccnc23)cn1]

N-(7-chloroquinolin-8-yl)-5-methoxypyrazine-2-sulfonamide. To a solution of 5-chloro-N-(7-chloroquinolin-8-yl)pyrazine-2-sulfonamide (150 mg, 0.422 mmol) in MeOH (3 mL) was added sodium methanolate (66 mg, 1.69 mmol) under nitrogen. The mixture was stirred at 60° C. for 2 h. The product was isolated and purified by standard methods to give N-(7-chloroquinolin-8-yl)-5-methoxypyrazine-2-sulfonamide (127.59 mg, 0.354 mmol, 84% yield, 97.3% purity). MS (ESI) m/z 351 [M+1]⁺.

### Example 6. N-(7-chloroquinolin-8-yl)-6-methoxypyrazine-2-sulfonamide

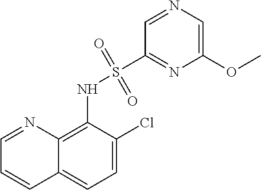 [SMILES: COc1cncc(S(=O)(=O)Nc2c(Cl)ccc3cccnc23)n1]

2-Chloro-6-((4-methoxybenzyl)thio)pyrazine. To a mixture of (4-methoxyphenyl)methanethiol (5.18 g, 33.6 mmol, 5 mL) in TIF (25 mL) was added sodium hydride (2.01 g, 50.3 mmol, 60% purity). The mixture was stirred at 25° C. for 10 min. Then to the mixture was added dropwise a solution of 2,6-dichloropyrazine (5.00 g, 33.6 mmol) in THE (15 mL) with stirring at 25° C. The resulting mixture was stirred at 25° C. for 3 h. The mixture was quenched with water (50 mL) at 0° C. The aqueous phase was extracted with ethyl acetate (50 mL×3). The combined organic layers were washed with brine, dried over sodium sulfate, filtered and concentrated under reduced pressure to give a residue. The residue was purified by silica gel chromatography to give 2-chloro-6-((4-methoxybenzyl)thio)pyrazine (6.33 g, 23.3 mmol, 69% yield, 98% purity).

2-Methoxy-6-((4-methoxybenzyl)thio)pyrazine. To a solution of 2-chloro-6-((4-methoxybenzyl)thio)pyrazine (3.00 g, 11.25 mmol) in MeOH (20 mL) was added sodium methanolate (911 mg, 16.9 mmol). The mixture was stirred at 60° C. for 16 h. The mixture was diluted with water (50 mL). The resulting mixture was extracted with ethyl acetate. The combined organic layers were washed with saturated brine, dried with anhydrous sodium sulfate, filtered, and concentrated in vacuo. The residue was purified by silica gel column chromatography to give 2-methoxy-6-((4-methoxybenzyl)thio)pyrazine (2.90 g, 11.1 mmol, 98% yield).

6-Methoxypyrazine-2-sulfonyl chloride. To a mixture of 2-methoxy-6-((4-methoxybenzyl)thio)pyrazine (1.50 g, 5.7 mmol) in DCM (21 mL), H₂O (6 mL) and acetic acid (3 mL) was added a solution of 1,3-dichloro-5,5-dimethyl-imidazolidine-2,4-dione (3.38 g, 17.2 mmol) in DCM (12 mL) dropwise at −5° C. over 1 h. Then the mixture was stirred at 0° C. for 1 h. The mixture was poured into water. The aqueous phase was extracted with DCM. The combined organic layers were washed with saturated sodium hydrogen carbonate, dried over anhydrous sodium sulfate, filtered and concentrated to remove most of solvent. The suspension was filtered and the filtrate was concentrated to give 6-methoxypyrazine-2-sulfonyl chloride (1.50 g, crude).

N-(7-chloroquinolin-8-yl)-6-methoxypyrazine-2-sulfonamide. To a mixture of 7-chloroquinolin-8-amine (300 mg, 1.68 mmol) in THF (10 mL) was added sodium hexamethyldisilazane (1 M, 5.6 mL) at −65° C. under N₂ protection. The mixture was stirred at 25° C. for 0.5 h. Then to the mixture was added a solution of 6-methoxypyrazine-2-sulfonyl chloride (1.17 g, 5.60 mmol) in THF (5 mL) at −65° C. Then the mixture was stirred at 25° C. for 1 h. The product was isolated and purified by standard methods to give N-(7-chloroquinolin-8-yl)-6-methoxypyrazine-2-sulfonamide (97.8 mg, 0.265 mmol, 5% yield, 95.1% purity). MS (ESI) m/z 351 [M+1]⁺.

### Example 7. 1-Ethyl-N-(7-methoxyquinolin-8-yl)-1H-imidazole-2-sulfonamide

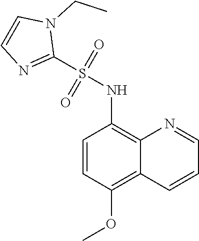 [SMILES: CCn1ccnc1S(=O)(=O)Nc1ccc(OC)c2cccnc12]

1-((1-Ethyl-1H-imidazol-2-yl)sulfonyl)-1H-benzo[d][1,2,3]triazole. To a mixture of 1-ethyl-1H-imidazole (15.0 g, 156 mmol) in THE (450 mL) was added a solution of n-butyllithium in hexane (2.5 N, 66 mL, 164 mmol) dropwise at −78° C. under nitrogen. The resulting reaction mixture was stirred at −78° C. for 1 h. Sulfur dioxide was bubbled to the above solution of the organometallic reagent in TUE at −78° C. until a sample to the solution gave a pH acid test. The mixture was stirred at that temperature for 15 minutes, then at room temperature for 1 h. N-chlorobenzotriazole (23.0 g, 156 mmol) was added in one portion and the mixture was stirred for 2 h at room temperature. TEA (39.4 g, 390 mmol) was added, followed by stirring at room temperature for 16 h. Water (300 mL) was added and extracted with ethyl acetate. The combined organic layers were washed with saturated aqueous ammonium chloride solution, dried over anhydrous sodium sulfate and filtered. The residue was recrystallized from ethyl acetate to afford the titled compound (23.0 g, 83.0 mmol, 53% yield).

7-Methoxyquinoline. To a slurry of sodium hydride (3.50 g, 86.2 mmol) in dry DMF (100 mL) at 0° C. was added quinolin-7-ol (5.0 g, 34.5 mmol) in dry DMF (200 mL). The mixture was stirred for 1 h and then allowed to warm to room temperature. Iodomethane (9.80 g, 69.0 mmol) was added and the mixture was stirred for 16 h. The reaction mixture was then poured into ice water and extracted with ethyl acetate. The combined extracts were washed with brine, dried and concentrated to give a crude product, which was purified by silica gel column chromatography to afford the titled compound (2.75 g, 17.3 mmol, 50% yield).

7-Methoxy-8-nitroquinoline. A cooled concentrated sulfuric acid (7.5 mL) was added to 7-methoxyquinoline (2.75 g, 17.29 mmol) at 0° C. Concentrated nitric acid (5.8 mL) was added dropwise keeping the temperature below 0° C. The mixture was stirred at room temperature for 1 h. The reaction mixture was poured into ice and neutralized with ammonium hydroxide. The yellow precipitate was filtered to give the desired product (2.80 g, 13.7 mmol, 79% yield).

7-Methoxyquinolin-8-amine. To a solution of 7-methoxy-8-nitroquinoline (1.00 g, 4.88 mmol) in MeOH (20 mL) and THF (20 mL) was added palladium on charcoal (200 mg, 10%). The reaction mixture was stirred at room temperature under hydrogen atmosphere for 2 h and filtered through celite. The filtrate was concentrated in vacuo to give the desired product (790 mg, 0.25 mmol, 92% yield).

1-Ethyl-N-(5-methoxyquinolin-8-yl)-1H-imidazole-2-sulfonamide. To a solution of 7-methoxyquinolin-8-amine (200 mg, 1.15 mmol) in THE (5 mL) was added a solution of sodium bis(trimethylsilyl)amide in THE (2M, 1.15 mL, 2.30 mmol) slowly at −78° C. under nitrogen. After the resulting reaction mixture was stirred for 1 h at the temperature, a solution of 1-((1-ethyl-1H-imidazol-2-yl)sulfonyl)-1H-benzo[d][1,2,3]triazole (400 mg, 1.44 mmol) in THE (8 mL) was added at −78° C. Then the resulting solution was stirred at room temperature for 1 h. The reaction mixture was quenched with aqueous ammonia chloride solution and extracted with ethyl acetate. The combined organic layers were washed with brine, dried over anhydrous sodium sulfate and concentrated. The residue was purified by prep-HPLC to give the title compound (102.2 mg, 0.308 mmol, 27% yield). MS (ESI) m/z 332.8 [M+1]⁺.

### Example 8. 1-Ethyl-N-(3-methylquinolin-8-yl)-1H-imidazole-2-sulfonamide

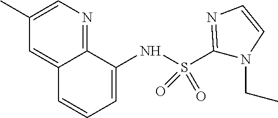 [SMILES: CCn1ccnc1S(=O)(=O)Nc1cccc2cc(C)cnc12]

1-((1-Ethyl-1H-imidazol-2-yl)sulfonyl)-1H-benzo[d][1,2,3]triazole. To a solution of mixture of 1-ethyl-1H-imidazole (15.0 g, 156 mmol) in THE (450 mL) was added a solution of n-BuLi in hexane (2.5 N, 66 mL, 164 mmol) dropwise at −78° C. under nitrogen. The resulting reaction mixture was stirred at −78° C. for 1 hour. Sulfur dioxide was bubbled into the solution of the organometallic reagent in THE at −78° C. until a sample to the solution gave a pH acid test. The mixture was stirred at that temperature for 15 minutes, then at room temperature for 1 h. N-chlorobenzotriazole (23.0 g, 156 mmol) was added in one portion and the mixture was stirred for 2 h at room temperature. Triethylamine (39.4 g, 390 mmol) was added, followed by stirring at room temperature for 16 hours. Water was added and extracted with ethyl acetate. The combined organic layers were washed with saturated aqueous ammonium chloride solution, dried over anhydrous sodium sulfate, filtered, and concentrated. The residue was recrystallized from ethyl acetate to afford the title compound (23.0 g, 83.0 mmol, 53% yield). MS (ESI) m/z 278.1 [M+H]⁺.

1-Ethyl-N-(3-methylquinolin-8-yl)-1H-imidazole-2-sulfonamide. To a solution of 3-methylquinolin-8-amine (200 mg, 1.26 mmol) in THE (5 mL) was added a solution of sodium bis(trimethylsilyl)amide in THE (2M, 1.25 mL, 2.50 mmol) slowly at −78° C. under nitrogen. After the resulting reaction mixture was stirred for 1 hour at the temperature, a solution of 1-ethyl-N-(4-methoxyquinolin-8-yl)-1H-imidazole-2-sulfonamide (400 mg, 1.44 mmol) in THF (8 mL) was added at −78° C. The resulting solution was stirred at room temperature for 1 hour. The product was isolated and purified by standard methods to give the title compound (59.6 mg, 0.189 mmol, 14% yield). ¹H NMR (400 MHz, DMSO-d₆) δ 8.36 (s, 1H), 8.18 (s, 1H), 7.67-7.56 (m, 2H), 7.38-7.32 (m, 2H), 6.62 (s, 1H) 4.42-4.39 (m, 2H), 2.24 (s, 3H), 1.42 (t, J=6.8 Hz, 3H). MS (ESI) m/z 316.8 [M+H]⁺. Purity=98.6% at 214 nm, Purity=96.6% at 254 nm.

### Example 9. N-(6-Fluoroquinolin-8-yl)-5-methylpyridine-2-sulfonamide

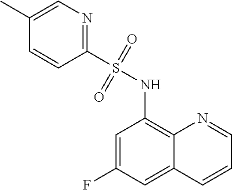 [SMILES: Cc1ccc(S(=O)(=O)Nc2cc(F)cc3cccnc23)nc1]

2-(Benzylthio)-5-methylpyridine. To a solution of phenylmethanethiol (23.3 g, 188 mmol) in anhydrous THF (200 mL) was added sodium hydride (60% in mineral oil, 8.28 g, 207 mmol) in portions at 0° C. After the result mixture was stirred at room temperature for 1 h, 2-chloro-5-methylpyridine (20.0 g, 157 mmol) was added portion-wise at 0° C. The result mixture was stirred at 70° C. for 30 h. The reaction was treated with water and the mixture was extracted with ethyl acetate. The combined organic layers were washed with brine, dried over anhydrous sodium sulfate and filtered. The filtrate was concentrated and the residue was purified by silica gel column chromatography (1% ethyl acetate in petroleum ether) to give the desired compound (10.5 g, 50% purity, 24.4 mmol, 16% yield).

5-Methylpyridine-2-sulfonyl chloride. Chlorine was bubbled into a solution of 2-(benzylthio)-5-methylpyridine (3.0 g, 50% purity crude, 6.98 mmol) in acetic acid (20 mL), DCM (20 mL) and water (7 mL) at 0° C. for 0.5 h. Then the resulting mixture was stirred at the temperature for 1 h. The reaction was diluted with DCM, washed with saturated aqueous sodium hydrogen carbonate and brine, dried over sodium sulfate and filtered. The filtrate was concentrated to afford the crude product (3.2 g), which was subjected to the next step without further purification.

6-Fluoroquinolin-8-amine. To a solution of 6-fluoro-8-nitroquinoline (100 mg, 0.52 mmol) in MeOH (5 mL) was added palladium on charcoal (10 mg, 10%). The resulting mixture was stirred at room temperature under hydrogen atmosphere overnight. The reaction mixture was filtered through celite and the filtrate was concentrated in vacuo to give crude product (110 mg, 80% purity), which was used for the next step without further purification.

N-(6-Fluoroquinolin-8-yl)-5-methylpyridine-2-sulfonamide. To a solution of 6-fluoroquinolin-8-amine (100 mg, crude) in THE (3 mL) was added a solution of sodium bis(trimethylsilyl)amide in THE (2M, 0.50 mL, 1.0 mmol) slowly at −78° C. under nitrogen. The resulting solution was stirred for 1 h at −78° C. Then the crude 5-methylpyridine-2-sulfonyl chloride (300 mg crude) was added to the above solution. The resulting solution was stirred at −78° C. and stirred at room temperature for 1 h. The reaction mixture was quenched with aqueous ammonia chloride solution and extracted with DCM. The combined organic layers were washed with brine, dried over anhydrous sodium sulfate and concentrated. The residue was purified by prep-HPLC to give the desired compound (26.1 mg, 0.0822 mmol, 16% yield over two steps). MS (ESI) m/z 317.8 [M+1]⁺.

### Example 10. N-(7-chloroquinolin-8-yl)-3-(dimethylamino)pyridine-2-sulfonamide

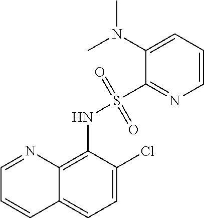 [SMILES: CN(C)c1cccnc1S(=O)(=O)Nc1c(Cl)ccc2cccnc12]

Lithium 3-fluoropyridine-2-sulfinate. To a solution of 2-bromo-3-fluoropyridine (2.00 g, 11.4 mmol) in diethyl ether (30 mL) was added butyllithium (2.5 M, 5.5 mL) at −70° C. The mixture was stirred at −70° C. for 1 h. Excess sulfur dioxide was purged and the mixture was stirred at −70° C. for 1 h. Yellow solid formed. The mixture was filtered and the filter cake was dried under vacuum to give lithium 3-fluoropyridine-2-sulfinate (1.90 g, crude).

3-Fluoropyridine-2-sulfonyl chloride. Lithium 3-fluoropyridine-2-sulfinate (1.90 g, 11.4 mmol) was added in portions to chloroform (40 mL) and water (50 mL) at 0° C. Then NCS (3.04 g, 22.7 mmol) was added into the above mixture in portions. The mixture was stirred at 0° C. for 1 h. The mixture was extracted with chloroform. The combined organic phase was dried over anhydrous sodium sulfate, filtered and concentrated under vacuum to give 3-fluoropyridine-2-sulfonyl chloride (2.00 g, 6.13 mmol, 54% yield, 60% purity).

N-(7-Chloroquinolin-8-yl)-3-methylpyrazine-2-sulfonamide. To a mixture of 7-chloroquinolin-8-amine (328 mg, 1.84 mmol) in THF (20 mL) was added sodium bis(trimethylsilyl)amide (1 M, 12.3 mL) drop-wise at −65° C. under nitrogen. The mixture was stirred at 25° C. for 0.5 h. Then 3-fluoropyridine-2-sulfonyl chloride (2.00 g, 6.13 mmol) in THE (10 mL) was added drop-wise into the above mixture at −65° C. under nitrogen. The mixture was stirred at 25° C. for 1 h. The mixture was added into saturated ammonium chloride. The aqueous phase was extracted with ethyl acetate. The combined organic phase was washed with brine, dried over anhydrous sodium sulfate, filtered and concentrated in vacuo. The residue was purified by silica gel chromatography to give N-(7-chloroquinolin-8-yl)-3-fluoropyridine-2-sulfonamide (500 mg, 1.32 mmol, 22% yield, 89% purity).

N-(7-Chloroquinolin-8-yl)-3-(dimethylamino)pyridine-2-sulfonamide. To a mixture of N-(7-chloroquinolin-8-yl)-3-fluoropyridine-2-sulfonamide (423 mg, 1.11 mmol) and N-methylmethanamine hydrogen chloride (545 mg, 6.69 mmol) in DMSO (5 mL) was added N-ethyl-N-isopropyl-propan-2-amine (1.44 g, 11.2 mmol). The mixture was stirred at 80° C. for 54 h. The product was isolated and purified by standard methods to give N-(7-chloroquinolin-8-yl)-3-(dimethylamino)pyridine-2-sulfonamide (137.92 mg, 0.371 mmol, 33% yield, 97.5% purity). MS (ESI): m/z 363.0 [M+1]⁺.

### Example 11. N-(6-Hydroxyquinolin-8-yl)pyridine-2-sulfonamide

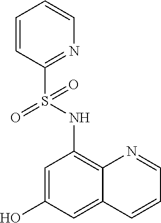 [SMILES: O=S(=O)(Nc1cc(O)cc2cccnc12)c1ccccn1]

N-(6-Methoxyquinolin-8-yl)pyridine-2-sulfonamide. To a solution of 6-methoxyquinolin-8-amine (200 mg, 1.14 mmol) in pyridine (6 mL) was added pyridine-2-sulfonyl chloride (305 mg, 1.17 mmol). The reaction mixture was heated in a microwave at 130° C. for 5 min. The reaction was cooled to room temperature and quenched with water. Solid product was filtered and washed with water and diethyl ether to afford the desired product (330 mg, 1.04 mmol, 91% yield).

N-(6-Hydroxyquinolin-8-yl)pyridine-2-sulfonamide. To a solution of N-(6-methoxyquinolin-8-yl)pyridine-2-sulfonamide (150 mg, 0.47 mmol) in dry DCM (2 mL) was added tribromoborane (297 mg, 1.19 mmol). The resulting reaction mixture was heated at reflux for 16 h. The product was isolated and purified by standard methods to afford the desired product (50 mg, 0.16 mmol, 35% yield) as a pink powder. MS (ESI) m/z 302 [M+1]⁺.

### Example 12. N-(6-Fluoroquinolin-8-yl)-3,5-dimethylbenzenesulfonamide

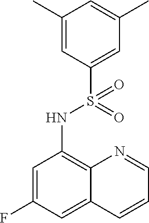 [SMILES: Cc1cc(C)cc(S(=O)(=O)Nc2cc(F)cc3cccnc23)c1]

6-Fluoro-8-nitroquinoline. Propane-1,2,3-triol (14 mL) was preheated to 160° C. for 1 h then cooled to 80° C. 4-Fluoro-2-nitroaniline (10.0 g, 64 mmol) and sodium iodide (200 mg, 1.28 mmol) were added and the mixture was heated to 150° C. before concentrated sulfuric acid (8.4 mL) was added dropwise. The reaction was stirred at 150° C. for 45 minutes. After the reaction was completed, the mixture was cooled to room temperature and partitioned between water and ethyl acetate. The organic layer was washed with saturated aqueous sodium carbonate solution, dried over sodium sulfate and concentrated. The residue was washed with MeOH/hexane (1:10) to give the desired product (4.0 g, 20.8 mmol, yield: 33%).

6-Fluoroquinolin-8-amine. To a suspension of 6-fluoro-8-nitroquinoline (4.0 g, 20.8 mmol) in EtOH (100 mL) was added tin dichloride (9.40 g, 41.7 mmol). The mixture was heated to reflux for 2 h. After the reaction was completed, sodium hydroxide (3.5 g, 83.4 mmol) in water (100 mL) was added and the reaction was extracted with ethyl acetate. The organic layer was dried over anhydrous sodium sulfate, concentrated and the residue was purified by silica gel column chromatography to afford the desired product as a yellow solid (1.2 g, 7.4 mmol, yield; 35%).

N-(6-Fluoroquinolin-8-yl)-3,5-dimethylbenzenesulfonamide. To a solution of 3,5-dimethylbenzene-1-sulfonyl chloride (180 mg, 1.0 mmol) in pyridine (5 mL) was added 6-fluoro-quinolin-8-ylamine (163 mg, 1.0 mmol). The mixture was stirred at room temperature overnight. The product was isolated and purified by standard methods to afford the desired product (28.4 mg, 0.86 mmol, yield: 8.6%). MS (ESI) m/z 331 [M+1]⁺.

### Example 13. 3-(N-(5-Morpholinoquinolin-8-yl)sulfamoyl)benzamide

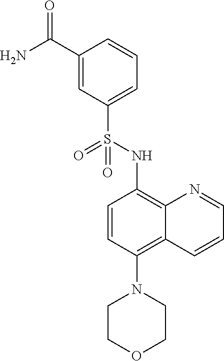 [SMILES: NC(=O)c1cccc(S(=O)(=O)Nc2ccc(N3CCOCC3)c3cccnc23)c1]

4-(8-Nitroquinolin-5-yl)morpholine. 5-Chloro-8-nitroquinoline (600 mg, 2.88 mmol) was suspended in morpholine (3 mL) and the mixture was irradiated by a microwave apparatus at 100° C. for 10 minutes. After cooling to room temperature, the residue was concentrated and washed with hexane to afford the desired product, which was used in the next step without further purification (480 mg, 64% yield, 1.84 mmol).

5-Morpholinoquinolin-8-amine. To a solution of 4-(8-nitroquinolin-5-yl)morpholine (480 mg, 1.84 mmol) in MeOH (10 mL) was added palladium on carbon (100 mg, 10%). The mixture was stirred at room temperature overnight under hydrogen atmosphere. The catalyst was filtered off and the filtrate was concentrated to give the desired product, which was used in the next step without further purification. (405 mg, yield: 95%, 1.76 mmol).

3-Cyano-N-(5-morpholinoquinolin-8-yl)benzenesulfonamide. To a solution of 5-morpholinoquinolin-8-amine (92 mg, 0.40 mmol) in pyridine (2 mL) was added 3-cyanobenzene-1-sulfonyl chloride (81 mg, 0.40 mmol), the reaction mixture was stirred at room temperature overnight. Concentrated in vacuo gave crude product, which was purified by silica gel column chromatography to give the desired product (118.7 mg, 0.30 mmol, 75% yield).

3-(N-(5-Morpholinoquinolin-8-yl)sulfamoyl)benzamide. To a solution of 3-cyano-N-(5-morpholinoquinolin-8-yl)benzenesulfonamide (118.7 mg, 0.30 mmol) in dimethyl sulfoxide (2 mL) was added potassium carbonate (83 mg, 0.60 mmol) and hydrogen peroxide (31 mg, 0.90 mmol). The reaction mixture was stirred at room temperature for 2 h. The product was isolated and purified by standard methods to give the title compound (71.7 mg, 0.17 mmol, 57% yield). MS (ESI) m/z 413.2 [M+1]⁺.

### Example 14. 4-Methyl-N-(6-methylquinolin-8-yl)benzenesulfonamide

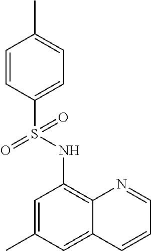 [SMILES: Cc1ccc(S(=O)(=O)Nc2cc(C)cc3cccnc23)cc1]

6-Methyl-8-nitroquinoline. Glycerol (3.25 mL, 44.5 mmol) was preheated at 16 5° C. for 1 h and cooled to 150° C. 4-methyl-2-nitroaniline (2.5 g, 16.5 mmol) and sodium iodide (100 mg, 0.7 mmol) was added followed by the addition of sulfuric acid (2.1 mL, 39 mmol) at 150° C. over a period of 10 minutes. The reaction mixture was stirred at 150° C. for 2 h. The resulting reaction mixture was carefully poured into ice-cold water, and the precipitate was collected by filtration. The solid obtained was recrystallized in 10% ethyl acetate and petroleum ether to afford the desired product (1 g, 90% purity by LC-MS, 5.31 mmol, 32% yield).

6-Methylquinolin-8-amine. To a solution of 6-methyl-8-nitroquinoline (1 g, 5.31 mmol) in EtOH (20 mL) was added stannous chloride (4.01 g, 21.27 mmol). The resulting reaction mixture was heated at reflux for 35 minutes then cooled to room temperature. The reaction was poured into 10% aqueous sodium hydroxide solution (15 mL) and extracted with ethyl acetate. The combined organic layer was washed with 10% sodium hydroxide solution, water, brine and the organic phase was dried over anhydrous sodium sulfate, filtered and concentrated in vacuo to afford the desired product (800 mg, 5.06 mmol, 94% yield, 90% pure by LC-MS).

4-Methyl-N-(6-methylquinolin-8-yl)benzenesulfonamide. To a solution of 6-methylquinolin-8-amine (200 mg, 1.26 mmol) in pyridine (6 mL) was added 4-methylbenzene-1-sulfonyl chloride (359 mg, 1.89 mmol). The resulting reaction mixture was heated in a microwave for 3 min at 130° C., cooled to room temperature, and quenched with water (18 mL). The product was isolated and purified by standard methods to afford the desired product (43 mg, 0.13 mmol, 11% yield). MS (ESI) m/z 312.9 [M+1]⁺.

### Example 15. N-(5-(4-Hydroxy-4-phenylpiperidin-1-yl)quinolin-8-yl)-4-methylbenzenesulfonamide

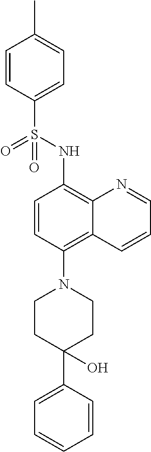 [SMILES: Cc1ccc(S(=O)(=O)Nc2ccc(N3CCC(O)(c4ccccc4)CC3)c3cccnc23)cc1]

N-(5-(4-Hydroxy-4-phenylpiperidin-1-yl)quinolin-8-yl)-4-methylbenzenesulfonamide. A mixture of N-(5-bromoquinolin-8-yl)-4-methylbenzenesulfonamide (500 mg, 1.33 mmol), 4-phenylpiperidin-4-ol (1.14 g, 6.65 mmol), tris(dibenzylideneacetone)dipalladium (61 mg, 0.066 mmol), racemic-2,2′-bis(diphenylphosphino)-1,1′-binaphthyl (43.7 mg, 0.066 mmol) and cesium carbonate (0.66 g, 2 mmol) in DMF (2 mL) was heated at 110° C. overnight under nitrogen atmosphere. The product was isolated and purified by standard methods to afford the desired product (16.0 mg, 0.032 mmol, 2.4% yield). MS (ESI) m/z 474.2 [M+1]⁺.

### Example 16. 4-Methyl-N-(3-methylquinolin-8-yl)benzenesulfonamide

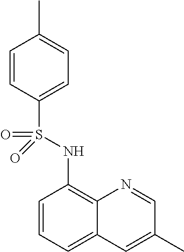 [SMILES: Cc1ccc(S(=O)(=O)Nc2cccc3cc(C)cnc23)cc1]

3-Methyl-8-nitroquinoline. To a solution of 3-methylquinoline (1 g, 6.99 mmol) in sulfuric acid (2 mL) was added slowly nitric acid (1 mL) at −5° C. The resulting solution was stirred at room temperature overnight. The reaction mixture was poured into ice-water and adjusted to pH 9 with ammonia. The mixture was filtered and the cake was dissolved in MeOH (5 mL), heated at reflux for 0.5 h and cooled to room temperature slowly. The mixture was filtered and the filtrate was concentrated and purified by silica gel column chromatography to afford the titled compound (309 mg, 1.6 mmol, 23% yield).

3-Methyl-quinolin-8-amine. A mixture of 3-methyl-8-nitroquinoline (300 mg, 1.60 mmol) and palladium on carbon (10%, 30 mg) in MeOH (10 mL) was stirred at 25° C. under hydrogen atmosphere for 3 h. The reaction mixture then was filtered over celite and the residue was washed with MeOH. The reaction was filtered through celite and evaporated in vacuo to give crude product (250 mg, 1.58 mmol, 99% yield), which was used for next step without further purification.

4-Methyl-N-(3-methylquinolin-8-yl)benzenesulfonamide. To a solution of 3-methyl-quinolin-8-amine. (172 mg, 1.09 mmol) in pyridine (10 mL) was added 4-methyl-benzenesulfonyl chloride (420 mg 2.2 mmol). The resulting mixture was heated at 100° C. overnight. The product was isolated and purified by standard methods to give the titled product (155.5 mg, 0.50 mmol, 46% yield). MS (ESI) m/z 313.1 [M+1]⁺.

### Example 17. N-(6-Fluoroquinolin-8-yl)-3,5-dimethoxybenzenesulfonamide

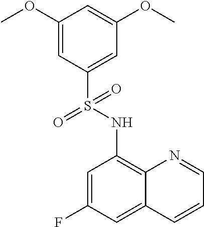 [SMILES: COc1cc(OC)cc(S(=O)(=O)Nc2cc(F)cc3cccnc23)c1]

3,5-Dimethoxybenzenesulfonyl chloride. A solution of 1-bromo-3,5-dimethoxy-benzene (1 g, 4.61 mmol) in THF was added a 2.5 N solution of n-butyllithium (2 mL, 5 mmol) dropwise at −78° C. and stirred at the same temperature for 1 hour. After that, sulfuryl chloride (0.42 mL, 5.0 mmol) was added and the mixture was warmed to room temperature and stirred overnight. The reaction was quenched with water, extracted with DCM and dried over sodium sulfate. Concentration under vacuum afforded the crude product (1.0 g, 50% purity by LC-MS), which was subjected to the next step without further purification.

N-(6-Fluoroquinolin-8-yl)-3,5-dimethoxybenzenesulfonamide. To a solution of 3,5-dimethoxy-benzenesulfonyl chloride (1.0 g, 4.23 mmol) in pyridine (2 mL) was added 6-fluoroquinolin-8-amine (684 mg, 4.23 mmol) and stirred at room temperature overnight. The product was isolated and purified by standard methods to afford the title product (167 mg, 0.461 mmol, 11% yield). MS (ESI) m/z 363.3 [M+H]⁺.

### Example 18. N-(6-Phenylquinolin-8-yl)pyridine-2-sulfonamide

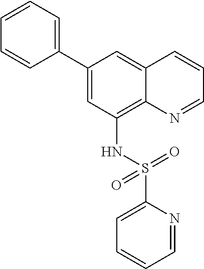 [SMILES: O=S(=O)(Nc1cc(-c2ccccc2)cc2cccnc12)c1ccccn1]

Pyridine-2-sulfonyl chloride. Pyridine-2-thiol (5.0 g, 45 mmol) was dissolved in 6 N aqueous hydrochloric acid (20 mL) and chlorine was bubbled into the mixture at 0° C. for 30 minutes. The mixture was extracted with diethyl ether. The extracts were dried over anhydrous sodium sulfate and evaporated at low temperature. The residue obtained was used directly in the next step (4.6 g, crude).

N-(6-Phenylquinolin-8-yl)pyridine-2-sulfonamide. To a solution of 6-phenylquinolin-8-amine (120 mg, 0.545 mmol) in pyridine (3 mL) was added pyridine-2-sulfonyl chloride (180 mg, crude). The mixture was stirred at room temperature overnight. The product was isolated and purified by standard methods to afford the title product (36 mg, 0.1 mmol, yield: 18%). MS(ESI) m/z 362.1 [M+H]⁺.

### Example 19. 6-Cyano-pyridine-2-sulfonic acid quinolin-8-ylamide

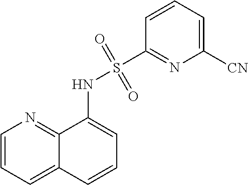 [SMILES: N#Cc1cccc(S(=O)(=O)Nc2cccc3cccnc23)n1]

2-Bromo-6-(4-methoxy-benzylsulfanyl)-pyridine. To a suspension of sodium hydride (60% in oil, 1.01 g, 25.32 mmol) in THE (20 mL) was added (4-methoxyphenyl)methanethiol (1.56 g, 10.13 mmol) at −15° C. A solution of 2,6-dibromopyridine (3.0 g, 12.66 mmol) in THE (15 mL) was added to the reaction mixture and stirred at 25° C. for 2 h. After completion, reaction mixture was diluted with water and extracted with ethyl acetate. Combined organic layer was washed with brine, dried over anhydrous Na₂SO₄, filtered, and concentrated under reduced pressure. Crude product was purified by column chromatography to afford 2-bromo-6-(4-methoxy-benzylsulfanyl)-pyridine (1.2 g, 30%). MS (ESI): m/z 309.9 [M+1]⁺.

6-Bromo-pyridine-2-sulfonic acid quinolin-8-ylamide. 2-Bromo-6-(4-methoxy-benzylsulfanyl)-pyridine (1.2 g, 3.87 mmol) was added drop wise to concentrated sulphuric acid (12 mL) at −30° C. and continued stirring at 25° C. until it became a clear solution. It was again cooled to −30° C. and NaOCl solution (10% aq, 36 mL) was added drop wise via addition funnel to the reaction mixture. The reaction mixture was slowly warmed to 25° C. and stirred for 2 h. The reaction mixture was diluted with ice cold water and extracted with ethyl acetate. Combined organic layer was washed with water and brine, dried over sodium sulphate, filtered, and concentrated under reduced pressure. Crude solution of sulphonyl chloride in DCM (10 mL) was added to the solution of quinolin-8-amine (279 mg, 1.93 mmol) in DCM (15 mL) and pyridine (3 mL, 38.71 mmol) at 0° C. and stirred for 12 h. After completion, the reaction mixture was diluted with water and extracted with ethyl acetate three times. The organic layer was washed with brine, dried over anhydrous Na₂SO₄, filtered, and concentrated under reduced pressure. Crude material was purified by column chromatography to afford product 6-bromo-pyridine-2-sulfonic acid quinolin-8-ylamide (930 mg, 66%). MS (ESI): m/z 363.8 [M+1]⁺.

6-Cyano-pyridine-2-sulfonic acid quinolin-8-ylamide. To a stirred degassed solution of 6-bromo-pyridine-2-sulfonic acid quinolin-8-ylamide (400 mg, 1.10 mmol) in DMA (5 ml) in sealed tube was added zinc cyanide (141.8 mg, 117.41 mmol) followed by TMEDA (0.052 ml, 0.329 mmol), Pd₂(dba)₃ (100 mg, 0.11 mmol), and Xantphos (63.54 mg, 0.11 mmol). Resulting mixture was heated at 80° C. for 5 h. The reaction mass was then cooled to 25° C., diluted with water and extracted with ethyl acetate. The combined organic layer was washed with brine and dried over anhydrous Na₂SO₄, filtered, and concentrated under reduced pressure. Crude product was purified by prep-HPLC to afford 6-Cyano-pyridine-2-sulfonic acid quinolin-8-ylamide (25 mg, 8%). MS (ESI): m/z 311.1 [M+1]⁺.

### Example 20. 6-Phenyl-N-(quinolin-8-yl)pyridine-2-sulfonamide

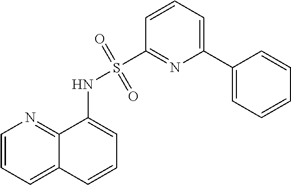 [SMILES: O=S(=O)(Nc1cccc2cccnc12)c1cccc(-c2ccccc2)n1]

2-Bromo-3-phenylpyridine. To a stirred solution of 2-bromo-3-iodopyridine (1.5 gm, 5.28 mmol) in toluene (10 ml) was added EtOH (5 ml), water (2 ml), and potassium carbonate (802 mg, 5.81 mmol) followed by phenyl boronic acid (773 mg, 6.34 mmol). Resulting mixture was degassed with argon for 15 min and Pd(PPh₃)₄ (305 mg, 0.26 mmol) was added under inert atmosphere. Resulting mixture was heated to 70° C. for 4 h under argon atmosphere. Reaction mixture was cooled to 25° C. and diluted with ethyl acetate. Organics were washed with water and brine, dried over anhydrous sodium sulphate, filtered, and concentrated under reduced pressure. Crude mass was purified by silica gel column chromatography to afford 2-bromo-3-phenylpyridine (250 mg, 20%). MS (ESI): m/z 233.8 [M+1]⁺.

2-{[(4-Methoxyphenyl)methyl]sulfanyl}-3-phenylpyridine. To a stirred solution of 4-methoxy-benzylamine (790 mg, 2.56 mmol) in DMSO (10 mL) was added CsF (779 mg, 5.13 mmol) followed by 2-bromo-3-phenylpyridine (600 mg, 2.56 mmol) at 25° C. Resulting mixture was heated at 80° C. for 3 h. After completion, reaction mixture was diluted with ice cold water and extracted with ethyl acetate. The combined organic layer was washed with brine, dried over anhydrous sodium sulphate, filtered, and evaporated under reduced pressure. Crude mass was purified by flash column chromatograph to afford 2-{[(4-methoxyphenyl)methyl]sulfanyl}-3-phenylpyridine (600 mg, 76%). MS (ESI): m/z 308.2 [M+1]⁺.

3-Phenylpyridine-2-sulfonyl chloride. A solution of 2-{[(4-methoxyphenyl)methyl]sulfanyl}-3-phenylpyridine (100 mg, 0.32 mmol) in H₂SO₄ (4 mL) was cooled to −10° C. and sodium hypochlorite (10% aqueous solution, 10 mL) was added and stirred for 0.5 h under cooling conditions. The reaction mixture was quenched with ice cooled water and extracted with DCM. The combined organic layer was washed with water and brine, dried over anhydrous sodium sulphate, filtered, and concentrated under reduced pressure to afford 3-phenylpyridine-2-sulfonyl chloride (70 mg, crude, 84%).

6-Phenyl-N-(quinolin-8-yl)pyridine-2-sulfonamide. To a stirred solution of quinolin-8-amine (22 mg, 0.154 mmol) in pyridine (0.3 mL), a solution of 3-phenylpyridine-2-sulfonyl chloride (65 mg, 0.26 mmol) in DCM (2.5 mL) was added drop wise at 0° C. under argon atmosphere. The reaction mixture was warmed to 25° C. and stirred for 16 h. After completion, the reaction mixture was diluted with DCM. The organic layer was washed with water and brine, dried over anhydrous sodium sulphate, filtered, and concentrated under reduced pressure. Crude mass was purified by prep HPLC to afford 6-phenyl-N-(quinolin-8-yl)pyridine-2-sulfonamide (30 mg, 32%). MS (ESI): m/z 362.1 [M+1]⁺.

### Example 21. 4-Cyclopropyl-N-(1, 5-naphthyridin-4-yl) pyridine-2-sulfonamide

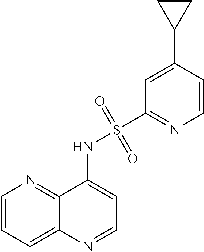 [SMILES: O=S(=O)(Nc1ccnc2cccnc12)c1cc(C2CC2)ccn1]

2-Chloro-4-cyclopropylpyridine. To a stirred solution of 4-bromo-2-chloropyridine (1 g, 5.19 mmol) in dioxane (25 mL) and water (2.5 mL) was added potassium carbonate (2.51 g, 18.19 mmol) followed by cyclopropylboronic acid (669 mg, 7.79 mmol). The resulting mixture was degassed with argon and Pd(dppf)Cl₂. DCM (381 mg, 0.52 mmol) was added under inert atmosphere. Resulting mixture was heated at 110° C. for 16 h. After completion, the reaction mixture was cooled to 25° C. and filtered through a short pad of celite. The filtrate was concentrated under reduced pressure and diluted with ethyl acetate. Organic layer was washed with water and brine, dried over anhydrous sodium sulphate, filtered, and evaporated under reduced pressure. Crude product was purified by column chromatography to afford 2-chloro-4-cyclopropylpyridine (700 mg, 88%). MS (ESI): m/z 154.2 [M+1]⁺.

4-Cyclopropyl-2-{[(4-methoxyphenyl) methyl] sulfanyl} pyridine. To a stirred solution of (4-methoxyphenyl)methanethiol (2.62 g, 16.99 mmol) in DMSO (20 mL) was added CsF (3.2 g, 21.24 mmol) followed by 2-chloro-4-cyclopropylpyridine (1.3 g, 8.50 mmol) at 25° C. Resulting mixture was heated at 80° C. for 16 h. After completion, reaction mixture was diluted with ice cold water and extracted with ethyl acetate. Combined organic layer was washed with brine, dried over anhydrous sodium sulphate, filtered, and evaporated under reduced pressure. Crude mass was purified by column chromatograph to afford 4-cyclopropyl-2-{[(4-methoxyphenyl) methyl] sulfanyl} pyridine (2.1 g, 91%). MS (ESI): m/z 271.9 [M+1]⁺.

4-cyclopropylpyridine-2-sulfonyl chloride. A solution of 4-cyclopropyl-2-{[(4-methoxyphenyl) methyl] sulfanyl} pyridine (100 mg, 0.37 mmol) in H₂SO₄ (4 mL) was cooled to −10° C. and sodium hypochlorite (10% aqueous solution, 10 mL) was added and stirred for 0.5 h under cooling condition. Reaction mixture was quenched with ice cooled water and extracted with DCM. Combined organic layer was washed with water and brine, dried over anhydrous sodium sulphate, filtered, and concentrated under reduced pressure to afford 4-cyclopropylpyridine-2-sulfonyl chloride (80 mg, crude, 99%).

4-Cyclopropyl-N-(1, 5-naphthyridin-4-yl) pyridine-2-sulfonamide. To a solution of 1, 5-naphthyridin-4-amine (60 mg, 0.41 mmol) in pyridine (3 mL) was added a solution of 4-cyclopropylpyridine-2-sulfonyl chloride (300 mg, 1.38 mmol) in DCM (3 mL) drop wise at 0° C. under argon atmosphere. The reaction mixture was warmed to 25° C. and stirred for 16 h. After completion, the reaction mixture was diluted DCM. The organic layer was washed with water and brine, dried over anhydrous sodium sulphate, filtered, and concentrated under reduced pressure. Crude mass was purified by prep HPLC to afford 4-cyclopropyl-N-(1, 5-naphthyridin-4-yl) pyridine-2-sulfonamide (15 mg, 3%). MS (ESI): m/z 327.0 [M+1]⁺.

### Example 22. N-(3-Cyclopropylquinolin-8-yl)-3-methylpyridine-2-sulfonamide

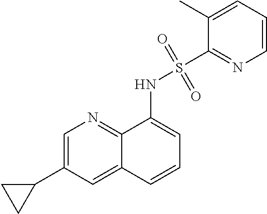 [SMILES: Cc1cccnc1S(=O)(=O)Nc1cccc2cc(C3CC3)cnc12]

3-Cyclopropylquinolin-8-amine. To a stirred solution of 3-bromoquinolin-8-amine (100 mg, 0.45 mmol) in toluene-water (10.5 mL, 20:1) in a sealed tube was added cyclopropyl boronic acid (116 mg, 1.34 mmol) and K₃PO₄ (333 mg, 1.57 mmol). The solution was degassed with argon for 10 min followed by addition of tricyclohexyl phosphine (19 mg, 0.07 mmol) and Pd(OAc)₂ (10 mg, 0.05 mmol) under inert atmosphere. Resulting mixture was heated at 110° C. for 16 h. After completion, the reaction mixture was cooled to 25° C., diluted with water, and extracted with ethyl acetate. The combined organic layer was washed with brine, dried over Na₂SO₄, filtered, and concentrated under reduced pressure. Crude compound was purified by column chromatography to afford 3-cyclopropylquinolin-8-amine (70 mg, 84%). MS (ESI): m/z 185.1 [M+1]⁺.

3-Methylpyridine-2-sulfonyl chloride. Concentrated H₂SO₄ (6.4 mL) was added drop wise to 2-{[(4-methoxyphenyl)methyl]sulfanyl}-3-methylpyridine (400 mg, 1.63 mmol) in a round bottom flask at −20° C. and continued stirring for 15 min. NaOCl (24.11 mL, 359.18 mmol) was added using an addition funnel over 30 min at −20° C. and then slowly warmed to 0° C. over 30 min. After completion, the reaction mixture was quenched with cold water and extracted with dry DCM. Combined organic layer was washed with brine, dried over MgSO₄, and filtered. Filtrate was concentrated under reduced pressure to a minimum volume and was used directly in the next step.

N-(3-Cyclopropylquinolin-8-yl)-3-methylpyridine-2-sulfonamide. To a stirred solution of 3-cyclopropylquinolin-8-amine (50 mg, 0.27 mmol) in pyridine (2 mL) at 0° C. was slowly added a solution of 3-methylpyridine-2-sulfonyl chloride (104 mg, 0.54 mmol) in DCM (4 mL). Resulting mixture was warmed to 25° C. and stirred for 16 h. After completion, the reaction mixture was diluted with DCM, washed with water and brine, dried over Na₂SO₄, filtered, and concentrated under reduced pressure. Crude compound was purified by prep-HPLC to afford N-(3-cyclopropylquinolin-8-yl)-3-methylpyridine-2-sulfonamide (24 mg, 26%). MS (ESI): m/z 340.3 [M+1]⁺.

### Example 23. N-(5-Methylquinolin-8-yl)furan-2-sulfonamide

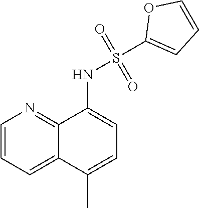 [SMILES: Cc1ccc(NS(=O)(=O)c2ccco2)c2ncccc12]

5-Methyl-8-nitroquinoline. Glycerol (6.5 mL, 89 mmol) was preheated at 165° C. for 1 h and cooled to 150° C. 5-methyl-2-nitroaniline (5 g, 33 mmol) and sodium iodide (100 mg, 0.7 mmol) were added. Sulphuric acid (4.2 mL, 78 mmol) was then added drop wise over the period of 10 min at the same temperature and the reaction mixture was stirred at 150° C. for 5 h. After being cooled to 25° C., the resulting reaction mixture was carefully poured into ice-cold water, and resulting solid product was filtered off. The collected solid was recrystallized from 10% ethyl acetate in petroleum ether to afford 5-methyl-8-nitro-quinoline (4 g, 90% purity, 21.27 mmol, 64.7% yield). MS (ESI) m/z 189.0 [M+H]⁺.

5-Methylquinolin-8-amine. To a solution of 5-methyl-8-nitroquinoline (2 g, 10.63 mmol) in EtOH (50 mL) was added stannous chloride (8.02 g, 42.55 mmol). The resulting reaction mixture was heated under reflux for 2 h and cooled to 25° C. Sodium hydroxide solution (10%, 50 mL) was added and the reaction mixture was extracted with ethyl acetate. The combined organic layers were washed with 10% sodium hydroxide solution, water and brine. The organic phase was dried over anhydrous sodium sulfate and filtered. Concentration under vacuum gave crude product (1.5 g, 9.49 mmol, 89.3% yield), which was subjected to the next step without further purification. MS (ESI) m/z 159.1 [M+H]⁺.

N-(5-Methylquinolin-8-yl)furan-2-sulfonamide. To a solution of 5-methylquinolin-8-amine (200 mg, 1.26 mmol) in pyridine (6 mL) was added furan-2-sulfonyl chloride (316 mg, 1.89 mmol). After being irradiated via microwave for 3 min at 130° C., the reaction mixture was cooled to 25° C., then quenched with water. The resulting product was filtered, washed with water and diethyl ether to afford the title compound (51 mg, 0.17 mmol, 13% yield). ¹H NMR (400 MHz, DMSO-d₆) δ 10.10 (br s, 1H), 8.77-8.76 (m, 1H), 8.27-8.25 (m, 1H), 7.83 (s, 1H), 7.56-7.51 (m, 2H), 7.17 (s, 1H), 6.55-6.54 (m, 1H), 2.44 (s, 3H); MS (ESI) m/z 289.0 [M+H]+; Purity: 97.4% at 214 nm, 90.2% at 254 nm.

### Example 24. N-(5-Fluoroquinolin-8-yl)furan-2-sulfonamide

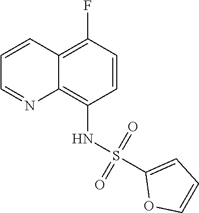 [SMILES: O=S(=O)(Nc1ccc(F)c2cccnc12)c1ccco1]

5-Fluoroquinoline. To a suspension of quinolin-5-amine (1.5 g, 10.4 mmol) in fluoroboric acid (10 mL w.t. %=48%) was added portion wise sodium nitrite (934 mg, 13.5 mmol) at −5° C. The mixture was stirred for 1 h and then poured into a solution of 50% ethyl acetate in petroleum ether. The resulting suspension was filtered and the filter cake was air dried. The resulting solid which was collected was added to refluxing xylene and stirred under reflux for 2 hours before cooling to 25° C. The xylene was decanted off and the residue was dissolved in hydrochloric acid (20 mL, 1N). After neutralization with sodium carbonate, the mixture was extracted with ethyl acetate. The organic extracts were dried over anhydrous sodium sulfate and concentrated under reduced pressure. The residue was subjected to silica gel chromatography to give the desired compound (1.1 g, 7.43 mmol, 71% yield). MS (ESI) m/z 148.0. [M+H]⁺.

5-Fluoro-8-nitroquinoline. 5-Fluoroquinoline (300 mg 2.02 mmol) was dissolved in concentrated sulfuric acid (2 mL) at 0° C., then nitric acid (1 mL, 65%) was added slowly at −5° C. The mixture was stirred at −5° C. for 1 h, and then allowed to stir at 25° C. overnight. The reaction mixture was poured into ice and the resulting mixture was basified with aqueous ammonium hydroxide (10 mL, 11 M) to pH 10 and extracted with ethyl acetate. After removal of all volatiles in vacuo, the residue was purified by silica gel column chromatography to provide the title product (168 mg, 0.87 mmol, 43% yield). ¹H NMR (400 MHz, Chloroform-d₃) δ 8.17-8.15 (m, 1H), 7.73 (d, J=8.4 Hz, 1H), 7.34-7.30 (m, 1H), 6.88-6.84 (m, 1H).

5-Fluoroquinolin-8-amine. To a suspension of 5-fluoro-8-nitroquinoline (192 mg 1.0 mmol) and iron dust (366 mg, 6.0 mmol) in water (8 mL) was added acetic acid (0.5 mL). The mixture was heated at 100° C. for 1.5 h, and then cooled to 25° C. The reaction mixture was basified with solid sodium hydroxide and then treated with ethyl acetate with continued stirring. The resulting solid which was collected was rinsed with ethyl acetate and the combined filtrate was dried over anhydrous sodium sulfate and concentrated under reduced pressure. The residue was subjected to silica gel chromatography to provide the desired product (130 mg, 0.80 mmol, 80% yield). MS (ESI) m/z 163.1 [M+H]⁺.

N-(5-Fluoroquinolin-8-yl)furan-2-sulfonamide. To a solution of 5-fluoroquinolin-8-amine (100 mg, 0.62 mmol) in pyridine (3 mL) was added furan-2-sulfonyl chloride (113 mg, 0.68 mmol), the reaction mixture was irritated at 110° C. by microwave for 15 min. After removal of all volatiles in vacuo, the residue was purified by silica gel column chromatography to give the desired product (54.3 mg, 0.19 mmol, 31% yield). ¹H NMR (400 MHz, CHLOROFORM-d) δ 9.10 (s, 1H), 8.84 (dd, J=4.4, 1.6 Hz, 1H), 8.40 (dd, J=8.8, 1.6 Hz, 1H), 7.80-7.77 (m, 1H), 7.54-7.51 (m, 1H), 7.39 (s, 1H), 7.17 (t, J=8.8 Hz, 1H), 7.07 (d, J=3.6 Hz, 1H), 6.35 (m, 1H); MS (ESI) m/z 293.0 [M+H]⁺; Purity=99.2% at 254 nm.

### Example 25. N-(5-Morpholinoquinolin-8-yl)-1H-pyrazole-4-sulfonamide

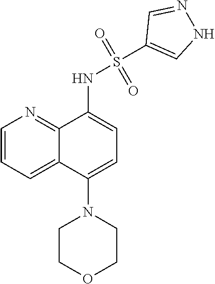 [SMILES: O=S(=O)(Nc1ccc(N2CCOCC2)c2cccnc12)c1cn[nH]c1]

4-(8-Nitroquinolin-5-yl)morpholine. 5-Chloro-8-nitroquinoline (600 mg, 2.88 mmol) was suspended in morpholine (3 mL) and the mixture was irritated by a microwave apparatus under 100° C. for 10 min. After cooling to 25° C., the residue was concentrated and washed with hexane (20 mL) to afford the desired product, which was used in the next step without further purification (480 mg, 64% yield, 1.84 mmol). MS (ESI) m/z 260.1 [M+H]⁺.

5-Morpholinoquinolin-8-amine. To a solution of 4-(8-nitroquinolin-5-yl)morpholine (480 mg, 1.84 mmol) in MeOH (10 mL) was added palladium on carbon (100 mg, 10%). The mixture was stirred at 25° C. overnight under an atmosphere of hydrogen. The catalyst was filtered off and the filtrate was concentrated to give the desired product, which was used in the next step without further purification (405 mg, yield: 95%, 1.76 mmol). MS (ESI) m/z 230.1 [M+H]⁺.

N-(5-Morpholinoquinolin-8-yl)-1H-pyrazole-4-sulfonamide. To a solution of 5-morpholinoquinolin-8-amine (80 mg, 0.35 mmol) in pyridine (2 mL) was added 1H-pyrazole-4-sulfonyl chloride (58 mg, 0.35 mmol) and the reaction mixture was stirred at 25° C. for 16 h. The solution was concentrated under vacuum to give crude product, which was purified by silica gel column chromatography to give the desired product (25.7 mg, 0.072 mmol, 21% yield). ¹H NMR (400 MHz, METHANOL-d) δ 8.93-8.88 (m, 2H), 7.85 (s, 2H), 7.75-7.71 (m, 1H), 7.59 (d, J=8.8 Hz, 1H), 7.26 (d, J=8.8 Hz, 1H), 3.95-3.93 (m, 4H), 3.09-3.07 (m, 4H); MS (ESI) m/z 360.1 [M+H]⁺; Purity: 96.5% at 214 nm.

### Example 26. N-(Quinazolin-8-yl)pyridine-2-sulfonamide

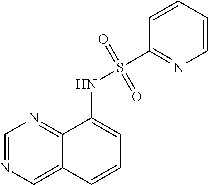 [SMILES: O=S(=O)(Nc1cccc2cncnc12)c1ccccn1]

N-(Quinazolin-8-yl)pyridine-2-sulfonamide. Quinazolin-8-amine (175 mg, 1.206 mmol), pyridine-2-sulfonyl chloride (257 mg, 1.447 mmol), and pyridine (5 mL, 61.8 mmol) were combined and stirred over 48 h at 25° C. The reaction was heated at 70° C. for 3 h. The crude material was purified via column chromatography. The crude material was dissolved in MeOH and filtered through a frit to remove insoluble material before purifying directly on a semi-prep HPLC. Product fractions were combined and condensed under reduced pressure. The product was dissolved in MeOH, and washed through a resin column, eluting with MeOH, and condensed to afford N-(quinazolin-8-yl)pyridine-2-sulfonamide (27.4 mg, 0.096 mmol, 7.94% yield). ¹H NMR (400 MHz, DMSO-d₆) δ ppm 10.34 (s, 1H), 9.60 (s, 1H), 9.23 (s, 1H), 8.59 (dt, J=4.69, 1.17 Hz, 1H), 7.98-8.09 (m, 3H), 7.89 (dd, J=8.20, 1.17 Hz, 1H), 7.68-7.74 (m, 1H), 7.59-7.65 (m, 1H); MS (ESI) m/z 287.0 [M+1]⁺

### Example 27. N-(Quinolin-8-yl)-[1,2,4]triazolo[4,3-a]pyridine-3-sulfonamide

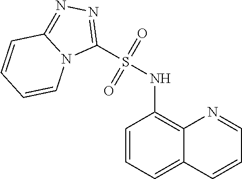 [SMILES: O=S(=O)(Nc1cccc2cccnc12)c1nnc2ccccn12]

[1,2,4]Triazolo[4,3-a]pyridine-3-sulfonyl chloride. To a solution of 3-bromo-[1,2,4]triazolo[4,3-a]pyridine (530 mg, 2.68 mmol) was added a solution of n-butyllithium in hexane (1.1 mL, 2.75 mmol, 2.5 N) dropwise at −78° C. under nitrogen. The mixture was stirred at −78° C. for 1 h before sulfuryl chloride (359 mg, 2.68 mmol) was added slowly. The resulting mixture was stirred at 25° C. for 1 h. The reaction was quenched with water and the aqueous layer was extracted with DCM. The organic layer was dried over sodium sulfate, filtered, and concentrated to afford the crude desired product (430 mg, crude), which was subjected to the next step without further purification. MS (ESI) m/z 218.0 [M+H]⁺.

N-(Quinolin-8-yl)-[1,2,4]triazolo[4,3-a]pyridine-3-sulfonamide. To a solution of quinolin-8-amine (70 mg, 0.48 mmol) in pyridine (1.5 mL) was added N-(quinolin-8-yl)-[1,2,4]triazolo[4,3-a]pyridine-3-sulfonamide (430 mg, crude) at 0° C. The reaction was stirred at 25° C. for 16 h. The reaction was concentrated and then the residue was purified by high performance liquid chromatography to give the desired product (5.6 mg, 0.017 mmol, 3.5%). ¹H NMR (400 MHz, DMSO-d₆) δ 9.00 (d, J=4.4 Hz, 1H), 8.59 (d, J=8.4 Hz, 1H), 8.32 (d, J=6.4 Hz, 1H), 7.97 (d, J=7.2 Hz, 1H), 7.73-7.52 (m, 4H), 7.38 (t, J=8.0 Hz, 1H), 6.70 (t, J=7.2 Hz, 1H). MS (ESI) m/z 325.8 [M+H]⁺. Purity=98.7% at 214 nm, Purity=97.3% at 254 nm.

### Example 28. N-(Quinolin-8-yl)pyrimidine-4-sulfonamide

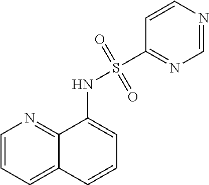 [SMILES: O=S(=O)(Nc1cccc2cccnc12)c1ccncn1]

Pyrimidine-4-thiol. A mixture of pyrimidin-4-ol (7.00 g, 72.9 mmol) and phosphorus pentasulfide (16.24 g, 73.15 mmol) in pyridine (105 mL) was stirred at reflux for 3 h. The reaction mixture was concentrated, and the residue was treated with water. The aqueous layer was extracted with ethyl acetate. The combined organic layers were washed with brine, dried over anhydrous sodium sulfate and concentrated to give the desired product (7.00 g, 62.5 mmol, 85% yield). ¹H NMR (400 MHz, DMSO-d₆) δ 14.11 (br, 1H), 8.29 (s, 1H), 7.86 (d, J=6.4 Hz, 1H), 7.18 (d, J=6.4 Hz, 1H). MS (ESI) m/z 113.0 [M+H]⁺.

N-(Quinolin-8-yl)pyrimidine-4-sulfonamide. To a stirred mixture of aqueous hydrochloride acid solution (2N, 89 mL) and DCM (118 mL) cooled to −5° C. (internal temperature) was added a pre-cooled (−5° C.) sodium hypochlorite (10% solution, 1.55 M, 78 mL, 122.7 mmol) at such a rate that the temperature was maintained below 0° C. Pyrimidine-2-thiol (4.00 g, 35.7 mmol) was added in small portions while the internal temperature was maintained at −10° C. to −5° C. The mixture was stirred for 20 minutes at −10° C. to −5° C. after the addition was completed. Excess chlorine was quenched by addition of a cold (0° C.) aqueous sodium sulfite solution (1M) until the yellow greenish color of the mixture disappeared and iodide paper (potassium iodide/starch) no longer gave a fast coloration. The reaction mixture was then transferred to a separating funnel (pre-cooled either in the freezer or with ice water) and the organic layer was rapidly separated and collected in a clean flask. The aqueous phase was quickly extracted with cold (−10° C.) DCM. The organic extracts were combined and dried over magnesium sulfate under nitrogen atmosphere cooled in a dry ice-acetone bath. To a solution of quinolin-8-ylamine (400 mg, 2.78 mmol) in THE (1 mL) was added a solution of sodium bis(trimethylsilyl)amide in THE (2M, 2.1 mL, 4.20 mmol) slowly at −78° C. under nitrogen and stirred for another hour. Then the pyrimidine-4-sulfonyl chloride solution in DCM obtained above was filtered quickly and added to the above solution. One hour later, the reaction mixture was quenched with aqueous ammonia chloride solution and extracted with DCM. The combined organic layers were washed with brine, dried over anhydrous sodium sulfate and concentrated. The residue was purified by high performance liquid chromatography to give the desired product (11.0 mg, 0.038 mmol, 1% yield). ¹H NMR (400 MHz, MeOH-d₄) δ 9.13, (s, 1H), 8.99 (d, J=4.8 Hz, 1H), 8.79 (dd, J=4.4 Hz, 1.2 Hz, 1H), 8.26 (d, J=8.0 Hz, 1H), 8.05 (d, J=4.8 Hz, 1H), 7.89 (d, J=7.6 Hz, 1H), 7.63 (d, J=8.4 Hz, 1H), 7.52-7.46 (m, 2H). MS (ESI) m/z 287.0 [M+H]⁺. Purity=97.4% at 214 nm, Purity=98.0% at 254 nm.

### Example 29. 1-Ethyl-N-(5-methoxyquinolin-8-yl)-1H-imidazole-2-sulfonamide

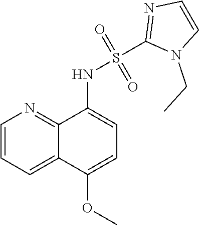 [SMILES: CCn1ccnc1S(=O)(=O)Nc1ccc(OC)c2cccnc12]

1-((1-Ethyl-1H-imidazol-2-yl)sulfonyl)-1H-benzo[d][1,2,3]triazole. To a solution of mixture of 1-ethyl-1H-imidazole (15.0 g, 156 mmol) in THE (450 mL) was added a solution of n-butyllithium in hexane (2.5 N, 66 mL, 164 mmol) dropwise at −78° C. under nitrogen. The resulting reaction mixture was stirred at −78° C. for 1 h. Sulfur dioxide was bubbled to the above solution of the organometallic reagent in THF at −78° C. until a sample of the solution gave a pH acid test. The mixture was stirred at that temperature for 15 min, then at 25° C. for 1 h. N-chlorobenzotriazole (23.0 g, 156 mmol) was added in one portion and the mixture was stirred for 2 h at 25° C. Triethylamine (39.4 g, 390 mmol) was added, followed by stirring at 25° C. for 16 h. Water was added and the solution was extracted with ethyl acetate. The combined organic layers were washed with saturated aqueous ammonium chloride solution, dried over anhydrous sodium sulfate and filtered. Concentrated and the residue was recrystallized from ethyl acetate to afford the title compound (23.0 g, 83.0 mmol, 53% yield). MS (ESI) m/z 278.1 [M+H]⁺.

1-Ethyl-N-(5-methoxyquinolin-8-yl)-1H-imidazole-2-sulfonamide. To a solution of 5-methoxyquinolin-8-amine (200 mg, 1.15 mmol) in THF (5 mL) was added a solution of sodium bis(trimethylsilyl)amide in THF (2M, 1.15 mL, 2.30 mmol) slowly at −78° C. under nitrogen. After the resulting reaction mixture was stirred for 1 h at the temperature, a solution of 1-((1-ethyl-1H-imidazol-2-yl)sulfonyl)-1H-benzo[d][1,2,3]triazole (400 mg, 1.44 mmol) in THF (8 mL) was added at −78° C. Then the resulting solution was stirred at 25° C. for 1 h. The reaction mixture was quenched with aqueous ammonia chloride solution and extracted with ethyl acetate. The combined organic layers were washed with brine, dried over anhydrous sodium sulfate and concentrated. The residue was purified by preparative high performance liquid chromatography to give the title compound (15.6 mg, 0.047 mmol, 4% yield). 1H NMR (400 MHz, DMSO-d₆) δ 10.09 (brs, 1H), 8.79 (dd, J₁=4.4 Hz, J₂=1.6 Hz, 1H), 8.49 (dd, J₁=8.0 Hz, J₂=1.2 Hz, 1H), 7.65 (d, J=8.4 Hz, 1H), 7.52 (dd, J₁=8.4 Hz, J₂=4.0 Hz, 1H), 7.39 (d, J=0.8 Hz, 1H), 7.03 (d, J=8.4 Hz, 1H), 6.85 (d, J=1.2 Hz, 1H), 4.30 (q, J=7.2 Hz, 2H), 3.96 (s, 3H), 1.31 (t, J=7.2 Hz, 3H). MS (ESI) m/z 332.8 [M+H]⁺. Purity=98.6% at 214 nm, Purity=98.6% at 254 nm.

### Example 30. 5-Methyl-N-(quinoxalin-5-yl)pyridine-2-sulfonamide

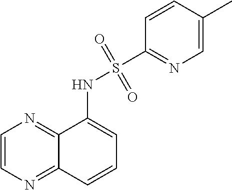 [SMILES: Cc1ccc(S(=O)(=O)Nc2cccc3nccnc23)nc1]

2-(Benzylthio)-5-methylpyridine. To a solution of phenylmethanethiol (23.3 g, 188 mmol) in anhydrous THF (200 mL) was added sodium hydride (60% in mineral oil, 8.28 g, 207 mmol) in portions at 0° C. The resulting mixture was stirred at 25° C. for 1 h and 2-chloro-5-methylpyridine (20.0 g, 157 mmol) was added portion-wise at 0° C. The resulting mixture was stirred at 70° C. for 30 h. The reaction was treated with water and the mixture was extracted with ethyl acetate. The combined organic layers were washed with brine, dried over anhydrous sodium sulfate and filtered. The filtrate was concentrated and the residue was purified by silica gel column chromatography to give the desired compound (10.5 g, 50% purity, 24.4 mmol, 16% yield). MS (ESI) m/z 216.1 [M+H]⁺.

5-Methylpyridine-2-sulfonyl chloride. Chlorine was bubbled into a solution of 2-(benzylthio)-5-methylpyridine (3.0 g, 50% purity crude, 6.98 mmol) in acetic acid (20 mL), DCM (20 mL) and water (7 mL) at 0° C. for 30 min. Then the resulting mixture was stirred at that temperature for 1 h. The reaction was diluted with DCM, washed with saturated aqueous sodium hydrogen carbonate and brine, dried over sodium sulfate and filtered. The filtrate was concentrated to afford the crude product (3.2 g), which was subjected to the next step without further purification. MS (ESI) m/z 191.9 [M+H]⁺.

5-Methyl-N-(quinoxalin-5-yl)pyridine-2-sulfonamide. To a solution of quinoxalin-5-amine (200 mg, 1.38 mmol) in anhydrous THE (3 mL) was added a solution of sodium bis(trimethylsilyl)amide in THE (2M, 1.38 mL, 2.76 mmol) slowly at −78° C. under nitrogen. The mixture was stirred at −78° C. for 1 h before the crude 5-methylpyridine-2-sulfonyl chloride (500 mg, crude) was added. The mixture was stirred at 25° C. for 1 h. The reaction was quenched with saturated aqueous ammonium chloride solution and extracted with DCM. The organic layer was dried over sodium sulfate, filtered and concentrated to afford the desired product, which was purified by preparative high performance liquid chromatography to afford the desired product (14.8 mg, 0.049 mmol, 4% yield). ¹H NMR (400 MHz, DMSO-d₆) δ 10.20 (brs, 1H), 8.98 (d, J=1.6 Hz, 1H), 8.87 (d, J=2.0 Hz, 1H), 8.43 (s, 1H), 7.95 (d, J=8.4 Hz, 1H), 7.85-7.77 (m, 4H), 2.32 (s, 3H). MS (ESI) m/z 300.8 [M+H]. Purity=98.3% at 214 nm, Purity=98.1% at 254 nm.

### Example 31. 3-Ethyl-N-(5-morpholino-8-quinolyl)imidazole-4-sulfonamide

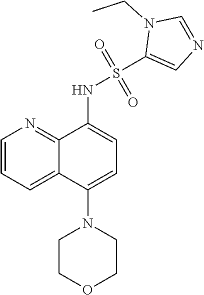 [SMILES: CCn1cncc1S(=O)(=O)Nc1ccc(N2CCOCC2)c2cccnc12]

(3-Ethylimidazol-4-yl)sulfinyloxylithium. To a solution of 1-ethylimidazole (10. g, 104.03 mmol) in diethyl ether (100 mL) was added n-butyllithium (54.27 mL, 124.83 mmol) slowly at −70° C. under nitrogen. The mixture was stirred at −70° C. for 30 min and 0° C. for 30 min. Then excess sulfur dioxide was bubbled and the mixture was stirred at −70° C. for 1 h. Then the mixture was warmed to 25° C., filtered, and the filter cake was dried under vacuum to give the crude (3-ethylimidazol-4-yl)sulfinyloxylithium (17 g, crude).

3-Ethylimidazole-4-sulfonyl chloride. To a solution of (3-ethylimidazol-4-yl)sulfinyloxylithium (17. g, crude) in chloroform (100 mL) and water (100 mL) was added NCS (13.66 g, 102.33 mmol) at 0° C., the mixture was stirred at 0° C. for 1 h. The mixture was diluted with cold water and the aqueous phase was extracted with chloroform. The combined organic phase was dried over anhydrous sodium sulfate, filtered and concentrated under vacuum. The crude product was purified by silica gel chromatography to give 3-ethylimidazole-4-sulfonyl chloride (3.5 g, 17.982 mmol, 17.573% yield). LCMS (ESI): m/z 195.0 [M+1].

3-Ethyl-N-(5-morpholino-8-quinolyl)imidazole-4-sulfonamide. To a solution of 5-morpholinoquinolin-8-amine (294.49 mg, 1.28 mmol) in pyridine (8 mL) was added a solution of 3-ethylimidazole-4-sulfonyl chloride (250. mg, 1.28 mmol) in DCM (2 mL). The mixture was stirred at 25° C. for 16 h. The reaction mixture was concentrated under reduced pressure to remove solvent. The residue was purified by flash silica gel chromatography to give the crude product, which was further purified by prep-HPLC followed by lyophilization to give 3-ethyl-N-(5-morpholino-8-quinolyl)imidazole-4-sulfonamide (63.21 mg, 0.1625 mmol, 12.651% yield, 99.6% purity). ¹H NMR (400 MHz, DMSO-d₆) δ 8.82 (dd, J₁=4.2, J₂=1.6 Hz, 1H), 8.50 (dd, J₁=8.4, J₂=1.5 Hz, 1H), 7.84 (s, 1H), 7.58-7.50 (m, 2H), 7.24 (d, J=1.0 Hz, 1H), 7.17 (d, J=8.2 Hz, 1H), 4.31 (q, J=7.1 Hz, 2H), 3.85-3.81 (m, 4H), 3.02-2.89 (m, 4H), 1.35 (t, J=7.2 Hz, 3H); LCMS (ESI): m/z 388.1 [M+1]⁻.

### Example 32. N-(5-Chloro-8-quinolyl)-3-isopropyl-imidazole-4-sulfonamide

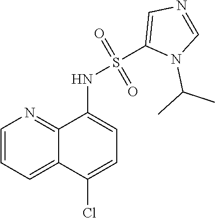 [SMILES: CC(C)n1cncc1S(=O)(=O)Nc1ccc(Cl)c2cccnc12]

(3-Isopropylimidazol-4-yl)sulfinyloxylithium. To a solution of 1-isopropylimidazole (10. g, 90.78 mmol) in diethyl ether (150 mL) was added n-butyllithium (47.36 mL, 108.93 mmol, 2.5 M in THF) slowly at −70° C. under nitrogen. The mixture was stirred at −70° C. for 30 min and 0° C. for 30 min. Then excess sulfur dioxide was bubbled, and the mixture was stirred at −70° C. for 1 h. Then the mixture was warmed to 25° C., which was filtered and the filter cake was dried under vacuum to give the crude (3-isopropylimidazol-4-yl)sulfinyloxylithium (16 g, crude).

3-Isopropylimidazole-4-sulfonyl chloride. To a solution of (3-isopropylimidazol-4-yl)sulfinyloxylithium (16. g, 88.81 mmol) in chloroform (100 mL) and water (100 mL) was added NCS (11.86 g, 88.81 mmol) at 0° C. The mixture was stirred at 0° C. for 1 h. The mixture was diluted with cold water and the aqueous phase was extracted with DCM. The combined organic phase was dried over anhydrous sodium sulfate, filtered, and concentrated under vacuum. The crude product was purified by silica gel chromatography to give 3-isopropylimidazole-4-sulfonyl chloride (1.4 g, 6.7092 mmol, 7.5541% yield). ¹H NMR (400 MHz, DMSO-d₆) δ 9.30 (d, J=1.5 Hz, 1H), 7.66 (d, J=1.5 Hz, 1H), 5.22-5.11 (m, 1H), 1.50 (d, J=6.8 Hz, 6H).

N-(5-Chloro-8-quinolyl)-3-isopropyl-imidazole-4-sulfonamide. To a solution of 5-chloroquinolin-8-amine (200. mg, 1.12 mmol) in pyridine (4 mL) was added a solution of 3-isopropylimidazole-4-sulfonyl chloride (233.65 mg, 1.12 mmol) in DCM (1 mL). The mixture was stirred at 25° C. for 16 h. The mixture was diluted with 10% of citric acid (50 mL) and the resulting mixture was extracted with ethyl acetate. The combined organic phase was washed with saturated sodium bicarbonate and brine, dried over sodium sulfate, filtered and concentrated at reduced pressure to give a residue, which was purified by prep-HPLC followed by lyophilization to give N-(5-chloro-8-quinolyl)-3-isopropyl-imidazole-4-sulfonamide (166.84 mg, 0.4670 mmol, 41.709% yield, 98.2% purity). ¹H NMR (400 MHz, DMSO-d₆) δ 10.65 (s, 1H), 8.96 (dd, J₁=4.2, J₂=1.6 Hz, 1H), 8.55 (dd, J₁=8.5, J₂=1.4 Hz, 1H), 8.07 (s, 1H), 7.79-7.73 (m, 2H), 7.72-7.66 (m, 1H), 7.37 (s, 1H), 5.04-4.92 (m, 1H), 1.34 (d, J=6.7 Hz, 6H); LCMS (ESI): m/z 351.1 [M+1]⁺.

### Example 33. 3-(Cyclopropylmethyl)-N-(5-morpholino-8-quinolyl)imidazole-4-sulfonamide

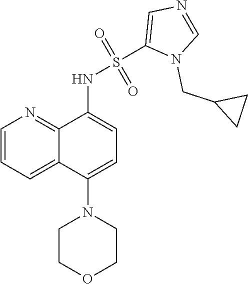 [SMILES: O=S(=O)(Nc1ccc(N2CCOCC2)c2cccnc12)c1cncn1CC1CC1]

1-(Cyclopropylmethyl)imidazole. To a solution of 1H-imidazole (20. g, 293.77 mmol) in ACN (250 mL) was added potassium carbonate (109.44 mL, 587.54 mmol), followed by bromomethylcyclopropane (51.56 g, 381.9 mmol). The mixture was stirred at 80° C. for 48 h. The reaction mixture was filtered and the filtrate was concentrated under reduced pressure to give a residue, which was purified by silica gel chromatography to give 1-(cyclopropylmethyl)imidazole (13 g, 106.41 mmol, 36.222% yield). ¹H NMR (400 MHz, CDCl₃) δ 7.32 (s, 1H), 6.84 (s, 1H), 6.78 (s, 1H), 3.57 (d, J=7.0 Hz, 2H), 1.06-0.91 (m, 1H), 0.52-0.39 (m, 2H), 0.19-0.07 (m, 2H).

[3-(Cyclopropylmethyl)imidazol-4-yl]sulfinyloxylithium. To a solution of 1-(cyclopropylmethyl)imidazole (13. g, 106.41 mmol) in diethyl ether (200 mL) was added n-butyllithium (55.52 mL, 127.69 mmol) slowly at −70° C. under nitrogen. The mixture was stirred at −70° C. for 30 min and 0° C. for 30 min. Excess sulfur dioxide was bubbled and the mixture was stirred at −70° C. for 1 h. Then the mixture was warmed to 25° C., which was filtered and the filtered cake was dried under vacuum to give the crude [3-(cyclopropylmethyl)imidazol-4-yl]sulfinyloxylithium (20 g, crude). (It contained [1-(cyclopropylmethyl)imidazol-4-yl]sulfinyloxylithium). It was used directly without further purification.

3-(Cyclopropylmethyl)imidazole-4-sulfonyl chloride. To a solution of [3-(cyclopropylmethyl)imidazol-4-yl]sulfinyloxylithium (20. g, crude) (contained [1-(cyclopropylmethyl)imidazol-4-yl]sulfinyloxylithium) in chloroform (100 mL) and water (100 mL) was added NCS (13.89 g, 104.08 mmol) at 0° C. The mixture was stirred at 0° C. for 1 h. The mixture was diluted with cold water and the aqueous phase was extracted with DCM. The combined organic phase was dried over anhydrous sodium sulfate, filtered and concentrated under vacuum. The crude product was purified by silica gel chromatography to give the crude 3-(cyclopropylmethyl)imidazole-4-sulfonyl chloride (1 g, crude). (it contained 1-(cyclopropylmethyl)imidazole-4-sulfonyl chloride). ¹H NMR (400 MHz, DMSO-d₆) δ 9.18 (d, J=1.5 Hz, 1H), 7.69 (d, J=1.6 Hz, 1H), 4.20 (d, J=7.5 Hz, 2H), 1.58-1.45 (m, 1H), 0.59-0.45 (m, 4H).

3-(Cyclopropylmethyl)-N-(5-morpholino-8-quinolyl)imidazole-4-sulfonamide. To a solution of 5-morpholinoquinolin-8-amine (220. mg, 0.9600 mmol) in Pyridine (4 mL) was added a solution of 3-(cyclopropylmethyl)imidazole-4-sulfonyl chloride (211.75 mg, 0.9600 mmol). (it contained 1-(cyclopropylmethyl)imidazole-4-sulfonyl chloride) in DCM (1 mL). The mixture was stirred at 25° C. for 16 hours. The mixture was diluted with 10% of citric acid (50 mL) and the resulting mixture was extracted with ethyl acetate. The combined organic phase was washed with saturated sodium bicarbonate and brine, dried over sodium sulfate, filtered and concentrated at reduced pressure to give a residue, which was purified by prep-HPLC followed by lyophilization to give 3-(cyclopropylmethyl)-N-(5-morpholino-8-quinolyl)imidazole-4-sulfonamide (42.14 mg, 0.0971 mmol, 10.122% yield, 95.3% purity). ¹H NMR (400 MHz, DMSO-d₆) δ 10.42-10.02 (m, 1H), 8.80 (dd, J₁=4.1, J₂=1.5 Hz, 1H), 8.49 (dd, J₁=8.5, J₂=1.5, Hz, 1H), 7.89 (s, 1H), 7.62 (d, J=8.1 Hz, 1H), 7.54 (dd, J₁=8.6, J₂=4.2, Hz, 1H), 7.23 (d, J=0.9 Hz, 1H), 7.18 (d, J=8.3 Hz, 1H), 4.12 (d, J=7.3 Hz, 2H), 3.88-3.80 (m, 4H), 3.03-2.92 (m, 4H), 1.42-1.29 (m, 1H), 0.57-0.48 (m, 2H), 0.38-0.34 (m, 2H); LCMS (ESI): m/z 414.2 [M+1]⁺

### Example 34. 1-(Cyclopropylmethyl)-N-(5-morpholino-8-quinolyl)imidazole-4-sulfonamide

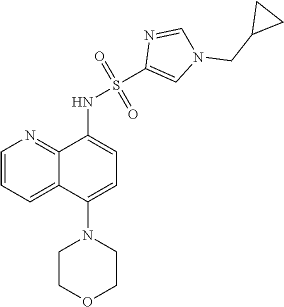 [SMILES: O=S(=O)(Nc1ccc(N2CCOCC2)c2cccnc12)c1cn(CC2CC2)cn1]

[1-(Cyclopropylmethyl)imidazol-4-yl]sulfinyloxylithium. To a solution of 1-(cyclopropylmethyl)imidazole (13 g, 106.41 mmol) in diethyl ether (200 mL) was added n-butyllithium (55.52 mL, 127.69 mmol) slowly at −70° C. under nitrogen. The mixture was stirred at −70° C. for 30 min and 0° C. for 30 min. Excess sulfur dioxide was bubbled and the mixture was stirred at −70° C. for 1 h. Then the mixture was warmed to 25° C., which was filtered and the filtered cake was dried under vacuum to give the crude [1-(cyclopropylmethyl)imidazol-4-yl]sulfinyloxylithium. (also contained [3-(cyclopropylmethyl)imidazol-4-yl]sulfinyloxylithium). It was used directly without further purification.

1-(Cyclopropylmethyl)imidazole-4-sulfonyl chloride. To a solution of [1-(cyclopropylmethyl)imidazol-4-yl]sulfinyloxylithium (20. g, crude) (contained [3-(cyclopropylmethyl)imidazol-4-yl]sulfinyloxylithium) in chloroform (100 mL) and water (100 mL) was added NCS (13.89 g, 104.08 mmol) at 0° C. The mixture was stirred at 0° C. for 1 h. The mixture was diluted with cold water and the aqueous phase was extracted with DCM. The combined organic phase was dried over anhydrous sodium sulfate, filtered and concentrated under vacuum. The crude product was purified by silica gel chromatography to give the crude 1-(cyclopropylmethyl)imidazole-4-sulfonyl chloride (1 g, crude). (also contained 3-(cyclopropylmethyl)imidazole-4-sulfonyl chloride). ¹H NMR (400 MHz, DMSO-d₆) δ 9.18 (d, J=1.5 Hz, 1H), 7.69 (d, J=1.6 Hz, 1H), 4.20 (d, J=7.5 Hz, 2H), 1.58-1.45 (m, 1H), 0.59-0.45 (m, 4H).

1-(Cyclopropylmethyl)-N-(5-morpholino-8-quinolyl)imidazole-4-sulfonamide. To a solution of 5-morpholinoquinolin-8-amine (220. mg, 0.9600 mmol) in Pyridine (4 mL) was added a solution of 1-(cyclopropylmethyl)imidazole-4-sulfonyl chloride (211.75 mg, 0.9600 mmol). (it contained 3-(cyclopropylmethyl)imidazole-4-sulfonyl chloride) in DCM (1 mL). The mixture was stirred at 25° C. for 16 hours. The mixture was diluted with 10% of citric acid (50 mL) and the resulting mixture was extracted with ethyl acetate. The combined organic phase was washed with saturated sodium bicarbonate and brine, dried over sodium sulfate, filtered, and concentrated at reduced pressure to give a residue, which was purified by prep-HPLC followed by lyophilization to give 1-(cyclopropylmethyl)-N-(5-morpholino-8-quinolyl)imidazole-4-sulfonamide (6.19 mg, 0.0143 mmol, 72.242% yield, 95.7% purity). ¹H NMR (400 MHz, MeOH-d₄) δ 8.80 (dd, J₁=4.1, J₂=1.6 Hz, 1H), 8.57 (dd, J₁=8.5, J₂=1.5 Hz, 1H), 7.80 (d, J=1.3 Hz, 1H), 7.76 (d, J=8.3 Hz, 1H), 7.63 (d, J=1.1 Hz, 1H), 7.51 (dd, J₁=8.5, J₂=4.2 Hz, 1H), 7.15 (d, J=8.3 Hz, 1H), 3.98-3.87 (m, 4H), 3.79 (d, J=7.3 Hz, 2H), 3.05-2.95 (m, 4H), 1.14-1.01 (m, 1H), 0.56-0.47 (m, 2H), 0.28-0.24 (m, 2H); LCMS (ESI): m/z 414.2 [M+1].

### Example 35. 1-Cyclopropyl-N-(5-morpholino-8-quinolyl)imidazole-2-sulfonamide

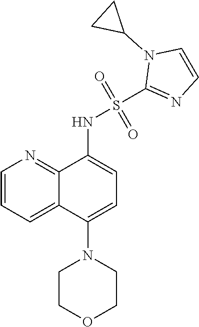 [SMILES: O=S(=O)(Nc1ccc(N2CCOCC2)c2cccnc12)c1nccn1C1CC1]

(1-Cyclopropylimidazol-2-yl)sulfinyloxylithium. To a solution of 1-cyclopropylimidazole (10. g, 92.47 mmol) in diethyl ether (150 mL) was added n-butyllithium (48.25 mL, 110.97 mmol) slowly at −70° C. under nitrogen. The mixture was stirred at −70° C. for 30 min and 0° C. for 30 min. Excess sulfur dioxide was bubbled and the mixture was stirred at −70° C. for 1 h. Then the mixture was warmed to 25° C., filtered, and the filtered cake was dried under vacuum to give the crude (1-cyclopropylimidazol-2-yl)sulfinyloxylithium (16 g, crude). (also containing (3-cyclopropylimidazol-4-yl)sulfinyloxylithium).

1-Cyclopropylimidazole-2-sulfonyl chloride. To a solution of (1-cyclopropylimidazol-2-yl)sulfinyloxylithium (16. g, crude) (contained (3-cyclopropylimidazol-4-yl)sulfinyloxylithium) in chloroform (100 mL) and water (100 mL) was added NCS (11.99 g, 89.82 mmol) at 0° C. The mixture was stirred at 0° C. for 1 h. The mixture was diluted with cold water and the aqueous phase was extracted with DCM. The combined organic phase was dried over anhydrous sodium sulfate, filtered and concentrated under vacuum. The crude product was purified by silica gel chromatography to give the crude 1-cyclopropylimidazole-2-sulfonyl chloride (1 g, crude). (contained 3-cyclopropylimidazole-4-sulfonyl chloride)

1-Cyclopropyl-N-(5-morpholino-8-quinolyl)imidazole-2-sulfonamide. To a solution of 5-morpholinoquinolin-8-amine (256.72 mg, 1.12 mmol) in pyridine (4 mL) was added a solution of 1-cyclopropylimidazole-2-sulfonyl chloride (277.66 mg, 1.34 mmol) (contained 3-cyclopropylimidazole-4-sulfonyl chloride) in DCM (1 mL). The mixture was stirred at 25° C. for 16 h. The mixture was diluted with 10% of citric acid (50 mL) and the resulting mixture was extracted with ethyl acetate. The combined organic phase was washed with saturated sodium bicarbonate and brine, dried over sodium sulfate, filtered and concentrated at reduced pressure to give a residue, which was purified by flash silica gel chromatography to give the crude product. It was further purified by prep-HPLC followed by lyophilization to give 1-cyclopropyl-N-(5-morpholino-8-quinolyl)imidazole-2-sulfonamide (23.42 mg, 0.0586 mmol, 5.2308% yield, 99.9% purity). ¹H NMR (400 MHz, Methanol-d₄) δ 8.86 (dd, J₁=4.4, J₂=1.5 Hz, 1H), 8.76 (dd, J₁=8.5, J₂=1.5 Hz, 1H), 7.79 (d, J=8.3 Hz, 1H), 7.64 (dd, J₁=8.5, J₂=4.4 Hz, 1H), 7.28 (s, 1H), 7.23 (d, J=8.3 Hz, 1H), 6.98 (s, 1H), 3.99-3.89 (m, 4H), 3.87-3.80 (m, 1H), 3.11-3.01 (m, 4H), 1.19-1.04 (m, 4H); LCMS (ESI): m/z 400.2 [M+1]⁺.

### Example 36. 3-Methyl-N-(2-methylisoindolin-4-yl)pyridine-2-sulfonamide

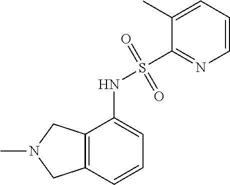 [SMILES: Cc1cccnc1S(=O)(=O)Nc1cccc2c1CN(C)C2]

2-Methyl-4-nitroisoindoline. To a solution of 1,2-bis(bromomethyl)-3-nitro-benzene (2.00 g, 6.47 mmol) and methylamine (2 M, 3.6 mL) was added TEA (1.44 g, 14.24 mmol). The mixture was stirred at 25° C. for 0.5 h under N₂. Methylamine (2 M, 15.0 mL) was added to the above mixture. The mixture was stirred at 25° C. for another 1 h under N₂. The mixture was concentrated in vacuo. The residue was poured into water and the aqueous phase was extracted with ethyl acetate. The combined organic phase was washed with brine, dried over anhydrous sodium sulfate, filtered and concentrated under vacuum. The residue was purified by column chromatography to give 2-methyl-4-nitro-isoindoline (200 mg, 1.05 mmol, 16% yield, 93.3% purity). MS (ESI): m/z 179.2 [M+1]⁺.

2-Methylisoindolin-4-amine. To a solution of 2-methyl-4-nitro-isoindoline (200 mg, 1.12 mmol) in 2,2,2-trifluoroethanol (10 mL) was added Pd/C (40 mg). The mixture was stirred at 45° C. for 3 h under H₂ (15 psi). The reaction mixture was filtered and the filtrate was concentrated in vacuo to give 2-methylisoindolin-4-amine (150 mg, crude). MS (ESI): m/z 149.0 [M+1]⁺.

3-Methyl-N-(2-methylisoindolin-4-yl)pyridine-2-sulfonamide. To a solution of 2-methylisoindolin-4-amine (143 mg, 0.966 mmol) in pyridine (3.83 g, 48.40 mmol) was added a mixture of 3-methylpyridine-2-sulfonyl chloride (400 mg, 1.61 mmol) in DCM (5 mL) at 0° C. under N₂. The mixture was stirred at 25° C. for 3 h under N₂. The mixture was concentrated. The residue was diluted with brine and the aqueous phase was extracted with DCM. The combined organic phase was washed with sodium bicarbonate solution, dried over anhydrous sodium sulfate, filtered and the filtrate was concentrated under reduced pressure to give the crude product. The crude product was purified by prep-HPLC to give 3-methyl-N-(2-methyl isoindolin-4-yl)pyridine-2-sulfonamide (120.74 mg, 0.330 mmol, 34% yield, 95.5% purity, FA). H NMR (400 MHz, CD3OD-d₄) δ 8.50-8.39 (m, 2H), 7.82 (dd, J₁=7.8, J₂=0.7 Hz, 1H), 7.48 (dd, J₁=7.7, J₂=4.6 Hz, 1H), 7.29-7.20 (m, 2H), 7.16 (d, J=6.6 Hz, 1H), 4.65 (s, 2H), 4.46 (s, 2H), 2.97 (s, 3H), 2.59 (s, 3H); MS (ESI): m/z 304.1 [M+1]⁺.

### Example 37. 3-Fluoro-N-(1,2,3,4-tetrahydroquinolin-8-yl)pyridine-2-sulfonamide

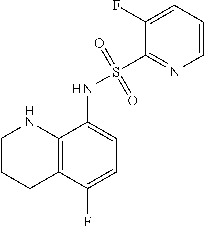 [SMILES: O=S(=O)(Nc1ccc(F)c2c1NCCC2)c1ncccc1F]

lithium 3-fluoropyridine-2-sulfinate. To a solution of 2-bromo-3-fluoropyridine (4.8 g, 27.27 mmol) in diethyl ether (100 mL) was added n-BuLi (2.5 M, 13.09 mL) at −70° C. The mixture was stirred at −70° C. for 0.5 h. Excess sulfur dioxide was bubbled and the mixture was stirred at −70° C. for 1 h. Then the mixture was warmed to 25° C. The mixture was filtered and the filter cake was collected and dried under vacuum to give lithium 3-fluoropyridine-2-sulfinate (4 g, crude).

3-Fluoropyridine-2-sulfonyl chloride. To a solution of lithium 3-fluoropyridine-2-sulfinate (4 g) in chloroform (50 mL) and water (50 mL) was added NCS (4.79 g, 35.87 mmol) in portions at 0° C. The mixture was stirred at 0° C. for 1 h. The mixture was diluted with cold water and the aqueous phase was extracted with chloroform. The combined organic phase was dried over anhydrous sodium sulfate, filtered and concentrated under vacuum to obtain 3-fluoropyridine-2-sulfonyl chloride (3 g, 13.19 mmol, 86% purity).

1,2,3,4-Tetrahydroquinolin-8-amine. To a solution of quinolin-8-amine (10 g, 69.36 mmol) in acetic acid (50 mL) was added platinum dioxide (0.4 g, 1.76 mmol) under nitrogen. The suspension was degassed under vacuum and purged with hydrogen several times. The mixture was stirred under hydrogen (20 psi) at 30° C. for 40 h. The mixture was filtered and the filtrate was concentrated at reduced pressure to give a residue. The residue was purified by silica gel chromatography to give 1,2,3,4-tetrahydroquinolin-8-amine (9.4 g, 63.43 mmol, 91.44% yield). ¹H NMR (400 MHz, DMSO-d₆) δ 6.34 (dd, J₁=6.0, J₂=1.2 Hz, 1H), 6.80 (t, J=7.6 Hz, 1H), 6.21 (d, J=7.2 Hz, 1H), 4.60 (brs, 2H), 3.20 (t, J=5.6 Hz, 2H), 2.62 (t, J=6.0 Hz, 2H), 1.79-1.73 (m, 2H).

3-Fluoro-N-(1,2,3,4-tetrahydroquinolin-8-yl)pyridine-2-sulfonamide. To a solution of 1,2,3,4-tetrahydroquinolin-8-amine (1.30 g, 8.79 mmol) in pyridine (10 mL) was added 3-fluoropyridine-2-sulfonyl chloride (2 g, 8.79 mmol) which was dissolved DCM (5 mL) slowly at 0° C. under nitrogen. The mixture was stirred at 25° C. for 16 h. The mixture was concentrated at reduced pressure to give a residue and the residue was diluted with water. The mixture was extracted with ethyl acetate and combined organic phase was washed with brine, dried over sodium sulfate, filtered and concentrated at reduced pressure to give a residue. The residue was purified by silica gel chromatography to give 3-fluoro-N-(1,2,3,4-tetrahydroquinolin-8-yl)pyridine-2-sulfonamide (1.6 g, crude). The crude product was triturated with MeOH for 30 min, then the mixture was filtered and the filtered cake was triturated with water which contained 20% of ACN for 10 minutes. The mixture was lyophilizated to give 3-fluoro-N-(1,2,3,4-tetrahydroquinolin-8-yl)pyridine-2-sulfonamide (150.21 mg, 482.87 umol, 14.84% yield, 98.8% purity). ¹H NMR (400 MHz, DMSO-d₆) δ 9.77 (s, 1H), 8.57 (d, J=4.8, 1H), 8.03-7.98 (m, 1H), 7.82-7.79 (m, 1H), 6.74 (d, J=7.6 Hz, 1H), 6.67 (d, J=7.6 Hz, 1H), 6.31 (t, J=7.6 Hz, 1H), 5.49 (s, 1H), 3.19 (t, J=5.6 Hz, 2H), 2.62 (t, J=6.0 Hz, 2H), 1.74-1.68 (m, 2H); MS (ESI): m/z 308.1 [M+1]⁺.

### Example 38. 3-Methyl-N-(1-methylindolin-7-yl)pyridine-2-sulfonamide

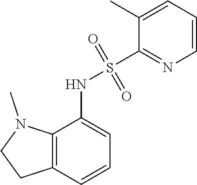 [SMILES: Cc1cccnc1S(=O)(=O)Nc1cccc2c1N(C)CC2]

Lithium 3-methylpyridine-2-sulfinate. To a solution of 2-bromo-3-methylpyridine (10 g, 58.13 mmol) in THF (100 mL) was added n-BuLi (2.5 M, 27.90 mL) slowly at −70° C. under nitrogen. The mixture was stirred at −70° C. for 1 h. Then excess sulfur dioxide was purged and the mixture was stirred at −70° C. for 0.5 h. Then the mixture was warmed to 20° C. The mixture was filtered and the filter cake was collected and dried under vacuum to give lithium 3-methylpyridine-2-sulfinate (12 g, crude).

3-Methylpyridine-2-sulfonyl chloride. To a mixture of lithium 3-methylpyridine-2-sulfinate (12 g, 1 eq) in chloroform (50 mL) and water (50 mL) was added NCS (14.73 g, 110.31 mmol) in portions at 0° C. The mixture was stirred at 0° C. for 1 h. The mixture was separated and the organic phase was dried over sodium sulfate, filtered and concentrated at reduced pressure to give 3-methylpyridine-2-sulfonyl chloride (6 g, crude).

1-Methyl-7-nitroindoline. To a solution of 1-methyl-7-nitro-1H-indole (5 g, 28.38 mmol) in acetic acid (50 mL) was added sodium cyanoborohydride (7.13 g, 113.53 mmol) at 25° C. in portions. The mixture was stirred at 70° C. for 48 h. The mixture was concentrated and the residue was diluted with water. The aqueous phase was adjusted to pH 8 using sodium hydroxide (2 M) and extracted with DCM. The combined organic layers were concentrated under reduced pressure to give 1-methyl-7-nitroindoline (7.07 g, crude).

1-Methylindolin-7-amine. To a solution of 1-methyl-7-nitroindoline (6.5 g, 1 eq) in MeOH (100 mL) was added Pd/C (600 mg, 10% purity) and Pd(OH)₂/C (600 mg, 4.27 mmol) under nitrogen. Then the mixture was stirred at 25° C. for 4 h under hydrogen (15 psi). The reaction mixture was filtered and the filtrate was concentrated under reduced pressure to give a residue. The residue was purified by prep-HPLC to afford 1-methylindol-7-amine (2.16 g, 14.34 mmol, 97% purity). ¹H NMR (400 MHz, DMSO-d₆) δ 6.54-6.50 (m, 1H), 6.44-6.42 (m, 2H), 4.39 (brs, 2H), 3.17 (t, J=8.4 Hz, 1H), 2.81 (t, J=8.0 Hz, 2H), 2.74 (s, 3H); compound 1-methylindolin-7-amine (0.72 g, 4.62 mmol, 95% purity) was obtained as red oil. ¹H NMR (400 MHz, DMSO-d₆) δ 7.05 (d, J=3.2 Hz, 1H), 6.82 (dd, J₁=7.6, J₂=0.4 Hz, 1H), 6.80 (d, J=7.6 Hz, 1H), 6.37 (d, J=7.2 Hz, 1H), 6.21 (d, J=3.2 Hz, 1H), 4.82 (s, 2H), 4.05 (s, 3H).

N-(1-Methylindolin-7-yl)acetamide. To a solution of 1-methylindol-7-amine (1.86 g, 12.31 mmol) in acetic acid (30 mL) was added sodium cyanoborohydride (3.87 g, 61.56 mmol) in portions. The mixture was stirred at 60° C. for 48 h. The mixture was concentrated and the residue was poured into water. The aqueous phase was adjusted to pH 9 and the mixture was extracted with DCM. The combined organic phase was dried over anhydrous sodium sulfate filtered and concentrated under vacuum. The residue was purified by silica gel chromatography to give N-(1-methylindolin-7-yl) acetamide (1.1 g, 5.78 mmol, 46.96% yield).

1-Methylindolin-7-amine. To a mixture of N-(1-methylindolin-7-yl)acetamide (0.9 g, 4.73 mmol) in MeOH (15 mL) was added thionyl chloride (450.26 mg, 3.78 mmol, 274.55 uL) under nitrogen. The mixture was stirred at 75° C. for 3 h. The mixture was concentrated to give a residue and saturated sodium carbonate was added to the residue and extracted with ethyl acetate. The combined organic phase was dried over sodium sulfate, filtered and concentrated at reduced pressure to give 1-methylindolin-7-amine (0.7 g). ¹H NMR (400 MHz, CDCl₃) δ 6.72-6.68 (m, 2H), 6.53-6.51 (m, 1H), 3.34 (t, J=8.4 Hz, 2H), 2.98 (t, J=8.0 Hz, 2H), 2.86 (s, 3H).

3-methyl-N-(1-methylindolin-7-yl)pyridine-2-sulfonamide. To a mixture of 1-methylindolin-7-amine (0.65 g, 4.39 mmol) in pyridine (10 mL) was added 3-methylpyridine-2-sulfonyl chloride (840.48 mg) in DCM (10 mL) slowly at 0° C. under nitrogen. The mixture was stirred at 25° C. for 16 h. The mixture was concentrated at reduced pressure to give a residue. The residue was purified by prep-HPLC to give 3-methyl-N-(1-methylindolin-7-yl)pyridine-2-sulfonamide (430.07 mg, 1.36 mmol, 31.03% yield, 96% purity). ¹H NMR (400 MHz, DMSO-d₆) δ 8.57 (d, J=3.2 Hz, 1H), 7.86 (d, J=6.8 Hz, 1H), 7.57 (dd, J₁=7.6, J₂=4.6 Hz, 1H), 6.88 (dd, J₁=7.0, J₂=0.8 Hz, 1H), 6.49 (d, J=8.0 Hz, 1H), 6.34 (t, J=7.2 Hz, 1H), 3.26 (t, J=8.8 Hz, 2H), 3.01 (s, 3H), 2.82 (t, J=8.4 Hz, 2H), 2.45 (s, 3H); MS (ESI): m/z 304.1 [M+1]⁺.

### Example 39. 3-Methyl-N-(2-methyl-1,2,3,4-tetrahydroisoquinolin-8-yl)pyridine-2-sulfonamide

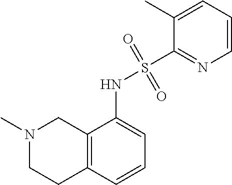 [SMILES: Cc1cccnc1S(=O)(=O)Nc1cccc2c1CN(C)CC2]

tert-Butyl 8-(3-methylpyridine-2-sulfonamido)-3,4-dihydroisoquinoline-2(1H)-carboxylate. To a solution of tert-butyl 8-amino-3,4-dihydroisoquinoline-2(1H)-carboxylate (1.2 g, 4.83 mmol) in pyridine (20 mL) was added 3-methylpyridine-2-sulfonyl chloride (926 mg) which was dissolved with DCM (10 mL). The mixture was stirred at 25° C. 16 h. The mixture was diluted with water and extracted with ethyl acetate. The combined organic phase was washed with brine, dried over sodium sulfate, filtered and concentrated at reduced pressure to give a residue. The residue was purified by silica gel chromatography to give tert-butyl 8-[(3-methyl-2-pyridyl)sulfonylamino]-3,4-dihydro-1H-isoquinoline-2-carboxylate (1.7 g).

3-Methyl-N-(1,2,3,4-tetrahydroisoquinolin-8-yl)pyridine-2-sulfonamide. To a solution of tert-butyl 8-[(3-methyl-2-pyridyl)sulfonylamino]-3,4-dihydro-1H-isoquinoline-2-carboxylate (0.9 g, 2.23 mmol) in DCM (15 mL) was added trifluoromethanesulfonic acid (4.62 g, 40.52 mmol, 3 mL) slowly at 0° C. under nitrogen. The mixture was stirred at 0° C. for 1 h. The mixture was concentrated at reduced pressure to give 3-methyl-N-(1,2,3,4-tetrahydroisoquinolin-8-yl)pyridine-2-sulfonamide (1 g, crude, trifluoromethanesulfonic acid salt).

3-Methyl-N-(2-methyl-1,2,3,4-tetrahydroisoquinolin-8-yl)pyridine-2-sulfonamide. To a mixture of 3-methyl-N-(1,2,3,4-tetrahydroisoquinolin-8-yl)pyridine-2-sulfonamide (1 g, crude, trifluoromethanesulfonic acid salt) in MeOH (10 mL) was added triethylamine (400 mg, 3.96 mmol, 550.55 uL). Then acetic acid (59 mg, 0.989 mmol) and formaldehyde (2.67 g, 32.96 mmol, 37% purity) were added. The mixture was stirred at 20° C. for 2 h. Then the mixture was cooled to 0° C. and sodium cyanoborohydride (621.40 mg, 9.89 mmol) was added in portions. The mixture was stirred at 25° C. for 14 h. The mixture was concentrated at reduced pressure to give a residue and saturated sodium carbonate was added. The mixture was extracted with ethyl acetate and the combined organic phase was dried over sodium sulfate, filtered and concentrated at reduced pressure to give a residue. The residue was purified by prep-HPLC to give 3-methyl-N-(2-methyl-3,4-dihydro-1H-isoquinolin-8-yl)pyridine-2-sulfonamide (556.67 mg, 1.70 mmol, 97% purity). ¹H NMR (400 MHz, DMSO-d₆) δ 8.52 (d, J=4.0 Hz, 1H), 7.87 (d, J=7.2 Hz, 1H), 7.56 (dd, J₁=7.8, J₂=4.4 Hz, 1H), 7.00-6.94 (m, 1H), 6.92 (d, J=7.2 Hz, 1H), 6.85 (d, J=7.6 Hz, 1H), 3.49 (s, 3H), 2.79 (t, J=5.6 Hz, 2H), 2.53 (t, J=5.6 Hz, 2H), 2.48 (s, 2H), 2.33 (s, 3H); MS (ESI): m/z 318.1 [M+1]⁺.

### Example 40. N-(1-Cyclopropyl-1,2,3,4-tetrahydroquinolin-8-yl)-3-(dimethylamino)pyridine-2-sulfonamide

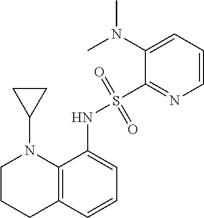 [SMILES: CN(C)c1cccnc1S(=O)(=O)Nc1cccc2c1N(C1CC1)CCC2]

N-(1-Cyclopropyl-1,2,3,4-tetrahydroquinolin-8-yl)-3-(dimethylamino)pyridine-2-sulfonamide. To a solution of 3-(dimethylamino)-N-(1,2,3,4-tetrahydroquinolin-8-yl)pyridine-2-sulfonamide (0.7 g, 2.11 mmol) and (1-ethoxycyclopropoxy)trimethylsilane (1.84 g, 10.53 mmol) in MeOH (20 mL) and acetic acid (20 mL) was added sodium cyanoborohydride (662 mg, 10.53 mmol). The mixture was stirred at 25° C. for 32 h. The mixture was concentrated. The residue was poured into sodium bicarbonate and the aqueous phase was extracted with ethyl acetate. The combined organic phase dried with anhydrous sodium sulfate, filtered and concentrated under vacuum. The residue was triturated with MeOH. The filter cake and filtrate were combined followed by concentration to give the crude product. The crude product was purified by prep-HPLC followed by lyophilization to give N-(1-cyclopropyl-1,2,3,4-tetrahydroquinolin-8-yl)-3-(dimethylamino)pyridine-2-sulfonamide (414.09 mg, 1.06 mmol, 50.42% yield, 95.5% purity). ¹H NMR (400 MHz, DMSO-d₆) 8.78 (br s, 1H), 8.21 (dd, J₁=4.3, J₂=1.1 Hz, 1H), 7.77 (dd, J=8.3, 12=1.1 Hz, 1H), 7.51 (dd, J=8.3, 12=4.3 Hz, 1H), 6.83 (d, J=7.0 Hz, 1H), 6.70-6.65 (m, 1H), 6.62-6.55 (m, 1H), 3.10 (t, J=6.1 Hz, 2H), 2.76 (s, 6H), 2.72-2.65 (m, 1H), 2.59 (t, J=6.7 Hz, 2H), 1.83 (q, J=6.4 Hz, 2H), 0.69-0.53 (m, 4H); MS (ESI): m/z 373.2 [M+1]⁺.

### Example 41. 1-Isopropyl-N-(2-methyl-1,2,3,4-tetrahydroisoquinolin-8-yl)-1H-pyrazole-5-sulfonamide

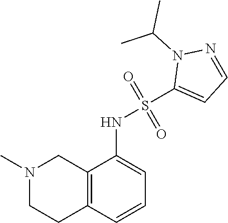 [SMILES: CC(C)n1nccc1S(=O)(=O)Nc1cccc2c1CN(C)CC2]

1,2,3,4-Tetrahydroisoquinolin-8-amine. To a solution of isoquinolin-8-amine (5 g, 34.68 mmol) in acetic acid (30 mL) was added platinum dioxide (0.2 g, 0.881 mmol) under nitrogen. The suspension was degassed under vacuum and purged with hydrogen several times. The mixture was stirred under hydrogen (50 psi) at 25° C. for 16 h. The mixture was filtered and the filtrate was concentrated at reduced pressure to give 1,2,3,4-tetrahydroisoquinolin-8-amine (10 g, crude, acetic acid). ¹H NMR (400 MHz, DMSO-d₆) δ 6.91 (t, J=7.7 Hz, 1H), 6.52 (d, J=7.9 Hz, 1H), 6.37 (d, J=7.4 Hz, 1H), 3.86 (s, 2H), 3.17 (t, J=5.4 Hz, 2H), 2.82 (t, J=5.5 Hz, 2H), 1.82 (s, 3H).

tert-Butyl 8-amino-3,4-dihydroisoquinoline-2(1H)-carboxylate. To a mixture of 1,2,3,4-tetrahydroisoquinolin-8-amine (9.3 g, crude, acetic acid) and sodium bicarbonate (15.01 g, 178.67 mmol) in water (50 mL) and THE (50 mL) was added di-tert-butyl dicarbonate (9.75 g, 44.67 mmol, 10.26 mL) slowly at 0° C. under nitrogen. The mixture was stirred at 15° C. for 2 h. The mixture was extracted with ethyl acetate and the combined organic phase was washed with brine, dried over sodium sulfate, filtered and concentrated at reduced pressure to give a residue. The residue was purified by silica gel chromatography to give tert-butyl 8-amino-3,4-dihydroisoquinoline-2(1H)-carboxylate (5.9 g, 23.76 mmol). ¹H NMR (400 MHz, CDCl₃) δ 7.01 (t, J=7.7 Hz, 1H), 6.66-6.51 (m, 2H), 4.36 (s, 2H), 3.71-3.47 (m, 4H), 2.81 (s, 2H), 1.52 (s, 9H).

tert-Butyl 8-(1-isopropyl-1H-pyrazole-5-sulfonamido)-3,4-dihydroisoquinoline-2(1H)-carboxylate. To a solution of tert-butyl 8-amino-3,4-dihydroisoquinoline-2(1H)-carboxylate (700 mg, 2.82 mmol) in pyridine (8 mL) was added 1-isopropyl-1H-pyrazole-5-sulfonyl chloride (588 mg, 2.82 mmol) which was dissolved with DCM (2 mL) slowly at 0° C. under nitrogen. The mixture was stirred at 15° C. for 16 h. The mixture was diluted with 10% citric acid and the resulting mixture was extracted with ethyl acetate. The combined organic phase was washed with saturated sodium bicarbonate and brine, dried over sodium sulfate, filtered and concentrated at reduced pressure to give a residue. The residue was purified by silica gel chromatography to give tert-butyl 8-(1-isopropyl-1H-pyrazole-5-sulfonamido)-3,4-dihydroisoquinoline-2(1H)-carboxylate (1.2 g, crude).

tert-Butyl 8-(1-isopropyl-1H-pyrazole-5-sulfonamido)-3,4-dihydroisoquinoline-2(1H)-carboxylate. To a solution of 8-(1-isopropyl-1H-pyrazole-5-sulfonamido)-3,4-dihydroisoquinoline-2(1H)-carboxylate (1.2 g, crude) in DCM (10 mL) was added trifluoroacetic acid (3.08 g, 27.01 mmol, 2.00 mL) slowly at 0° C. under nitrogen. The mixture was stirred at 0° C. for 0.5 h. The mixture was concentrated at reduced pressure to give tert-butyl 8-(1-isopropyl-1H-pyrazole-5-sulfonamido)-3,4-dihydroisoquinoline-2(1H)-carboxylate (1.2 g, crude, trifluroroacetic acid).

1-Isopropyl-N-(2-methyl-1,2,3,4-tetrahydroisoquinolin-8-yl)-1H-pyrazole-5-sulfonamide. To a mixture of tert-butyl 8-(1-isopropyl-1H-pyrazole-5-sulfonamido)-3,4-dihydroisoquinoline-2(1H)-carboxylate (1.2 g, crude, trifluroroacetic acid) and formaldehyde (2.24 g, 27.60 mmol, 12.32 mL, 37% purity) in MeOH (20 mL) was added TEA (307.46 mg, 3.04 mmol) and acetic acid (24.88 mg, 0.414 mmol). The mixture was stirred at 15° C. for 30 min, then sodium cyanoborohydride (520.75 mg, 8.29 mmol) was added in portions and the mixture was stirred at 15° C. for 2 h. The mixture was concentrated at reduced pressure to give a residue. The residue was diluted with saturated sodium bicarbonate and the resulting mixture was extracted with ethyl acetate. The combined organic phase was dried over sodium sulfate, filtered and concentrated at reduced pressure to give a residue. The residue was purified by prep-HPLC to give 1-isopropyl-N-(2-methyl-1,2,3,4-tetrahydroisoquinolin-8-yl)-1H-pyrazole-5-sulfonamide (177.35 mg, 0.509 mmol, 96% purity). ¹H NMR (400 MHz, DMSO-d₆) δ 8.14 (s, 1H), 7.47 (d, J=1.8 Hz, 1H), 7.02-6.98 (m, 1H), 6.78 (t, J=8.2 Hz, 2H), 6.47 (d, J=1.8 Hz, 1H), 5.15-5.05 (m, 1H), 3.80 (s, 2H), 3.03-3.00 (m, 2H), 2.91-2.88 (m, 2H), 2.67 (s, 3H), 1.31 (d, J=6.6 Hz, 6H); MS (ESI): m/z 335.3 [M+1]⁺.

### Example 42. N-(1-Cyclopropyl-1,2,3,4-tetrahydroquinolin-8-yl)-3-methylpyridine-2-sulfonamide

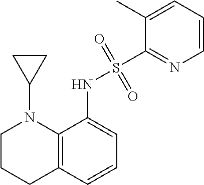 [SMILES: Cc1cccnc1S(=O)(=O)Nc1cccc2c1N(C1CC1)CCC2]

3-Methyl-N-(1,2,3,4-tetrahydroquinolin-8-yl)pyridine-2-sulfonamide. To a solution of 1,2,3,4-tetrahydroquinolin-8-amine (773.37 mg, 5.22 mmol) in pyridine (8 mL) was added 3-methylpyridine-2-sulfonyl chloride (1 g, 5.22 mmol) which was dissolved with DCM (5 mL) slowly at 0° C. under nitrogen. The mixture was stirred at 15° C. for 16 h. The mixture was diluted with 10% citric acid and the resulting mixture was extracted with ethyl acetate. The combined organic phase was washed with saturated sodium bicarbonate and brine, dried over sodium sulfate, filtered and concentrated at reduced pressure to give a residue. The residue was purified by silica gel chromatography to give 3-methyl-N-(1,2,3,4-tetrahydroquinolin-8-yl)pyridine-2-sulfonamide (1 g, 3.30 mmol, 63.17% yield).

N-(1-Cyclopropyl-1,2,3,4-tetrahydroquinolin-8-yl)-3-methylpyridine-2-sulfonamide. To a mixture of 3-methyl-N-(1,2,3,4-tetrahydroquinolin-8-yl)pyridine-2-sulfonamide (1 g, 3.30 mmol) and (1-ethoxycyclopropoxy)trimethylsilane (1.72 g, 9.89 mmol) in MeOH (20 mL), DCM (5 mL) and acetic acid (20 mL) was added sodium cyanoborohydride (621.40 mg, 9.89 mmol) in portions at 0° C. under nitrogen. The mixture was stirred at 40° C. for 16 h. The mixture was concentrated at reduced pressure to give a residue and the residue was diluted with ethyl acetate and the resulting mixture was washed with saturated sodium bicarbonate and brine, dried over sodium sulfate, filtered and concentrated at reduced pressure to give a residue. The residue was purified by silica gel chromatography followed by prep-HPLC to give N-(1-cyclopropyl-1,2,3,4-tetrahydroquinolin-8-yl)-3-methylpyridine-2-sulfonamide (203.15 mg, 0.562 mmol, 17.05% yield, 95% purity). ¹H NMR (400 MHz, DMSO-d₆) δ 9.03 (s, 1H), 8.52 (dd, J₁=4.5, J₂=0.9 Hz, 1H), 7.83 (d, J=7.7 Hz, 1H), 7.54 (dd, J₁=7.7, J₂=4.5 Hz, 1H), 6.75 (dd, J₁=7.6, J₂=2.8 Hz, 2H), 6.55-6.51 (m, 1H), 3.04 (t, J=6.4 Hz, 2H), 2.85-2.80 (m, 1H), 2.38 (s, 3H), 1.77-1.70 (m, 2H), 0.71-0.66 (m, 2H), 0.48-0.45 (m, 2H); MS (ESI): m/z 344.1 [M+1]⁺.

### Example 43. N-(2-Cyclopropylisoindolin-4-yl)-1-isopropyl-1H-imidazole-2-sulfonamide

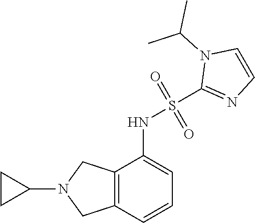 [SMILES: CC(C)n1ccnc1S(=O)(=O)Nc1cccc2c1CN(C1CC1)C2]

1,2-bis(Bromomethyl)-3-nitrobenzene. To a solution of 1,2-dimethyl-3-nitrobenzene (9.5 g, 62.85 mmol) in tetrachloromethane (50 mL) were added benzoyl peroxide (456.69 mg, 1.89 mmol) and 1-bromopyrrolidine-2,5-dione (22.37 g, 125.69 mmol) at 10° C. The reaction mixture was stirred at 80° C. for 16 h. The reaction mixture was filtered and the filtrate was concentrated to give 1,2-bis(bromomethyl)-3-nitrobenzene (18 g, crude).

2-Cyclopropyl-4-nitroisoindoline. To a solution of 1,2-bis(bromomethyl)-3-nitro-benzene (18 g, 58.26 mmol) and TEA (11.79 g, 116.52 mmol, 16.22 mL) in THF (80 mL) was added a mixture of cyclopropanamine (3.33 g, 58.26 mmol, 4.04 mL) in THF (20 mL). The reaction mixture was stirred at 10° C. for 2 hours. The reaction mixture was concentrated to give crude product. The crude was purified by silica gel chromatography twice to give 2-cyclopropyl-4-nitroisoindoline (1.5 g, crude). ¹H NMR (400 MHz, DMSO-d₆) 8.04 (d, J=7.9 Hz, 1H), 7.69 (d, J=7.4 Hz, 1H), 7.51 (t, J=7.8 Hz, 1H), 4.40 (s, 2H), 4.09 (s, 2H), 2.15-2.07 (m, 1H), 0.52-0.46 (m, 2H), 0.46-0.41 (m, 2H); MS (ESI): m/z 205.0 [M+1]⁺.

2-Cyclopropylisoindolin-4-amine. To a solution of 2-cyclopropyl-4-nitroisoindoline (1.4 g, crude) in MeOH (20 mL) was added palladium/carbon (500 mg, 10% purity). The suspension was degassed under vacuum and purged with hydrogen three times and stirred at 45° C. for 1 hour under hydrogen (15 psi). The reaction mixture was filtered and the filtrate was concentrated to give residue. The residue was purified by silica gel chromatography to give 2-cyclopropylisoindolin-4-amine (0.7 g, 3.62 mmol, 52.74% yield, 90% purity). ¹H NMR (400 MHz, CDCl₃) 7.03 (t, J=7.7 Hz, 1H), 6.65 (d, J=7.4 Hz, 1H), 6.53 (d, J=7.9 Hz, 1H), 4.07 (s, 2H), 3.97 (s, 2H), 3.52 (s, 2H), 2.10-2.03 (m, 1H), 0.55-0.50 (m, 4H); MS (ESI): m/z 175.1 [M+1]⁺.

N-(2-Cyclopropylisoindolin-4-yl)-1-isopropyl-1H-imidazole-2-sulfonamide.

To a mixture of 2-cyclopropylisoindolin-4-amine (0.35 g, 1.81 mmol) in pyridine (5 mL) was added a solution of 1-isopropyl-1H-imidazole-2-sulfonyl chloride (452.68 mg, 2.17 mmol) in DCM (3 mL) drop-wise at 0° C. The mixture was stirred at 20° C. for 16 h. The reaction mixture was concentrated and the residue was diluted DCM, washed with 10% citric acid, saturated sodium bicarbonate and brine, dried over anhydrous sodium sulfate, filtered and concentrated in vacuum to give a crude product. The crude product was triturated with MeOH followed by lyophilization to give N-(2-cyclopropylisoindolin-4-yl)-1-isopropyl-1H-imidazole-2-sulfonamide (150.66 mg, 0.426 mmol, 23.57% yield, 98% purity). ¹H NMR (400 MHz, DMSO-d₆) 10.26 (br s, 1H), 7.62 (s, 1H), 7.15-7.08 (m, 2H), 7.07-6.97 (m, 2H), 4.80-4.87 (m, 1H), 3.90 (s, 2H), 3.77 (s, 2H), 1.93-1.97 (m, 1H), 1.25 (d, J=6.6 Hz, 6H), 0.47-0.41 (m, 2H), 0.37-0.31 (m, 2H); MS (ESI): m/z 347.1 [M+1]⁺.

### Example 44. N-(7-Chloro-2-methyl-1,2,3,4-tetrahydroisoquinolin-8-yl)-3-methylpyridine-2-sulfonamide

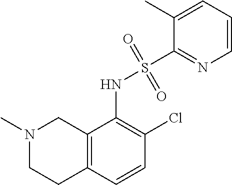 [SMILES: Cc1cccnc1S(=O)(=O)Nc1c(Cl)ccc2c1CN(C)CC2]

tert-Butyl 8-amino-5-bromo-3,4-dihydroisoquinoline-2(1H)-carboxylate. To a solution of tert-butyl 8-amino-3,4-dihydroisoquinoline-2(1H)-carboxylate (1 g, 4.03 mmol) in DMF (10 mL) was added N-bromosuccinimide (716.75 mg, 4.03 mmol) which was dissolved with DMF (3 mL) slowly at 0° C. under nitrogen. The mixture was stirred at 15° C. for 2 h. The mixture was diluted with water and the resulting mixture was extracted with ethyl acetate. The combined organic phase was washed with brine, dried over sodium sulfate, filtered and concentrated at reduced pressure to give a residue. The residue was purified by silica gel chromatography to give tert-butyl 8-amino-5-bromo-3,4-dihydroisoquinoline-2(1H)-carboxylate (1.2 g, 3.67 mmol, 91.07% yield). ¹H NMR (400 MHz, CDCl₃) δ 7.24 (d, J=8.4 Hz, 1H), 6.48 (d, J=8.4 Hz, 1H), 4.33 (s, 2H), 3.64 (t, J=5.7 Hz, 2H), 3.57 (s, 2H), 2.81 (t, J=5.6 Hz, 2H), 1.51 (s, 9H).

tert-Butyl 8-amino-5-bromo-7-chloro-3,4-dihydroisoquinoline-2(1H)-carboxylate. A mixture of tert-butyl 8-amino-5-bromo-3,4-dihydroisoquinoline-2(1H)-carboxylate (1.2 g, 3.67 mmol) and N-chlorosuccinimide (538.68 mg, 4.03 mmol) in DMF (15 mL) was stirred at 85° C. for 3 h. The mixture was diluted with water and the resulting mixture was extracted with ethyl acetate. The combined organic phase was washed with brine, dried over sodium sulfate, filtered and concentrated at reduced pressure to give a residue. The residue was purified by silica gel chromatography to give tert-butyl 8-amino-5-bromo-7-chloro-3,4-dihydroisoquinoline-2(1H)-carboxylate (1 g, 2.77 mmol, 75.40% yield). 1H NMR (400 MHz, CDCl₃) δ 7.40 (s, 1H), 4.34 (s, 2H), 3.98 (s, 2H), 3.63 (t, J=5.6 Hz, 2H), 2.78 (t, J=5.7 Hz, 2H), 1.50 (s, 9H).

tert-Butyl 8-amino-7-chloro-3,4-dihydroisoquinoline-2(1H)-carboxylate. A mixture of tert-butyl 8-amino-5-bromo-7-chloro-3,4-dihydroisoquinoline-2(1H)-carboxylate (0.42 g, 1.16 mmol) and lithium aluminum hydride (66.11 mg, 1.74 mmol) in THF (8 mL) was stirred at 25° C. for 16 h. Sodium sulfate decahydrate (0.1 g, 0.31 mmol) was added slowly to the reaction mixture and the resulting mixture was stirred at 15° C. for 30 min. Then the mixture was filtered and the filtrate was concentrated at reduced pressure to give tert-butyl 8-amino-7-chloro-3,4-dihydroisoquinoline-2(1H)-carboxylate (0.3 g, 1.06 mmol, 91.36% yield). MS (ESI): m/z 227.0 [M−55]+.

tert-Butyl 7-chloro-8-(3-methylpyridine-2-sulfonamido)-3,4-dihydroisoquinoline-2(1H)-carboxylate. To a solution of tert-butyl 8-amino-7-chloro-3,4-dihydroisoquinoline-2(1H)-carboxylate (0.15 g, 0.53 mmol, crude) in THE (10 mL) was added sodium bis(trimethylsilyl)amide (1 M, 0.8 mL) slowly at −70° C. under nitrogen. The mixture was stirred at 15° C. for 30 min, then 3-methylpyridine-2-sulfonyl chloride (101.66 mg, 0.53 mmol) which was dissolved with THF (2 mL) was added slowly at −70° C. and the resulting mixture was stirred at 15° C. for 2 h. The mixture was quenched with saturated ammonium chloride (5 mL) slowly at −20° C. and the mixture was diluted with water. The resulting mixture was extracted with ethyl acetate and the combined organic phase was washed with brine, dried over sodium sulfate, filtered and concentrated at reduced pressure to give a residue. The residue was purified by silica gel chromatography to give tert-butyl 7-chloro-8-(3-methylpyridine-2-sulfonamido)-3,4-dihydroisoquinoline-2(1H)-carboxylate (0.1 g, crude). MS (ESI): m/z 438.0 [M+1]⁺.

N-(7-Chloro-1,2,3,4-tetrahydroisoquinolin-8-yl)-3-methylpyridine-2-sulfonamide (trifluoroacetic acid salt). To a solution of tert-butyl 7-chloro-8-(3-methylpyridine-2-sulfonamido)-3,4-dihydroisoquinoline-2(1H)-carboxylate (0.18 g, 0.41 mmol, crude) in DCM (4 mL) was added trifluoroacetic acid (3.08 g, 27.01 mmol, 2 mL) slowly at 0° C. The mixture was stirred at 15° C. for 2 h. The mixture was concentrated at reduced pressure to give N-(7-chloro-1,2,3,4-tetrahydroisoquinolin-8-yl)-3-methylpyridine-2-sulfonamide (trifluoroacetic acid salt). MS (ESI): m/z 338.0 [M+1]⁺.

N-(7-Chloro-2-methyl-1,2,3,4-tetrahydroisoquinolin-8-yl)-3-methylpyridine-2-sulfonamide. To a solution of N-(7-chloro-1,2,3,4-tetrahydroisoquinolin-8-yl)-3-methylpyridine-2-sulfonamide (0.2 g, crude) (trifluoroacetic acid salt) in MeOH (10 mL) was added TEA (71.89 mg, 0.71 mmol) slowly at 0° C. Then formaldehyde (480.43 mg, 5.92 mmol, 0.44 mL, 37% purity), acetic acid (7.11 mg, 0.12 mmol) and sodium cyanoborohydride (111.61 mg, 1.78 mmol) were added and the mixture was stirred at 15° C. for 16 h. The mixture was concentrated at reduced pressure to give a residue which was diluted with saturated sodium carbonate and the resulting mixture was extracted with ethyl acetate. The combined organic phase was washed with brine, dried over sodium sulfate, filtered and concentrated at reduced pressure to give a residue. The residue was purified by prep-HPLC to give N-(7-chloro-2-methyl-3,4-dihydro-1H-isoquinolin-8-yl)-3-methyl-pyridine-2-sulfonamide (14.01 mg, 0.04 mmol, 6.46% yield, 96% purity). ¹H NMR (400 MHz, CDCl₃) δ 8.53 (d, J=3.9 Hz, 1H), 7.64 (d, J=7.8 Hz, 1H), 7.40 (dd, J₁=7.7, J₂=4.5 Hz, 1H), 6.99 (d, J=8.4 Hz, 1H), 6.92 (d, J=8.4 Hz, 1H), 3.80 (s, 2H), 2.94 (t, J=6.1 Hz, 2H), 2.73 (t, J=6.2 Hz, 2H), 2.54 (s, 3H), 2.51 (s, 3H); MS (ESI): m/z 352.1 [M+1]⁺.

### Example 45. 1-(2,2,2-trifluoroethyl)-N-[5-[3-(trifluoromethyl)phenoxy]-8-quinolyl]imidazole-2-sulfonamide

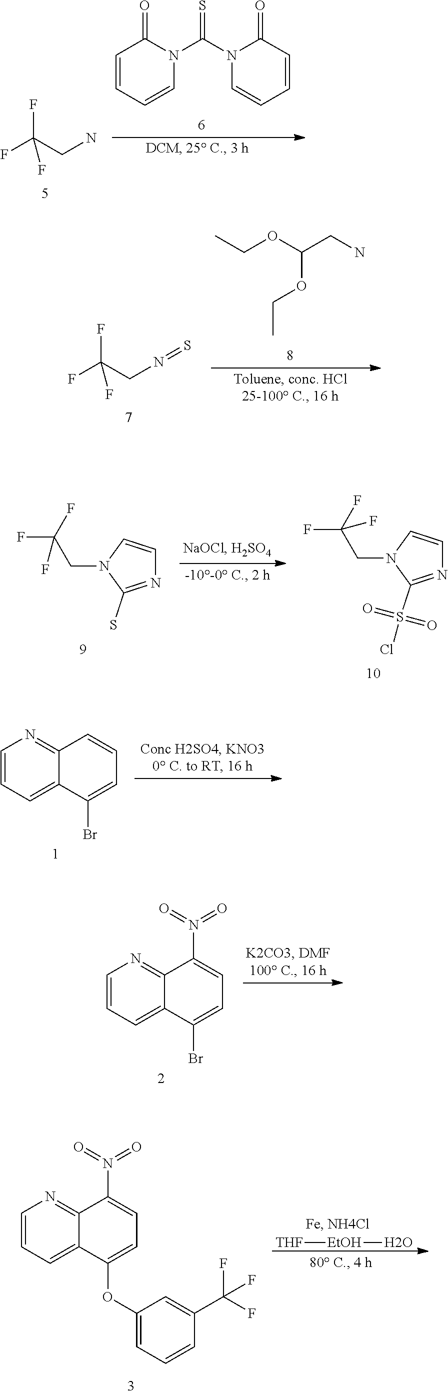

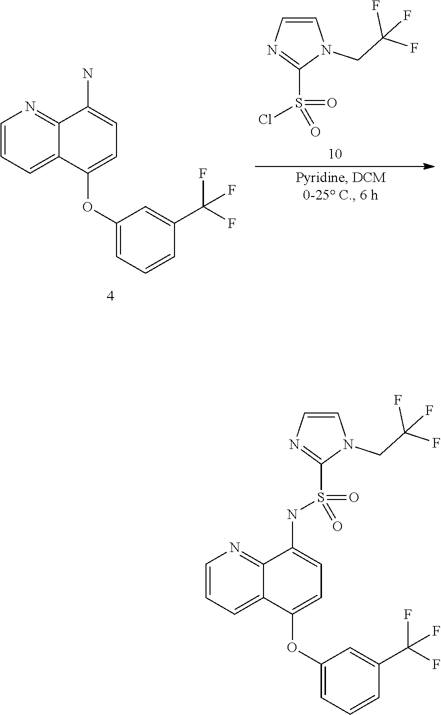 [SMILES: Nc1ccc(Oc2cccc(C(F)(F)F)c2)c2cccnc12 ; O=S(=O)(Cl)c1nccn1CC(F)(F)F ; O=S(=O)(Nc1ccc(Oc2cccc(C(F)(F)F)c2)c2cccnc12)c1nccn1CC(F)(F)F]

5-Bromo-8-nitro-quinoline. A solution of 5-bromoquinoline (10 g, 48.1 mmol) in concentrated H₂SO₄ (40 mL) was cooled to 0° C. and potassium nitrate (7.77 g, 77 mmol) was added slowly in portions under inert atmosphere. Resulting reaction mixture was allowed to attain room temperature and continued stirring for 16 h. Reaction mixture was poured in crushed ice. Precipitate thus formed was filtered, washed with water and dried to afford 5-bromo-8-nitro-quinoline (9 g, 74%) as a light yellow solid. MS (ESI): m/z 253.0 [M+1]⁺.

8-Nitro-5-[3-(trifluoromethyl)phenoxy]quinoline. To a stirred solution of 5-bromo-8-nitro-quinoline (5 g, 19.84 mmol) in dry DMF (50 ml) was added K₂CO₃ (6.85 g, 49.5 mmol) followed by addition of 3-trifluoromethyl phenol (6.4 g, 39.68 mmol) at room temperature under argon atmosphere. Resulting mixture was heated at 100° C. for 16 h. It was cooled to room temperature, quenched with water and extracted with ethyl acetate. Organic layer was dried over anhydrous sodium sulphate, filtered and concentrated under reduce pressure. Crude product was purified by column chromatography (10-20% EtOAc in hexane) to afford 8-nitro-5-[3-(trifluoromethyl)phenoxy]quinoline (3.5 g, 53%) as yellowish solid. MS (ESI): m/z 335.37 [M+1]⁺.

5-[3-(trifluoromethyl)phenoxy]quinolin-8-amine. To a stirred solution of 8-nitro-5-[3-(trifluoromethyl)phenoxy]quinoline (1 g, 2.99 mmol) in THF-EtOH-H₂O (24 mL, 3:2:1 ratio) NH₄Cl (238 mg, 4.49 mmol) and Fe powder (838 mg, 14.97 mmol) were added. Resulting mixture was heated at 80° C. for 4 h. After completion, reaction mixture was filtered through a short pad of celite and washed with ethyl acetate. Filtrate was concentrated under reduced pressure and crude product was purified by combiflash chromatography (20-25% EtOAc in hexane) to afford 5-[3-(trifluoromethyl)phenoxy]quinolin-8-amine (700 mg, 77%) as yellow solid. MS (ESI): m/z 305.4 [M+1]⁺.

1-(2,2,2-Trifluoroethyl)-1H-imidazole-2-thiol. To a stirred solution of 2,2,2-trifluoroethan-1-amine (5 g, 50.5 mmol) in DCM (50 mL) and 1-[(2-oxo-1,2-dihydropyridin-1-yl)carbothioyl]-1,2-dihydropyridin-2-one (12.8 g, 55.6 mmol) was added in portions at 0° C. Resulting mixture was warmed to 10° C. and stirred for 3 h. 2,2-Diethoxyethan-1-amine (10 g, 75.2 mmol) was added under cooling condition and stirred at RT for 1 h. The reaction mixture was evaporated under reduced pressure, diluted with toluene (50 mL) and concentrated HCl (5 mL) was added. Resulting mixture was heated at 110° C. for 16 h. After completion, reaction mixture was concentrated under reduced pressure, diluted with water, adjusted pH-8 with aqueous 1(N) NaOH solution and extracted with ethyl acetate. Combined organic layer was washed with brine, dried over anhydrous sodium sulphate, filtered and concentrated under reduced pressure. Crude mass was purified by column chromatography (25-30% EtOAc-Hex) to afford 1-(2,2,2-trifluoroethyl)-1Himidazole-2-thiol (3 g, 33% over two steps) as white solid. MS (ESI): m/z 183.3 [M+1]⁺.

1-(2,2,2-Trifluoroethyl)-1H-imidazole-2-sulfonyl chloride. 1-(2,2,2-Trifluoroethyl)-1H-imidazole-2-thiol (1.5 g, 8.2 mmol) was taken in a two necked round bottom flask and cooled to −10° C. Concentrated H₂SO₄ (40 mL) was added slowly under cooling condition. NaOCl (70 mL) was added at −15° C. over a period of 30 min and continued stirring for additional 0.5 h. After completion, reaction mixture was quenched with ice water and extracted with DCM. Combined organic layer was washed with brine, dried over anhydrous MgSO₄, filtered and partially concentrated under reduced pressure to afford 1-(2,2,2-trifluoroethyl)-1H-imidazole-2-sulfonyl chloride as DCM solution which was used immediately in the next step without further purification. MS (ESI): m/z 249.3 [M+1]⁺.

1-(2,2,2-Trifluoroethyl)-N-[5-[3-(trifluoromethyl)phenoxy]-8-quinolyl]imidazole-2-sulfonamide. To a stirred solution of 5-[3-(trifluoromethyl)phenoxy]quinolin-8-amine (700 mg, 2.31 mmol) in pyridine (4 mL, 49.6 mmol) and DCM (10 ml) at 0° C., 1-(2,2,2-trifluoroethyl)-1H-imidazole-2-sulfonyl chloride (DCM solution) was added drop wise under argon atmosphere. Reaction mixture was slowly warmed to RT and stirred for 2 h. After completion, reaction mixture was diluted with water and extracted with DCM. Combined organic layer was washed with brine, dried over anhydrous sodium sulphate, filtered and concentrated under reduced pressure. Crude product was purified by combiflash chromatography (25-30% ethyl acetate in hexane) to afford 1-(2,2,2-trifluoroethyl)-N-[5-[3-(trifluoromethyl)phenoxy]-8-quinolyl]imidazole-2-sulfonamide (680 mg, 62%). ¹H NMR (400 MHz, DMSO-d₆) δ 5.44 (q, J=8.9 Hz, 2H), 7.00 (d, J=1.2 Hz, 1H), 7.19 (d, J=8.4 Hz, 1H), 7.31 (dd, J=2.4, 8.3 Hz, 1H), 7.42 (s, 1H), 7.47 (s, 1H), 7.54 (d, J=7.8 Hz, 1H), 7.57-7.68 (m, 2H), 7.79 (d, J=8.4 Hz, 1H), 8.44 (dd, J=1.6, 8.5 Hz, 1H), 8.91 (dd, J=1.7, 4.2 Hz, 1H), 10.72 (brs, 1H). MS (ESI): m/z 517.2 [M+1]⁺.

### Example 46. N-(5-Cyano-8-quinolyl)-1-ethyl-imidazole-2-sulfonamide

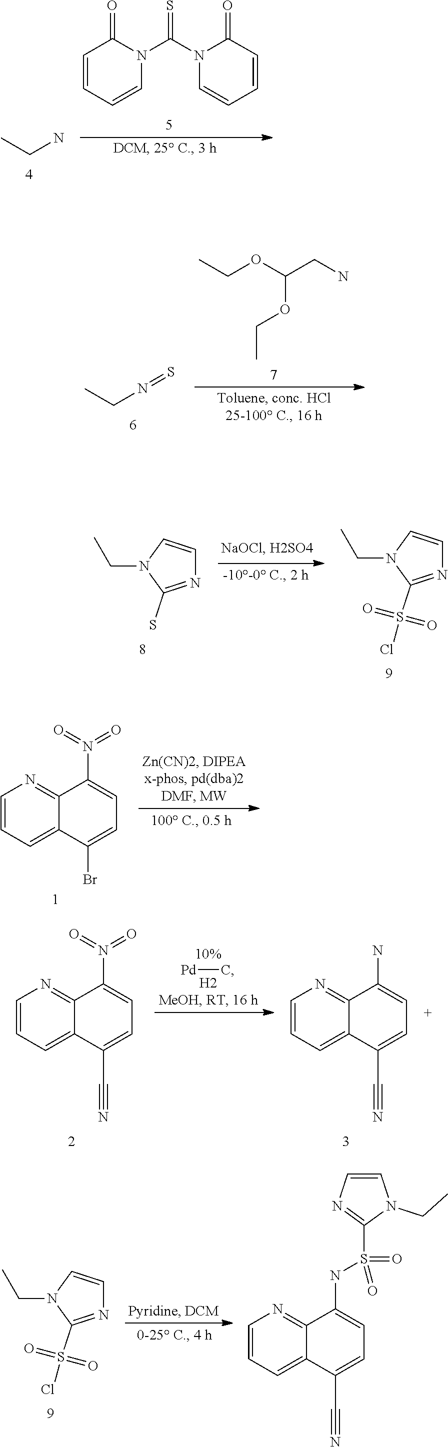

8-Nitroquinoline-5-carbonitrile. To a stirred degassed solution of 5-bromo-8-nitro-quinoline (200 mg, 0.791 mmol) in DMF (4 ml) in zinc cyanide (371 mg, 3.16 mmol) was added followed by DIPEA (0.2 ml, 1.18 mmol), Pd(dba)₂ (136 mg, 0.24 mmol) and Xphos (113 mg, 0.24 mmol). Resulting mixture was heated under MW irradiation at 100° C. for 0.5 h. Reaction mixture was cooled to ambient temperature, diluted with water and extract with ethyl acetate. Combined organic layer was washed with brine and dried over anhydrous Na₂SO₄, filtered and concentrated under reduced pressure. Crude product was purified by combiflash chromatography (20-25% EtOAc in hexane) to afford 8-nitroquinoline-5-carbonitrile (75 mg, 47%) as off white solid. MS (ESI): m/z 200.3 [M+1]⁺.

8-Aminoquinoline-5-carbonitrile. To a stirred solution of 8-nitroquinoline-5-carbonitrile (85 mg, 0.43 mmol) in MeOH (5 mL) was added 10% Pd—C (33 mg). Resulting mixture was hydrogenated under balloon pressure at room temperature for 16 h. After completion, reaction mixture was filtered through a short pad of celite and filtrate was concentrated under reduced pressure. Crude mass was purified combiflash chromatography (20-25% EtOAc in hexane) to afford 8-aminoquinoline-5-carbonitrile (40 mg, 55%) as colourless gum. MS (ESI): m/z 170.18 [M+1]⁺.

Isothiocyanatoethane. To a stirred solution of ethylamine (5 g, 55.5 ml, 110.92 mmol, 2M in THF) in DCM (300 mL), 1-[(2-oxo-1,2-dihydropyridin-1-yl)carbothioyl]-1,2-dihydropyridin-2-one (28.4 g, 122 mmol) was added in portions and stirred at RT for 3 h. After completion, the reaction mixture was evaporated to dryness to afford isothiocyanatoethane (7.7 g, crude) as brownish solid, which was used directly for the next step without further purification.

1-Ethylimidazole-2-thiol. To a stirred solution of isothiocyanatoethane (7.7 g, 88.70 mmol, crude) in toluene (40 mL), 2,2-diethoxyethan-1-amine (19.3 ml, 133.06 mmol) was added drop wise at RT and stirred for 2 h. Then conc. HCl (7.5 mL) was added to the reaction mixture and stirred at 110° C. for 3 h. After completion, reaction mixture was evaporated to dryness and the residue was diluted with water (40 mL). Aqueous part was basified with saturated sodium bicarbonate solution to pH-8 and extracted with ethyl acetate. Combined organic part was washed with brine, dried over sodium sulphate, filtered and concentrated under reduced pressure. Crude product was purified by silica gel (100-200 mesh) column chromatography (eluted at 30-50% ethyl acetate in hexane) to afford 1-ethylimidazole-2-thiol (10 g, 70%_2 steps) as brown solid. MS (ESI): m/z 129.2 [M+1]⁻.

1-Ethylimidazole-2-sulfonyl chloride. 1-Ethylimidazole-2-thiol (150 mg, 1.72 mmol) was taken in a two neck round bottom flask and cooled to −10° C. Conc. H₂SO₄ (3 mL) was added slowly drop wise with constant stirring and stirred at −10° C. for another 10 min. Reaction mixture was cooled to −15° C. and NaOCl (9 mL) was added drop wise over 30 min. It was stirred at −10° C. for another 30 min. After completion, reaction mixture was quenched with ice water and extracted with DCM. Combined organic layer was washed with cold brine, dried over MgSO₄ (pre-cooled flask), filtered on a sintered funnel and partially concentrated at low temper to afford 1-ethylimidazole-2-sulfonyl chloride as a DCM solution which was used directly for the next step without further purification.

N-(5-Cyano-8-quinolyl)-1-ethyl-imidazole-2-sulfonamide. To a stirred solution of 8-aminoquinoline-5-carbonitrile (50 mg, 0.296 mmol) in pyridine (1 mL, 0.6 mmol) and DCM (4 mL) at 0° C., 1-ethylimidazole-2-sulfonyl chloride (crude, DCM solution) was added drop wise under argon atmosphere. Then it was stirred at 0° C.-RT for 2 h. After completion, it was diluted with water and extracted with DCM. Combined organic layer was washed with brine, dried over anhydrous sodium sulphate, filtered and concentrated under reduced pressure. Crude product was purified by prep HPLC to afford N-(5-cyano-8-quinolyl)-1-ethyl-imidazole-2-sulfonamide (20 mg, 21%). ¹H NMR (400 MHz, DMSO-d₆) δ 1.32 (t, J=7.2 Hz, 3H), 4.40 (q, J=7.2 Hz, 2H), 7.01 (s, 1H), 7.50 (s, 1H), 7.84 (dd, J=4.6, 8.4 Hz, 2H), 8.15 (d, J=7.9 Hz, 1H), 8.46 (d, J=8.4 Hz, 1H), 9.03 (d, J=3.4 Hz, 1H) [NH proton was not seen]. MS (ESI): m/z 328.0 [M+1]⁺.

### Example 47. 1-Phenyl-N-[5-(trifluoromethyl)-8-quinolyl]imidazole-2-sulfonamide

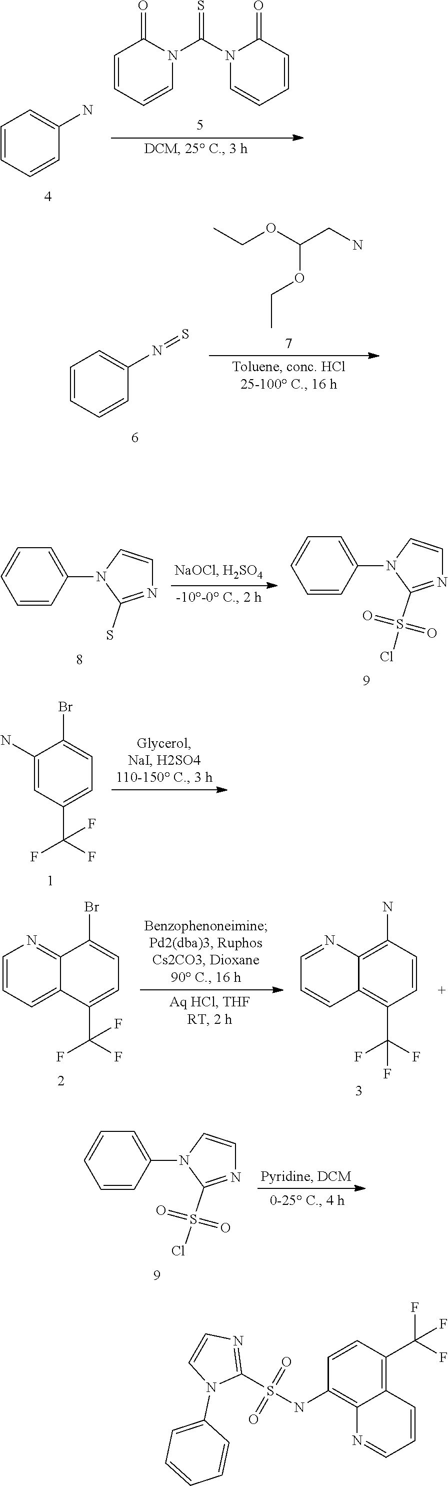

8-Bromo-5-(trifluoromethyl)quinoline. Glycerol (1.5 g, 16.3 mmol) was heated to 160° C. for 1 h. It was cooled down to 110° C. and 2-bromo-5-(trifluoromethyl)aniline (2.0 g, 8.33 mmol), sodium iodide (25 mg, 0.16 mmol) were added. Resulting mixture was vigorously stirred and heated to 150° C. and sulfuric acid (95-98%, 1.8 g, 18.4 mmol) was added drop wise and heating continued at 150° C. for 45 min. Reaction mixture was allowed to cooled to ambient temperature, diluted with water and extract with ethyl acetate. Combined organic layer was washed with brine, dried over anhydrous Na₂SO₄, filtered and concentrated under reduced pressure. Crude product was purified by combiflash chromatography (20-25% EtOAc in hexane) to afford 8-bromo-5-(trifluoromethyl)quinoline (600 mg, 26%) as colourless gum. MS (ESI): m/z 276.0 [M+1]⁺.

5-(Trifluoromethyl)quinolin-8-amine. To a stirred degassed solution of 8-bromo-5-(trifluoromethyl)quinoline (500 mg, 1.82 mmol) in toluene (8 ml) was added benzophenone imine (395 mg, 2.18 mmol) followed by Cs₂CO₃ (289 mg, 2.72 mmol), Pd(OAc)₂ (41 mg, 0.18 mmol) and xantphos (210 mg, 0.36 mmol). Resulting mixture was heated at 100° C. for 16 h. Reaction mixture was cooled to ambient temperature, filtered through a short pad of celite and washed with DCM. Solvent was removed under reduced pressure and the residue was dissolved in EtOH-THF (10 mL, 1:1) and acidified to pH 1 with 1 M aqueous HCl solution. Resulting mixture was stirred at room temperature for 3 h. It was then basified with saturated aqueous NaHCO₃ solution and extracted with ethyl acetate. Combined organic layer was washed with brine and dried over anhydrous Na₂SO₄, filtered and concentrated under reduced pressure. Crude product was purified by combiflash chromatography (20-25% EtOAc in hexane) to afford 5-(trifluoromethyl)quinolin-8-amine (310 mg, 80%) as orange solid. MS (ESI): m/z 200.3 [M+1]⁺.

Isothiocyanatobenzene. To a stirred solution of aniline (1 g, 10.73 mmol) in DCM (30 mL), 1-[(2-oxo-1,2-dihydropyridin-1-yl)carbothioyl]-1,2-dihydropyridin-2-one (2.9 g, 12.90 mmol) was added in portions and stirred at RT for 3 h. After completion, the reaction mixture was concentrated under reduced pressure to afford isothiocyanatobenzene (1.5 g, crude) as brownish solid, which was used directly in the next step without further purification.

1-Phenylimidazole-2-thiol. To a stirred solution of isothiocyanatobenzene (1.5 g, 11.12 mmol, crude) in toluene (15 mL), 2,2-diethoxyethan-1-amine (2.2 g, 16.66 mmol) was added drop wise at RT and stirred for 2 h. Then conc. HCl (1 mL) was added to the reaction mixture and stirred at 110° C. for 3 h. After completion, reaction mixture was evaporated to dryness and the residue was diluted with water (40 mL). Aqueous part was basified with saturated sodium bicarbonate solution to pH-8 and extracted with ethyl acetate. Combined organic part was washed with brine, dried over sodium sulphate, filtered and concentrated under reduced pressure. Crude product was purified by silica gel (100-200 mesh) column chromatography (eluted at 30-50% ethyl acetate in hexane) to afford 1-Phenylimidazole-2-thiol (1.2 g, 63%_2 steps) as colourless gum. MS (ESI): m/z 177.0 [M+1]⁺.

1-Phenylimidazole-2-sulfonyl chloride. 1-Phenylimidazole-2-thiol (300 mg, 1.70 mmol) was taken in a two neck round bottom flask and cooled to −10° C. Conc. H₂SO₄ (5 mL) was added slowly drop wise with constant stirring and stirred at −10° C. for another 10 min. Reaction mixture was cooled to −15° C. and NaOCl (15 mL) was added drop wise over 30 min. It was stirred at −10° C. for another 30 min. After completion, reaction mixture was quenched with ice water and extracted with DCM. Combined organic layer was washed with cold brine, dried over MgSO₄ (pre-cooled flask), filtered on a sintered funnel and partially concentrated at low temp to afford 1-phenylimidazole-2-sulfonyl chloride as a DCM solution which was used directly in the next step without further purification.

1-Phenyl-N-[5-(trifluoromethyl)-8-quinolyl]imidazole-2-sulfonamide. To a stirred solution of 5-(trifluoromethyl)quinolin-8-amine (200 mg, 0.943 mmol) in pyridine (1 mL, 12.6 mmol) and DCM (5 mL) at 0° C., 1-phenylimidazole-2-sulfonyl chloride (crude, DCM solution) was added drop wise under argon atmosphere. Then it was stirred at 0° C.-RT for 2 h. After completion, it was diluted with water (25 mL) and extracted with DCM (2×50 mL). Combined organic layer was washed with brine, dried over anhydrous sodium sulphate, filtered and concentrated. Crude product was purified by combiflash chromatography (20-25% EtOAc in hexane) to afford 1-phenyl-N-[5-(trifluoromethyl)-8-quinolyl]imidazole-2-sulfonamide (75 mg, 19%) as off white solid. MS (ESI): m/z 418.8 [M+1]⁺.

### Example 48. 1-Ethyl-N-(4-phenyl-8-quinolyl)imidazole-2-sulfonamide

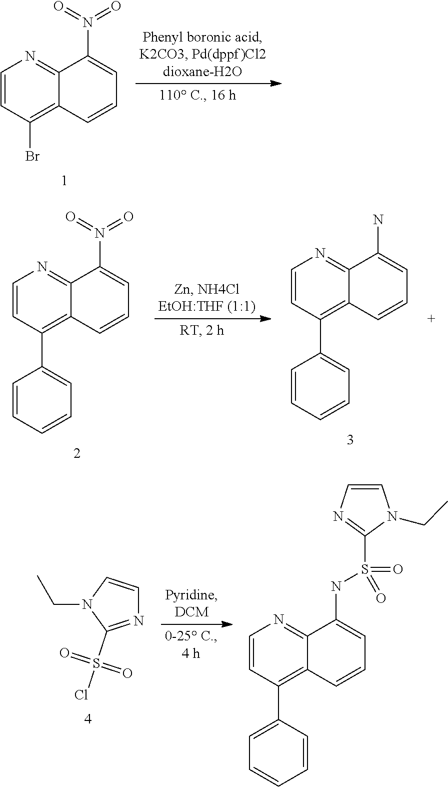 [SMILES: O=[N+]([O-])c1cccc2c(Br)ccnc12 ; O=[N+]([O-])c1cccc2c(-c3ccccc3)ccnc12 ; Nc1cccc2c(-c3ccccc3)ccnc12 ; CCn1ccnc1S(=O)(=O)Cl ; CCn1ccnc1S(=O)(=O)Nc1cccc2c(-c3ccccc3)ccnc12]

8-Nitro-4-phenyl-quinoline. To a stirred degassed solution of 4-bromo-8-nitro-quinoline (200 mg, 0.791 mmol) in dioxane-H₂O (5.5 mL, 10:1) was added K₂CO₃ (82 mg, 0.59 mmol) followed by phenylboronic acid (58 mg, 0.48 mmol), Pd(dppf)Cl₂ (15 mg, 0.02 mmol) under argon atmosphere. Resulting mixture was heated at 100° C. for 16 h. Reaction mixture was cooled to ambient temperature, diluted with water and extract with ethyl acetate. Combined organic layer was washed with brine and dried over anhydrous Na₂SO₄, filtered and concentrated under reduced pressure. Crude product was purified by combiflash chromatography (80-100% EtOAc in hexane) to afford 8-nitro-4-phenyl-quinoline (75 mg, 76%) as off white solid. MS (ESI): m/z 251.0 [M+1]⁺

4-Phenylquinolin-8-amine. To a stirred solution of 8-nitro-4-phenyl-quinoline (140 mg, 0.56 mmol) in THF-EtOH (24 mL, 1:1 ratio), NH₄C₁ (297 mg, 5.6 mmol) and zinc dust (182 mg, 2.8 mmol) were added and stirred at RT for 2 h. After completion, the reaction mixture was filtered through a short pad of celite and washed with ethyl acetate. Filtrate was concentrated under reduced pressure and crude product was purified by combiflash chromatography (8-10% EtOAc in hexane) to afford 4-phenylquinolin-8-amine (100 mg, 81%) as grey solid. MS (ESI): m/z 221.3 [M+1]⁺.

1-Ethyl-N-(4-phenyl-8-quinolyl)imidazole-2-sulfonamide. To a stirred solution of 4-phenylquinolin-8-amine (80 mg, 0,363 mmol) in pyridine (1 mL, 12.6 mmol) and DCM (4 mL) at 0° C., 1-ethylimidazole-2-sulfonyl chloride (crude, DCM solution) was added drop wise under argon atmosphere. Then it was stirred at 0° C.-RT for 2 h. After completion, it was diluted with water and extracted with DCM. Combined organic layer was washed with brine, dried over anhydrous sodium sulphate, filtered and concentrated under reduced pressure. Crude product was purified by prep HPLC to afford 1-ethyl-N-(4-phenyl-8-quinolyl)imidazole-2-sulfonamide (70 mg, 51%) as off white solid. MS (ESI): m/z 379.2 [M+1]⁺.

### Example 49. 1-Ethyl-N-(5-methylsulfonyl-8-quinolyl)imidazole-2-sulfonamide

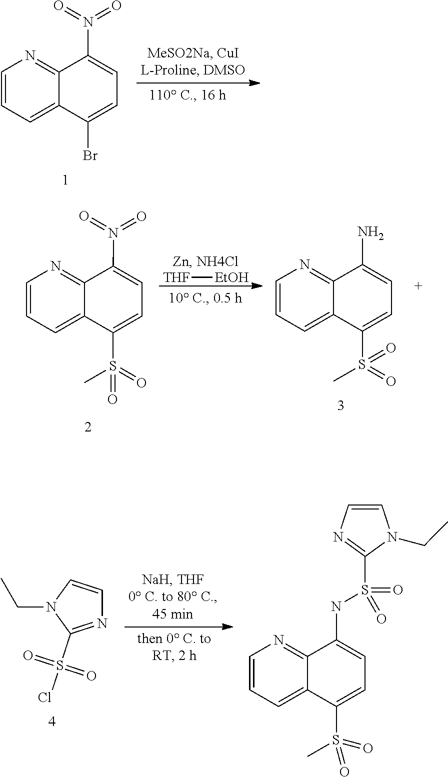

5-Methylsulfonyl-8-nitro-quinoline. To a stirred degassed solution of 5-bromo-8-nitro-quinoline (150 mg, 0.595 mmol) in DMSO (8 mL) was added sodium methanesulfinate (73 mg, 0.71 mmol) followed by cupper iodide (11 mg, 0.06 mmol) and L-proline (14 mg, 0.12 mmol) under inert atmosphere. Resulting mixture was heated at 100° C. for 16 h in a sealed tube. Reaction mass was cooled to ambient temperature, diluted with water and extract with ethyl acetate. Combined organic layer was washed with brine and dried over anhydrous Na₂SO₄, filtered and concentrated under reduced pressure. Crude product was purified by combiflash chromatography (5-10% EtOAc in hexane) to afford 5-methylsulfonyl-8-nitro-quinoline (45 mg, 30%) as off white solid. MS (ESI): m/z 253.2 [M+1]⁺.

5-Methylsulfonylquinolin-8-amine. To a stirred solution of 5-methylsulfonyl-8-nitro-quinoline (90 mg, 0.35 mmol) in THF-EtOH (10 mL, 1:1 ratio), NH₄Cl (95 mg, 1.78 mmol) and zinc dust (117 mg, 1.78 mmol) were added and stirred at 10° C. for 0.5 h. After completion, the reaction mixture was filtered through a short pad of celite bed and washed with ethyl acetate. Filtrate was concentrated under reduced pressure and crude product was purified by combiflash chromatography (30-35% EtOAc in hexane) to afford 5-methylsulfonylquinolin-8-amine (75 mg, 94%) as grey solid. MS (ESI): m/z 223.2 [M+1]⁺.

1-Ethyl-N-(5-methylsulfonyl-8-quinolyl)imidazole-2-sulfonamide. To a stirred solution of 5-methylsulfonylquinolin-8-amine (75 mg, 0.34 mmol) in THE (5 mL) at 0° C. was added NaH (27.0 mg, 0.676 mmol) and stirred at same temperature for 15 min. Resulting mixture was heated at 80° C. for 30 min and cooled to 0° C. 1-Ethylimidazole-2-sulfonyl chloride (crude, DCM solution) was added drop wise under argon atmosphere. Reaction mixture was stirred at 0° C.-RT for 2 h. After completion, it was diluted with water and extracted with DCM. Combined organic layer was washed with brine, dried over anhydrous sodium sulphate, filtered and concentrated under reduced pressure. Crude product was purified by prep HPLC to afford 1-ethyl-N-(5-methylsulfonyl-8-quinolyl)imidazole-2-sulfonamide (10 mg, 8%). ¹H NMR (400 MHz, DMSO-d₆) δ 1.33 (t, J=7.0 Hz, 3H), 4.37 (q, J=7.0 Hz, 2H), 6.93 (s, 1H), 7.46 (s, 1H), 7.63-7.83 (m, 2H), 7.92 (d, J=8.0 Hz, 1H), 8.49 (d, J=8.8 Hz, 1H), 8.90-8.97 (m, 1H), 10.63 (brs, 1H). MS (ESI): m/z 381.1 [M+1]⁺.

### Example 50. N-(6-Fluoro-8-quinolyl)-1-[4-(trifluoromethyl)phenyl]imidazole-2-sulfonamide

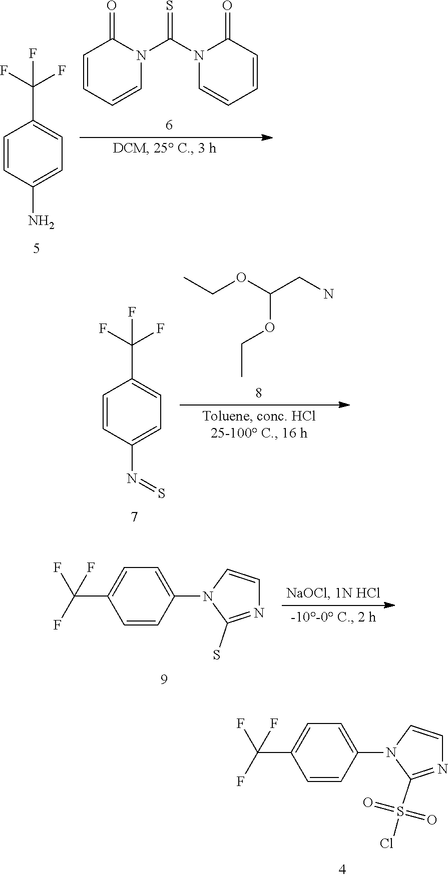

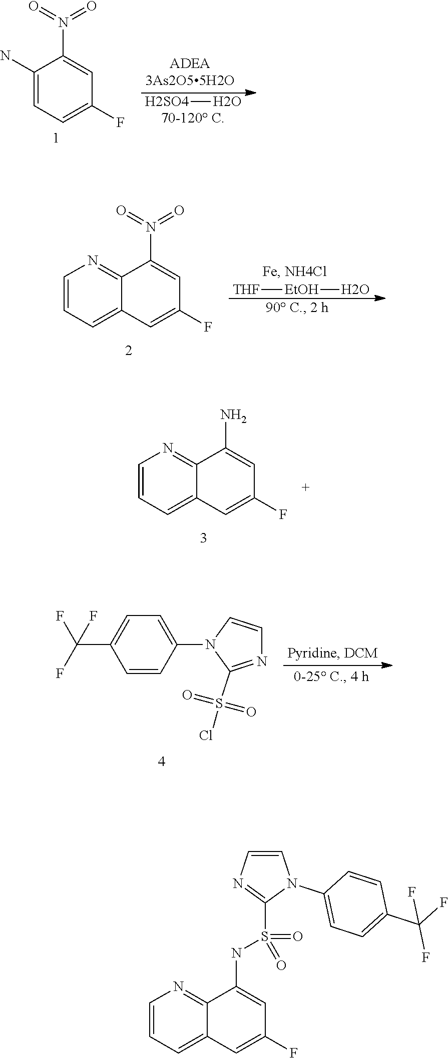

6-Fluoro-8-nitro-quinoline. 4-Fluoro-2-nitro-aniline (10.0 g, 64.06 mmol) and arsenic pentoxide hydrate (10.0 g, 12.82 mmol) were dissolved in a mixture of sulfuric acid (32.0 mL, ˜70%) and water (20.0 mL). Resulting mixture was heated to 80° C. and ADEA (15.0 ml, 96.086 mmol) was added dropwise over 1 h. Reaction mixture was then heated to 120° C. for 90 min. It was allowed to cooled to ambient temperature and poured into an ice/water mixture (200 ml) and filtered. Aqueous ammonia was added dropwise to adjust pH-6. and extracted with DCM. Combined organic layer was washed with brine and dried over anhydrous Na₂SO₄, filtered and concentrated under reduced pressure. Crude product was purified by combiflash chromatography (10% EtOAc in hexane) to afford 6-fluoro-8-nitro-quinoline (6.5 g, 53%) as yellow solid. MS (ESI): m/z 193.2 [M+1]⁺.

6-Fluoroquinolin-8-amine. To a stirred solution of 6-fluoro-8-nitro-quinoline (3.43 g, 17.86 mmol) in THF-EtOH-H₂O (42 mL, 3:2:1 ratio), NH₄Cl (1.43 g, 26.8 mmol) and Fe powder (4.98 g, 89.3 mmol) were added and stirred at 90° C. for 2 h. After completion, the reaction mixture was filtered through a short pad of celite and washed with ethyl acetate. Filtrate was concentrated under reduced pressure and crude product was purified by combiflash chromatography (10-20% EtOAc in hexane) to afford 6-fluoroquinolin-8-amine (1.5 g, 52%) as brown solid. MS (ESI): m/z 163.3 [M+1]⁺.

1-Isothiocyanato-4-(trifluoromethyl)benzene. To a stirred solution of 4-(trifluoromethyl)aniline (1 g, 6.21 mmol) in DCM (20 mL), 1-[(2-oxo-1,2-dihydropyridin-1-yl)carbothioyl]-1,2-dihydropyridin-2-one (1.58 g, 6.83 mmol) was added in portions and stirred at RT for 3 h. After completion, the reaction mixture was concentrated under reduced pressure to afford 1-isothiocyanato-4-(trifluoromethyl)benzene (1.2 g, crude) as brownish solid. It was used directly in the next step without further purification.

1-[4-(Trifluoromethyl)phenyl]imidazole-2-thiol. To a stirred solution of 1-isothiocyanato-4-(trifluoromethyl)benzene (1.2 g, 5.91 mmol, crude) in toluene (10 mL), 2,2-diethoxyethan-1-amine (1.28 g, 8.87 mmol) was added drop wise at RT and stirred for 2 h. Conc. HCl (2 mL) was added to the reaction mixture and stirred at 100° C. for 16 h. After completion, reaction mixture was evaporated to dryness and the residue was diluted with water. Aqueous part was basified with saturated sodium bicarbonate solution to pH˜8 and extracted with ethyl acetate. Combined organic part was washed with brine, dried over sodium sulphate, filtered and concentrated under reduced pressure. Crude product was purified by silica gel (100-200 mesh) column chromatography (30-40% ethyl acetate in hexane) to afford 1-[4-(trifluoromethyl)phenyl]imidazole-2-thiol (1.2 g, 83%_2 steps) as off white solid. MS (ESI): m/z 245.3 [M+1]⁺.

1-[4-(Trifluoromethyl)phenyl]imidazole-2-sulfonyl chloride. 1-[4-(trifluoromethyl)phenyl]imidazole-2-thiol (300.0 mg, 1.23 mmol) was cooled to −10° C. and 1 (N) HCl (10 ml) was added dropwise with constant stirring and stirred for 10 min. NaOCl (18 ml) was added dropwise over 30 min and continued stirring for additional 0.5 h. It was quenched with ice water and extracted with DCM. Combined organic layer was washed with cold brine, dried over MgSO₄ (cooled flask), filtered on a sintered funnel and partially concentrated at low temperature. DCM solution of the 1-[4-(trifluoromethyl)phenyl]imidazole-2-sulfonyl chloride was directly used in the next step without further purification.

N-(6-Fluoro-8-quinolyl)-1-[4-(trifluoromethyl)phenyl]imidazole-2-sulfonamide. To a stirred solution of 6-fluoroquinolin-8-amine (100 mg, 0.617 mmol) in pyridine (2 mL, 25.2 mmol) and DCM (10 mL) at 0° C., 1-[4-(trifluoromethyl)phenyl]imidazole-2-sulfonyl chloride (crude, DCM solution) was added drop wise under argon atmosphere. It was stirred at 0° C.-RT for 2 h. After completion, reaction mixture was diluted with water and extracted with DCM. Combined organic layer was washed with brine, dried over anhydrous sodium sulphate, filtered and concentrated under reduced pressure. Crude product was purified by prep HPLC to afford N-(6-fluoro-8-quinolyl)-1-[4-(trifluoromethyl)phenyl]imidazole-2-sulfonamide (12 mg, 5%). ¹H NMR (400 MHz, DMSO-d₆) δ 7.19 (s, 1H), 7.43-7.55 (m, 2H), 7.65-7.67 (m, 4H), 7.82 (d, J=8.3 Hz, 2H), 8.40 (d, J=8.1 Hz, 1H), 8.80 (dd, J=1.6, 4.2 Hz, 1H), 10.60 (brs, 1H). MS (ESI): m/z 437.2 [M+1]⁺.

### Example 51. N-[5-(4-Fluorophenyl)quinolin-8-yl]-1-(2,2,2-trifluoroethyl)-1H-imidazole-2-sulfonamide

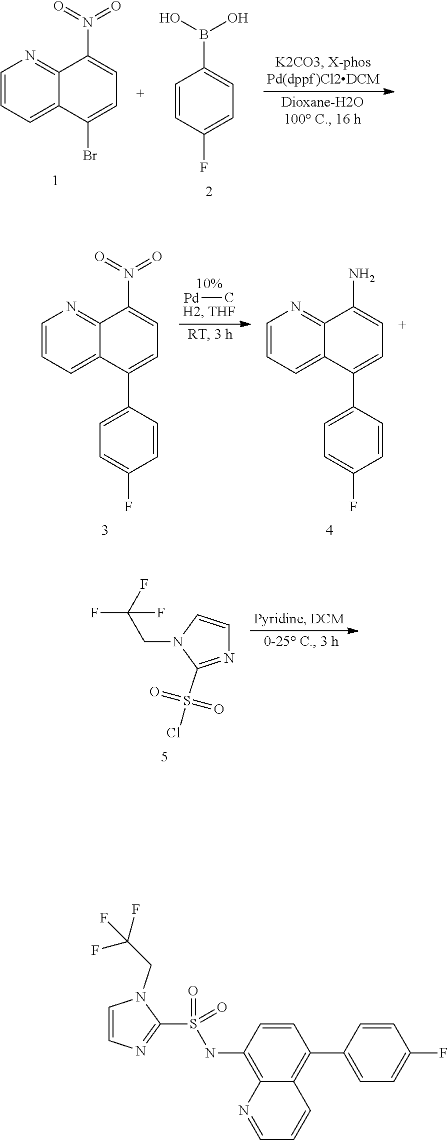

5-(4-Fluorophenyl)-8-nitroquinoline. To a stirred solution of 5-bromo-8-nitroquinoline (1 g, 3.97 mmol) in 1,4-dioxane (20 mL) and water (2 mL) K₂CO₃ (1.37 g, 9.92 mmol) and (4-fluorophenyl)boronic acid (667 mg, 4.76 mmol) were added. Resulting mixture was purged with argon for 5 min. X-phos (296 mg, 0.4 mmol) and Pd(dppf)Cl₂.DCM (162 mg, 0.2 mmol) were added under inert atmosphere. Resulting mixture was heated at 100° C. for 16 h. After completion, the reaction mixture was cooled to room temperature, filtered through a short pad of celite and washed with ethyl acetate. Filtrate part was diluted with water and extracted with ethyl acetate. Combined organic layer was washed with brine, dried over anhydrous sodium sulphate, filtered and concentrated under reduced pressure. Crude product was purified by combiflash column chromatography (35-40% EtOAc in hexane) to afford 5-(4-fluorophenyl)-8-nitroquinoline (700 mg, 66%) as yellow solid. MS (ESI). m/z 268.9 [M+1]⁺.

5-(4-Fluorophenyl)quinolin-8-amine. To a stirred degassed solution of 5-(4-fluorophenyl)-8-nitroquinoline (700 mg, 2.61 mmol) in THF (20 mL) was added 10% Pd—C (700 mg) and hydrogenated under balloon atmosphere at RT for 3 h. After completion, the reaction mixture was filtered through short pad of celite and washed with ethyl acetate. Filtrate was evaporated under reduced pressure and crude product was purified by combiflash column chromatography (25-30% EtOAc in hexane) to afford 5-(4-fluorophenyl)quinolin-8-amine (350 mg, 56%) as yellow solid. MS (ESI): m/z 238.6 [M+1]⁺.

N-[5-(4-Fluorophenyl)quinolin-8-yl]-1-(2,2,2-trifluoroethyl)-1H-imidazole-2-sulfonamide. To a stirred solution of 5-(4-fluorophenyl)quinolin-8-amine (100 mg, 0.42 mmol) in pyridine (2 mL) and DCM (4 mL) at 0° C., 1-(2,2,2-trifluoroethyl)-1H-imidazole-2-sulfonyl chloride (230 mg, 0.92 mmol), dissolved in DCM (8 ml) was added dropwise under inert atmosphere. Resulting mixture was warmed to room temperature and stirred for 3 h. After completion, it was diluted with water and extracted with DCM. Combined organic layer was washed with brine, dried over anhydrous sodium sulphate, filtered and concentrated under reduced pressure. Crude product was purified by combiflash column chromatography (65-70% EtOAc in hexane) to afford N-[5-(4-fluorophenyl)quinolin-8-yl]-1-(2,2,2-trifluoroethyl)-1H-imidazole-2-sulfonamide (55 mg, 29%). ¹H NMR (400 MHz, DMSO-d₆) δ 5.48 (q, J=8.8 Hz, 2H), 7.08 (d, J=0.8 Hz, 1H), 7.31-7.42 (m, 2H), 7.46-7.56 (m, 4H), 7.59 (dd, J=4.2, 8.6 Hz, 1H), 7.89 (d, J=7.9 Hz, 1H), 8.18 (dd, J=1.6, 8.6 Hz, 1H), 8.90 (dd, J=1.6, 4.2 Hz, 1H), 10.83 (brs, 1H). MS (ESI): m/z 451.2 [M+1]⁺.

### Example 52. N-5-Bromoquinolin-8-yl-1-ethyl-1H-imidazole-2-sulfonamide

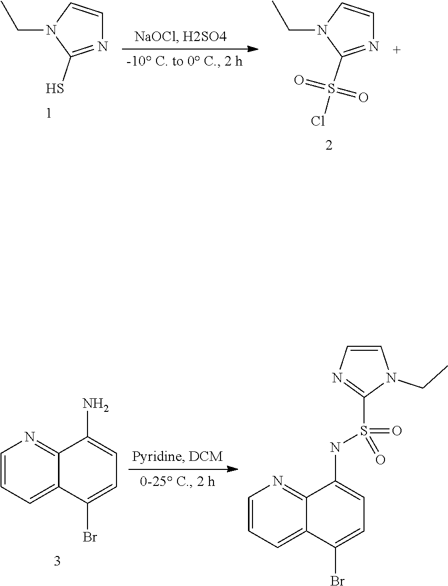

1-Ethylimidazole-2-sulfonyl chloride. 1-Ethylimidazole-2-thiol (300 mg, 3.44 mmol) was taken in a two neck round bottom flask and cooled to −10° C. Conc. H₂SO₄ (5 mL) was added slowly drop wise with constant stirring and stirred at −10° C. for another 10 min. Reaction mixture was cooled to −15° C. and NaOCl (15 mL) was added drop wise over 30 min and stirred at −10° C. for another 30 min. After completion, reaction mixture was quenched with ice water and extracted with cold DCM. Combined organic layer was washed with cold brine, dried over MgSO₄ (pre-cooled flask), filtered on a sintered funnel and partially concentrated at low temp to afford 1-ethylimidazole-2-sulfonyl chloride as a DCM solution which was used directly in the next step without further purification.

N-(5-Bromoquinolin-8-yl)-1-ethyl-1H-imidazole-2-sulfonamide. To a stirred solution of 5-bromoquinolin-8-amine (100 mg, 0.45 mmol) in DCM (3 mL) at 0° C., was added pyridine (2 mL) and stirred at 0° C. for 10 min. 1-Ethyl-1H-imidazole-2-sulfonyl chloride (193 mg, 0.99 mmol in DCM) was added under cooling condition and resulting mixture was stirred at room temperature for 2 h. After completion, reaction mixture was diluted with water and extracted with DCM. Combined organic layer was washed with brine, dried over anhydrous sodium sulphate, filtered and concentrated under reduced pressure. Crude product was purified by prep HPLC to afford N-(5-bromoquinolin-8-yl)-1-ethyl-1H-imidazole-2-sulfonamide (30 mg, 17%). ¹H NMR (400 MHz, DMSO-d₆) δ 1.33 (t, J=7.0 Hz, 3H), 4.37 (q, J=7.0 Hz, 2H), 6.93 (s, 1H), 7.46 (s, 1H), 7.63-7.83 (m, 2H), 7.92 (d, J=8.0 Hz, 1H), 8.49 (d, J=8.8 Hz, 1H), 8.90-8.97 (m, 1H), 10.63 (brs, 1H). MS (ESI): m/z 381.1 [M+1]⁺.

### Example 53. N-(3-Acetylquinolin-8-yl)-1-ethyl-1H-imidazole-2-sulfonamide

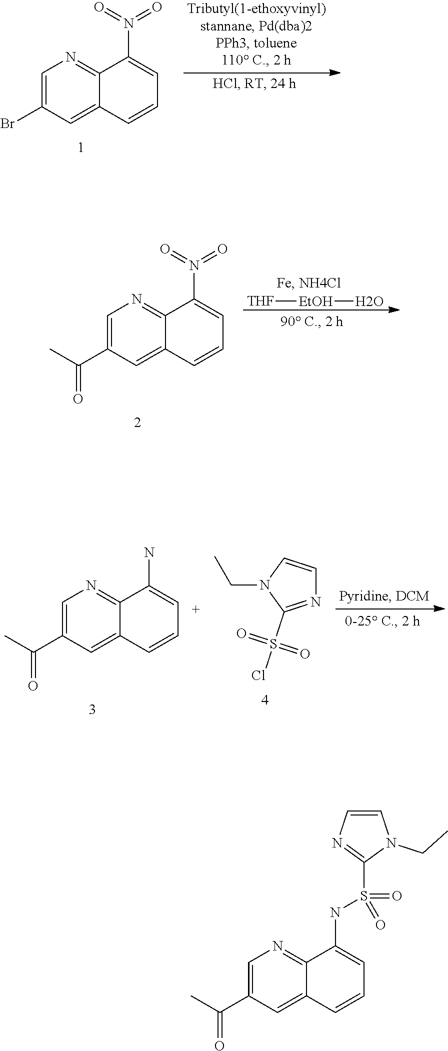

1-(8-Nitroquinolin-3-yl)ethan-1-one. To a stirred solution of 3-bromo-8-nitroquinoline (252 mg, 1 mmol) in toluene (3 mL) was added triphenylphosphine (21 mg, 0.08 mmol) under argon atmosphere followed by Pd(dba)₂ (23 mg, 0.04 mmol) and stirred at room temperature for 15 min. A solution of tributyl(1-ethoxyvinyl)stannane (0.34 mL, 1 mmol) in toluene (2 mL) was added and resulting mixture was heated at 110° C. for 1 h. It was cooled to room temperature and 1(N) HCl (10 mL) was added and resulting mixture was at room temperature for 24 h. After completion, it was neutralized with 1(N) NaOH and extracted with ethyl acetate. Combined organic layer was washed with brine, dried over anhydrous sodium sulphate, filtered and concentrated under reduced pressure. Crude product was purified by combiflash column chromatography (30-35% EtOAc in hexane) to afford 1-(8-nitroquinolin-3-yl)ethan-1-one (160 mg, 74%) as yellowish solid. MS (ESI): m/z 217.08 [M+1]⁺.

1-(8-Aminoquinolin-3-yl)ethan-1-one. To a stirred solution of 1-(8-nitroquinolin-3-yl)ethan-1-one (110 mg, 0.51 mmol) in a mixture of THF-EtOH-water (3:3:1, 10 mL) was added ammonium chloride (41 mg, 0.76 mmol) followed by iron powder (143 mg, 2.55 mmol). Resulting mixture was stirred at 90° C. for 2 h. After completion, reaction mixture was filtered through short pad of celite and washed with dichloromethane. Filtrate part was evaporated under reduced pressure, diluted with dichloromethane, water and the layers were separated. Aqueous layer was back extracted with dichloromethane. Combined organic layer was washed with aqueous NaHCO₃ solution, brine, dried over anhydrous sodium sulphate, filtered and concentrated under reduced pressure. Crude product was purified by combiflash column chromatography (15-20% EtOAc in hexane) to afford 1-(8-aminoquinolin-3-yl)ethan-1-one (80 mg, 84%) as off white solid. MS (ESI): m/z 187.23 [M+1]⁺.

N-(3-Acetylquinolin-8-yl)-1-ethyl-1H-imidazole-2-sulfonamide. To a stirred solution of 1-(8-aminoquinolin-3-yl)ethan-1-one (70 mg, 0.38 mmol) in DCM (3 mL) at 0° C., was added pyridine (2 mL) and stirred at 0° C. for 10 min. 1-Ethyl-1H-imidazole-2-sulfonyl chloride (110 mg, 0.56 mmol in DCM) was added under cooling condition and resulting reaction mixture was stirred at room temperature for 2 h. After completion, reaction mixture was diluted with water and extracted with DCM. Combined organic layer was washed with brine, dried over anhydrous sodium sulphate, filtered and concentrated under reduced pressure. Crude product was purified by combiflash column chromatography (25-30% EtOAc in hexane) to afford N-(3-acetylquinolin-8-yl)-1-ethyl-1H-imidazole-2-sulfonamide (46 mg, 35%). ¹H NMR (400 MHz, DMSO-d₆) δ 1.32 (t, J=7.1 Hz, 3H), 2.72 (s, 3H), 4.37 (q, J=6.8 Hz, 2H), 6.92 (s, 1H), 7.41 (s, 1H), 7.64 (t, J=8.0 Hz, 1H), 7.88 (d, J=7.2 Hz, 2H), 9.03 (s, 1H), 9.22 (s, 1H), 10.60 (brs, 1H). MS (ESI): m/z 345.2 [M+1]⁺.

### Example 54. N-[3-(dimethylamino)quinolin-8-yl]-1-ethyl-1H-imidazole-2-sulfonamide

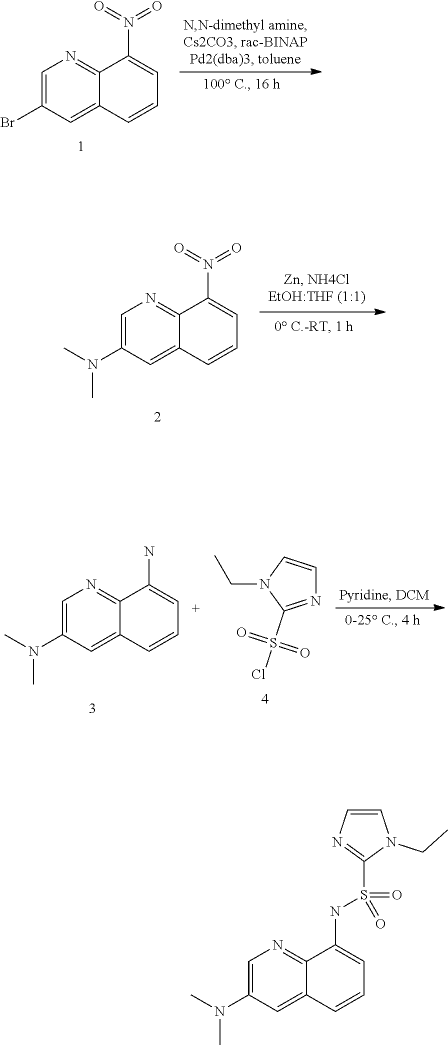 [SMILES: O=[N+]([O-])c1cccc2cc(Br)cnc12 ; CN(C)c1cnc2c([N+](=O)[O-])cccc2c1 ; CN(C)c1cnc2c(N)cccc2c1 ; CCn1ccnc1S(=O)(=O)Cl ; CCn1ccnc1S(=O)(=O)Nc1cccc2cc(N(C)C)cnc12]

N,N-Dimethyl-8-nitroquinolin-3-amine. To a stirred degassed solution of 3-bromo-8-nitroquinoline (300 mg, 1.19 mmol) in toluene (8 mL) in a sealed tube was added cesium carbonate (773 mg, 2.37 mmol), rac-BINAP (111 mg, 0.18 mmol) followed by Pd₂(dba)₃ (109 mg, 0.12 mmol) and purged with argon for additional 5 min. N,N-Dimethylamine (2M in THF, 6 ml, 12 mmol) was added and stirred at 100° C. for 16 h. After completion, reaction mixture was cooled to room temperature, quenched with water and extracted with ethyl acetate. Combined organic layer was washed with brine, dried over anhydrous sodium sulphate, filtered and concentrated under reduced pressure. Crude product was purified by combiflash column chromatography (25-30% EtOAc in hexane) to afford N,N-dimethyl-8-nitroquinolin-3-amine (220 mg, 85%) as yellow solid. MS (ESI): m/z 218.36 [M+1]⁺.

3-N,3-N-Dimethylquinoline-3,8-diamine. To a stirred solution of N,N-dimethyl-8-nitroquinolin-3-amine (100 mg, 0.46 mmol) in EtOH (5 mL) and THF (5 mL) at 10° C., was added ammonium chloride (247 mg, 4.61 mmol) and stirred for 5 min. Zn dust (151 mg, 2.3 mmol) was added and reaction mixture was stirred at RT for 1 h. After completion, reaction mixture was filtered through a short pad of celite and washed with ethyl acetate. Filtrate was concentrated under reduced pressure. Residue thus obtained was diluted with water, ethyl acetate and the layers were separated. Aqueous layer was back extracted with ethyl acetate. Combined organic layer was washed with brine, dried over anhydrous sodium sulphate, filtered and concentrated under reduced pressure. Crude product was purified by combiflash column chromatography (20-25% EtOAc in hexane) to afford 3-N,3-N-dimethylquinoline-3,8-diamine (50 mg, 58%) as deep brown solid. MS (ESI): m/z 188.34 [M+1]⁺.

N-[3-(Dimethylamino)quinolin-8-yl]-1-ethyl-1H-imidazole-2-sulfonamide. To a stirred solution of 3-N,3-N-dimethylquinoline-3,8-diamine (100 mg, 0.54 mmol) in DCM (2 mL) pyridine (2 mL) was added at 0° C. and stirred for 10 min. 1-Ethyl-1H-imidazole-2-sulfonyl chloride (260 mg, 1.34 mmol in DCM), was added at 0° C. and resulting mixture was stirred at room temperature for 4 h. After completion, reaction mixture was diluted with water and extracted with DCM. Combined organic layer was washed with brine, dried over anhydrous sodium sulphate, filtered and concentrated under reduced pressure. Crude product was purified by combiflash column chromatography (25-30% EtOAc in hexane) to afford N-[3-(dimethylamino)quinolin-8-yl]-1-ethyl-1H-imidazole-2-sulfonamide (50 mg, 27%). ¹H NMR (400 MHz, DMSO-d₆) δ 1.30 (t, J=7.2 Hz, 3H), 3.05 (s, 6H), 4.33 (q, J=7.2 Hz, 2H), 6.95 (d, J=0.7 Hz, 1H), 7.27-7.38 (m, 3H), 7.42-7.49 (m, 2H), 8.65 (d, J=2.9 Hz, 1H), 10.15 (brs, 1H). MS (ESI): m/z 346.2 [M+1]⁺.

### Example 55. N-(4-Acetylquinolin-8-yl)-1-ethyl-1H-imidazole-2-sulfonamide

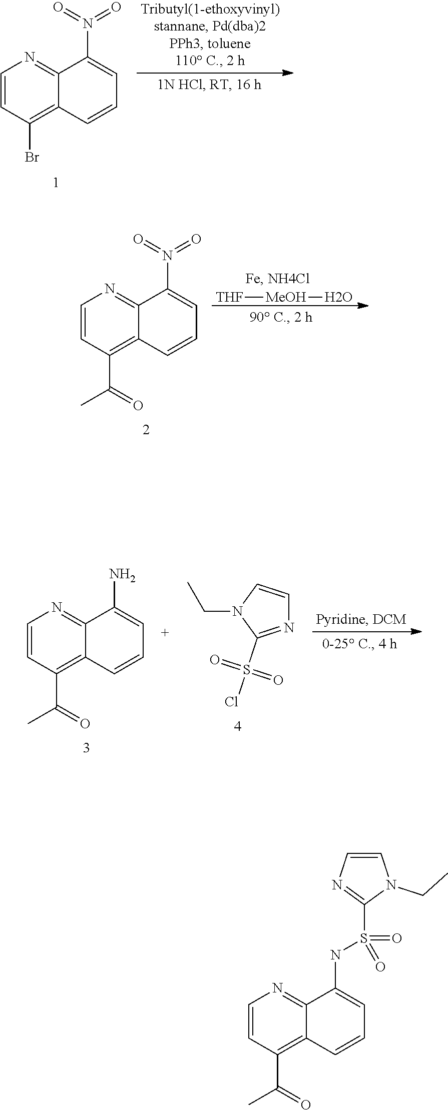

1-(8-Nitroquinolin-4-yl)ethan-1-one. To a stirred degassed solution of 4-bromo-8-nitroquinoline (252 mg, 1 mmol) in toluene (3 mL) was added triphenylphosphine (21 mg, 0.08 mmol) followed by Pd(dba)₂ (23 mg, 0.04 mmol) under inert atmosphere and stirred for 15 min. A solution of tributyl(1-ethoxyvinyl)stannane (0.34 mL, 1 mmol) in toluene (2 mL) was added and resulting mixture was heated at 110° C. for 1 h. It was cooled to room temperature and 1(N) HCl (10 mL) was added. Resulting mixture was stirred at room temperature for 24 h. After completion, reaction mixture was neutralized with 1N NaOH and extracted with ethyl acetate. Combined organic layer was washed with brine, dried over anhydrous sodium sulphate, filtered and concentrated under reduced pressure. Crude product was purified by combiflash column chromatography (35-40% EtOAc in hexane) to afford 1-(8-nitroquinolin-4-yl)ethan-1-one (150 mg, 69%) as off white solid. MS (ESI): m/z 217.2 [M+1]⁺.

1-(8-Aminoquinolin-4-yl)ethan-1-one. To a stirred solution of 1-(8-nitroquinolin-4-yl)ethan-1-one (365 mg, 1.69 mmol) in a mixture of THE (6 mL), ethanol (6 mL) and water (2 mL), was added ammonium chloride (136 mg, 2.54 mmol) followed by iron powder (472 mg, 8.45 mmol). Resulting mixture was heated at 90° C. for 2 h. After completion, reaction mixture was filtered through short pad of celite and washed with dichloromethane. Filtrate part was evaporated under reduced pressure. The residue was diluted with dichloromethane, water and the layers were separated. Organic layer was washed with aqueous NaHCO₃ solution, brine, dried over anhydrous sodium sulphate, filtered and concentrated under reduced pressure. Crude product was purified by combiflash column chromatography (15-20% EtOAc in hexane) to afford 1-(8-aminoquinolin-4-yl)ethan-1-one (240 mg, 76%) as grey solid. MS (ESI): m/z 187.2 [M+1]⁺.

N-(4-Acetylquinolin-8-yl)-1-ethyl-1H-imidazole-2-sulfonamide. To a stirred solution of 1-(8-aminoquinolin-4-yl)ethan-1-one (100 mg, 0.54 mmol) in DCM (3 mL) pyridine (2 mL) was added at 0° C. and stirred for 10 min. 1-Ethyl-1H-imidazole-2-sulfonyl chloride (125 mg, 0.64 mmol in DCM), was added under cooling condition and the resulting mixture was stirred at room temperature for 2 h. After completion, reaction mixture was diluted with water and extracted with DCM. Combined organic layer was washed with brine, dried over anhydrous sodium sulphate, filtered and concentrated under reduced pressure. Crude product was purified by combiflash column chromatography (25-30% EtOAc in hexane) to afford N-(4-acetylquinolin-8-yl)-1-ethyl-1H-imidazole-2-sulfonamide (55 mg, 29%). ¹H NMR (400 MHz, DMSO-d₆) δ 1.33 (t, J=7.2 Hz, 3H), 2.73 (s, 3H), 4.36 (q, J=7.0 Hz, 2H), 6.92 (s, 1H), 7.46 (s, 1H), 7.61 (t, J=8.0 Hz, 1H), 7.80 (d, J=7.1 Hz, 1H), 7.98 (d, J=4.3 Hz, 1H), 8.05 (d, J=8.2 Hz, 1H), 9.01 (d, J=4.3 Hz, 1H), 10.50 (brs, 1H). MS (ESI): m/z 345.2 [M+1]⁺.

### Example 56. N-(6-Benzoylquinolin-8-yl)-1-ethyl-1H-imidazole-2-sulfonamide

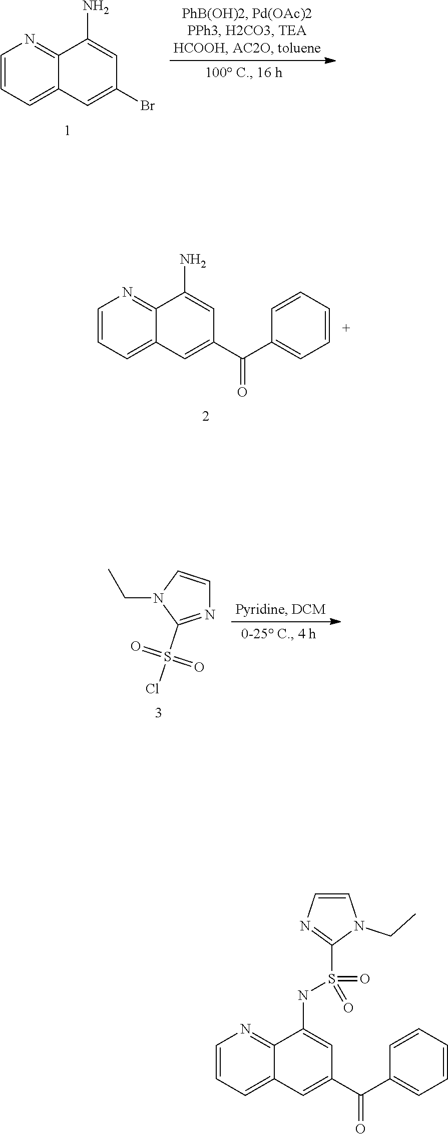 [SMILES: Nc1cc(Br)cc2cccnc12 ; Nc1cc(C(=O)c2ccccc2)cc2cccnc12 ; CCn1ccnc1S(=O)(=O)Cl ; CCn1ccnc1S(=O)(=O)Nc1cc(C(=O)c2ccccc2)cc2cccnc12]

6-Benzoylquinolin-8-amine. To a stirred solution of 6-bromoquinolin-8-amine (250 mg, 1.13 mmol) in toluene (15 mL) in a sealed tube K₂CO₃ (467 mg, 3.38 mmol) was added. Resulting mixture was degassed with argon for 5 min. Triphenyl phosphine (18 mg, 0.07 mmol) and Pd(OAc)₂ (8 mg, 0.04 mmol) were added under inert atmosphere. Phenyl boronic acid (165 mg, 1.35 mmol) was added followed by triethyl amine (0.7 mL, 4.51 mmol) and again purged with argon for 5 min. Formic acid (0.13 mL, 3.38 mmol) and acetic anhydride (0.32 mL, 3.38 mmol) were added and resulting reaction mixture was heated at 100° C. for 16 h. After completion, it was cooled to ambient temperature, filtered through a short pad of celite and washed with ethyl acetate. Filtrate part was washed with water, brine, dried over anhydrous sodium sulphate, filtered and concentrated under reduced pressure. Crude product was purified by combiflash column chromatography (15-20% EtOAc in hexane) to afford 6-benzoylquinolin-8-amine (120 mg, 43%) as grey solid. MS (ESI): m/z 249.2 [M+1]⁺.

N-(6-Benzoylquinolin-8-yl)-1-ethyl-1H-imidazole-2-sulfonamide. To a stirred solution of 6-benzoylquinolin-8-amine (120 mg, 0.35 mmol) in DCM (4 mL) pyridine (2.5 mL) was added at 0° C. and stirred for 10 min. 1-Ethyl-1H-imidazole-2-sulfonyl chloride (235 mg, 1.21 mmol in DCM), was added at 0° C. and resulting reaction mixture was stirred at room temperature for 2 h. After completion, reaction mixture was diluted with water and extracted with DCM. Combined organic layer was washed with brine, dried over anhydrous sodium sulphate, filtered and concentrated under reduced pressure. Crude product was purified by combiflash column chromatography (65-70% EtOAc in hexane) to afford N-(6-benzoylquinolin-8-yl)-1-ethyl-1H-imidazole-2-sulfonamide (20 mg, 14%). ¹H NMR (400 MHz, DMSO-d₆) δ 1.33 (t, J=7.2 Hz, 3H), 4.38 (q, J=7.2 Hz, 2H), 6.94 (s, 1H), 7.48 (s, 1H), 7.59 (t, J=7.6 Hz, 2H), 7.67-7.77 (m, 2H), 7.78 (d, J=7.3 Hz, 2H), 8.10 (s, 1H), 8.14 (brs, 1H), 8.59 (d, J=8.1 Hz, 1H), 9.01 (dd, J=1.6, 4.2 Hz, 1H), 10.70 (brs, 1H). MS (ESI): m/z 407.2 [M+1]⁺.

### In Vitro Parasite Motility Assays

Parasite motility assays. Adult and microfilariae B. malayi and B. pahangi parasites, harvested from infected jirds, were procured from the NIAID/NIH Filariasis Research Reagent Resource Center (FR3). Adult and microfilariae of L. sigmodontis were procured from TRS labs Inc. (Athens, GA). Adult worms were plated in 24-well plates with 2 mL of Advanced RPMI 1640 medium (Invitrogen) supplemented with 25 mM HEPES, 2 mM L-Glutamine (Invitrogen), 100 U/mL Penicillin (Invitrogen), 100 g/mL Streptomycin (Invitro-gen), 2.5 g/mL Amphotericin B solution (Invitrogen), and 5% heat inactivated fetal bovine serum and placed in a 37° C. humidified incubator with 5% C₀₂. After 24 h, adult worms were selected based upon motility as described below. After scoring for motility, 4-6 highly motile worms were selected for each treatment group and were transferred to new plates. Microfilariae were centrifuged at 5000×g for 5 min, and re-suspended in 2 ml of media. Microfilarial density was determined using a hemocytometer and were plated in a 96-well plate at 80 microfilariae/well with 200 μL of complete media. Treatment groups received compounds (0.1% DMSO) at 1 μM and 100 nM with 0.1% DMSO as a vehicle control. Cultures were incubated at 37° C. in a humidified incubator with 5% C₀₂. Worms were transferred into a new plate containing fresh media and drug every 48 h. Parasite and microfilariae motility were given a score from 0 to 4 with 4, rapid movement and largely coiled; 3, moderated movement and uncoiled; 2, slow movement and uncoiled; 1, twitching movement and uncoiled; 0, no motility (dead). The motility of the worms and microfilariae were evaluated every 24 h and analyzed by a one sided unpaired Student's t-test using Microsoft Excel. Experiments were performed 2-3 times with similar results. Onchocerciasis: in vitro screening model Onchocerca gutturosa

Parasite and cell cultures. Onchocerca gutturosa adult male worms were obtained by dissection from the nuchal ligament connective tissues of naturally infected cattle, from Gambia, W Africa.

The worms were maintained for at least 24 h in culture before use in Eagles Minimum Essential Medium with Earl's Salts (Gibco, UK)+10% heat inactivated new born calf serum (Gibco, UK)+antibiotic cover of 200 units/ml penicillin, 200 μg/ml streptomycin and 0.5 μg/ml amphotericin B (Sigma, UK). Only normally active specimens were used in the test. All cultures and assays were conducted at 37° C. under an atmosphere of 5% C₀₂ in air.

Drug sensitivity assays. Compound stock solutions were prepared in 100% DMSO unless otherwise indicated and diluted into the medium. Any unused compound stocks were stored at −20° C. Assays were performed in sterile 24-well (2 ml) plates (Falcon, UK). Worms were then transferred individually to each well of the plate using fine forceps. Worm viability was assessed using 2 parameters:
The measurement of mean worm motility scores on a scale of 0 (immotile) to 10 (maximum) every 24 h, terminating at 120 h, using an Olympus inverted microscope.
The biochemical evaluation of worm viability using MTT/formazan colorimetry. The MTT assay was carried out after the last motility reading (120 h). Single intact worms were placed in each well of a 48-well plate (Falcon, UK) containing 0.5 ml of a solution consisting of 0.5 mg/ml MTT (Sigma UK) in phosphate buffered saline, and then incubated for 30 min at 37° C. The worms were removed, blotted carefully, and individually transferred to separate wells of a 96-well microtiter plate, each containing 200 μl of DMSO to solubilize the formazan. After 1 h the plate was gently agitated to disperse the color evenly and the absorbance value (optical density) of the resulting formazan solution was determined at 490 nm using a multi-well scanning spectrophotometer (Elisa-reader, Dynatech, UK). Inhibition of formazan formation was correlated with worm damage or death.

Primary screen, New compounds were usually tested at 1.25×10⁻⁵ M. Also expressed in μg/ml. Test drugs (2 worms/group) were compared to untreated controls (6 worms/group) and a positive control (standard drug, 6 worms/group). The standard used was Immiticide (Merial): this drug produces a reduction in motility of 100%, and mean inhibition of formazan formation of ˜-85%. The approximate motility EC₅₀ for Immiticide was 3×10⁻⁷ M, and for ivermectin was 1×10 M. The readouts are: Motility score (mean % reduction at 120 h) MTT colorimetry (mean inhibition of formazan formation).

A test compound was considered active if there was a 50% or greater reduction in motility score and/or a 50% or greater inhibition of formazan formation compared to untreated controls.

Compounds were classified as moderately active if there was a 50-99% reduction in motility and/or inhibition of formazan, or highly active at 100%/lower concentrations.

Secondary screen. All active compounds were re-tested. Serial 1 in 4 drug dilutions was carried out to find activity endpoint and EC₅₀ values for motility reduction and inhibition of formazan formation were produced. EC₅₀ values were determined using Excel or Origin V7 scientific graphing and data analysis software.

Heartworm Screen Dirofilaria immitis (D. immitis)

Dirofilaria immitis, Microfilaria (DiMF) Assay. Compounds were dissolved and serially diluted in DMSO. Aliquots were spotted to the empty wells of assay plates. Media and microfilariae of Dirofilaria immitis were added to each well to dilute the test compounds to the desired concentrations. Assay plates were incubated for approximately 72 hours, and the larvae in each well were observed microscopically for drug effect. Microfilariae in each well were assessed subjectively for survival or paralysis, and results were reported as Minimum Effective Dose (MED).

Dirofilaria immitis, L4 stage (DiL4) Assay. Compounds were dissolved and serially diluted in DMSO. Aliquots were spotted to the empty wells of assay plates. Media and 4th stage larvae (L4) of Dirofilaria immitis were added to each well to dilute the test compounds to the desired concentrations. Assay plates were incubated for approximately 72 hours, and the larvae in each well were observed microscopically for drug effect. Larvae in each well were assessed subjectively for survival or paralysis, and results were reported as Minimum Effective Dose (MED).

The compounds described herein demonstrated nematocidal activity against either Dirofilaria immitis (Larva stage 4 (DiL4)) and/or Dirofilaria immitis (microfilaria (DiMF)) as determined by reductions in nematode motility either by paralysis or death. Active and selective (DiL4 vs. DiMF potency) example compounds were subsequently evaluated in heartworm positive dog studies to correlate the in vitro selectivity profile with in vivo effects on circulating microfilariae.

Activity of the Sulfonamide Compounds in the parasite motility assays is shown in Table 1, Table 2 and Table 3. Further activity of the Sulfonamide Compounds in the parasite motility assays is shown in Table 1, Table 2, Table 3, and Table 4.

### In Vivo Filariasis Assays

L. sigmodontis in vivo assays. The infection of mice and jirds can be either initiated by the natural route, exposure of mites containing infective third stage larvae (L3) of L. sigmodontis, or via the injection (subcutaneous, intraperitoneal or intravenous) of a known number of L3 larvae (G. Karadjian et al., Migratory phase of Litomosoides sigmodontis filarial infective larvae is associated with pathology and transient increase of S100A9 expressing neutrophils in the lung, PLoS Negl Trop Dis 11, e0005596 (2017)). Upon infection L3 larvae migrate from the site of inoculation within 2-6 days via the lymphatics to the thoracic cavity, where they molt around 10 days post infection (dpi) into 4th stage larvae and around 30 dpi into adult worms. Approximately 56 dpi adult female worms start to release microfilariae that enter the peripheral blood. In BALB/c mice, adult worm burden starts to decline around 70 dpi and by 100 dpi at which most of the adult worms are cleared. Jirds harbor the adult worms for more than one year.

L. sigmodontis mouse model. The L. sigmodontis mouse model allows the analysis of the activity of compounds on the adult worm or the development into adult worms.

L. sigmodontis jird model. In order to assess the efficacy of drug candidates during chronic, patient infection the L. sigmodontis jird model was used. In general, treatment with drug candidates was initiated 12 weeks post infection and only microfilariae-positive jirds were included in the experiments. Necropsies were performed in general 8-16 weeks post treatment. This extended time between initiation of treatment and necropsy allowed to identify the macrofilaricidal (adult worm killing) efficacy of slow acting compounds. The jird model allowed the assessment of the in vivo impact of compounds on microfilariae over time. Compounds with strong microfilaricidal efficacy clear the microfilariae from peripheral blood within a short period of time. Compounds with an adult worm sterilizing or macrofilaricidal efficacy (lacking a microfilaricidal efficacy) lead to a delayed reduction of the microfilaremia that exceeds 4 weeks post treatment start. Additional analysis at the time of necropsy included the quantification of adult worms, ratios of female and male adult worms, and motility of adult worms at the time of necropsy. Remaining female adult worms were assessed for their embryogenesis and therefore sterilizing effects of compounds. Embryograms from female adult worms included the quantification of early developmental stages (egg/morulae) and later stages (pretzel stage & stretched microfilariae) according to (S. Ziewer et al., Immunization with L. sigmodontis Microfilariae Reduces Peripheral Microfilaraemia after Challenge Infection by Inhibition of Filarial Embryogenesis, PLoS Negl Trop Dis 6, e1558 (2012)). Lack of early and/or later developmental embryonic stages suggested a sterilizing effect of the compounds. Additional histological and TEM analysis was applied to analyze any tissue damages caused by the drug candidates that may be associated with permanent sterilization.

The L. sigmodontis jird model assessed the macrofilaricidal efficacy of compounds, their impact on microfilaremia, female worm embryogenesis and sterilization.

The Sulfonamide Compounds provided herein were tested and showed activity in both L. sigmodontis mouse and L. sigmodontis jird model assays performed as described herein, with some compounds showing macrofilaricidal activity and some compounds showing macrofilaricidal selectivity.

In some embodiments, the compounds disclosed herein surprisingly presented distinct activity between parasitic nematodes in adult and juvenile stage. In some such embodiments, the compounds disclosed herein were found to be selectively effective against adult filarial nematodes (i.e., were macroselective). Therefore, the compounds disclosed herein have the potential to be potent anti-filarial drugs.

In some embodiments, the compounds disclosed herein surprisingly presented distinct activity between parasitic nematodes in adult and juvenile stage. In some such embodiments, the compounds disclosed herein were found to be selectively effective against adult filarial nematodes (i.e., were macroselective). Therefore, the compounds disclosed herein have the potential to be potent anti-filarial drugs.

Heartworm Dog Studies. Dogs with pre-existing heartworm infections, via surgical transplantation were used for these studies. To confirm that the dogs had circulating microfilariae, blood samples were taken from each dog and examined for microfilariae by using the modified Knott's method. All dog cohorts included in the studies exhibited average microfilariae counts of at least 15,000 MF/mL of the blood (pre-dose). On approximately Day −7, dogs were randomly allocated to treatments (three animals per treatment group) based on Day −7 MF counts. Dogs were fasted overnight prior to dosing and fed immediately following dosing of the test articles. Compounds were administered by point dosing in oral liquid-filled capsules on Day 0. Blood samples were collected to measure MF counts on Days 0 (pre-dose and 2 hours post-dose), 1, 2, 7, 21 and 28. Clinical observations were conducted by a suitably experienced veterinarian on days −7, 0 (immediately prior to treatment, 1-2 hours post-treatment), 1 and 2 whereby any abnormal clinical signs were documented using standard veterinary medical terminology. Additionally, general health observations were conducted throughout the study including (but not limited to) general physical appearance and behavior, abnormalities of food and water consumption, vomiting/regurgitation, appearance of urine and feces and any sign of MF anaphylaxis.

The Sulfonamide Compounds provided herein were tested and showed activity, or will be shown to have activity, on circulating microfilariae in vivo.

### Activity Table

Each of the compounds in Table 1, Table 2, Table 3, and Table 4 was tested in at least one of the in vitro filarial motility assays and was found to have activity therein, with all of the Sulfonamide Compounds of formula (I), formula (Ia), and formula (II), having an IC₅₀ below or at 5 μM in one or more of the assays, with some compounds having an IC₅₀ between 0.5 μM and 5 μM (activity level A), some having an IC₅₀ between 0.2 μM and 0.5 μM (activity level B), and some having an IC₅₀ below 0.2 μM (activity level C). Sulfonamide Compounds of formula (I), formula (Ia), and formula (II), were tested in one or more of the assays and were shown to have activity therein, with some of the Sulfonamide Compounds of formula (I), formula (Ia), and formula (II), having activity against microfilaria at compound concentrations below 1 μM (activity level D) with some compounds having activity against adult filaria at compound concentrations below 1 μM (activity level E).

TABLE 1

| | | | | | | | L. sig- | |
|---|---|---|---|---|---|---|---|---|
| | | | | O. gutt | B. malayi | B. phangi | modontis | D. immitis |
| Compound | | | | Activity | Activity | Activity | Activity | Activity |
| ID | Name | Structure | M+ | class | class | class | class | class |
| 1 | 1-ethyl-N-(5- morpholino- quinolin-8-yl)- 1H-imidazole- 5-sulfonamide | 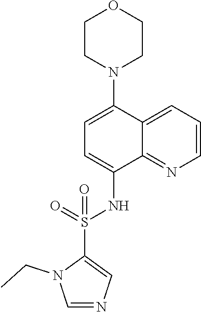 [SMILES: CCn1cncc1S(=O)(=O)Nc1ccc(N2CCOCC2)c2cccnc12] | 388.1 | | | | | |
| 2 | N-(5-chloro- quinolin-8-yl)- 1-isopropyl- 1H-imidazole- 5-sulfonamide | 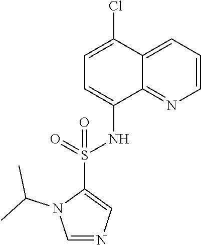 [SMILES: CC(C)n1cncc1S(=O)(=O)Nc1ccc(Cl)c2cccnc12] | 351.1 | | | | | |
| 3 | 1-isopropyl- N-(5-morpho- linoquinolin- 8-yl)-1H- imidazole- 5-sulfonamide | 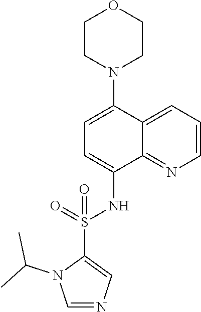 [SMILES: CC(C)n1cncc1S(=O)(=O)Nc1ccc(N2CCOCC2)c2cccnc12] | 402.1 | | | | | |
| 4 | N-(5-chloro- quinolin-8- yl)-1-(cyclo- propylmeth- yl)-1H- imidazole-5- sulfonamide | 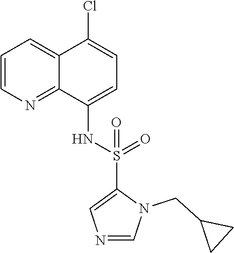 [SMILES: O=S(=O)(Nc1ccc(Cl)c2cccnc12)c1cncn1CC1CC1] | 363 | | | | | |
| 5 | 1-(cyclo- propylmeth- yl)-N-(5- morpholino- quinolin-8- yl)-1H- imidazole-5- sulfonamide | 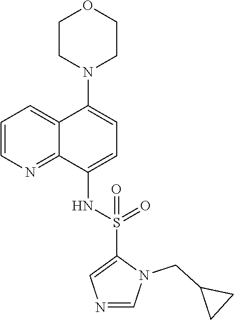 [SMILES: O=S(=O)(Nc1ccc(N2CCOCC2)c2cccnc12)c1cncn1CC1CC1] | 414.2 | | | | | |
| 6 | 1-cyclopropyl- N-(5-morpho- linoquinolin- 8-yl)-1H- imidazole-2- sulfonamide | 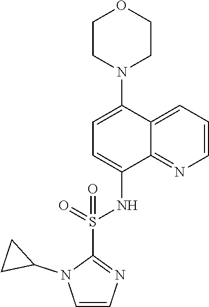 [SMILES: O=S(=O)(Nc1ccc(N2CCOCC2)c2cccnc12)c1nccn1C1CC1] | 400.2 | | | | | |
| 7 | 1-(cyclo- propylmeth- yl)-N-(5- morpholino- quinolin-8- yl)-1H- imidazole-4- sulfonamide | 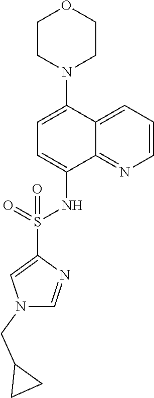 [SMILES: O=S(=O)(Nc1ccc(N2CCOCC2)c2cccnc12)c1cn(CC2CC2)cn1] | 414.2 | | | | | |
| 8 | N-(4-methyl- quinolin-8-yl)- 6-(trifluoro- methyl)pyr- idine-3-sulfon- amide | 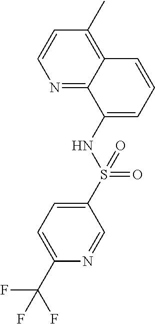 [SMILES: Cc1ccnc2c(NS(=O)(=O)c3ccc(C(F)(F)F)nc3)cccc12] | 368.1 | | | | | B |
| 9 | N-(4-fluoro- quinolin-8-yl)- 6-(trifluoro- methyl)pyr- idine-3- sulfonamide | 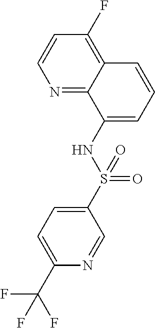 [SMILES: O=S(=O)(Nc1cccc2c(F)ccnc12)c1ccc(C(F)(F)F)nc1] | 372.1 | | | | | |
| 10 | N-(4-meth- oxyquinolin- 8-yl)-6- (trifluoro- methyl)pyr- idine-3- sulfonamide | 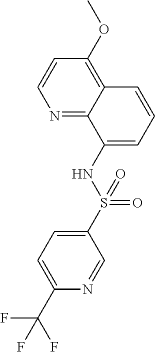 [SMILES: COc1ccnc2c(NS(=O)(=O)c3ccc(C(F)(F)F)nc3)cccc12] | 384.3 | | | | | A |
| 11 | N-(3-meth- oxyquinolin- 8-yl)-6- (trifluoro- methyl)pyr- idine-3- sulfonamide | 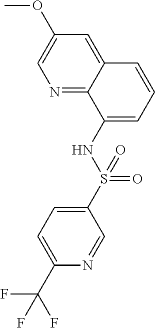 [SMILES: COc1cnc2c(NS(=O)(=O)c3ccc(C(F)(F)F)nc3)cccc2c1] | 384.1 | | | | | A |
| 12 | N-(4-fluoro- quinolin-8-yl)- 3-methylpyr- idine-2- sulfonamide | 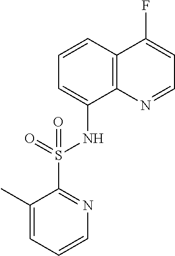 [SMILES: Cc1cccnc1S(=O)(=O)Nc1cccc2c(F)ccnc12] | 318.3 | B | | | | |
| 13 | 3-methyl-N- (4-(trifluoro- methyl)quin- olin-8-yl)pyr- idine-2- sulfonamide | 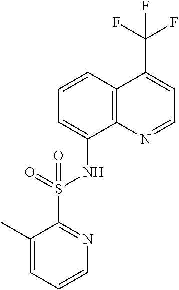 [SMILES: Cc1cccnc1S(=O)(=O)Nc1cccc2c(C(F)(F)F)ccnc12] | 368.1 | C | | | | B |
| 14 | N-(4-meth- oxyquinolin- 8-yl)-3-meth- ylpyridine-2- sulfonamide | 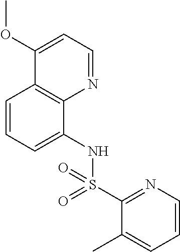 [SMILES: COc1ccnc2c(NS(=O)(=O)c3ncccc3C)cccc12] | 330.2 | A | | | | A |
| 15 | 3-methyl-N- (4-methyl- quinolin-8- yl)pyridine- 2-sulfonamide | 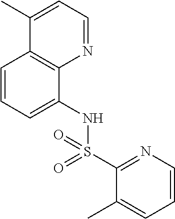 [SMILES: Cc1cccnc1S(=O)(=O)Nc1cccc2c(C)ccnc12] | 314.3 | C | | | | A |
| 16 | N-(7-(cyclo- propylmeth- oxy)quinolin- 8-yl)-3-meth- ylpyridine-2- sulfonamide | 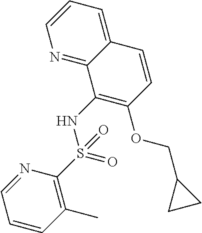 [SMILES: Cc1cccnc1S(=O)(=O)Nc1c(OCC2CC2)ccc2cccnc12] | 370.3 | A | | | | C |
| 17 | N-(7-cyclo- propylquin- olin-8-yl)-3- methylpyr- idine-2- sulfonamide | 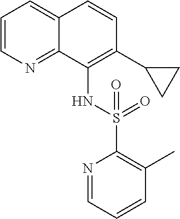 [SMILES: Cc1cccnc1S(=O)(=O)Nc1c(C2CC2)ccc2cccnc12] | 340.4 | B | | | | B |
| 18 | 3-methyl-N- (7-morpho- linoquinolin- 8-yl)pyridine- 2-sulfonamide | 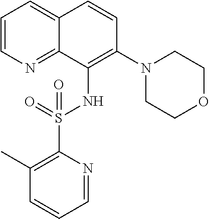 [SMILES: Cc1cccnc1S(=O)(=O)Nc1c(N2CCOCC2)ccc2cccnc12] | 385.4 | B | | | | B |
| 19 | N-(4-iso- propoxyquin- olin-8-yl)-3- methylpyr- idine-2- sulfonamide | 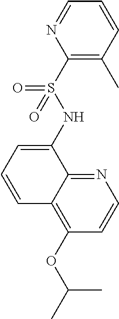 [SMILES: Cc1cccnc1S(=O)(=O)Nc1cccc2c(OC(C)C)ccnc12] | 358.4 | A | | | | |
| 20 | N-(3-(azet- idin-1-yl)- quinolin-5- yl)-3-meth- ylpyridine-2- sulfonamide | 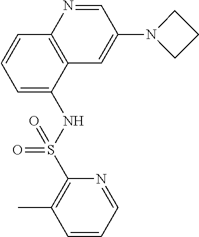 [SMILES: Cc1cccnc1S(=O)(=O)Nc1cccc2ncc(N3CCC3)cc12] | 355 | B | | | D | |
| 21 | 3-methyl-N- (3-morpho- linoquinolin- 8-yl)pyridine- 2-sulfonamide | 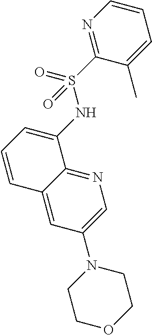 [SMILES: Cc1cccnc1S(=O)(=O)Nc1cccc2cc(N3CCOCC3)cnc12] | 385.4 | C | | | D, E | A |
| 22 | 3-methyl-N- (2-phenyl- quinolin-8- yl)pyridine- 2-sulfonamide | 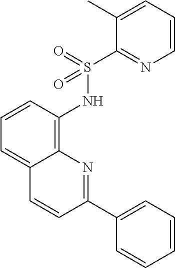 [SMILES: Cc1cccnc1S(=O)(=O)Nc1cccc2ccc(-c3ccccc3)nc12] | 376.2 | A | | | | |
| 23 | N-(2-(azet- idin-1-yl)- quinolin-8- yl)-3-meth- ylpyridine-2- sulfonamide | 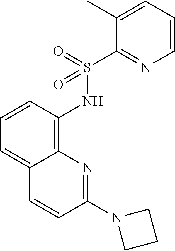 [SMILES: Cc1cccnc1S(=O)(=O)Nc1cccc2ccc(N3CCC3)nc12] | 355 | A | | | | |
| 24 | 3-methyl-N- (3-morpho- linoquinolin- 5-yl)pyridine- 2-sulfonamide | 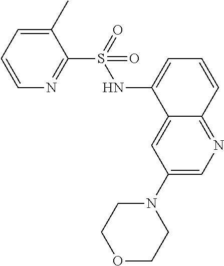 [SMILES: Cc1cccnc1S(=O)(=O)Nc1cccc2ncc(N3CCOCC3)cc12] | 385.1 | A | | | | |
| 25 | 3-methyl-N- (2-morpho- linoquinolin- 8-yl)pyridine- 2-sulfonamide | 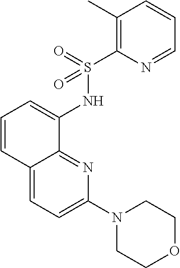 [SMILES: Cc1cccnc1S(=O)(=O)Nc1cccc2ccc(N3CCOCC3)nc12] | 385.2 | | | | | |
| 26 | N-(2-cyclo- propylquin- olin-8-yl)-3- methylpyr- idine-2- sulfonamide | 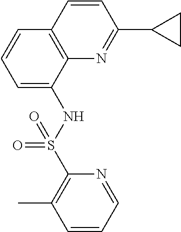 [SMILES: Cc1cccnc1S(=O)(=O)Nc1cccc2ccc(C3CC3)nc12] | 340 | A | | | | A |
| 27 | N-(4-cyclo- propylquin- olin-8-yl)-3- methylpyr- idine-2- sulfonamide | 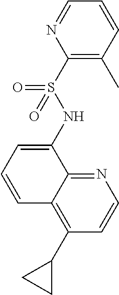 [SMILES: Cc1cccnc1S(=O)(=O)Nc1cccc2c(C3CC3)ccnc12] | 340.4 | A | | | | A |
| 28 | N-(4-(cyclo- propylmeth- oxy)quinolin- 8-yl)-3-meth- ylpyridine-2- sulfonamide | 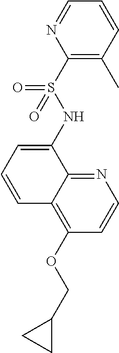 [SMILES: Cc1cccnc1S(=O)(=O)Nc1cccc2c(OCC3CC3)ccnc12] | 370.3 | A | | | | A |
| 29 | N-(3-cyclo- propylquin- olin-8-yl)-3- methylpyr- idine-2- sulfonamide | 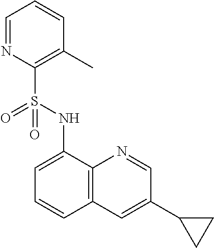 [SMILES: Cc1cccnc1S(=O)(=O)Nc1cccc2cc(C3CC3)cnc12] | 340.3 | A | | | | A |
| 30 | 3-methyl-N- (4-phenyl- quinolin-8- yl)pyridine-2- sulfonamide | 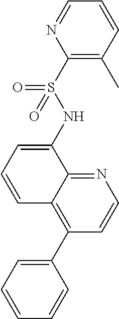 [SMILES: Cc1cccnc1S(=O)(=O)Nc1cccc2c(-c3ccccc3)ccnc12] | 376.2 | A | | | | A |
| 31 | 4-cyclopropyl- N-(6-methoxy- 1,5-naphthyr- idin-4-yl)pyr- idine-2- sulfonamide | 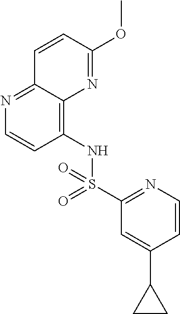 [SMILES: COc1ccc2nccc(NS(=O)(=O)c3cc(C4CC4)ccn3)c2n1] | 357 | | | | | |
| 32 | N-(7-chloro- 2-methyl- 1,2,3,4-tetra- hydroisoquin- olin-8-yl)-3- methylpyr- idine-2- sulfonamide | 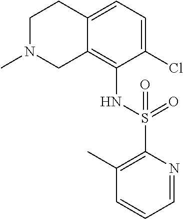 [SMILES: Cc1cccnc1S(=O)(=O)Nc1c(Cl)ccc2c1CN(C)CC2] | 352.1 | | | E | D | |
| 33 | N-(2-cyclo- propyliso- indolin-4-yl)- 1-isopropyl- 1H-imidazole- 2-sulfonamide | 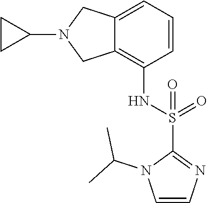 [SMILES: CC(C)n1ccnc1S(=O)(=O)Nc1cccc2c1CN(C1CC1)C2] | 347.1 | B | | | D | |
| 34 | 1-isopropyl- N-(2-methyl- 1,2,3,4-tetra- hydroisoquin- olin-8-yl)- 1H-imidazole- 2-sulfonamide | 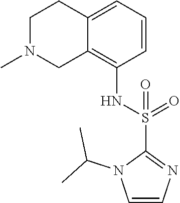 [SMILES: CC(C)n1ccnc1S(=O)(=O)Nc1cccc2c1CN(C)CC2] | 335.3 | | | | D | |
| 35 | 1-isopropyl- N-(6-meth- ylquinolin- 8-yl)-1H- imidazole-2- sulfonamide | 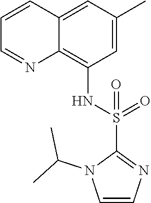 [SMILES: Cc1cc(NS(=O)(=O)c2nccn2C(C)C)c2ncccc2c1] | 331.2 | B | D | E | D | B |
| 36 | N-(2-cyclo- propyl- 1,2,3,4-tetra- hydroisoquin- olin-8-yl)-3- methylpyr- idine-2- sulfonamide | 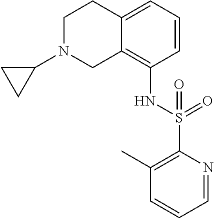 [SMILES: Cc1cccnc1S(=O)(=O)Nc1cccc2c1CN(C1CC1)CC2] | 344.3 | A | | E | D | |
| 37 | 3-cyclo- propyl-N-(2- methyl- 1,2,3,4-tetra- hydroisoquin- olin-8-yl)pyr- idine-2- sulfonamide | 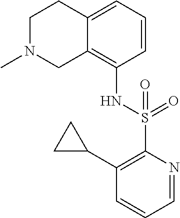 [SMILES: CN1CCc2cccc(NS(=O)(=O)c3ncccc3C3CC3)c2C1] | 344.3 | B | | | D | |
| 38 | N-(1-cyclo- propyl- 1,2,3,4-tetra- hydroquin- olin-8-yl)-3- methylpyr- idine-2- sulfonamide | 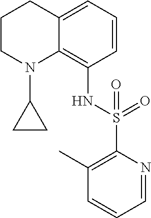 [SMILES: Cc1cccnc1S(=O)(=O)Nc1cccc2c1N(C1CC1)CCC2] | 344.1 | A | | | D | |
| 39 | 1-isopropyl- N-(2-methyl- 1,2,3,4-tetra- hydroisoquin- olin-8-yl)- 1H-pyrazole- 5-sulfonamide | 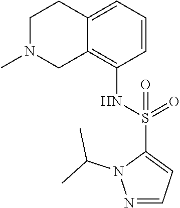 [SMILES: CC(C)n1nccc1S(=O)(=O)Nc1cccc2c1CN(C)CC2] | 335.3 | | | | D | |
| 40 | N-(2-cyclo- propyliso- indolin-4-yl)- 1-isopropyl- 1H-pyrazole- 5-sulfonamide | 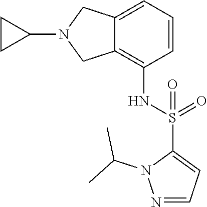 [SMILES: CC(C)n1nccc1S(=O)(=O)Nc1cccc2c1CN(C1CC1)C2] | 347.1 | A | | | | |
| 41 | N-(2-cyclo- propyliso- indolin-4-yl)- 3-methylpyr- idine-2- sulfonamide | 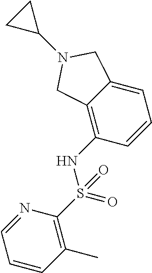 [SMILES: Cc1cccnc1S(=O)(=O)Nc1cccc2c1CN(C1CC1)C2] | 330.1 | A | | E | D | |
| 42 | N-(1-cyclo- propyl- 1,2,3,4-tetra- hydroquin- olin-8-yl)-3- (dimethyl- amino)pyr- idine-2- sulfonamide | 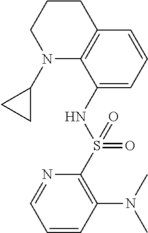 [SMILES: CN(C)c1cccnc1S(=O)(=O)Nc1cccc2c1N(C1CC1)CCC2] | 373.2 | A | | E | D | A |
| 43 | 3-methyl-N- (2-methyl- 1,2,3,4-tetra- hydroisoquin- olin-8-yl)pyr- idine-2- sulfonamide | 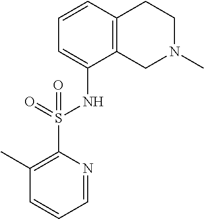 | 318.1 | C | | | D | |
| 44 | 3-methyl-N- (1-methyl- indolin-7-yl)- pyridine-2- sulfonamide | 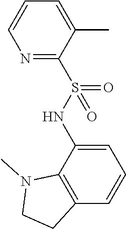 [SMILES: Cc1cccnc1S(=O)(=O)Nc1cccc2c1N(C)CC2] | 304.1 | A | | E | D | |
| 45 | 3-(dimethyl- amino)-N- (1,2,3,4-tetra- hydroquin- olin-8-yl)- pyridine-2- sulfonamide | 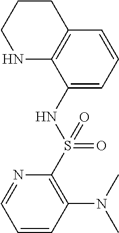 [SMILES: CN(C)c1cccnc1S(=O)(=O)Nc1cccc2c1NCCC2] | 333.1 | A | | | D | A |
| 46 | 3-fluoro-N- (1,2,3,4-tetra- hydroquin- olin-8-yl)- pyridine-2- sulfonamide | 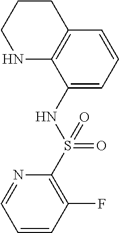 [SMILES: O=S(=O)(Nc1cccc2c1NCCC2)c1ncccc1F] | 308.1 | A | | | D | |
| 47 | N-(2-cyclo- propyliso- indolin-4-yl)- 3-(dimethyl- amino)pyr- idine-2- sulfonamide | 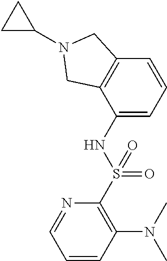 [SMILES: CN(C)c1cccnc1S(=O)(=O)Nc1cccc2c1CN(C1CC1)C2] | 359.1 | A | | | D | |
| 48 | 3-cyclo- propyl-N-(5- fluoroquin- olin-8-yl)- pyridine-2- sulfonamide | 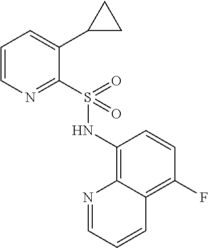 [SMILES: O=S(=O)(Nc1ccc(F)c2cccnc12)c1ncccc1C1CC1] | 344.1 | C | D | E | D, E | A |
| 49 | N-(7-chloro- quinolin-8- yl)-5-(dimeth- ylamino)pyr- azine-2- sulfonamide | 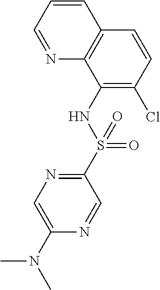 [SMILES: CN(C)c1cnc(S(=O)(=O)Nc2c(Cl)ccc3cccnc23)cn1] | 364 | A | | | D | |
| 50 | N-(7-chloro- quinolin-8- yl)-6-(dimeth- ylamino)pyr- azine-2- sulfonamide | 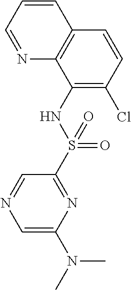 [SMILES: CN(C)c1cncc(S(=O)(=O)Nc2c(Cl)ccc3cccnc23)n1] | 364 | A | | | D | |
| 51 | 3-methyl-N- (2-methyl- isoindolin-4- yl)pyridine-2- sulfonamide | 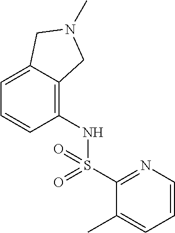 [SMILES: Cc1cccnc1S(=O)(=O)Nc1cccc2c1CN(C)C2] | 304.1 | C | | E | | |
| 52 | N-(7-chloro- quinolin-8- yl)-5-methyl- pyrazine-2- sulfonamide | 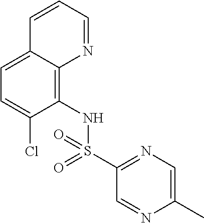 [SMILES: Cc1cnc(S(=O)(=O)Nc2c(Cl)ccc3cccnc23)cn1] | 335.1 | | | | | |
| 53 | N-(7-chloro- quinolin-8- yl)-3,6- dimethyl- pyrazine-2- sulfonamide | 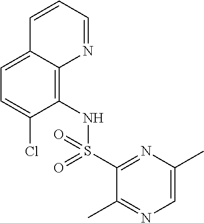 [SMILES: Cc1cnc(C)c(S(=O)(=O)Nc2c(Cl)ccc3cccnc23)n1] | 349 | A | | | | A |
| 54 | N-(iso- indolin-4-yl)- 3-methyl- pyridine-2- sulfonamide | 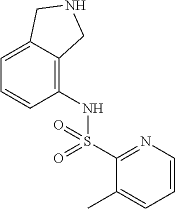 [SMILES: Cc1cccnc1S(=O)(=O)Nc1cccc2c1CNC2] | 290.1 | | D | | | |
| 55 | 3-methyl-N- (1-methyl- 1,2,3,4-tetra- hydroquin- olin-8-yl)- pyridine-2- sulfonamide | 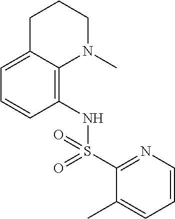 [SMILES: Cc1cccnc1S(=O)(=O)Nc1cccc2c1N(C)CCC2] | 318.1 | A | | E | | A |
| 56 | 3-methyl-N- (1,2,3,4-tetra- hydroquinolin- 8-yl)pyridine- 2-sulfonamide | 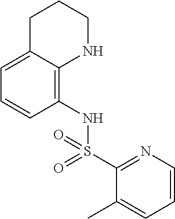 [SMILES: Cc1cccnc1S(=O)(=O)Nc1cccc2c1NCCC2] | 304.2 | A | | | D | |
| 57 | 1-ethyl-N-(5- methoxyquin- olin-8-yl)-1H- imidazole-2- sulfonamide | 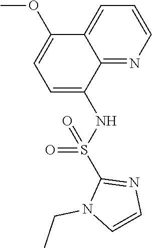 [SMILES: CCn1ccnc1S(=O)(=O)Nc1ccc(OC)c2cccnc12] | 332.8 | A | | | | |
| 58 | 5-methyl-N- (5-methyl- quinolin-8- yl)pyridine- 2-sulfonamide | 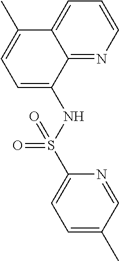 [SMILES: Cc1ccc(S(=O)(=O)Nc2ccc(C)c3cccnc23)nc1] | 313.9 | B | | | | A |
| 59 | N-(5-fluoro- quinolin-8- yl)-1-meth- yl-1H-pyr- azole-4- sulfonamide | 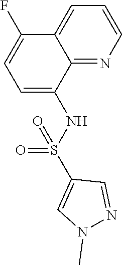 [SMILES: Cn1cc(S(=O)(=O)Nc2ccc(F)c3cccnc23)cn1] | 307 | A | | | | |
| 60 | N-(5-meth- oxyquinolin- 8-yl)furan-2- sulfonamide | 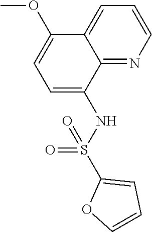 [SMILES: COc1ccc(NS(=O)(=O)c2ccco2)c2ncccc12] | 305 | A | | | | |
| 61 | N-(5-morph- olinoquin- olin-8-yl)- furan-2- sulfonamide | 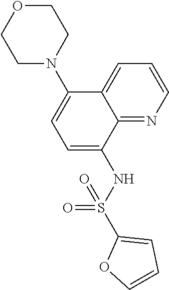 [SMILES: O=S(=O)(Nc1ccc(N2CCOCC2)c2cccnc12)c1ccco1] | 360 | A | | | | |
| 62 | N-(5-morph- olinoquin- olin-8-yl)- 1H-pyrazole- 4-sulfonamide | 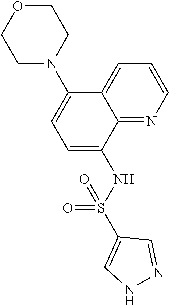 [SMILES: O=S(=O)(Nc1ccc(N2CCOCC2)c2cccnc12)c1cn[nH]c1] | 360.1 | A | D, E | | E | |
| 63 | N-(5-fluoro- quinolin-8- yl)-1H- pyrazole-4- sulfonamide | 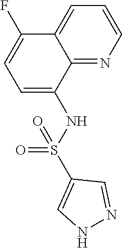 [SMILES: O=S(=O)(Nc1ccc(F)c2cccnc12)c1cn[nH]c1] | 293 | A | E | | | |
| 64 | N-(5-fluoro- quinolin-8- yl)furan-2- sulfonamide | 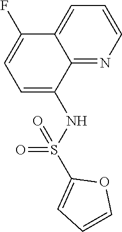 [SMILES: O=S(=O)(Nc1ccc(F)c2cccnc12)c1ccco1] | 293 | C | D, E | | D, E | |
| 65 | N-(5-methyl- quinolin-8- yl)furan-2- sulfonamide | 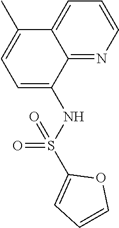 [SMILES: Cc1ccc(NS(=O)(=O)c2ccco2)c2ncccc12] | 289 | A | | | | |
| 66 | 3-methyl-N- (3-methyl- quinolin-8- yl)pyridine-2- sulfonamide | 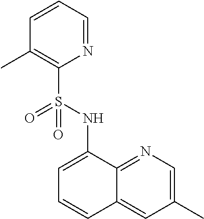 | 314.1 | | | | | A |
| 67 | 3-methyl-N- (2-methyl- quinolin-8- yl)pyridine- 2-sulfonamide | 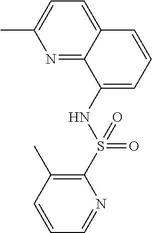 [SMILES: Cc1ccc2cccc(NS(=O)(=O)c3ncccc3C)c2n1] | 314.3 | | | | | A |
| 68 | N-(7-chloro- quinolin-8- yl)-3-iso- propylpyr- idine-2- sulfonamide | 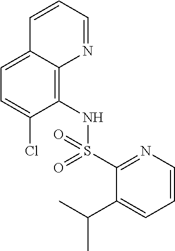 [SMILES: CC(C)c1cccnc1S(=O)(=O)Nc1c(Cl)ccc2cccnc12] | 362 | C | D, E | E | D, E | A |
| 69 | N-(7-chloro- quinolin-8- yl)-3-cyclo- propylpyr- idine-2- sulfonamide | 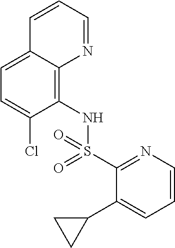 [SMILES: O=S(=O)(Nc1c(Cl)ccc2cccnc12)c1ncccc1C1CC1] | 360.1 | C | D, E | E | D, E | |
| 70 | N-(7-chloro- quinolin-8- yl)-3-methyl- pyrazine-2- sulfonamide | 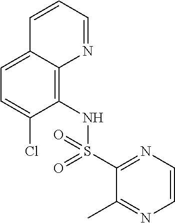 [SMILES: Cc1nccnc1S(=O)(=O)Nc1c(Cl)ccc2cccnc12] | 355.1 | A | | D | | |
| 71 | N-(7-chloro- quinolin-8- yl)-4-cyano- pyridine-2- sulfonamide | 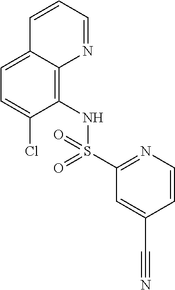 [SMILES: N#Cc1ccnc(S(=O)(=O)Nc2c(Cl)ccc3cccnc23)c1] | 345.1 | C | | E | D | |
| 72 | 3-cyano-N- (5-fluoroquin- olin-8-yl)ben- zenesulfon- amide | 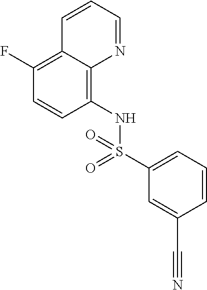 [SMILES: N#Cc1cccc(S(=O)(=O)Nc2ccc(F)c3cccnc23)c1] | 328.1 | C | E | E | D | A |
| 73 | 3-chloro-N- (7-chloro- quinolin-8- yl)pyridine- 2-sulfonamide | 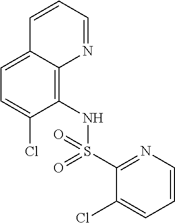 [SMILES: O=S(=O)(Nc1c(Cl)ccc2cccnc12)c1ncccc1Cl] | 354 | C | | E | E | A |
| 74 | N-(7-chloro- quinolin-8- yl)-6-(pyrrol- idin-1-yl)pyr- azine-2- sulfonamide | 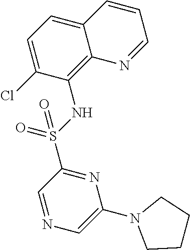 [SMILES: O=S(=O)(Nc1c(Cl)ccc2cccnc12)c1cncc(N2CCCC2)n1] | 390.1 | B | | E | | |
| 75 | 3-methyl-N- (5-morpho- linoquinolin- 8-yl)pyrazine- 2-sulfonamide | 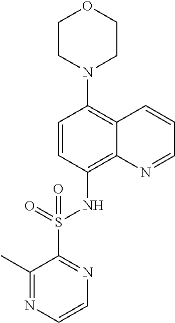 [SMILES: Cc1nccnc1S(=O)(=O)Nc1ccc(N2CCOCC2)c2cccnc12] | 386.1 | C | D | E | | A |
| 76 | N-(7-chloro- quinolin-8-yl)- 3-(dimethyl- amino)pyr- azine-2- sulfonamide | 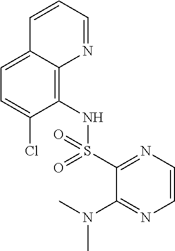 [SMILES: CN(C)c1nccnc1S(=O)(=O)Nc1c(Cl)ccc2cccnc12] | 364 | A | D | D | | A |
| 77 | N-(7-chloro- quinolin-8- yl)-1-iso- propyl-1H- pyrazole-5- sulfonamide | 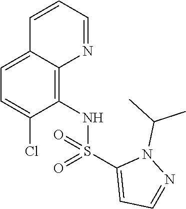 [SMILES: CC(C)n1nccc1S(=O)(=O)Nc1c(Cl)ccc2cccnc12] | 351 | B | | E | | A |
| 78 | N-(7-chloro- quinolin-8- yl)-5-meth- oxypyrazine- 2-sulfonamide | 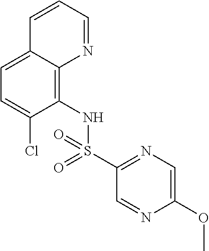 [SMILES: COc1cnc(S(=O)(=O)Nc2c(Cl)ccc3cccnc23)cn1] | 351 | A | | | | |
| 79 | N-(4-chloro- quinolin-8- yl)-1-iso- propyl-1H- pyrazole-5- sulfonamide | 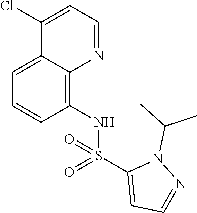 [SMILES: CC(C)n1nccc1S(=O)(=O)Nc1cccc2c(Cl)ccnc12] | 351.1 | C | | E | E | |
| 80 | 3-cyano-N- (5-morpho- linoquinolin- 8-yl)ben- zenesulfon- amide | 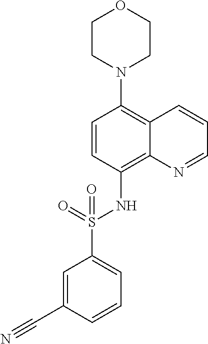 [SMILES: N#Cc1cccc(S(=O)(=O)Nc2ccc(N3CCOCC3)c3cccnc23)c1] | 300.1 | C | | | D | A |
| 81 | N-(7-chloro- quinolin-8- yl)-6-meth- oxypyrazine- 2-sulfonamide | 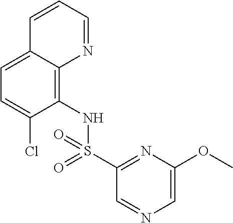 [SMILES: COc1cncc(S(=O)(=O)Nc2c(Cl)ccc3cccnc23)n1] | 351 | A | D | | | |
| 82 | N-(7-chloro- quinolin-8- yl)-3-cyano- benzene sulfonamide | 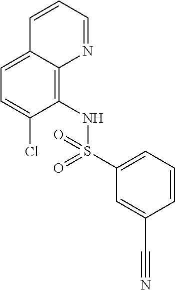 [SMILES: N#Cc1cccc(S(=O)(=O)Nc2c(Cl)ccc3cccnc23)c1] | 344.1 | A | | | | |
| 83 | 1-ethyl-N-(5- methylquin- olin-8-yl)-1H- imidazole-2- sulfonamide | 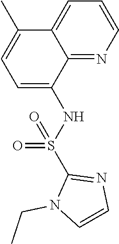 [SMILES: CCn1ccnc1S(=O)(=O)Nc1ccc(C)c2cccnc12] | 316.8 | C | D | | | B |
| 84 | 1-ethyl-N-(7- methoxyquin- olin-8-yl)-1H- imidazole-2- sulfonamide | 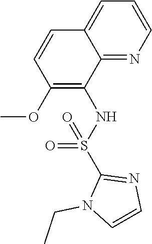 [SMILES: CCn1ccnc1S(=O)(=O)Nc1c(OC)ccc2cccnc12] | 332.8 | C | D | | D, E | B |
| 85 | N-(4-chloro- quinolin-8- yl)-1-ethyl- 1H-imidazole- 2-sulfonamide | 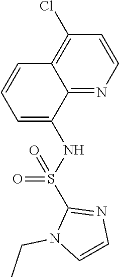 [SMILES: CCn1ccnc1S(=O)(=O)Nc1cccc2c(Cl)ccnc12] | 336.8 | | D | | | A |
| 86 | 1-ethyl-N-(3- methoxyquin- olin-8-yl)-1H- imidazole-2- sulfonamide | 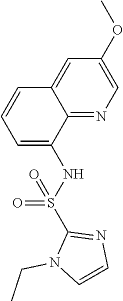 [SMILES: CCn1ccnc1S(=O)(=O)Nc1cccc2cc(OC)cnc12] | 332.8 | C | E | | | B |
| 87 | N-(3-fluoro- quinolin-8- yl)-5-methyl- pyridine-2- sulfonamide | 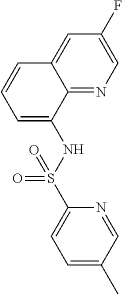 [SMILES: Cc1ccc(S(=O)(=O)Nc2cccc3cc(F)cnc23)nc1] | 317.8 | C | | | D | B |
| 88 | N-(6-fluoro- quinolin-8- yl)-5-methyl- pyridine-2- sulfonamide | 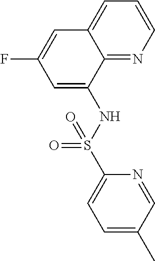 [SMILES: Cc1ccc(S(=O)(=O)Nc2cc(F)cc3cccnc23)nc1] | 317.8 | A | D | | | A |
| 89 | N-(6-chloro- quinolin-8- yl)-1-methyl- 1H-imidazole- 2-sulfonamide | 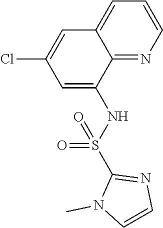 [SMILES: Cn1ccnc1S(=O)(=O)Nc1cc(Cl)cc2cccnc12] | 323 | | D, E | | D, E | |
| 90 | 1-methyl-N- (6-(trifluoro- methyl)quin- olin-8-yl)-1H- imidazole-2- sulfonamide | 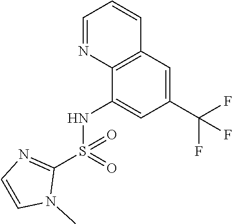 [SMILES: Cn1ccnc1S(=O)(=O)Nc1cc(C(F)(F)F)cc2cccnc12] | 357 | | D | | | |
| 91 | N-(6-fluoro- quinolin-8- yl)-3,5- dimethyl- benzene- sulfonamide | 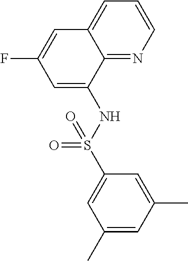 [SMILES: Cc1cc(C)cc(S(=O)(=O)Nc2cc(F)cc3cccnc23)c1] | 331 | C | | | | |
| 92 | 3-(N-(5- morpholino- quin-olin-8- yl)sulfamoyl)- benzamide | 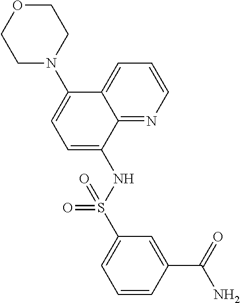 [SMILES: NC(=O)c1cccc(S(=O)(=O)Nc2ccc(N3CCOCC3)c3cccnc23)c1] | 413.2 | A | E | | | |
| 93 | 4-methyl-N- (6-methyl- quinolin-8- yl)benzene- sulfonamide | 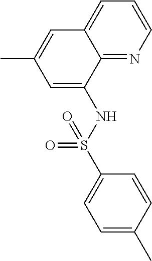 | 321.9 | B | | | | |
| 94 | N-(6-hydroxy- quinolin-8- yl)pyridine- 2-sulfonamide | 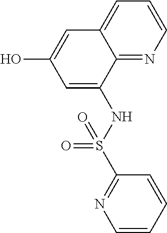 [SMILES: O=S(=O)(Nc1cc(O)cc2cccnc12)c1ccccn1] | 302 | A | D | | | |
| 95 | N-(5-(4- hydroxy-4- phenylpiper- idin-1-yl)- quinolin-8- yl)-4-methyl- benzene- sulfonamide | 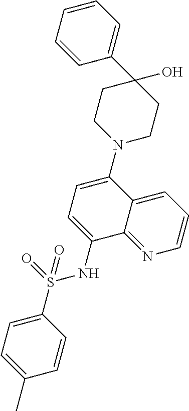 [SMILES: Cc1ccc(S(=O)(=O)Nc2ccc(N3CCC(O)(c4ccccc4)CC3)c3cccnc23)cc1] | 474.2 | A | D | | | |
| 96 | 4-methyl-N- (3-methyl- quinolin-8- yl)benzene- sulfonamide | 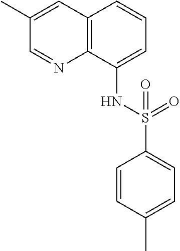 [SMILES: Cc1ccc(S(=O)(=O)Nc2cccc3cc(C)cnc23)cc1] | 313.1 | C | D | | | |
| 97 | N-(6-phenyl- quinolin-8- yl)pyridine-2- sulfonamide | 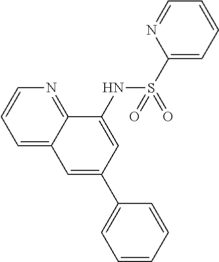 [SMILES: O=S(=O)(Nc1cc(-c2ccccc2)cc2cccnc12)c1ccccn1] | 362.1 | A | D | | | |
| 98 | N-(7-chloro- quinolin-8- yl)-3-fluoro- pyridine-2- sulfonamide | 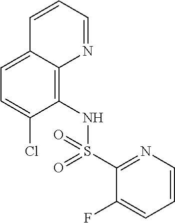 [SMILES: O=S(=O)(Nc1c(Cl)ccc2cccnc12)c1ncccc1F] | 338.1 | C | | E | D | A |
| 99 | N-(7-chloro- quinolin-8-yl)- 3-(dimethyl- amino)pyridine- 2-sulfonamide | 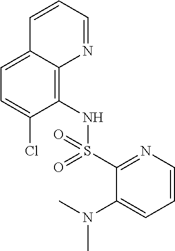 [SMILES: CN(C)c1cccnc1S(=O)(=O)Nc1c(Cl)ccc2cccnc12] | 360 | A | | E | | A |
| 100 | N-(7-chloro- quinolin-8-yl)- 3-methylpyr- idine-2-sulfon- amide | 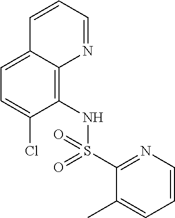 [SMILES: Cc1cccnc1S(=O)(=O)Nc1c(Cl)ccc2cccnc12] | 334 | C | | E | | A |
| 101 | N-(7-chloro- quinolin-8-yl)- 3-methoxypyr- azine-2-sulfon- amide | 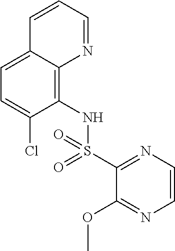 [SMILES: COc1nccnc1S(=O)(=O)Nc1c(Cl)ccc2cccnc12] | 351.1 | A | | E | | |
| 102 | 1-ethyl-N-(7- methylquin- olin-8-yl)-1H- imidazole-2- sulfonamide | 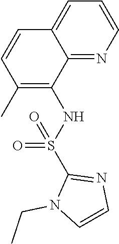 [SMILES: CCn1ccnc1S(=O)(=O)Nc1c(C)ccc2cccnc12] | 316.8 | C | | | | B |
| 103 | 1-ethyl-N-(6- methylquin- olin-8-yl)-1H- imidazole-2- sulfonamide | 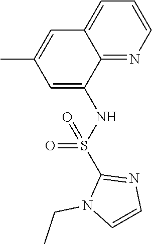 [SMILES: CCn1ccnc1S(=O)(=O)Nc1cc(C)cc2cccnc12] | 316.8 | A | D | | | B |
| 104 | 1-ethyl-N-(4- methylquin- olin-8-yl)-1H- imidazole-2- sulfonamide | 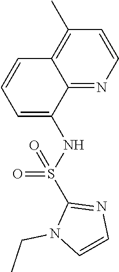 [SMILES: CCn1ccnc1S(=O)(=O)Nc1cccc2c(C)ccnc12] | 316.8 | | D, E | | | B |
| 105 | 1-ethyl-N-(3- methylquin- olin-8-yl)-1H- imidazole-2- sulfonamide | 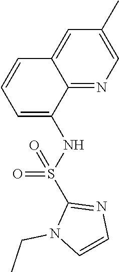 [SMILES: CCn1ccnc1S(=O)(=O)Nc1cccc2cc(C)cnc12] | 316.8 | C | D | | | A |
| 106 | 1-ethyl-N-(2- methylquin- olin-8-yl)-1H- imidazole-2- sulfonamide | 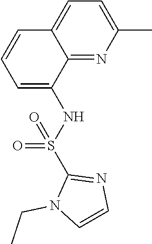 [SMILES: CCn1ccnc1S(=O)(=O)Nc1cccc2ccc(C)nc12] | 317 | A | | | | B |
| 107 | 1-ethyl-N-(6- fluoroquinolin- 8-yl)-1H- imidazole-2- sulfonamide | 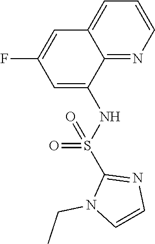 [SMILES: CCn1ccnc1S(=O)(=O)Nc1cc(F)cc2cccnc12] | 320.8 | C | | | | B |
| 108 | 1-ethyl-N-(5- fluoroquinolin- 8-yl)-1H- imidazole-2- sulfonamide | 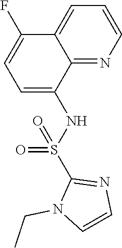 [SMILES: CCn1ccnc1S(=O)(=O)Nc1ccc(F)c2cccnc12] | 320.8 | A | D | | | C |
| 109 | 1-ethyl-N-(3- fluoroquinolin- 8-yl)-1H- imidazole-2- sulfonamide | 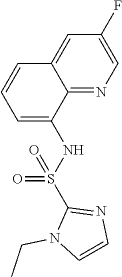 [SMILES: CCn1ccnc1S(=O)(=O)Nc1cccc2cc(F)cnc12] | 321.8 | C | D | | | B |
| 110 | N-(5-chloro- quinolin-8-yl)- 1-ethyl-1H- imidazole-2- sulfonamide | 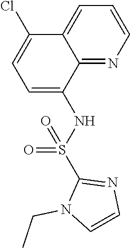 [SMILES: CCn1ccnc1S(=O)(=O)Nc1ccc(Cl)c2cccnc12] | 337.8 | B | D | | | B |
| 111 | N-(7-methoxy- quinolin-8-yl)- 5-methylpyr- idine-2- sulfonamide | 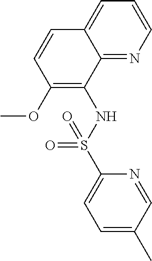 | 329.8 | | D | | | A |
| 112 | 5-methyl-N- (7-methyl- quinolin-8-yl)- pyridine-2- sulfonamide | 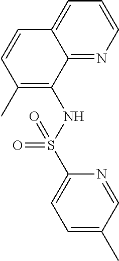 [SMILES: Cc1ccc(S(=O)(=O)Nc2c(C)ccc3cccnc23)nc1] | 314 | B | D | | | A |
| 113 | 5-methyl-N- (3-methyl- quinolin-8-yl)- pyridine-2- sulfonamide | 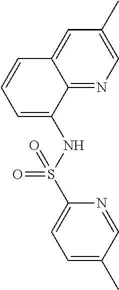 [SMILES: Cc1ccc(S(=O)(=O)Nc2cccc3cc(C)cnc23)nc1] | 313.8 | C | D | | | B |
| 114 | N-(7-chloro- quinolin-8-yl)- 5-methylpyr- idine-2-sulfon- amide | 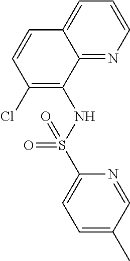 [SMILES: Cc1ccc(S(=O)(=O)Nc2c(Cl)ccc3cccnc23)nc1] | 335.7 | C | | | | A |
| 115 | N-(6-chloro- quinolin-8-yl)- 5-methylpyr- idine-2-sulfon- amide | 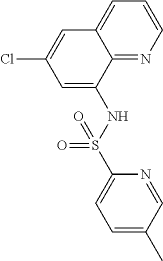 [SMILES: Cc1ccc(S(=O)(=O)Nc2cc(Cl)cc3cccnc23)nc1] | 333.8 | | D | | | A |
| 116 | N-(3-chloro- quinolin-8-yl)- 1-ethyl-1H- imidazole-2- sulfonamide | 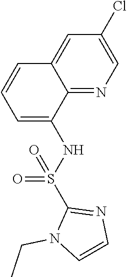 [SMILES: CCn1ccnc1S(=O)(=O)Nc1cccc2cc(Cl)cnc12] | 336.8 | B | | | | B |
| 117 | 1-ethyl-N-(6- methoxyquin- olin-8-yl)-1H- imidazole-2- sulfonamide | 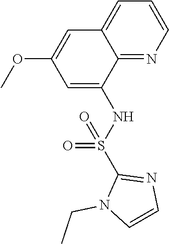 [SMILES: CCn1ccnc1S(=O)(=O)Nc1cc(OC)cc2cccnc12] | 332.8 | A | | | | B |
| 118 | N-(5-chloro- quinolin-8-yl)- 5-methylpyr- idine-2-sulfon- amide | 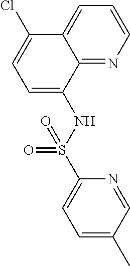 [SMILES: Cc1ccc(S(=O)(=O)Nc2ccc(Cl)c3cccnc23)nc1] | 321.8 | C | | | | A |
| 119 | N-(4-chloro- quinolin-8-yl)- 5-methylpyr- idine-2-sulfon- amide | 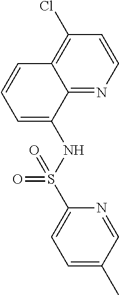 [SMILES: Cc1ccc(S(=O)(=O)Nc2cccc3c(Cl)ccnc23)nc1] | 333.8 | C | E | | D | A |
| 120 | N-(3-chloro- quinolin-8-yl)- 5-methylpyr- idine-2-sulfon- amide | 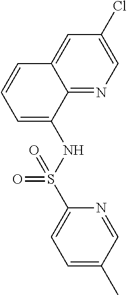 [SMILES: Cc1ccc(S(=O)(=O)Nc2cccc3cc(Cl)cnc23)nc1] | 333.7 | B | | | | B |
| 121 | N-(3-methoxy- quinolin-8-yl)- 5-methylpyr- idine-2-sulfon- amide | 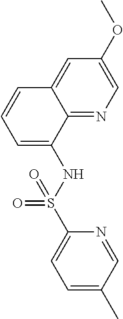 [SMILES: COc1cnc2c(NS(=O)(=O)c3ccc(C)cn3)cccc2c1] | 329.8 | C | | | D | A |
| 122 | 1-ethyl-N-(4- methoxyquin- olin-8-yl)-1H- imidazole-2- sulfonamide | 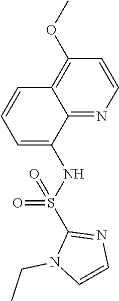 [SMILES: CCn1ccnc1S(=O)(=O)Nc1cccc2c(OC)ccnc12] | 332.8 | C | D | | D | A |
| 123 | N-(5-methoxy- quinolin-8-yl)- 5-methylpyr- idine-2-sulfon- amide | 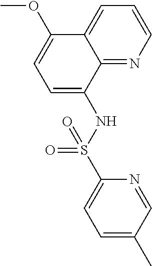 [SMILES: COc1ccc(NS(=O)(=O)c2ccc(C)cn2)c2ncccc12] | 329.8 | C | D | | | A |
| 124 | N-(4-methoxy- quinolin-8-yl)- 5-methylpyr- idine-2-sulfon- amide | 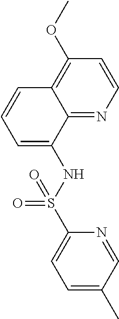 [SMILES: COc1ccnc2c(NS(=O)(=O)c3ccc(C)cn3)cccc12] | 329.8 | C | | | | A |
| 125 | N-(6-methoxy- quinolin-8-yl)- 5-methylpyr- idine-2-sulfon- amide | 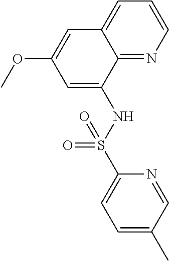 | 329.8 | C | | | | A |
| 126 | N-(5-fluoro- quinolin-8-yl)- 5-methylpyr- idine-2-sulfon- amide | 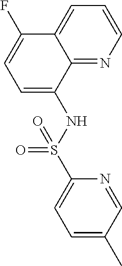 [SMILES: Cc1ccc(S(=O)(=O)Nc2ccc(F)c3cccnc23)nc1] | 317.8 | A | | | | B |
| 127 | 5-methyl-N- (6-methylquin- olin-8-yl)pyr- idine-2-sulfon- amide | 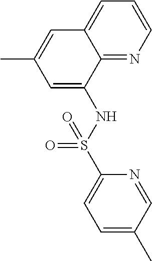 [SMILES: Cc1ccc(S(=O)(=O)Nc2cc(C)cc3cccnc23)nc1] | 313.9 | C | D, E | | | B |
| 128 | N-(6-fluoro- quinolin-8-yl)- 3,5-dimethoxy- benzenesulfon- amide | 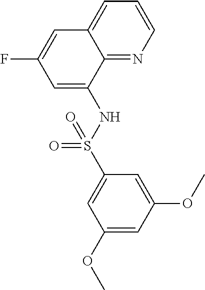 [SMILES: COc1cc(OC)cc(S(=O)(=O)Nc2cc(F)cc3cccnc23)c1] | 363.3 | C | | | | |
| 129 | 1-methyl-N- (6-methyl- quinolin-8- yl)-1H- imidazole-2- sulfonamide | 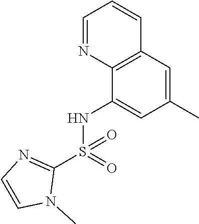 [SMILES: Cc1cc(NS(=O)(=O)c2nccn2C)c2ncccc2c1] | 303.1 | C | D | | D | |
| 130 | N-(5-ethyl- quinolin-8- yl)-1-methyl- 1H-imidazole- 2-sulfonamide | 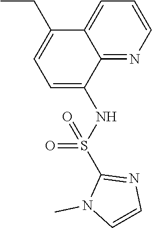 [SMILES: CCc1ccc(NS(=O)(=O)c2nccn2C)c2ncccc12] | 317.1 | A | | | | |
| 131 | N-(6-cyclo- hexylquinolin- 8-yl)pyridine- 2-sulfonamide | 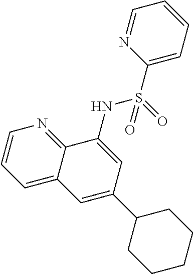 [SMILES: O=S(=O)(Nc1cc(C2CCCCC2)cc2cccnc12)c1ccccn1] | 368.1 | A | | | | |
| 132 | N-(6-methyl- quinolin-8- yl)pyridine-2- sulfonamide | 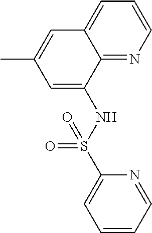 [SMILES: Cc1cc(NS(=O)(=O)c2ccccn2)c2ncccc2c1] | 300.1 | B | | | | |
| 133 | N-(5-fluoro- quinolin-8-yl)- 1-methyl-1H- imidazole-2- sulfonamide | 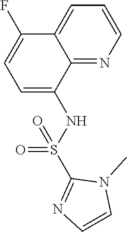 [SMILES: Cn1ccnc1S(=O)(=O)Nc1ccc(F)c2cccnc12] | 307 | C | D | | D, E | |
| 134 | N-(6-fluoro- quinolin-8-yl)- 1-methyl-1H- imidazole-2- sulfonamide | 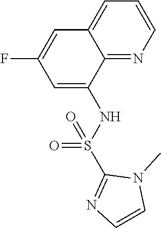 [SMILES: Cn1ccnc1S(=O)(=O)Nc1cc(F)cc2cccnc12] | 307 | C | D, E | | D, E | |
| 135 | N-(6-fluoro- quinolin-8-yl)- pyridine-2- sulfonamide | 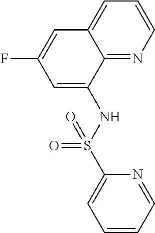 [SMILES: O=S(=O)(Nc1cc(F)cc2cccnc12)c1ccccn1] | 304 | A | | | | |
| 136 | N-(5,6- difluoroquin- olin-8-yl)-4- methylben- zenesulfon- amide | 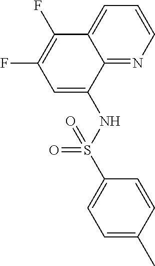 [SMILES: Cc1ccc(S(=O)(=O)Nc2cc(F)c(F)c3cccnc23)cc1] | 335 | B | D | | | |
| 137 | N-(5-methyl- quinolin-8- yl)pyridine-2- sulfonamide | 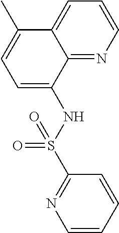 [SMILES: Cc1ccc(NS(=O)(=O)c2ccccn2)c2ncccc12] | 300 | C | D | | | |
| 138 | 4-methyl-N- (6-(trifluoro- methyl)quin- olin-8-yl)- benzene- sulfonamide | 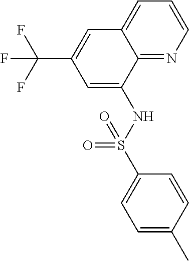 [SMILES: Cc1ccc(S(=O)(=O)Nc2cc(C(F)(F)F)cc3cccnc23)cc1] | 366.9 | B | | | | |
| 139 | N-(7-chloro- 5-morpholino- quinolin-8-yl)- 3-cyclopropyl- pyridine-2- sulfonamide | 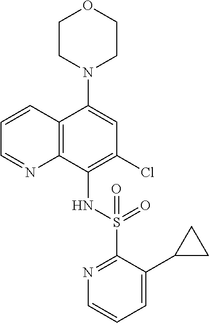 [SMILES: O=S(=O)(Nc1c(Cl)cc(N2CCOCC2)c2cccnc12)c1ncccc1C1CC1] | 445 | C | | | | |
| 140 | N-(7-chloro-5- fluoroquinolin- 8-yl)-3-cyclo- propylpyridine- 2-sulfonamide | 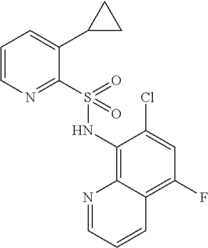 [SMILES: O=S(=O)(Nc1c(Cl)cc(F)c2cccnc12)c1ncccc1C1CC1] | 378 | | | | | A |
| 141 | 3-cyclopropyl- N-(7-methoxy- quinolin-8-yl)- pyridine-2- sulfonamide | 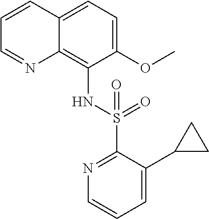 [SMILES: COc1ccc2cccnc2c1NS(=O)(=O)c1ncccc1C1CC1] | 356.1 | C | | | | |

TABLE 2

| | | | | | | | L. | |
|---|---|---|---|---|---|---|---|---|
| | | | | | B. | B. | sigmod- | D. |
| Com- | | | | O. gutt | malayi | phangi | ontis | immitis |
| pound | | | | Activity | Activity | Activity | Activity | Activity |
| ID | Name | Structure | M+ | class | class | class | class | class |
| 142 | 6-cyclopropyl- N-(quinolin-8- yl)pyridine-3- sulfonamide | 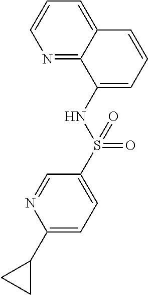 [SMILES: O=S(=O)(Nc1cccc2cccnc12)c1ccc(C2CC2)nc1] | 326.1 | | | | | A |
| 143 | 6-chloro-N- (quinolin-8- yl)pyridine-3- sulfonamide | 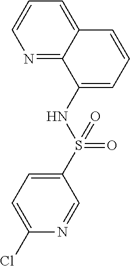 [SMILES: O=S(=O)(Nc1cccc2cccnc12)c1ccc(Cl)nc1] | 320.3 | | | | | |
| 144 | 6-methoxy-N- (quinolin-8- yl)pyridine-3- sulfonamide | 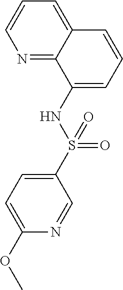 [SMILES: COc1ccc(S(=O)(=O)Nc2cccc3cccnc23)cn1] | 316.3 | | | | | A |
| 145 | N-(quinolin-8- yl)-5- (trifluoromethyl) pyridine-2- sulfonamide | 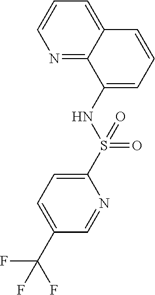 [SMILES: O=S(=O)(Nc1cccc2cccnc12)c1ccc(C(F)(F)F)cn1] | 354.1 | | | | | B |
| 146 | N-(quinolin-8- yl)-2- (trifluoromethyl) pyridine-3- sulfonamide | 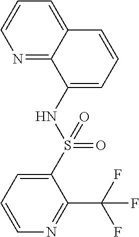 [SMILES: O=S(=O)(Nc1cccc2cccnc12)c1cccnc1C(F)(F)F] | 354.1 | | | | | A |
| 147 | N-(quinolin-8- yl)-4- (trifluoromethyl) pyridine-3- sulfonamide | 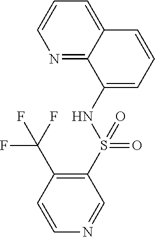 [SMILES: O=S(=O)(Nc1cccc2cccnc12)c1cnccc1C(F)(F)F] | 354.3 | | | | | A |
| 148 | 4-methyl-N- (quinolin-8- yl)pyridine-3- sulfonamide | 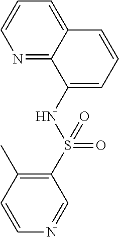 [SMILES: Cc1ccncc1S(=O)(=O)Nc1cccc2cccnc12] | 300.1 | | | | | A |
| 149 | 3-methyl-N- (quinolin-8- yl)pyridine-4- sulfonamide | 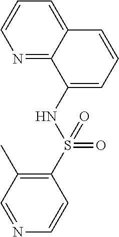 [SMILES: Cc1cnccc1S(=O)(=O)Nc1cccc2cccnc12] | 300.1 | | | | | A |
| 150 | 6- (cyanomethyl)- N-(quinolin-8- yl)pyridine-2- sulfonamide | 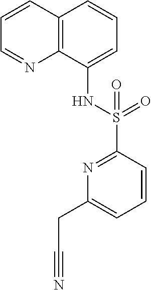 [SMILES: N#CCc1cccc(S(=O)(=O)Nc2cccc3cccnc23)n1] | 325.1 | B | D | | | A |
| 151 | 2-amino-N- (quinolin-8- yl)pyridine-3- sulfonamide | 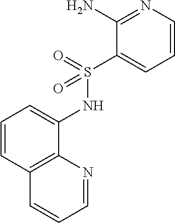 [SMILES: Nc1ncccc1S(=O)(=O)Nc1cccc2cccnc12] | 301.3 | A | | | | A |
| 152 | 2-cyclopropyl- N-(quinolin-8- yl)pyridine-4- sulfonamide | 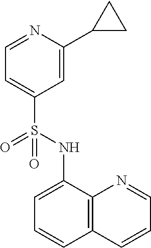 [SMILES: O=S(=O)(Nc1cccc2cccnc12)c1ccnc(C2CC2)c1] | 326.3 | C | D | | | A |
| 153 | 6- (difluoromethyl)- N-(quinolin- 8-yl)pyridine- 3-sulfonamide | 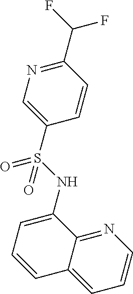 [SMILES: O=S(=O)(Nc1cccc2cccnc12)c1ccc(C(F)F)nc1] | 336.3 | A | D | | | A |
| 154 | 2-methyl-N- (quinolin-8- yl)pyridine-4- sulfonamide | 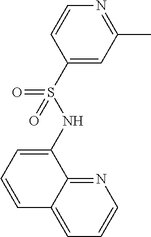 [SMILES: Cc1cc(S(=O)(=O)Nc2cccc3cccnc23)ccn1] | 300.1 | B | | | | A |
| 155 | 2-methyl-N- (quinolin-8- yl)pyridine-3- sulfonamide | 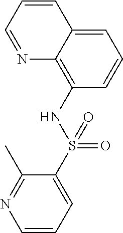 [SMILES: Cc1ncccc1S(=O)(=O)Nc1cccc2cccnc12] | 300.3 | A | | | | A |
| 156 | N-(quinolin-8- yl)-6- (trifluoromethyl) pyridine-2- sulfonamide | 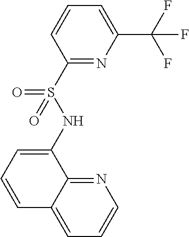 [SMILES: O=S(=O)(Nc1cccc2cccnc12)c1cccc(C(F)(F)F)n1] | 354.2 | C | | | | A |
| 157 | 6-methyl-N- (quinolin-8- yl)pyridine-3- sulfonamide | 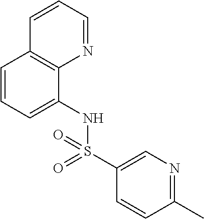 [SMILES: Cc1ccc(S(=O)(=O)Nc2cccc3cccnc23)cn1] | 300.1 | A | | | | A |
| 158 | N-(quinolin-8- yl)-2- (trifluoromethyl) pyridine-4- sulfonamide | 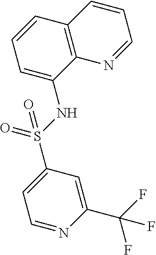 [SMILES: O=S(=O)(Nc1cccc2cccnc12)c1ccnc(C(F)(F)F)c1] | 354.2 | B | | | | A |
| 159 | 6-tert-butyl-N- (quinolin-8- yl)pyridine-3- sulfonamide | 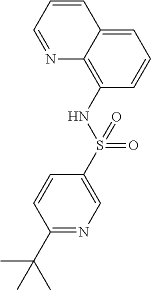 [SMILES: CC(C)(C)c1ccc(S(=O)(=O)Nc2cccc3cccnc23)cn1] | 342.3 | C | | | | B |
| 160 | 4-fluoro-N- (quinolin-8- yl)pyridine-2- sulfonamide | 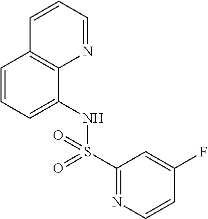 [SMILES: O=S(=O)(Nc1cccc2cccnc12)c1cc(F)ccn1] | 304.1 | C | | | | A |
| 161 | 6-phenyl-N- (quinolin-8- yl)pyridine-2- sulfonamide | 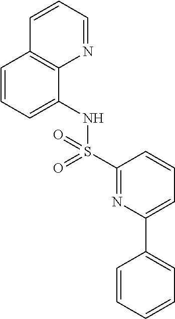 [SMILES: O=S(=O)(Nc1cccc2cccnc12)c1cccc(-c2ccccc2)n1] | 362.1 | A | | | | |
| 162 | 6-cyano-N- (quinolin-8- yl)pyridine-2- sulfonamide | 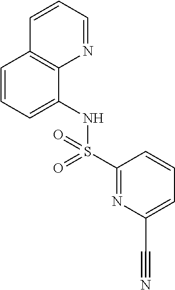 [SMILES: N#Cc1cccc(S(=O)(=O)Nc2cccc3cccnc23)n1] | 311.1 | A | | | | |
| 163 | 3-methyl-N- (quinolin-8-yl)- 6- (trifluoromethyl) pyridine-2- sulfonamide | 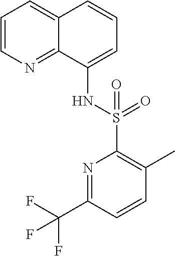 [SMILES: Cc1ccc(C(F)(F)F)nc1S(=O)(=O)Nc1cccc2cccnc12] | 368 | | | | | A |
| 164 | 4,6-dimethyl- N-(quinolin-8- yl)pyridine-3- sulfonamide | 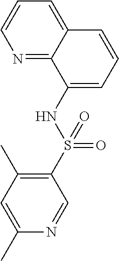 [SMILES: Cc1cc(C)c(S(=O)(=O)Nc2cccc3cccnc23)cn1] | 314.3 | | | | | A |
| 165 | 6-methyl-N- (quinolin-8-yl)- 3- (trifluoromethyl) pyridine-2- sulfonamide | 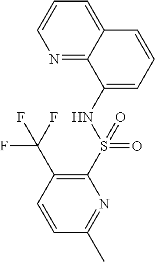 [SMILES: Cc1ccc(C(F)(F)F)c(S(=O)(=O)Nc2cccc3cccnc23)n1] | 368.1 | | | | | B |
| 166 | 4-methyl-N- (quinolin-8-yl)- 6- (trifluoromethyl) pyridine-3- sulfonamide | 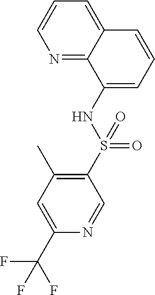 [SMILES: Cc1cc(C(F)(F)F)ncc1S(=O)(=O)Nc1cccc2cccnc12] | 368.3 | | | | | A |
| 167 | 4-cyano-6- methyl-N- (quinolin-8- yl)pyridine-2- sulfonamide | 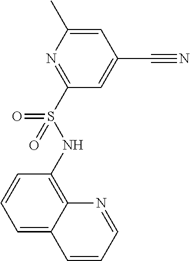 [SMILES: Cc1cc(C#N)cc(S(=O)(=O)Nc2cccc3cccnc23)n1] | 325.3 | B | | | D | A |
| 168 | N-(quinolin-8- yl)-1-(1- (trifluoromethyl) cyclopropyl)- 1H-imidazole- 2-sulfonamide | 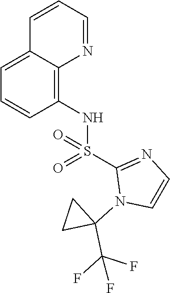 [SMILES: O=S(=O)(Nc1cccc2cccnc12)c1nccn1C1(C(F)(F)F)CC1] | 383.3 | C | | | D, E | B |
| 169 | 6-chloro-2- cyano-N- (quinolin-8- yl)pyridine-3- sulfonamide | 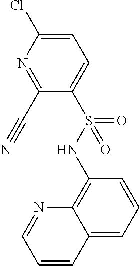 [SMILES: N#Cc1nc(Cl)ccc1S(=O)(=O)Nc1cccc2cccnc12] | 345 | A | | | | A |
| 170 | 2-(pyridin-2- yl)-N- (quinolin-8- yl)thiazole-5- sulfonamide | 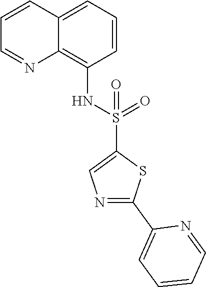 [SMILES: O=S(=O)(Nc1cccc2cccnc12)c1cnc(-c2ccccn2)s1] | 369.3 | C | | | D | |
| 171 | N-(quinolin-8- yl)-1-(2- (trifluoromethyl) cyclopropyl)- 1H-imidazole- 2-sulfonamide | 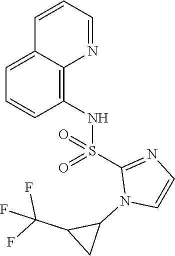 [SMILES: O=S(=O)(Nc1cccc2cccnc12)c1nccn1C1CC1C(F)(F)F] | 383.2 | C | | | D, E | B |
| 172 | 6-(oxazol-5- yl)-N- (quinolin-8- yl)pyridine-3- sulfonamide | 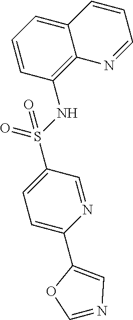 [SMILES: O=S(=O)(Nc1cccc2cccnc12)c1ccc(-c2cnco2)nc1] | 353.1 | A | | | D | |
| 173 | 2-methyl-N- (quinolin-8-yl)- 6- (trifluoromethyl) pyridine-3- sulfonamide | 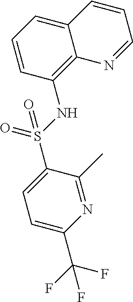 [SMILES: Cc1nc(C(F)(F)F)ccc1S(=O)(=O)Nc1cccc2cccnc12] | 368.2 | A | | | D | |
| 174 | 1-(2,2- difluorocyclo- propyl)-N- (quinolin-8-yl)- 1H-imidazole- 2-sulfonamide | 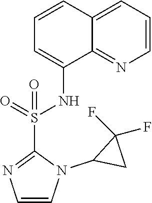 [SMILES: O=S(=O)(Nc1cccc2cccnc12)c1nccn1C1CC1(F)F] | 351.3 | C | | | D, E | B |
| 175 | 5-chloro-3- cyano-4,6- dimethyl-N- (quinolin-8- yl)pyridine-2- sulfonamide | 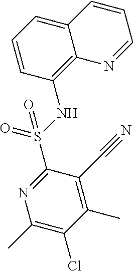 [SMILES: Cc1nc(S(=O)(=O)Nc2cccc3cccnc23)c(C#N)c(C)c1Cl] | 373.3 | B | | | | |
| 176 | 6-chloro-N- (quinolin-8-yl)- 2- (trifluoromethyl) pyridine-3- sulfonamide | 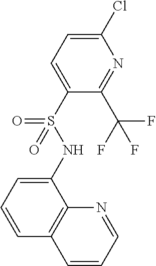 [SMILES: O=S(=O)(Nc1cccc2cccnc12)c1ccc(Cl)nc1C(F)(F)F] | 388 | B | | | | A |
| 177 | 2-chloro-4,6- dimethyl-N- (quinolin-8- yl)pyridine-3- sulfonamide | 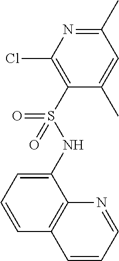 [SMILES: Cc1cc(C)c(S(=O)(=O)Nc2cccc3cccnc23)c(Cl)n1] | 348.1 | A | | | | A |
| 178 | 6-(4- cyanophenyl)- N-(quinolin-8- yl)pyridine-2- sulfonamide | 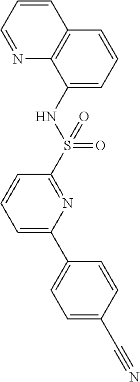 [SMILES: N#Cc1ccc(-c2cccc(S(=O)(=O)Nc3cccc4cccnc34)n2)cc1] | 387.3 | C | | | | A |
| 179 | 6-chloro-2- methyl-N- (quinolin-8- yl)pyridine-3- sulfonamide | 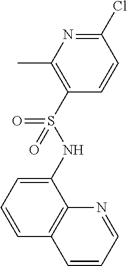 [SMILES: Cc1nc(Cl)ccc1S(=O)(=O)Nc1cccc2cccnc12] | 334.3 | C | | | D, E | A |
| 180 | 5-chloro-6- methyl-N- (quinolin-8- yl)pyridine-3- sulfonamide | 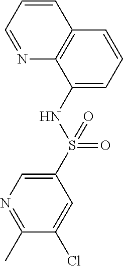 [SMILES: Cc1ncc(S(=O)(=O)Nc2cccc3cccnc23)cc1Cl] | 334.3 | A | | | | A |
| 181 | 5-chloro-2- methyl-N- (quinolin-8- yl)pyridine-3- sulfonamide | 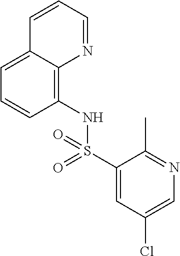 [SMILES: Cc1ncc(Cl)cc1S(=O)(=O)Nc1cccc2cccnc12] | 334.3 | A | | | | B |
| 182 | 6-(4- fluorophenyl)- N-(quinolin-8- yl)pyridine-3- sulfonamide | 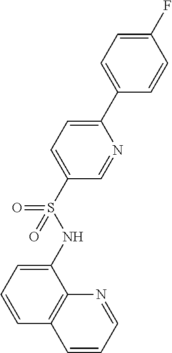 [SMILES: O=S(=O)(Nc1cccc2cccnc12)c1ccc(-c2ccc(F)cc2)nc1] | 380.3 | C | | | | A |
| 183 | 3-cyano-4,6- dimethyl-N- (quinolin-8- yl)pyridine-2- sulfonamide | 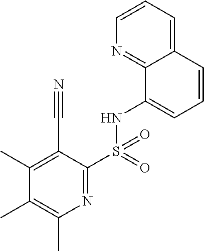 [SMILES: Cc1nc(S(=O)(=O)Nc2cccc3cccnc23)c(C#N)c(C)c1C] | 339.3 | B | | | D | A |
| 184 | 5-chloro-6- methyl-N- (quinolin-8- yl)pyridine-2- sulfonamide | 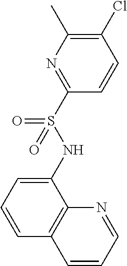 [SMILES: Cc1nc(S(=O)(=O)Nc2cccc3cccnc23)ccc1Cl] | 334.3 | C | | | D | B |
| 185 | (S)-1-(1- cyclopropyl- ethyl)-N- (quinolin-8-yl)- 1H-imidazole- 2-sulfonamide | 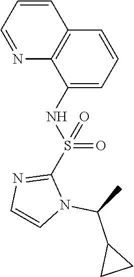 [SMILES: C[C@@H](C1CC1)n1ccnc1S(=O)(=O)Nc1cccc2cccnc12] | 343.3 | C | | | D, E | B |
| 186 | 2,6-dimethyl- N-(quinolin-8- yl)pyridine-4- sulfonamide | 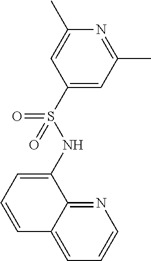 [SMILES: Cc1cc(S(=O)(=O)Nc2cccc3cccnc23)cc(C)n1] | 314.3 | B | | | | A |
| 187 | 2,6-dimethyl- N-(quinolin-8- yl)pyridine-3- sulfonamide | 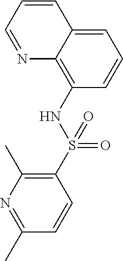 [SMILES: Cc1ccc(S(=O)(=O)Nc2cccc3cccnc23)c(C)n1] | 314.1 | A | | | | A |
| 188 | 2-chloro-6- methyl-N- (quinolin-8- yl)pyridine-3- sulfonamide | 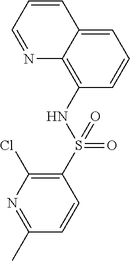 [SMILES: Cc1ccc(S(=O)(=O)Nc2cccc3cccnc23)c(Cl)n1] | 334.3 | C | | | | A |
| 189 | 6-phenyl-N- (quinolin-8- yl)pyridine-3- sulfonamide | 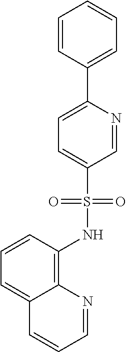 [SMILES: O=S(=O)(Nc1cccc2cccnc12)c1ccc(-c2ccccc2)nc1] | 362.3 | B | | | | A |
| 190 | N-(quinolin-8- yl)-6- (trifluoromethyl) pyridine-3- sulfonamide | 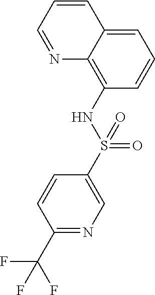 [SMILES: O=S(=O)(Nc1cccc2cccnc12)c1ccc(C(F)(F)F)nc1] | 354.2 | C | | | | C |
| 191 | 1-ethyl-4- methyl-N- (quinolin-8-yl)- 1H-imidazole- 2-sulfonamide | 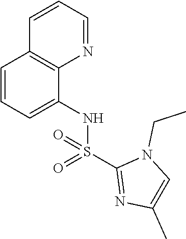 [SMILES: CCn1cc(C)nc1S(=O)(=O)Nc1cccc2cccnc12] | 317.3 | C | | | | B |
| 192 | 1-methyl-3- morpholino-N- (quinolin-8-yl)- 1H-pyrazole-5- sulfonamide | 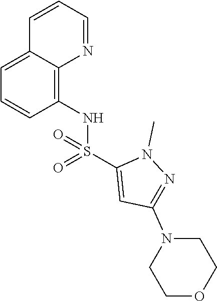 [SMILES: Cn1nc(N2CCOCC2)cc1S(=O)(=O)Nc1cccc2cccnc12] | 374.1 | A | | | | |
| 193 | 1-ethyl-N- (quinolin-8-yl)- 1H-pyrazole-3- sulfonamide | 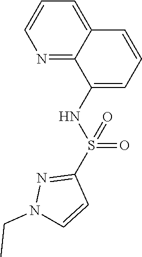 [SMILES: CCn1ccc(S(=O)(=O)Nc2cccc3cccnc23)n1] | 302.9 | B | | | | A |
| 194 | 3-methyl-N- (1,6- naphthyridin-8- yl)pyridine-2- sulfonamide | 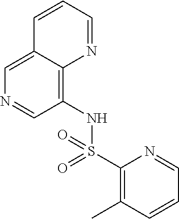 [SMILES: Cc1cccnc1S(=O)(=O)Nc1cncc2cccnc12] | 301.2 | B | | | | |
| 195 | 4-cyclopropyl- N-(1,5- naphthyridin-4- yl)pyridine-2- sulfonamide | 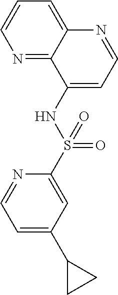 [SMILES: O=S(=O)(Nc1ccnc2cccnc12)c1cc(C2CC2)ccn1] | 327 | C | D | | D, E | |
| 196 | 5-methyl-N- (quinoxalin-5- yl)pyridine-2- sulfonamide | 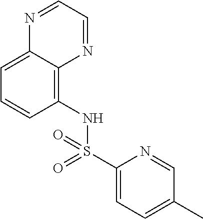 [SMILES: Cc1ccc(S(=O)(=O)Nc2cccc3nccnc23)nc1] | 300.8 | B | D | | | B |
| 197 | 2-methoxy-N- (quinolin-8- yl)pyrimidine- 4-sulfonamide | 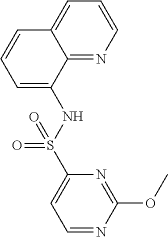 [SMILES: COc1nccc(S(=O)(=O)Nc2cccc3cccnc23)n1] | 316.8 | B | D | | | A |
| 198 | 5-methyl-N- (quinolin-8- yl)pyrimidine- 2-sulfonamide | 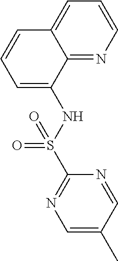 [SMILES: Cc1cnc(S(=O)(=O)Nc2cccc3cccnc23)nc1] | 300.8 | B | | | | |
| 199 | N-(quinolin-8- yl)pyrimidine- 4-sulfonamide | 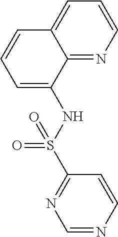 [SMILES: O=S(=O)(Nc1cccc2cccnc12)c1ccncn1] | 287 | A | | | | |
| 200 | N-(quinolin-8- yl)pyrimidine- 2-sulfonamide | 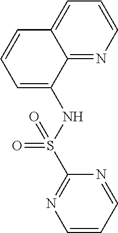 [SMILES: O=S(=O)(Nc1cccc2cccnc12)c1ncccn1] | 287 | C | D | | | |
| 201 | N-(quinolin-8- yl)- [1,2,4]triazolo [4,3-a]pyridine- 3-sulfonamide | 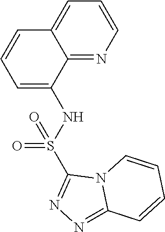 [SMILES: O=S(=O)(Nc1cccc2cccnc12)c1nnc2ccccn12] | 325.8 | A | | | | |
| 202 | N-(quinazolin- 8-yl)pyridine- 2-sulfonamide | 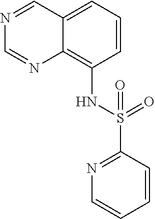 [SMILES: O=S(=O)(Nc1cccc2cncnc12)c1ccccn1] | 287 | A | | | | |
| 203 | N-(1,5- naphthyridin-4- yl)pyridine-2- sulfonamide | 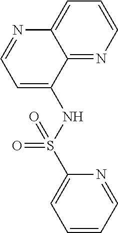 [SMILES: O=S(=O)(Nc1ccnc2cccnc12)c1ccccn1] | 287 | A | | | | |
| 204 | N-(quinolin-8- yl)-1H- pyrrolo[2,3- b]pyridine-3- sulfonamide | 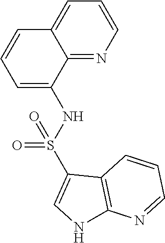 [SMILES: O=S(=O)(Nc1cccc2cccnc12)c1c[nH]c2ncccc12] | 325 | A | D | | | |
| 205 | N-(quinolin-8- yl)piperidine-1- sulfonamide | 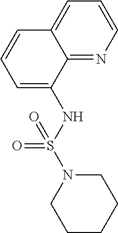 [SMILES: O=S(=O)(Nc1cccc2cccnc12)N1CCCCC1] | 294.1 | A | D | | | |
| 206 | 1-isopropyl-N- (quinolin-8-yl)- 1H-pyrazole-5- sulfonamide | 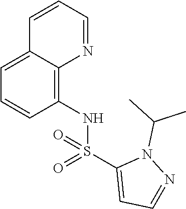 [SMILES: CC(C)n1nccc1S(=O)(=O)Nc1cccc2cccnc12] | 317.3 | B | D | E | D | A |
| 207 | N,N-dimethyl- 2-(N-quinolin- 8- ylsulfamoyl) nicotinamide | 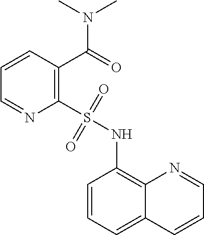 [SMILES: CN(C)C(=O)c1cccnc1S(=O)(=O)Nc1cccc2cccnc12] | 357.1 | A | | | | A |
| 208 | 3- (dimethylamino)- N-(quinolin- 8-yl)pyridine- 2-sulfonamide | 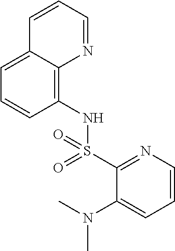 [SMILES: CN(C)c1cccnc1S(=O)(=O)Nc1cccc2cccnc12] | 329.1 | C | E | | D,E | A |
| 209 | 4-methyl-N- (quinolin-8- yl)piperazine- 1-sulfonamide | 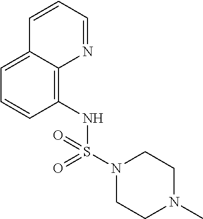 [SMILES: CN1CCN(S(=O)(=O)Nc2cccc3cccnc23)CC1] | 307.2 | | D | | | |
| 210 | 3-methyl-N- (quinolin-8- yl)pyrazine-2- sulfonamide | 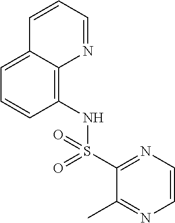 [SMILES: Cc1nccnc1S(=O)(=O)Nc1cccc2cccnc12] | 301 | C | D,E | | D,E | |
| 211 | 3-isopropyl-N- (quinolin-8- yl)pyridine-2- sulfonamide | 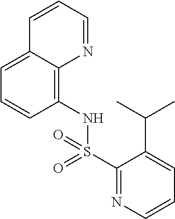 [SMILES: CC(C)c1cccnc1S(=O)(=O)Nc1cccc2cccnc12] | 328.2 | C | | E | D,E | A |
| 212 | 2-(N-quinolin- 8- ylsulfamoyl) nicotinamide | 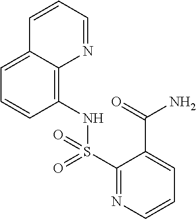 [SMILES: NC(=O)c1cccnc1S(=O)(=O)Nc1cccc2cccnc12] | 329.1 | | | E | D | |
| 213 | 3-methyl-N- (quinolin-8- yl)pyridine-2- sulfonamide | 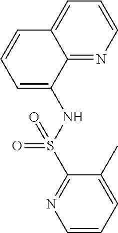 [SMILES: Cc1cccnc1S(=O)(=O)Nc1cccc2cccnc12] | 300.1 | C | D,E | E | D,E | A |
| 214 | 3-cyclopropyl- N-(quinolin-8- yl)pyridine-2- sulfonamide | 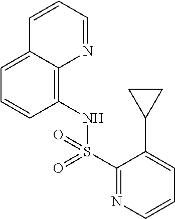 [SMILES: O=S(=O)(Nc1cccc2cccnc12)c1ncccc1C1CC1] | 326.1 | B | | E | D | |
| 215 | 3-chloro-N- (quinolin-8- yl)pyridine-2- sulfonamide | 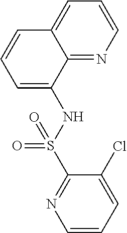 [SMILES: O=S(=O)(Nc1cccc2cccnc12)c1ncccc1Cl] | 320.1 | C | E | | E | A |
| 216 | 1-methyl-N- (quinolin-8- yl)piperidine-4- sulfonamide | 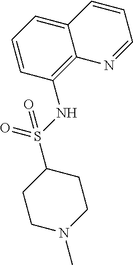 [SMILES: CN1CCC(S(=O)(=O)Nc2cccc3cccnc23)CC1] | 306.1 | A | | E | | |
| 217 | N-(4-oxo-3,4- dihydro- quinazolin-8- yl)pyridine-2- sulfonamide | 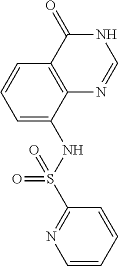 [SMILES: O=c1[nH]cnc2c(NS(=O)(=O)c3ccccn3)cccc12] | 303 | | D | | | |
| 218 | 5-hydroxy-N- (quinolin-8- yl)pyridine-2- sulfonamide | 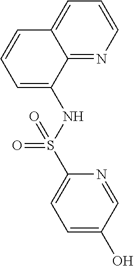 [SMILES: O=S(=O)(Nc1cccc2cccnc12)c1ccc(O)cn1] | 301.8 | A | | | | |
| 219 | 3-hydroxy-N- (quinolin-8- yl)pyridine-2- sulfonamide | 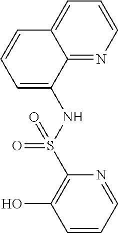 [SMILES: O=S(=O)(Nc1cccc2cccnc12)c1ncccc1O] | 301.8 | A | | | | |
| 220 | 4-chloro-N- (quinolin-8- yl)pyridine-2- sulfonamide | 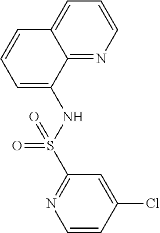 [SMILES: O=S(=O)(Nc1cccc2cccnc12)c1cc(Cl)ccn1] | 321.7 | C | D,E | | D | C |
| 221 | 6-chloro-N- (quinolin-8- yl)pyridine-2- sulfonamide | 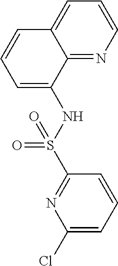 [SMILES: O=S(=O)(Nc1cccc2cccnc12)c1cccc(Cl)n1] | 321.9 | C | D | | D | A |
| 222 | 4-methoxy-N- (quinolin-8- yl)pyridine-2- sulfonamide | 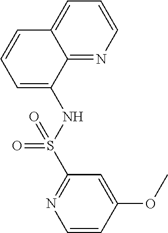 [SMILES: COc1ccnc(S(=O)(=O)Nc2cccc3cccnc23)c1] | 315.8 | C | E | | D | B |
| 223 | 3-methoxy-N- (quinolin-8- yl)pyridine-2- sulfonamide | 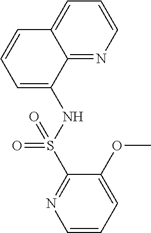 [SMILES: COc1cccnc1S(=O)(=O)Nc1cccc2cccnc12] | 316 | C | | | | |
| 224 | 6-methoxy-N- (quinolin-8- yl)pyridine-2- sulfonamide | 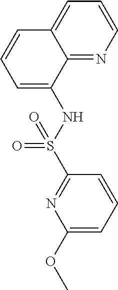 [SMILES: COc1cccc(S(=O)(=O)Nc2cccc3cccnc23)n1] | 316 | B | | | | |
| 225 | N-(quinolin-8- yl)isoquinoline- 1-sulfonamide | 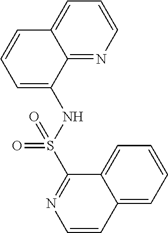 [SMILES: O=S(=O)(Nc1cccc2cccnc12)c1nccc2ccccc12] | 335.8 | C | D,E | | | A |
| 226 | 1-ethyl-4,5- dimethyl-N- (quinolin-8-yl)- 1H-imidazole- 2-sulfonamide | 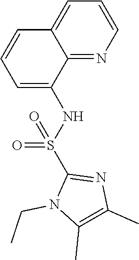 [SMILES: CCn1c(S(=O)(=O)Nc2cccc3cccnc23)nc(C)c1C] | 330.9 | C | | | D | |
| 227 | 1-ethyl-N- (quinolin-8-yl)- 1H-pyrrole-2- sulfonamide | 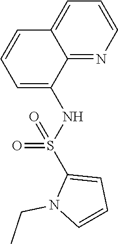 [SMILES: CCn1cccc1S(=O)(=O)Nc1cccc2cccnc12] | 302 | C | | | | |
| 228 | 4-hydroxy-N- (quinolin-8- yl)pyridine-2- sulfonamide | 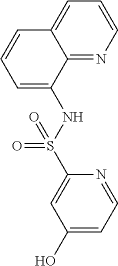 [SMILES: O=S(=O)(Nc1cccc2cccnc12)c1cc(O)ccn1] | 301.8 | | | | | |
| 229 | N-(quinolin-8- yl)-1,6- naphthyridine- 5-sulfonamide | 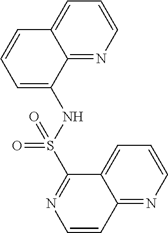 [SMILES: O=S(=O)(Nc1cccc2cccnc12)c1nccc2ncccc12] | 337 | C | D | | | |
| 230 | N-(quinolin-8- yl)imidazo[1,2- a]pyridine-5- sulfonamide | 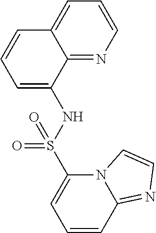 [SMILES: O=S(=O)(Nc1cccc2cccnc12)c1cccc2nccn12] | 324.8 | | | | | |
| 231 | 1,4-dimethyl- N-(quinolin-8- yl)-1H- imidazole-2- sulfonamide | 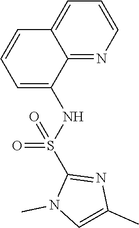 [SMILES: Cc1cn(C)c(S(=O)(=O)Nc2cccc3cccnc23)n1] | 302.8 | A | D,E | | | C |
| 232 | N-(quinolin-8- yl)-1H- benzo[d] imidazole-2- sulfonamide | 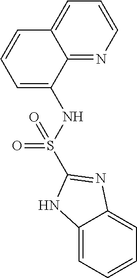 [SMILES: O=S(=O)(Nc1cccc2cccnc12)c1nc2ccccc2[nH]1] | 324.8 | | D | | | A |
| 233 | 1,5-dimethyl- N-(quinolin-8- yl)-1H- imidazole-2- sulfonamide | 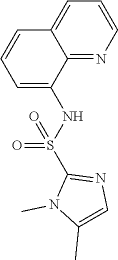 [SMILES: Cc1cnc(S(=O)(=O)Nc2cccc3cccnc23)n1C] | 302.8 | | D | | | B |
| 234 | 4-methyl-N- (quinolin-8- yl)pyridine-2- sulfonamide | 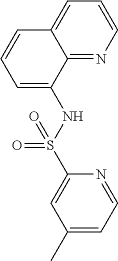 [SMILES: Cc1ccnc(S(=O)(=O)Nc2cccc3cccnc23)c1] | 300 | C | E | | | |
| 235 | 5-methoxy-N- (quinolin-8- yl)pyridine-2- sulfonamide | 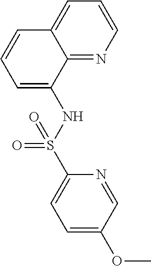 [SMILES: COc1ccc(S(=O)(=O)Nc2cccc3cccnc23)nc1] | 316.1 | C | D,E | | | |
| 236 | 5-methyl-N- (quinolin-5- yl)pyridine-2- sulfonamide | 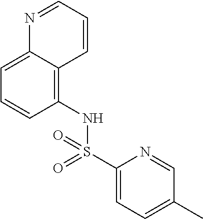 [SMILES: Cc1ccc(S(=O)(=O)Nc2cccc3ncccc23)nc1] | 299.8 | | | | | |
| 237 | N-(isoquinolin- 8-yl)-5- methylpyridine- 2-sulfonamide | 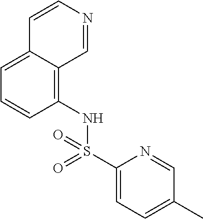 [SMILES: Cc1ccc(S(=O)(=O)Nc2cccc3ccncc23)nc1] | 299.8 | | | | | |
| 238 | N-(isoquinolin- 5-yl)-5- methylpyridine- 2-sulfonamide | 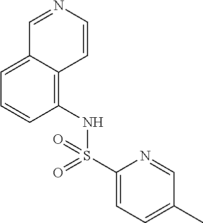 [SMILES: Cc1ccc(S(=O)(=O)Nc2cccc3cnccc23)nc1] | 299.8 | | D | | | |
| 239 | N-(quinolin-8- yl)quinoline-2- sulfonamide | 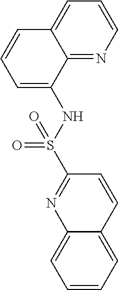 [SMILES: O=S(=O)(Nc1cccc2cccnc12)c1ccc2ccccc2n1] | 335.8 | C | D | | D | |
| 240 | 1-ethyl-N- (quinolin-8-yl)- 1H-imidazole- 2-sulfonamide | 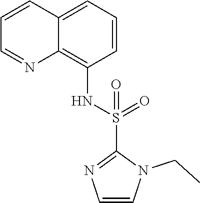 [SMILES: CCn1ccnc1S(=O)(=O)Nc1cccc2cccnc12] | 303.4 | C | | | | |
| 241 | 1-isopropyl-N- (quinolin-8-yl)- 1H-imidazole- 2-sulfonamide | 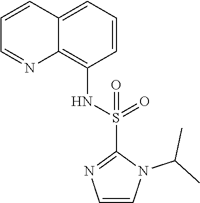 [SMILES: CC(C)n1ccnc1S(=O)(=O)Nc1cccc2cccnc12] | 317.1 | C | | | | |
| 242 | 5-isopropoxy- N-(quinolin-8- yl)pyridine-2- sulfonamide | 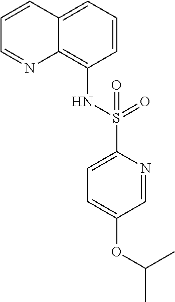 [SMILES: CC(C)Oc1ccc(S(=O)(=O)Nc2cccc3cccnc23)nc1] | 344.1 | C | E | | | B |
| 243 | N-(quinolin-8- yl)-1H- imidazole-2- sulfonamide | 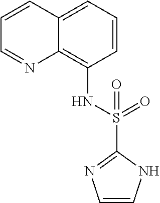 [SMILES: O=S(=O)(Nc1cccc2cccnc12)c1ncc[nH]1] | 275 | A | | | D | |
| 244 | 5-phenyl-N- (quinolin-8- yl)pyridine-2- sulfonamide | 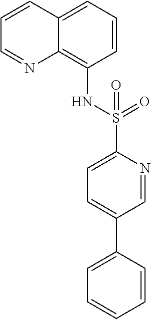 [SMILES: O=S(=O)(Nc1cccc2cccnc12)c1ccc(-c2ccccc2)cn1] | 362 | A | D | | | |
| 245 | 4-isopropoxy- N-(quinolin-8- yl)pyridine-2- sulfonamide | 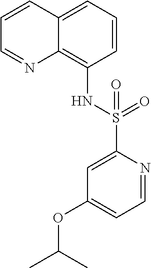 [SMILES: CC(C)Oc1ccnc(S(=O)(=O)Nc2cccc3cccnc23)c1] | 344 | C | E | | | B |
| 246 | N-(quinolin-7- yl)pyridine-2- sulfonamide | 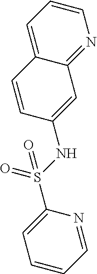 [SMILES: O=S(=O)(Nc1ccc2cccnc2c1)c1ccccn1] | 286 | | | | | |
| 247 | 5-fluoro-N- (quinolin-8- yl)pyridine-2- sulfonamide | 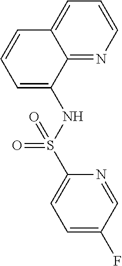 [SMILES: O=S(=O)(Nc1cccc2cccnc12)c1ccc(F)cn1] | 304 | C | | | | |
| 248 | 5-methyl-N- (quinolin-8- yl)pyridine-2- sulfonamide | 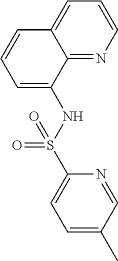 [SMILES: Cc1ccc(S(=O)(=O)Nc2cccc3cccnc23)nc1] | 300.1 | A | | | | B |
| 249 | 2-cyano-N- (quinolin-8- yl)benzene- sulfonamide | 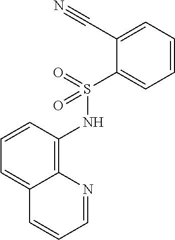 [SMILES: N#Cc1ccccc1S(=O)(=O)Nc1cccc2cccnc12] | 309.9 | B | | | D,E | |
| 250 | 3-cyano-N- (quinolin-8- yl)benzene- sulfonamide | 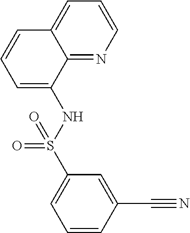 [SMILES: N#Cc1cccc(S(=O)(=O)Nc2cccc3cccnc23)c1] | 310.2 | A | D,E | | D,E | |
| 251 | 1-methyl-N- (quinolin-8-yl)- 1H-imidazole- 2-sulfonamide | 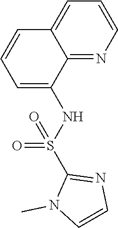 [SMILES: Cn1ccnc1S(=O)(=O)Nc1cccc2cccnc12] | 288.8 | C | E | | | |
| 252 | 1-methyl-N- (quinolin-8-yl)- 1H-pyrazole-5- sulfonamide | 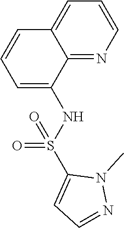 [SMILES: Cn1nccc1S(=O)(=O)Nc1cccc2cccnc12] | 288.8 | A | E | | | |
| 253 | N-(quinolin-8- yl)-4H-1,2,4- triazole-3- sulfonamide | 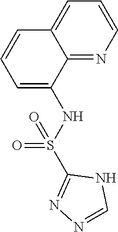 [SMILES: O=S(=O)(Nc1cccc2cccnc12)c1nnc[nH]1] | 276.0 | | E | | E | |
| 254 | 3,5-dimethyl- N-(quinolin-8- yl)benzene- sulfonamide | 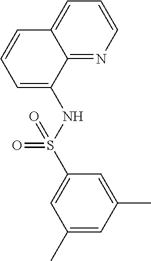 [SMILES: Cc1cc(C)cc(S(=O)(=O)Nc2cccc3cccnc23)c1] | | C | | | | |
| 255 | 1-methyl-N- (quinolin-8-yl)- 1H-pyrazole-3- sulfonamide | 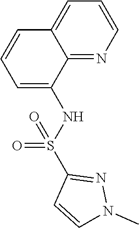 [SMILES: Cn1ccc(S(=O)(=O)Nc2cccc3cccnc23)n1] | | B | | | | |
| 256 | N-methyl-N- (quinolin-8- yl)pyridine-2- sulfonamide | 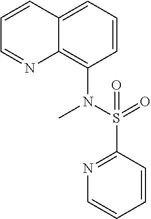 [SMILES: CN(c1cccc2cccnc12)S(=O)(=O)c1ccccn1] | A | | | | | |
| 257 | 4-methyl-N- (quinolin-8- yl)pyridine-2- sulfonamide | 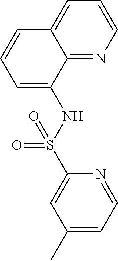 [SMILES: Cc1ccnc(S(=O)(=O)Nc2cccc3cccnc23)c1] | 300 | C | E | | | |
| 258 | 5-methoxy-N- (quinolin-8- yl)pyridine-2- sulfonamide | 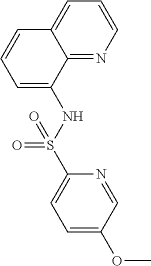 [SMILES: COc1ccc(S(=O)(=O)Nc2cccc3cccnc23)nc1] | 316 | C | | | | |
| 259 | 5-chloro-N- (quinolin-8- yl)pyridine-2- sulfonamide | 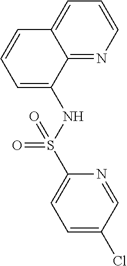 [SMILES: O=S(=O)(Nc1cccc2cccnc12)c1ccc(Cl)cn1] | 320 | A | D | | | |
| 260 | 5-phenyl-N- (quinolin-8- yl)pyridine-2- sulfonamide | 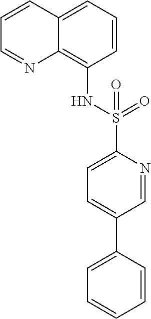 [SMILES: O=S(=O)(Nc1cccc2cccnc12)c1ccc(-c2ccccc2)cn1] | 362 | A | E | | | |
| 261 | 4-isopropoxy- N-(quinolin-8- yl)pyridine-2- sulfonamide | 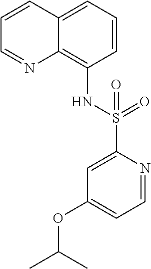 [SMILES: CC(C)Oc1ccnc(S(=O)(=O)Nc2cccc3cccnc23)c1] | 344 | C | | | | |
| 262 | N-(quinolin-7- yl)pyridine-2- sulfonamide | 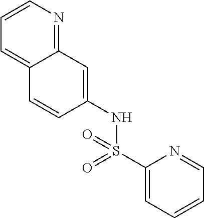 [SMILES: O=S(=O)(Nc1ccc2cccnc2c1)c1ccccn1] | 386 | A | | | | |

TABLE 3

| | | | | | | L. | |
|---|---|---|---|---|---|---|---|
| | | | | | | sigmodon- | D. |
| Com- | | | O. gutt | B. malayi | B. phange | tis | immitis- |
| pound | | | Activity | Activity | Activity | Activity | Activity |
| ID | Name | Structure | class | class | class | class | class |
| 263 | N-(quinolin-8- yl)-5- (trifluoromethyl) pyridine-3- sulfonamide | 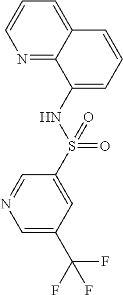 [SMILES: O=S(=O)(Nc1cccc2cccnc12)c1cncc(C(F)(F)F)c1] | | | | | B |
| 264 | 6-amino-N- (quinolin-8- yl)pyridin-2- sulfonamide | 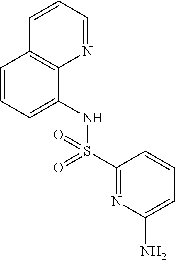 [SMILES: Nc1cccc(S(=O)(=O)Nc2cccc3cccnc23)n1] | A | | | | A |
| 265 | 4-methyl-N- (quinolin-8- yl)pyridine-2- sulfonamide | 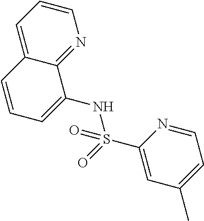 [SMILES: Cc1ccnc(S(=O)(=O)Nc2cccc3cccnc23)c1] | C | | | | B |
| 266 | 2-methyl-N- (quinolin-8- yl)benzenesulfon- amide | 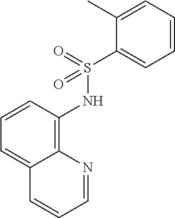 [SMILES: Cc1ccccc1S(=O)(=O)Nc1cccc2cccnc12] | | | | | A |
| 267 | N-(quinolin-8- yl)-1H- pyrazol-4- sulfonamide | 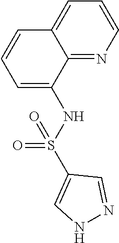 [SMILES: O=S(=O)(Nc1cccc2cccnc12)c1cn[nH]c1] | | A | | | |
| 268 | 1-methyl-N- (quinolin-8-yl)- 1H-pyrazole-4- sulfonamide | 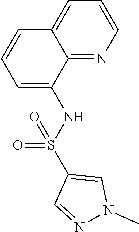 [SMILES: Cn1cc(S(=O)(=O)Nc2cccc3cccnc23)cn1] | | A | D | | |
| 269 | 3-fluoro-N- (quinolin-8- yl)pyridine-2- sulfonamide | 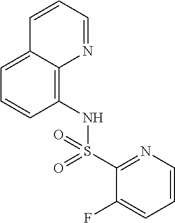 [SMILES: O=S(=O)(Nc1cccc2cccnc12)c1ncccc1F] | C | E | E | D, E | A |
| 270 | N-(quinolin-8- yl)isoquinoline- 3-sulfonamide | 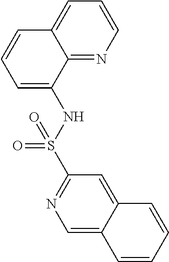 [SMILES: O=S(=O)(Nc1cccc2cccnc12)c1cc2ccccc2cn1] | C | D, E | | | B |
| 271 | N-(quinolin-8- yl)pyrazine-2- sulfonamide | 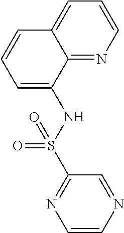 [SMILES: O=S(=O)(Nc1cccc2cccnc12)c1cnccn1] | C | | | D, E | A |
| 272 | 6-methyl-N- (quinolin-8- yl)pyridine-2- sulfonamide | 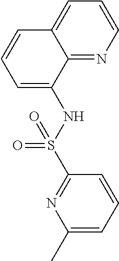 [SMILES: Cc1cccc(S(=O)(=O)Nc2cccc3cccnc23)n1] | C | | | | A |
| 273 | 3,4-dimethyl- N-(quinolin-8- yl)benzenesulfon- amide | 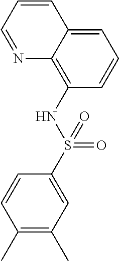 [SMILES: Cc1ccc(S(=O)(=O)Nc2cccc3cccnc23)cc1C] | C | | | | |
| 274 | N-(quinolin-8- yl)-2- (trifluoromethyl) benzene- sulfonamide | 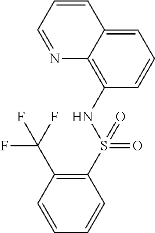 [SMILES: O=S(=O)(Nc1cccc2cccnc12)c1ccccc1C(F)(F)F] | C | | | | |
| 275 | 2,4-difluoro-N- (quinolin-8- yl)benzene- sulfonamide | 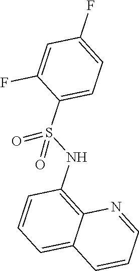 [SMILES: O=S(=O)(Nc1cccc2cccnc12)c1ccc(F)cc1F] | B | | | | |
| 276 | 5-methyl-N- (quinolin-8- yl)furan-2- sulfonamide | 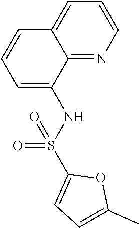 [SMILES: Cc1ccc(S(=O)(=O)Nc2cccc3cccnc23)o1] | B | | | D | B |
| 277 | N-(quinolin-8- yl)pyridine-3- sulfonamide | 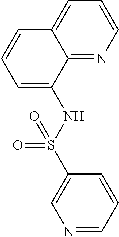 [SMILES: O=S(=O)(Nc1cccc2cccnc12)c1cccnc1] | A | | | | |
| 278 | 2,4-dichloro-N- (quinolin-8- yl)benzene- sulfonamide | 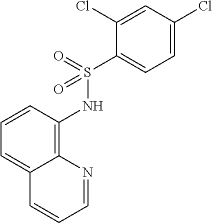 [SMILES: O=S(=O)(Nc1cccc2cccnc12)c1ccc(Cl)cc1Cl] | B | | | | |
| 279 | 4-cyano-N- (quinolin-8- yl)benzene- sulfonamide | 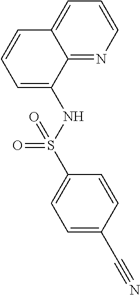 [SMILES: N#Cc1ccc(S(=O)(=O)Nc2cccc3cccnc23)cc1] | B | | | | |
| 280 | N-(quinolin-8- yl)pyridine-2- sulfonamide | 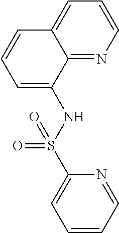 [SMILES: O=S(=O)(Nc1cccc2cccnc12)c1ccccn1] | A | | | | B |
| 281 | N-(quinolin-8- yl)furan-2- sulfonamide | 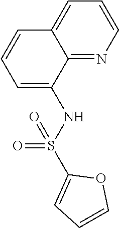 [SMILES: O=S(=O)(Nc1cccc2cccnc12)c1ccco1] | A | | | | |
| 282 | N-(quinolin-8- yl)naphthalene- 2-sulfonamide | 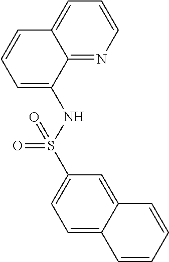 [SMILES: O=S(=O)(Nc1cccc2cccnc12)c1ccc2ccccc2c1] | B | D | | | |
| 283 | 3-fluoro-N- (quinolin-8- yl)benzene- sulfonamide | 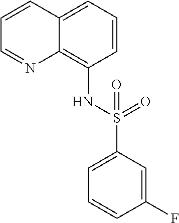 [SMILES: O=S(=O)(Nc1cccc2cccnc12)c1cccc(F)c1] | B | D | | | |
| 284 | 4-methyl-N- (quinolin-8- yl)benzene- sulfonamide | 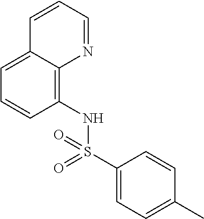 [SMILES: Cc1ccc(S(=O)(=O)Nc2cccc3cccnc23)cc1] | C | D | | | |
| 285 | 4-chloro-N- (quinolin-8- yl)benzene- sulfonamide | 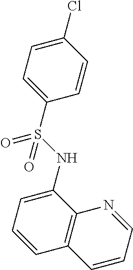 [SMILES: O=S(=O)(Nc1cccc2cccnc12)c1ccc(Cl)cc1] | C | D | | | |

TABLE 4

| | | | D. |
|---|---|---|---|
| | | | immitis |
| Compound | | | Activity |
| ID | Name | Structure | class |
| 286 | N-(3-(dimethylamino)quinolin-8- yl)-1-ethyl-1H-imidazole-2- sulfonamide | 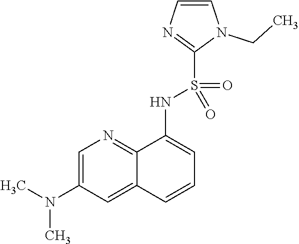 [SMILES: CCn1ccnc1S(=O)(=O)Nc1cccc2cc(N(C)C)cnc12] | B |
| 287 | N-(3-acetylquinolin-8-yl)-1-ethyl- 1H-imidazole-2-sulfonamide | 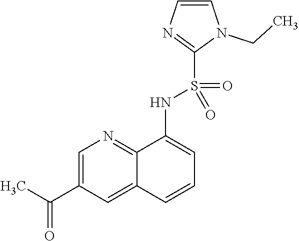 [SMILES: CCn1ccnc1S(=O)(=O)Nc1cccc2cc(C(C)=O)cnc12] | B |
| 288 | N-(4-acetylquinolin-8-yl)-1-ethyl- 1H-imidazole-2-sulfonamide | 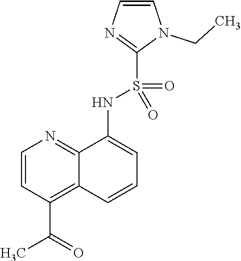 [SMILES: CCn1ccnc1S(=O)(=O)Nc1cccc2c(C(C)=O)ccnc12] | B |
| 289 | 1-phenyl-N-(5- (trifluoromethyl)quinolin-8-yl)- 1H-imidazole-2-sulfonamide | 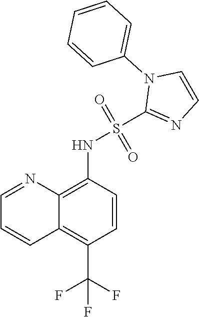 [SMILES: O=S(=O)(Nc1ccc(C(F)(F)F)c2cccnc12)c1nccn1-c1ccccc1] | C |
| 290 | N-(5-cyanoquinolin-8-yl)-1-ethyl- 1H-imidazole-2-sulfonamide | 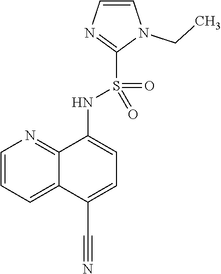 [SMILES: CCn1ccnc1S(=O)(=O)Nc1ccc(C#N)c2cccnc12] | B |
| 291 | 1-ethyl-N-(4-phenylquinolin-8-yl)- 1H-imidazole-2-sulfonamide | 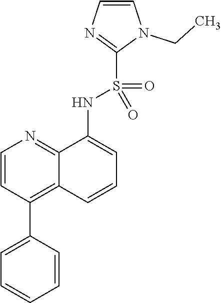 [SMILES: CCn1ccnc1S(=O)(=O)Nc1cccc2c(-c3ccccc3)ccnc12] | C |
| 292 | 1-(2,2,2-trifluoroethyl)-N-(5-(3- (trifluoromethyl)phenoxy)quinolin- 8-yl)-1H-imidazole-2-sulfonamide | 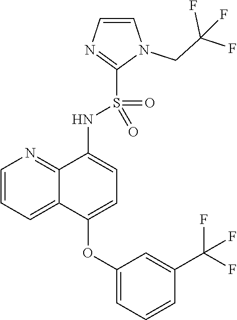 [SMILES: O=S(=O)(Nc1ccc(Oc2cccc(C(F)(F)F)c2)c2cccnc12)c1nccn1CC(F)(F)F] | C |
| 293 | N-(5-bromoquinolin-8-yl)-1-ethyl- 1H-imidazole-2-sulfonamide | 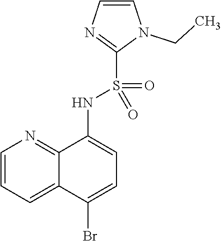 [SMILES: CCn1ccnc1S(=O)(=O)Nc1ccc(Br)c2cccnc12] | C |
| 294 | 1-ethyl-N-(5- (methylsulfonyl)quinolin-8-yl)-1H- imidazole-2-sulfonamide | 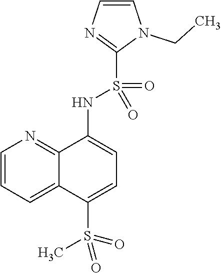 [SMILES: CCn1ccnc1S(=O)(=O)Nc1ccc(S(C)(=O)=O)c2cccnc12] | B |
| 295 | N-(6-fluoroquinolin-8-yl)-1-(4- (trifluoromethyl)phenyl)-1H- imidazole-2-sulfonamide | 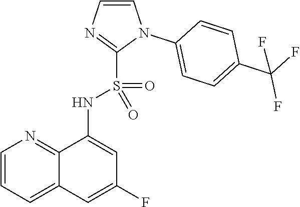 [SMILES: O=S(=O)(Nc1cc(F)cc2cccnc12)c1nccn1-c1ccc(C(F)(F)F)cc1] | C |
| 296 | N-(5-(4-fluorophenyl)quinolin-8- yl)-1-(2,2,2-trifluoroethyl)-1H- imidazol-2-sulfonamide | 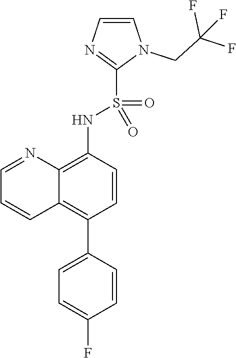 [SMILES: O=S(=O)(Nc1ccc(-c2ccc(F)cc2)c2cccnc12)c1nccn1CC(F)(F)F] | C |
| 297 | N-(6-benzoylquinolin-8-yl)-1- ethyl-1H-imidazole-2-sulfonamide | 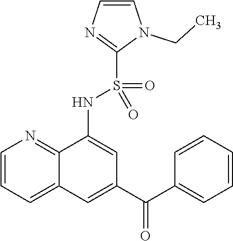 [SMILES: CCn1ccnc1S(=O)(=O)Nc1cc(C(=O)c2ccccc2)cc2cccnc12] | B |

A number of references have been cited, the disclosures of which are incorporated herein by reference in their entirety.

What is claimed is:

1. A compound of Formula (I): 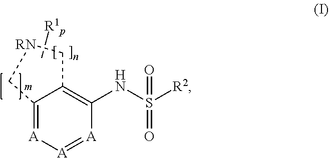
and pharmaceutically acceptable salts, tautomers, isotopologues, or
stereoisomers thereof,
wherein:
- - - is a single or double bond;
each A is independently N or CR¹;
each R¹ is independently H, halogen, alkyl, alkenyl, alkynyl, cycloalkyl, aryl, heterocyclyl, heteroaryl, aralkyl, heterocyclylalkyl, heteroarylalkyl; hydroxy; alkoxy;
cycloalkyloxy, aryloxy, heterocyclyloxy, heteroaryloxy, cycloalkylalkyloxy, aralkyloxy, heterocyclylalkyloxy, heteroarylalkyloxy; amino, alkylamino, cycloalkylamino, arylamino, heterocyclylamino, heteroarylamino; imino; imido; amidino; enamino; acylamino; sulfonylamino; urea, alkoxyamino; aralkoxyamino; thio (—SH) sulfonyl; alkyl sulfonyl, aminosulfonyl; acyl; formyl; carboxy; ester; carbamate; amido; or cyano; each optionally further substituted;
R² is substituted or unsubstituted alkyl, cycloalkyl, heterocyclyl, aryl or heteroaryl;
R is absent, H, substituted or unsubstituted alkyl, cycloalkyl, heterocyclyl, or CO (substituted or unsubstituted alkyl, cycloalkyl, aryl, heterocyclyl, or heteroaryl);
m is 0-3;
n is 0-3; and
p is 0-3;
provided that m and n are not both 0; and
wherein when a group described above is said to be “substituted,” it may be substituted with one or more substituents selected from: halogen; alkyl, alkenyl, alkynyl, cycloalkyl, aryl, heterocyclyl, heteroaryl, cycloalkylalkyl, aralkyl, heterocyclylalkyl, heteroarylalkyl, hydroxy; alkoxy; cycloalkyloxy, aryloxy, heterocyclyloxy, heteroaryloxy, cycloalkylalkyloxy, aralkyloxy, heterocyclylalkyloxy, heteroarylalkyloxy; oxo (═O); oxide; amino, alkylamino, cycloalkylamino, arylamino, heterocyclylamino, heteroarylamino; imino; imido; amidino; guanidino; enamino; acylamino; sulfonylamino; urea, nitrourea; oxime; hydroxylamino; alkoxyamino; aralkoxyamino; hydrazino; hydrazido; hydrazono; azido; nitro; thio (—SH), alkylthio; ═S; sulfinyl; sulfonyl; aminosulfonyl; phosphonate; phosphinyl; acyl; formyl; carboxy; ester; carbamate; amido; cyano; isocyanato; isothiocyanato; cyanato; thiocyanato; and —B(OH)₂; each optionally further substituted.

2. The compound of claim 1, wherein m is 2, n is 1, and A is CR¹.

3. The compound of claim 1, wherein m is 1, n is 1, and A is CR¹.

4. The compound of claim 1, wherein m is 3, n is 0, and A is CR¹.

5. The compound of claim 1, wherein m is 2, n is 0, and A is CR¹.

6. The compound of any one of claims 1-5, wherein - - - is a single bond.

7. The compound of any one of claims 1-6, wherein R¹ is H and p is 0.

8. The compound of any one of claims 1-7, wherein R² is
a. 2-pyridyl, substituted with one or more substituents independently selected from halogen, CN, substituted or unsubstituted C₁₋₄ alkyl, substituted or unsubstituted C₃₋₆ cycloalkyl, —OR, and —NR₂;
b. 2-imidazolyl substituted with one or more substituents independently selected from substituted or unsubstituted C₁₋₄ alkyl, substituted or unsubstituted C₃₋₆ cycloalkyl, (C₁₋₃ alkyl) (substituted or unsubstituted C₃₋₆ cycloalkyl), and substituted or unsubstituted aryl;
c. pyrazyl, substituted with one or more substituents independently selected from substituted or unsubstituted C₁₋₄ alkyl, substituted or unsubstituted 3-6 membered heterocyclyl, and —OR;
d. pyrazolyl, unsubstituted or substituted with substituted or unsubstituted C₁₋₄ alkyl;
e. 2-furanyl unsubstituted or substituted with one or more C₁₋₄ alkyl.

9. The compound of any one of claims 1-8, wherein R is H or substituted or unsubstituted C₁₋₄ alkyl, or substituted or unsubstituted C₃₋₆ cycloalkyl.

10. A compound of Formula (Ia) 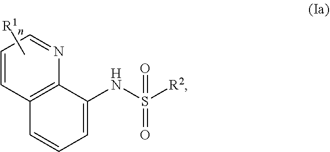
and pharmaceutically acceptable salts, tautomers, isotopologues, or stereoisomers thereof,
wherein:
each R¹ is independently halogen, —CN, substituted or unsubstituted C₁₋₄ alkyl, substituted or unsubstituted C₃₋₇ cycloalkyl, substituted or unsubstituted acyl, substituted or unsubstituted C₁₋₄ sulfonyl, substituted or unsubstituted 3-6 membered heterocyclyl, substituted or unsubstituted aryl, or —OR;
R² is
a. 2-pyridyl or 3-pyridyl, substituted with one or more substituents independently selected from halogen, CN, substituted or unsubstituted C₁₋₄ alkyl, substituted or unsubstituted C₃₋₆ cycloalkyl, —OR, and —NR₂;
b. 2-imidazolyl substituted with one or more substituents independently selected from substituted or unsubstituted C₁₋₄ alkyl, substituted or unsubstituted C₃₋₆ cycloalkyl, (C₁₋₃ alkyl) (substituted or unsubstituted C₃₋₆ cycloalkyl), and substituted or unsubstituted aryl;
c. 5-imidazolyl, substituted with one or more substituents independently selected from substituted or unsubstituted C₁₋₄ alkyl, substituted or unsubstituted C₃₋₆ cycloalkyl, and (C₁₋₃ alkyl) (substituted or unsubstituted C₃₋₆ cycloalkyl);
d. pyrazyl, substituted with one or more substituents independently selected from substituted or unsubstituted C₁₋₄ alkyl, substituted or unsubstituted 3-6 membered heterocyclyl, —OR, and NR₂;
e. pyrazolyl, unsubstituted or substituted with one or more substituted or unsubstituted C₁₋₄ alkyl;
f. 2-furanyl unsubstituted or substituted with one or more C₁₋₄ alkyl;
each R is independently H and substituted or unsubstituted C₁₋₄ alkyl, (C₁₋₃ alkyl), (substituted or unsubstituted C₃₋₆ cycloalkyl), or substituted or unsubstituted aryloxy;
n is 1-3;
provided the compound is not 5-cyano-N-[5-(trifluoromethyl)-8-quinolinyl]-2-pyridinesulfonamide.

11. The compound of claim 10, wherein each R is independently —CH₃, —CH₂CH₃, —CH₂CH₂CH₃, —CH₂CH(CH₃)₂, —CH(CH₃)₂, or —CH₂(cyclopropyl).

12. The compound of any one of claim 10 or 11, wherein n is 1 or 2.

13. The compound of any one of claims 10-12, wherein each R¹ is independently F, Cl, Br, CN, —CH₃, —CH₂CH₃, —CH₂CH₂CH₃, —CH₂CH(CH₃)₂, —CF₃, cyclopropyl, cylobutyl, cyclopentyl, cyclohexyl, —OCH₃, —OCH₂CH₃, —OCH(CH₃)₂, —OCH₂(cyclopropyl), azetidinyl, —N(CH₃)₂, —C(O)CH₃, benzoyl, methyl sulfonyl, phenyl, —O-(m-trifluormethyl)phenyl, or p-fluorophenyl, pyrrolidyl, piperidyl, piperazinyl or morpholinyl.

14. The compound of claim any one of claims 10-13, wherein each R¹ is independently F, Cl, Br, CN, —CH₃, —CH₂CH₃, —CF₃, cyclopropyl, cyclohexyl, —OCH₃, —OCH(CH₃)₂, —OCH₂(cyclopropyl), azetidinyl, —N(CH₃)₂, —C(O)CH₃, benzoyl, methyl sulfonyl, phenyl, —O-(m-trifluormethyl)phenyl, or p-fluorophenyl, or morpholinyl.

15. The compound of any one of claims 10-14, wherein R² is 2-pyridyl, substituted with one or more substituents independently selected from F, Cl, —CN, —CH₃, —CH₂CH₃, —CH₂CH₂CH₃, —CH(CH₃)₂, cyclopropyl, cyclobutyl, cyclopentyl, —OCH₃, —OCH₂CH₂CH₃, —OCH₂CH(CH₃)₂, —NH₂, —NHCH₃, and —N(CH₃)₂.

16. The compound of any one of claims 10-15, wherein R² is 2-pyridyl, substituted with one or more substituents independently selected from F, Cl, —CN, —CH₃, —CH(CH₃)₂, —CH₂CH(CH₃)₂, cyclopropyl, —OCH₃, —OCH₂CH(CH₃)₂, and —N(CH₃)₂.

17. The compound of any one of claims 10-16, wherein R² is 3-pyridyl substituted with one or more substituents independently selected from F, Cl, —CN, —CH₃, —CH₂CH₃, and —CF₃.

18. The compound of any one of claims 10-17, wherein R² is 3-pyridyl substituted with —CF₃.

19. The compound of any one of claims 10-18, wherein R² is 2-imidazolyl, substituted with one or more substituents independently selected —CH₃, —CH₂CH₃, —CH₂CF₃, cyclopropyl, —CH₂CH(CH₃)₂, phenyl, and p-trifluoromethyl phenyl.

20. The compound of any one of claims 10-19, wherein R² is 2-imidazolyl, substituted with one or more substituents independently selected from —CH₃, —CH₂CH₃, cyclopropyl, and —CH₂CH(CH₃)₂.

21. The compound of any one of claims 10-20, wherein R² is 5-imidazolyl, substituted with one or more substituents independently selected from —CH₃, —CH₂CH₃, —CH₂CH₂CH₃, —CH(CH₃)₂, cyclopropyl, and CH₂-cyclopropyl.

22. The compound of any one of claims 10-21, wherein R² is 2-pyrazyl, unsubstituted or substituted with one or more substituents independently selected from —CH₃, —CH₂CH₃, —CH₂CH₂CH₃, —CH₂CH(CH₃), —OCH₃, —OCH₂CH₃, —OCH₂CH₂CH₃, —OCH₂CH(CH₃)₂, —N(CH₃)₂, pyrrolidyl, piperidyl, piperazinyl and morpholinyl.

23. The compound of any one of claims 10-22, wherein R² is 2-pyrazyl, unsubstituted or substituted with one or more substituents independently selected from —CH₃, —OCH₃, and pyrrolidyl.

24. The compound of any one of claims 10-22, wherein R² is 2-pyrazolyl, unsubstituted or substituted with one or more substituents independently selected from —CH₃, —CH₂CH₃, —CH₂CH₂CH₃, and —CH(CH₃)₂.

25. The compound of claim 10, wherein each R¹ is independently F, Cl, —CH₃, —CH₂CH₂CH₃, CF₃, cyclohexyl, —OCH₃, —OCH(CH₃)₂, —OCH₂(cyclopropyl), azetidinyl, phenyl, or morpholinyl, and R² is 2-pyridyl, substituted with one or more substituents independently selected from F, Cl, CN, —CH₃, —CH₂CH₃, —CH₂CH₂CH₃, —CH₂CH(CH₃)₂, cyclopropyl, cyclobutyl, cyclopentyl, —OCH₃, —OCH₂CH₃, —OCH₂CH₂CH₃, —OCH₂CH(CH₃)₂, —NH₂, —NHCH₃, and —N(CH₃)₂.

26. The compound of claim 25, wherein R² is 2-pyridyl, substituted with one or more substituents independently selected from F, Cl, CN, —CH₃, —CH₂CH(CH₃)₂, cyclopropyl, —OCH₃, —OCH₂CH(CH₃)₂, and —N(CH₃)₂.

27. The compound of claim 25, wherein each R¹ is independently F, Cl, —CH₃, cyclopropyl, cyclohexyl, —OCH₃, —OCH(CH₃)₂, —OCH₂(cyclopropyl), azetidinyl, phenyl, or morpholinyl.

28. The compound of claim 10, wherein each R¹ is independently F, —CH₃, or —OCH₃, and R² is 3-pyridyl, substituted with —CF₃.

29. The compound of claim 10, wherein each R¹ is independently F, Cl, Br, CN, —CH₃, —CH₂CH₃, —CF₃, cyclohexyl, —OCH₃, —N(CH₃)₂, —C(O)CH₃, benzoyl, methyl sulfonyl, morpholinyl, phenyl, —O-(m-trifluormethyl)phenyl, or p-fluorophenyl, and R² is 2-imidazolyl, substituted with one or more substituents independently selected from —CH₃, —CH₂CH₃, —CH₂CF₃, —CH₂CH₂CH₃, cyclopropyl, —CH₂CH(CH₃)₂, phenyl, and p-trifluoromethyl phenyl.

30. The compound of claim 29, wherein R² is 2-imidazolyl, substituted with one or more substituents independently selected from —CH₃, —CH₂CH₃, —CH₂CF₃, cyclopropyl, —CH₂CH(CH₃)₂, phenyl, and p-trifluoromethyl phenyl.

31. The compound of claim 10, wherein each R¹ is independently C₁, or morpholinyl, and R² is 5-imidazolyl, substituted with one or more substituents independently selected from —CH(CH₃)₂, or —CH₂-cyclopropyl.

32. The compound of claim 10, wherein each R¹ is independently F, Cl, —CH₃, —CH₂CH₃, —CF₃, cyclohexyl, —OCH₃, or morpholinyl, and R² is 2-pyrazyl, substituted with one or more substituents independently selected from —CH₃, —CH₂CH₃, —CH₂CH₂CH₃, —CH₂CH(CH₃)₂, —OCH₃, —OCH₂CH₃, —OCH₂CH₂CH₃, —OCH₂CH(CH₃)₂, —N(CH₃)₂, pyrrolidyl, piperidyl, piperazinyl and morpholinyl.

33. The compound of claim 32, wherein R² is 2-pyrazyl, substituted with one or more substituents independently selected from —CH₃, —OCH₃, —N(CH₃)₂, and pyrrolidyl.

34. The compound of claim 32, wherein each R¹ is independently C₁ or morpholinyl.

35. The compound of claim 10, wherein each R¹ is independently F or morpholinyl and R² is 2-pyrazolyl, unsubstituted or substituted with one or more substituents independently selected from —CH₃ and —CH(CH₃)₂.

36. The compound of any of one of claims 1-10, wherein the compound is selected from Table 1.

37. A compound of Table 2.

38. A compound of Table 4.

39. A pharmaceutical composition comprising an effective amount of a compound of any one of claims 1-38, or a pharmaceutically acceptable salt, tautomer, isotopologue, or stereoisomer thereof, and a pharmaceutically acceptable carrier, excipient or vehicle.

40. A method of killing a filarial worm, comprising contacting the filarial worm with a compound of any one of claims 1-38, or a pharmaceutically acceptable salt, tautomer, isotopologue, or stereoisomer thereof, in an amount effective to kill the filarial worm.

41. A method of inhibiting growth or molt of a filarial worm, comprising contacting the filarial worm with a compound of any one of claims 1-38, or a pharmaceutically acceptable salt, tautomer, isotopologue, or stereoisomer thereof, in an amount effective to inhibit growth or molt of the filarial worm.

42. A method of killing a filarial worm, comprising contacting the filarial worm with a compound of Table 3, or a pharmaceutically acceptable salt, tautomer, isotopologue, or stereoisomer thereof, in an amount effective to kill the filarial worm.

43. A method of inhibiting growth or molt of a filarial worm, comprising contacting the filarial worm with a compound of Table 3, or a pharmaceutically acceptable salt, tautomer, isotopologue, or stereoisomer thereof, in an amount effective to inhibit growth or molt of the filarial worm.

44. A method of inhibiting motility of a filarial worm, comprising contacting the filarial worm with a compound of any one of claims 1-37, or a pharmaceutically acceptable salt, tautomer, isotopologue, or stereoisomer thereof, in an amount effective to inhibit motility of the filarial worm.

45. A method of inhibiting motility of a filarial worm, comprising contacting the filarial worm with a compound of Table 3, or a pharmaceutically acceptable salt, tautomer, isotopologue, or stereoisomer thereof, in an amount effective to inhibit motility of the filarial worm.

46. A method for the treatment or prevention of helminthic infections and diseases, the methods comprising administering to a subject an effective amount of a compound of any one of claims 1-38, or a pharmaceutically acceptable salt, tautomer, isotopologue, or stereoisomer thereof.

47. The method of claim 46, wherein the helminthic infection is a filarial worm infection.

48. A method for the treatment or prevention of helminthic infections and diseases, the methods comprising administering to a subject an effective amount of a compound of Table 3, or a pharmaceutically acceptable salt, tautomer, isotopologue, or stereoisomer thereof.

49. The method of claim 48, wherein the helminthic infection is a filarial worm infection.

50. A method for the treatment or prevention of helminthic infections and diseases, the methods comprising administering to a subject an effective amount of a compound of any one of claims 1-38, or a pharmaceutically acceptable salt, tautomer, isotopologue, or stereoisomer thereof in combination with one or more antihelminthic agent.

51. The method of claim 50, wherein the helminthic infection is a filarial worm infection.

52. A method for the treatment or prevention of helminthic infections and diseases, the methods comprising administering to a subject an effective amount of a compound of Table 3, or a pharmaceutically acceptable salt, tautomer, isotopologue, or stereoisomer thereof in combination with one or more antihelminthic agent.

53. The method of claim 52, wherein the helminthic infection is a filarial worm infection.

54. The method of claim 50 or 52, wherein the antihelminthic agent is selected from flubendazole, albendazole, mebendazole, thiabendazole, fenbendazole, triclabendazole, ivermectin, abamectin, diethylcarbamazine (DEC), suramin, pyrantel pamoate, levamisole, niclosamide, nitazoxanide, oxyclozanide, praziquantel, emodepside, monepantel, derquantel, or pelletierine sulphate.

55. The method of claim 50 or 52, wherein the antihelminthic agent is a Wolbachia targeting agent.

56. The method of claim 55, wherein the Wolbachia targeting agent is doxycycline.
